SEQUENCE LISTING (31 sequences)

SEQ ID NO: 1 - DNA, 20 bp, synthetic construct
  source 1..20: mol_type = other DNA
cttccagccaccatagagcg

SEQ ID NO: 2 - DNA, 20 bp, synthetic construct
  source 1..20: mol_type = other DNA
ggtccaagtccgaaaatatg

SEQ ID NO: 3 - DNA, 20 bp, synthetic construct
  source 1..20: mol_type = other DNA
tctgccgtcgcttactatga

SEQ ID NO: 4 - DNA, 20 bp, synthetic construct
  source 1..20: mol_type = other DNA
ttctatgaagaaccgccgca

SEQ ID NO: 5 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 69: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
cttccagccaccatagagcggttttagagctagaaatagcaagttaaaataaggctagtccgttatcaacttgaaaaagtggcaccgagtcggtgctttt

SEQ ID NO: 6 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 69: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
ggtccaagtccgaaaatatggttttagagctagaaatagcaagttaaaataaggctagtccgttatcaacttgaaaaagtggcaccgagtcggtgctttt

SEQ ID NO: 7 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 69: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
tctgccgtcgcttactatgagttttagagctagaaatagcaagttaaaataaggctagtccgttatcaacttgaaaaagtggcaccgagtcggtgctttt

SEQ ID NO: 8 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 69: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
ttctatgaagaaccgccgcagttttagagctagaaatagcaagttaaaataaggctagtccgttatcaacttgaaaaagtggcaccgagtcggtgctttt

SEQ ID NO: 9 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 69: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
gcaggcggttcaagactcatgttttagagctagaaatagcaagttaaaataaggctagtccgttatcaacttgaaaaagtggcaccgagtcggtgctttt

SEQ ID NO: 10 - DNA, 25 bp, synthetic construct
  source 1..25: mol_type = other DNA
cttgagaactgagggagaaaatcac

SEQ ID NO: 11 - DNA, 26 bp, synthetic construct
  source 1..26: mol_type = other DNA
gaattggaagagtctgtcctaaaagg

SEQ ID NO: 12 - DNA, 14 bp, synthetic construct
  source 1..14: mol_type = other DNA
ctgccacgctctat

SEQ ID NO: 13 - DNA, 14 bp, synthetic construct
  source 1..14: mol_type = other DNA
cttgcaggaagcct

SEQ ID NO: 14 - DNA, 20 bp, synthetic construct
  source 1..20: mol_type = other DNA
tgacctcccccttcctgatt

SEQ ID NO: 15 - DNA, 25 bp, synthetic construct
  source 1..25: mol_type = other DNA
cccagtagtgttaaagctggtcttt

SEQ ID NO: 16 - DNA, 16 bp, synthetic construct
  source 1..16: mol_type = other DNA
caagtccgaaaatatg

SEQ ID NO: 17 - DNA, 15 bp, synthetic construct
  source 1..15: mol_type = other DNA
ttctccctgaagctc

SEQ ID NO: 18 - DNA, 22 bp, synthetic construct
  source 1..22: mol_type = other DNA
tgtgcagacatgtaggcttgct

SEQ ID NO: 19 - DNA, 23 bp, synthetic construct
  source 1..23: mol_type = other DNA
gttgaacaggtttgggtggttag

SEQ ID NO: 20 - DNA, 13 bp, synthetic construct
  source 1..13: mol_type = other DNA
ccgtgcggcggtt

SEQ ID NO: 21 - DNA, 18 bp, synthetic construct
  source 1..18: mol_type = other DNA
cgcattaaagggagaaag

SEQ ID NO: 22 - DNA, 22 bp, synthetic construct
  source 1..22: mol_type = other DNA
gaaaactgtccgtcccttcagt

SEQ ID NO: 23 - DNA, 19 bp, synthetic construct
  source 1..19: mol_type = other DNA
cctcacctgttccgtgcat

SEQ ID NO: 24 - DNA, 17 bp, synthetic construct
  source 1..17: mol_type = other DNA
cagctgccctcatagta

SEQ ID NO: 25 - DNA, 16 bp, synthetic construct
  source 1..16: mol_type = other DNA
tggtgtgctgtcctgt

SEQ ID NO: 26 - DNA, 18 bp, synthetic construct
  source 1..18: mol_type = other DNA
gaaccgcatcgagctgaa

SEQ ID NO: 27 - DNA, 21 bp, synthetic construct
  source 1..21: mol_type = other DNA
tagacgttgtggctgttgtag

SEQ ID NO: 28 - DNA, 24 bp, synthetic construct
  source 1..24: mol_type = other DNA
atcgacttcaaggaggacggcaac

SEQ ID NO: 29 - DNA, 20 bp, synthetic construct
  source 1..20: mol_type = other DNA
agaaaagagactgtccctag

SEQ ID NO: 30 - DNA, 21 bp, synthetic construct
  source 1..21: mol_type = other DNA
gtgaacgtcatgagatccaaa

SEQ ID NO: 31 - DNA, 20 bp, synthetic construct
  source 1..20: mol_type = other DNA
cccacatttccaggcagaag

(19) United States
(12) Patent Application Publication
(10) Pub. No.: US 2025/0001013 A1
(43) Pub. Date: Jan. 2, 2025

(54) MODULATORS OF ADENO-ASSOCIATED VIRUS TRANSDUCTION AND USES THEREOF

(71) Applicant: Novartis AG, Basel (CH)

(72) Inventors: Eloise Hudry, Somerville, MA (US); Katie Kubek-Luck, Stoneham, MA (US); Keith Mansfield, Shrewsbury, MA (US)

(21) Appl. No.: 18/712,515

(22) PCT Filed: Nov. 23, 2022
(86) PCT No.: PCT/IB2022/061355

§ 371 Date: May 22, 2024

Related U.S. Application Data
(60) Provisional application No. 63/283,072, filed on Nov. 24, 2021.

(51) Int. Cl. A61K 48/00; C12N 15/86

(52) U.S. Cl. CPC A61K 48/0066; A61K 48/0033; C12N 15/86; C12N 2750/14143; C12N 2750/14145; C12N 2750/14152; C12N 2750/14162

(57) ABSTRACT

The disclosure provides compositions and methods for modulating transduction efficiency of AAV particles. Specifically, the disclosure provides AAV transduction modulators that modulate genes or gene products associated with AAV transduction efficiency in gene therapies.

### CROSS-REFERENCE TO RELATED APPLICATIONS

This application claims the benefit of U.S. Provisional Application No. 63/283,072, filed Nov. 24, 2021. The contents of the aforementioned application are hereby incorporated by reference in their entirety.

### FIELD OF THE DISCLOSURE

The disclosure relates to use of adeno-associated virus (AAV) transduction modulators to alter AAV transduction efficiency in gene therapies.

### BACKGROUND OF THE DISCLOSURE

AAVs are non-enveloped viruses that can be engineered to deliver a therapeutic payload to target cells. The ability to generate recombinant AAV particles lacking certain viral genes and containing therapeutic genes of interest provides a safe platform for gene therapy delivery across different therapeutic areas. AAV vectors have been used in clinical trials for various diseases, achieving promising results. Despite the recent progress, there exists a need for novel compositions and methods for modulating the transduction efficiency of AAV in gene therapies.

### SUMMARY OF THE DISCLOSURE

In an aspect, the disclosure features a method of modulating transduction efficiency of an AAV particle, comprising contacting a cell with an AAV transduction modulator, thereby modulating the transduction efficiency of the AAV particle.

In some embodiments, the modulator modulates (e.g., increases or decreases) a gene or gene product associated with AAV transduction efficiency. In some embodiments, the gene or gene product is a mammalian (e.g., human) gene or gene product.

In some embodiments, the modulator inhibits the gene or gene product, e.g., a gene or gene product that is associated with a decreased transduction efficiency of an AAV particle. In some embodiments, the modulator activates the gene or gene product, e.g., a gene or gene product that is associated with an increased transduction efficiency of an AAV particle.

In some embodiments, the gene or gene product is associated with an increased transduction efficiency of an AAV particle (e.g., AAV-R or GPR108). In some embodiments, the gene or gene product is associated with an increased transduction efficiency of an AAV particle of a first serotype (e.g., AAV2), an increased transduction efficiency of an AAV particle of a second serotype (e.g., AAV9), or both.

In some embodiments, the gene or gene product is associated with a decreased transduction efficiency of an AAV particle (e.g., WDR11 or MRE11). In some embodiments, the gene or gene product is associated with a decreased transduction efficiency of an AAV particle of a first serotype (e.g., AAV2), a decreased transduction efficiency of an AAV particle of a second serotype (e.g., AAV9), or both.

In some embodiments, the gene or gene product, when modulated, increases the transduction efficiency of an AAV particle (e.g., AAV-R or GPR108). In some embodiments, the gene or gene product, when modulated, increases the transduction efficiency of an AAV particle of a first serotype (e.g., AAV2), the transduction efficiency of an AAV particle of a second serotype (e.g., AAV9), or both.

In some embodiments, the gene or gene product, when modulated, decreases the transduction efficiency of an AAV particle (e.g., WDR11 or MRE11). In some embodiments, the gene or gene product, when modulated, decreases the transduction efficiency of an AAV particle of a first serotype (e.g., AAV2), the transduction efficiency of an AAV particle of a second serotype (e.g., AAV9), or both.

In some embodiments, the gene product is an RNA (e.g., an mRNA). In some embodiments, the gene product is a protein. In some embodiments, the gene or gene product is preferentially expressed in a target tissue (e.g., brain or liver). The following dependent claims describe the AAV transduction modulator based on its mechanism. In some embodiments, the modulator alters (e.g., increases or decreases) the expression of the gene. In some embodiments, the modulator alters the structure of the gene. In some embodiments, the modulator alters (e.g., increases or decreases) an activity (e.g., an enzymatic activity) of the gene product. In some embodiments, the modulator alters (e.g., increases or decreases) the level (e.g., abundance) of the gene product. In some embodiments, the modulator alters (e.g., increases or decreases) the stability of the gene product. In some embodiments, the modulator alters (e.g., increases or decreases) transduction efficiency of an AAV particle, e.g., as determined by an assay described herein (e.g., an FACS analysis, e.g., as described in Examples 1 or 2).

In some embodiments, the modulator increases transduction efficiency of an AAV particle. In some embodiments, the transduction efficiency is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference level of transduction efficiency. In some embodiments, the modulator increases transduction to at least 10, 20, 30, 40, 50, or 60 viral copies per genome. In some embodiments, the modulator increases transduction to at least 10³, 10⁴, 10⁵, 10⁶, or 10⁷ viral copies per μg DNA.

In some embodiments, the modulator decreases transduction efficiency of an AAV particle. In some embodiments, the transduction efficiency is decreased by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of transducation efficiency. In some embodiments, the modulator decreases transduction to no more than 0, 1, 2, 3, 4, 5, 6, 7, 8, 9, or 10 viral copies per genome. In some embodiments, the modulator decreases transduction to no more than 10², 10³, or 10⁴ viral copies per μg DNA.

In some embodiments, the reference level of transduction efficiency is the level of transduction efficiency in a cell that has not been contacted with the AAV transduction modulator. In some embodiments, the reference level of transduction efficiency is the level of transduction efficiency before the cell is contacted with the AAV transduction modulator.

In some embodiments, the gene or gene product increases the transduction efficiency of an AAV2 particle, optionally wherein the gene or gene product or gene or gene product does not increase, or does not substantially increase, the transduction efficiency of an AAV9 particle. In some embodiments, the gene or gene product is necessary for the transduction of an AAV2 particle, optionally wherein the gene or gene product is not necessary for the transduction of an AAV9 particle.

In some embodiments, the gene or gene product is AAV-R. In some embodiments, the gene or gene product is GRP108.

In some embodiments, the gene or gene product is chosen from EXT1, EXT2, NDST1, BCL10, OAF, PDCD10, LMO4, MALT1, ZNF644, CHUK, GLCE, EP300, ARID4B, RAB21, MED13, DHX15, WASHC5, IKBKB, USP24, HSPA14, CXXC1, UBE2A, INTS8, CDC42, NFKB1, SIN3A, USP7, ARF5, CARD10, JAK1, SLC35C2, PAXK1, ZC3H11A, FAM20B, FAM72A, DHX36, INTS12, RPRD1B, or a combination thereof.

In some embodiments, the gene or gene product is associated with endosomal sorting (e.g., RAB21, WASHC5, or USP7). In some embodiments, the gene or gene product is associated with vesicle-mediated transport (e.g., CDC42 or ARF5). In some embodiments, the gene or gene product is associated with NFKB signaling (e.g., BCL10, MALT1, NFKB1, IKBKB, CHUK, or CARD10).

In some embodiments, the gene or gene product is associated with heparan sulfate biosynthesis (e.g., EXT1, EXT2, NDST1, GLCE, or FAM20B). In some embodiments, the gene or gene product is associated with H3K9 methylation (e.g., ZNF644). In some embodiments, the gene or gene product is associated with pre-mRNA processing (e.g., DHX15). In some embodiments, the gene or gene product is associated with transcription (e.g., LMO4, EP300, MED13, SIN3, ARID4B, ZC3H11A, CXXC1, RPRD1B, or UBE2A). In some embodiments, the gene or gene product is associated with integrator complex and/or RNA polymerase II function (e.g., INTS8 or INST12). In some embodiments, the gene or gene product is associated with DNA repair and/or AAV genome resolution (e.g., DHX36, ARID4B, UBE2A, or USP24). In some embodiments, the gene or gene product is associated with cell proliferation and/or apoptosis (e.g., PDCD10). In some embodiments, the gene or gene product is associated with spondylocarpotarsal synostosis syndrome (e.g., OAF). In some embodiments, the gene or gene product is associated with protein folding (e.g., HSPA14). In some embodiments, the gene or gene product is associated with helicase activity (e.g., DHX36). In some embodiments, the gene or gene product is associated with fucosylation of Notch and/or transport of GCP-fucose (e.g., SLC35C2).

In some embodiments, the gene or gene product increases the transduction efficiency of an AAV9 particle, optionally wherein the gene or gene product does not increase, or does not substantially increase, the transduction efficiency of an AAV2 particle. In some embodiments, the gene or gene product is necessary for the transduction of an AAV9 particle, optionally wherein the gene or gene product is not necessary for the transduction of an AAV2 particle. In some embodiments, the gene or gene product is AAV-R. In some embodiments, the gene or gene product is GRP108.

In some embodiments, the gene or gene product is chosen from TMPRSS11B, HTT, SNRNP70, BRD7, DMXL1, RAB10, CNGA1, KLHDC3, CHP1, CYP3A5, ELOVL4, PNISR, ZCCHC14, AZGP1, TPST1, SC5D, ELP2, ELP3, IP09, RAB14, WDR7, XRCC4, GDI2, or a combination thereof.

In some embodiments, the gene or gene product is associated with microtubule-mediated transport or vesicle function (e.g., HT). In some embodiments, the gene or gene product is an ion channel (e.g., CNGA1). In some embodiments, the gene or gene product is associated with nuclear protein import (e.g., IP09). In some embodiments, the gene or gene product is associated with O-sulfation of a tyrosine residue within an acidic motif of a polypeptide (e.g., TPST1). In some embodiments, the gene or gene product is associated with protection of viral RNA (e.g., ZCCHC14).

In some embodiments, the gene or gene product is associated with RNA binding by an AAV protein (e.g., PNISR). In some embodiments, the gene or gene product is associated with calcium ion-regulated exocytosis (e.g., CHP1). In some embodiments, the gene or gene product is associated with intracellular trafficking (e.g., RAB10, RAB14, GDI2, WDR7, or DMXL1). In some embodiments, the gene or gene product is a subunit (e.g., a catalytic tRNA acetyltransferase subunit) of an elongator complex, e.g., of an RNA polymerase (e.g., RNA polymerase II), e.g., ELP2 or ELP3. In some embodiments, the gene or gene product binds to chromatin (e.g., KLHDC3). In some embodiments, the gene or gene product is associated with cholesterol biosynthesis (e.g., SC5D).

In some embodiments, the gene or gene product increases the transduction efficiency of AAV2 and AAV9 particles. In some embodiments, the gene or gene product is necessary for the transduction of AAV2 and AAV9 particles. In some embodiments, the gene or gene product is AAV-R. In some embodiments, the gene or gene product is GRP108.

In some embodiments, the gene or gene product is chosen from KIAA0319L, BAMBI, TM9SF2, PHIP, DCLRE1C, VPS35, GPR108, CYREN, ACP2, F8A2, F8A1, F8A3, RBPJ, ATP2C1, ICMT, RABIF, IER3IP1, RBM10, SMG7, GTF2I, ELAVL1, MEPCE, RAB4A, IER3IP1, SAMD1, or a combination thereof.

In some embodiments, the gene or gene product is associated with influenza infection (e.g., ACP2). In some embodiments, the gene or gene product binds to unmethylated CGIs (e.g., SAMD1). In some embodiments, the gene or gene product is associated with intracellular vesicular transport (e.g., RABIF). In some embodiments, the gene or gene product is associated with posttranslational modification of cysteine residues (e.g., C-terminal cysteine residues), e.g., in a target protein (e.g., ICMT). In some embodiments, the gene or gene product binds to a capsid protein of AAV (e.g., KIAA0319L). In some embodiments, the gene or gene product is associated with inhibition of a TLR (e.g., TLR9), e.g., GPR108. In some embodiments, the gene or gene product is associated with endosome trafficking, e.g., of AAV-R (e.g., VPS35). In some embodiments, the gene or gene product is a calcium ATPase pump (e.g., ATP2C1). In some embodiments, the gene or gene product is associated with Notch signaling (e.g., RBPJ). In some embodiments, the gene or gene product is associated with HSPG metabolism (e.g., TM9SF2). In some embodiments, the gene or gene product is associated with inhibition of NHEJ (e.g., CYREN). In some embodiments, the gene or gene product is associated with viral hairpin resolution (e.g., DCLRE1C). In some embodiments, the gene or gene product is associated with Glc transporter translocation, optionally wherein the gene or gene product binds to insulin receptor substrate 1 protein (e.g., PHIP).

In some embodiments, the gene or gene product decreases the transduction efficiency of an AAV2 particle, optionally wherein the gene or gene product, when modulated, does not decrease, or does not substantially decrease, the transduction efficiency of an AAV9 particle. In some embodiments, the gene or gene product inhibits or prevents the transduction of an AAV2 particle, optionally wherein the gene or gene product does not inhibit or prevent the transduction of an AAV9 particle. In some embodiments, the gene or gene product is WRD11. In some embodiments, the gene or gene product is MRE11.

In some embodiments, the gene or gene product is chosen from GREB1L, BIRC2, TYMSOS, MSL3, SIK3, GTF2H5, PIGW, ATRAID, PPP4R1, ZFC3H1, SETDB1, SMCHD1, TMEM123, UHRF1, CLUL1, SMAD5, SETX, STEEP1, DENND6A, RTN4RL2, MTMR9, STRADA, PSIP1, BMPR1A, HIKESHI, AP3M1, MEN1, MYL12B, CREB1, RAB12, SMTNL1, MRGBP, WTAP, SUMO2, TCN1, ALDH3B1, ARL14EP, MYL12A, RHOD, PGAP2, EIF4A1, PIGN, DAXX, HDLBP, GOLGA1, SARNP, UBE2L6, TRIM49, SIK2, SOX4, OR6Q1, OR4D6, UNC93B1, CATSPERZ, PIGT, PYM1, MICOS13, RIN1, DOT1L, TRIM49C, MAPK20, COL8A1, FASN, KDM3B, PIGF, PIGA, PIGY, GPC3, TRAPPC2L, RAB31, ELP3, or a combination thereof.

In some embodiments, the gene or gene product is associated with a retinoic acid receptor activity (e.g., GREB1L). In some embodiments, the gene or gene product is a thymidylate synthetase (e.g., TYMSOS). In some embodiments, the gene or gene product is associated with chromatin remodeling (e.g., SMCHD1). In some embodiments, the gene or gene product is associated with histone H4 acetylation (e.g., MSL3). In some embodiments, the gene or gene product is associated with transcription and/or DNA repair (e.g., GTF2H5). In some embodiments, the gene or gene product is a histone methyltransferase (e.g., SETDB1). In some embodiments, the gene or gene product is associated with exosomal degradation of polyadenylated RNA (e.g., ZFC3H1). In some embodiments, the gene or gene product is associated with histone deacetylase recruitment to a DNA (e.g., UHRF1). In some embodiments, the gene or gene product is associated with oxidative stress-induced DNA double strand break response (e.g., SETX). In some embodiments, the gene or gene product is associated with histone modification (e.g., MEN1). In some embodiments, the gene or gene product is associated with nucleosome/DNA interaction (e.g., MRGBP). In some embodiments, the gene or gene product is associated with GPI biosynthesis (e.g., PIGW, PIGN, PIGT, PIGF, PIGA, or PIGY). In some embodiments, the gene or gene product is associated with maturation of GPI anchors (e.g., PGAP2). In some embodiments, the gene or gene product is associated with vesicle-mediated endocytosis (e.g., DENND6A, GOLGA1, MTMR9, GPC3, PPP4R1, RAB12, RAB31, RHOD, RIN1, TMEM123, or TRAPPC2L). In some embodiments, the gene or gene product is associated with clathrin-coated vesicle-mediated endocytosis (e.g., AP3M1). In some embodiments, the gene or gene product is associated with a BMP receptor kinase (e.g., SMAD5, BMPR1A, or CREB1). In some embodiments, the gene or gene product is associated with transcription (e.g., PSIP1). In some embodiments, the gene or gene product is associated with nuclear import of an HSP70 protein (e.g., HIKESHI). In some embodiments, the gene or gene product is associated with a Ser/Thr kinase (e.g., SIK2 or SIK3). In some embodiments, the gene or gene product is associated with SUMOylation of a protein (e.g., SUMO2, UBE2L6).

In some embodiments, the gene or gene product decreases the transduction efficiency of an AAV9 particle, optionally wherein the gene or gene product does not decrease, or does not substantially decrease, the transduction efficiency of an AAV2 particle. In some embodiments, the gene or gene product inhibits or prevents the transduction of an AAV9 particle, optionally wherein the gene or gene product does not inhibit or prevent the transduction of an AAV2 particle. In some embodiments, the gene or gene product is WRD11. In some embodiments, the gene or gene product is MRE11.

In some embodiments, gene or gene product is chosen from AP2A1, AP2B1, TM9SF4, ACTR5, PTMA, PAPOLA, PDHA1, PDHB, SLC25A19, GAK, AUNIP, FCHO2, ACTB, LIPT1, UCHL5, INO80E, ECHS1, NELFB, EDC4, PRMT1, PDS5B, PELI3, FBXL20, SLC35A1, or a combination thereof.

In some embodiments, the gene or gene product binds to ATP, a chaperone protein, and/or clathrin (e.g., GAK). In some embodiments, the gene or gene product is associated with clathrin-coated vesicle trafficking (e.g., AP2B1 or AP2A1). In some embodiments, the gene or gene product is associated with clathrin-mediated endocytosis (e.g., FCHO2). In some embodiments, the gene or gene product is associated with vesicular trafficking (e.g., ACTB). In some embodiments, the gene or gene product is associated with an innate immune response (e.g., PELI3 or FBXL20). In some embodiments, the gene or gene product is associated with glucose metabolism (e.g., PDHB or PDHA1). In some embodiments, the gene or gene product is associated with DNA resection and/or homologous recombination, e.g., after DNA damage (e.g., AUNIP). In some embodiments, the gene or gene product is associated with DNA repair, e.g., after DNA damage (e.g., UCHL5, INO80E, PDS5B, ACTR5, or PRMT1). In some embodiments, the gene or gene product is associated with transport of CMP-sialic acid from cytosol to a Golgi vesicle (e.g., SLC35A1). In some embodiments, the gene or gene product is associated with localization of polypeptides comprising glycine-rich transmembrane domains, e.g., to the cell surface (e.g., TM9SF4). In some embodiments, the gene or gene product is associated with poly(A) tail synthesis (e.g., PAPOLA). In some embodiments, the gene or gene product is PTMA. In some embodiments, the gene or gene product is associated with elongation of an mRNA by RNA polymerase II (e.g., NELFB). In some embodiments, the gene or gene product is associated with mRNA degradation (e.g., EDC4). In some embodiments, the gene or gene product encodes a mitochondrial lipoyltransferase (e.g., LIPT1). In some embodiments, the gene or gene product is associated with mitochondrial fatty acid beta-oxidation (e.g., ECHS1). In some embodiments, the gene or gene product encodes a mitochondrial thiamine pyrophosphate carrier (e.g., SLC25A19).

In some embodiments, the gene or gene product decreases the transduction efficiency of AAV2 and AAV9 particles. In some embodiments, the gene or gene product inhibits or prevents the transduction of AAV2 and AAV9 particles. In some embodiments, the gene or gene product is WRD11. In some embodiments, the gene or gene product is MRE11.

In some embodiments, the gene or gene product is selected from the group consisting of: PITPNB, PITP, FAM91A1, WDR11, AP1G1, APIM1, APIS1, APIS3, HEATR5B, STX16, APIB1, PIAS1, DENR, ARL1, ZFAT, TBC1D23, LIN37, RALGAPB, B3GNT2, ELOVL1, AP2B1, KIAA2013, PTEN, MCTS1, NBN, HELZ, SLC38A10, FBXL20, TGIF1, KDSR, CPD, CHD7, USP9X, SIMC1, TAF11, VAPA, MTMR6, RAB1B, SLF2, MRE11, VTI1A, MBOAT7, PPP6R3, or a combination thereof.

In some embodiments, the gene or gene product is associated with the long-chain FA elongation cycle (e.g., ELOVL1). In some embodiments, the gene or gene product is associated with lipoic acid biosynthesis (e.g., PIAS1). In some embodiments, the gene or gene product is associated with a protein phosphatase catalytic subunit (e.g., PPP6R3). In some embodiments, the gene or gene product is associated with the WDR11 pathway (e.g., WDR11, FAM91A1, AP1G1, AP1S1, AP1M1, AP1S3, or TBC1D23). In some embodiments, the gene or gene product is associated with retrograde transport from the Golgi apparatus to the endoplasmic reticulum, e.g., of PI and/or PC (e.g., PITP). In some embodiments, the gene or gene product is associated with vesicular transport from a late endosome to a trans-Golgi network (e.g., STX16). In some embodiments, the gene or gene product is associated with an activity or recruitment of a golgin, arfaptin, and/or Arf-GEF to a trans-Golgi network (e.g., ARL1). In some embodiments, the gene or gene product encodes a component of a clathrin-dependent vesicle and/or is associated with AP1G1/AP-1-mediated protein trafficking (e.g., HEATR5B).

In some embodiments, the gene or gene product is AAV-R, GPR108, WDR11 or MRE11. In some embodiments, the gene or gene product is AAV-R. In some embodiments, the gene or gene product is GPR108. In some embodiments, the gene or gene product is WDR11. In some embodiments, the gene or gene product is MRE11.

In some embodiments, the modulator is: (a) a gene editing system targeted to one or more sites within the gene or a regulatory element thereof; (b) a nucleic acid encoding one or more components of the gene editing system; or (c) a combination thereof. In some embodiments, the gene editing system is a CRISPR/Cas system, a zinc finger nuclease system, a TALEN system, and a meganuclease system.

In some embodiments, the gene editing system binds to a target sequence in an early (e.g., the first, second, or third) exon or intron of the gene. In some embodiments, the gene editing system binds a target sequence of the gene, and the target sequence is upstream of exon 4, e.g., in exon 1, exon 2, or exon 3. In some embodiments, the gene editing system binds to a target sequence in a late exon or intron of the gene. In some embodiments, the gene editing system binds a target sequence of the gene, and the target sequence is downstream of a preantepenultimate exon, e.g., is in an antepenultimate exon, a penultimate exon, or a last exon. In some embodiments, the gene editing system is a CRISPR/Cas system comprising a guide RNA (gRNA) molecule comprising a targeting sequence which hybridizes to a target sequence of the gene. In some embodiments, the CRISPR/Cas system is a CRISPR/Cas9 system. In some embodiments, the CRISPR/Cas system is a CRISPR/Cas12a system.

In some embodiments, the modulator is a small interfering RNA (siRNA) or small hairpin (shRNA) specific for the gene, or a nucleic acid encoding the siRNA or shRNA. In some embodiments, the siRNA or shRNA comprises a nucleotide sequence complementary to a nucleotide sequence of an mRNA encoded by the gene. In some embodiments, the modulator is a guide RNA (gRNA) specific for the gene, or a nucleic acid encoding the gRNA. In some embodiments, the gRNA comprises a nucleotide sequence complementary to a nucleotide sequence of the gene.

In some embodiments, the modulator is an antisense oligonucleotide (ASO) specific for the gene, or a nucleic acid encoding the ASO. In some embodiments, the ASO comprises a nucleotide sequence complementary to a nucleotide sequence of an mRNA encoded by the gene.

In some embodiments, the modulator is a small molecule (e.g., a compound described herein, e.g., in FIG. 7). In some embodiments, the small molecule is a protein degrader.

In some embodiments, the modulator is a protein or a peptide, or a nucleic acid encoding the protein or peptide. In some embodiments, the modulator is an antibody molecule (e.g., an scFv or sdAb). In some embodiments, the modulator is a dominant negative binding partner of a protein encoded by the gene, or a nucleic acid encoding the dominant negative binding partner. In some embodiments, the modulator is a dominant negative variant (e.g., catalytically inactive) of a protein encoded by the gene, or a nucleic acid encoding said dominant negative variant.

In some embodiments, the AAV particle comprises an AAV genome. In some embodiments, the AAV particle comprises an AAV-like particle. In some embodiments, the AAV particle comprises a capsid. In some embodiments, the AAV particle has a serotype of AAV1, AAV2, AAV3, AAV4, AAV5, AAV6, AAV7, AAV8, AAV9, AAV10, AAV11, or a combination thereof. In some embodiments, the AAV particle is an AAV2 particle. In some embodiments, the AAV particle is an AAV9 particle.

In some embodiments, the AAV particle has a tropism towards the CNS (e.g., AAV1, AAV2, AAV4, AAV5, AAV8, or AAV9). In some embodiments, the AAV particle has a tropism towards heart (e.g., AAV1, AAV8, or AAV9). In some embodiments, the AAV particle has a tropism towards kidney (e.g., AAV2). In some embodiments, the AAV particle has a tropism towards liver (e.g., AAV7, AAV8, or AAV9). In some embodiments, the AAV particle has a tropism towards lung (e.g., AAV4, AAV5, AAV6, or AAV9). In some embodiments, the AAV particle has a tropism towards pancreas (e.g., AAV8). In some embodiments, the AAV particle has a tropism towards a photoreceptor cell (e.g., AAV2, AAV5, or AAV8). In some embodiments, the AAV particle has a tropism towards retinal pigment epithelium (RPE) (e.g., AAV1, AAV2, AAV4, AAV5, or AAV8). In some embodiments, the AAV particle has a tropism towards skeletal muscle (e.g., AAV1, AAV6, AAV7, AAV8, or AAV9).

In some embodiments, the method results in a high gene modulating (e.g., gene editing) efficiency (e.g., at least 60%, 65%, 70%, 75%, 80%, 85%, 90%, 95%, or 98%) in a first tissue, with limited impact (e.g., a gene modulating (e.g., gene editing) efficiency of 20%, 15%, 10%, 5%, 2%, or less) in a second tissue. In some embodiments, the method results in a high modulating (e.g., gene editing) efficiency (e.g., at least 60%, 65%, 70%, 75%, 80%, 85%, 90%, 95%, or 98%) in liver, with limited impact (e.g., a gene modulating (e.g., gene editing) efficiency of 20%, 15%, 10%, 5%, 2%, or less) in skeletal muscle, bone marrow, or both.

In some embodiments, the AAV particle comprises a nucleotide sequence encoding a therapeutic protein, e.g., a protein associated with a disorder described herein. In some embodiments, the AAV particle comprises a nucleotide sequence encoding NGF, APOE2 (e.g., hAPOE2), TERT (e.g., hTERT), MAPT, GAD, AADC, NTN, GDNF, GCase, HTT, SMN, SMN2, SOD1, or C9orf72. In some embodiments, the AAV particle comprises a nucleotide sequence encoding a therapeutic nucleic acid, e.g., a nucleic acid targeting a nucleotide sequence encoding a protein associated with a disorder described herein.

In some embodiments, the cell is a brain cell, a liver cell, a spinal cord cell, a dorsal root ganglion (DRG) cell, a spleen cell, a lymph node cell, a kidney cell, a lung cell, a heart cell, a muscle cell (e.g., a skeletal muscle cell, a femur muscle cell), a diaphragm cell, a bone marrow cell, or a gonad cell. In some embodiments, the cell is a central nervous system (CNS) cell. In some embodiments, the CNS cell is an astrocyte, an oligodendrocyte, a microglial cell, or an ependymal cell. In some embodiments, the cell is a brain cell. In some embodiments, the brain cell is a neuron or glial cell. In some embodiments, the cell is a DRG cell. In some embodiments, the cell is a liver cell. In some embodiments, the liver cell is a hepatocyte, hepatic stellate cell, a Kupffer cell, or a liver sinusoidal endothelial cell.

In some embodiments, the cell is contacted with the AAV transduction modulator in vitro. In some embodiments, the cell is contacted with the AAV transduction modulator ex vivo. In some embodiments, the cell is contacted with the AAV transduction modulator in vivo.

In some embodiments, the method comprises contacting the cell with a plurality of AAV transduction modulators or an AAV modulator that modulates a plurality of genes or gene products associated with AAV transduction efficiency.

In another aspect, the disclosure provides a method of modulating transduction efficiency of an AAV particle, the method comprising administering to a subject in need thereof an effective amount of an AAV transduction modulator, thereby modulating the transduction efficiency of the AAV particle.

In some embodiments, the modulator modulates (e.g., increases or decreases) a gene or gene product associated with AAV transduction efficiency. In some embodiments, the gene or gene product is a mammalian (e.g., human) gene or gene product.

In some embodiments, the modulator inhibits the gene or gene product, e.g., a gene or gene product that is associated with a decreased transduction efficiency of an AAV particle. In some embodiments, the modulator activates the gene or gene product, e.g., a gene or gene product that is associated with an increased transduction efficiency of an AAV particle.

In some embodiments, the gene or gene product is associated with an increased transduction efficiency of an AAV particle. In some embodiments, the gene or gene product is associated with an increased transduction efficiency of an AAV particle of a first serotype (e.g., AAV2), an increased transduction efficiency of an AAV particle of a second serotype (e.g., AAV9), or both. In some embodiments, the gene or gene product is AAV-R. In some embodiments, the gene or gene product is GRP108.

In some embodiments, the gene or gene product is associated with a decreased transduction efficiency of an AAV particle. In some embodiments, the gene or gene product is associated with a decreased transduction efficiency of an AAV particle of a first serotype (e.g., AAV2), a decreased transduction efficiency of an AAV particle of a second serotype (e.g., AAV9), or both. In some embodiments, the gene or gene product is WRD11. In some embodiments, the gene or gene product is MRE11.

In some embodiments, the gene or gene product, when modulated, increases the transduction efficiency of an AAV particle. In some embodiments, the gene or gene product, when modulated, increases the transduction efficiency of an AAV particle of a first serotype (e.g., AAV2), the transduction efficiency of an AAV particle of a second serotype (e.g., AAV9), or both.

In some embodiments, the gene or gene product, when modulated, decreases the transduction efficiency of an AAV particle. In some embodiments, the gene or gene product, when modulated, decreases the transduction efficiency of an AAV particle of a first serotype (e.g., AAV2), the transduction efficiency of an AAV particle of a second serotype (e.g., AAV9), or both.

In some embodiments, the gene product is an RNA (e.g., an mRNA). In some embodiments, the gene product is a protein. In some embodiments, the gene or gene product is preferentially expressed in a target tissue (e.g., brain or liver). The following dependent claims describe the AAV transduction modulator based on its mechanism. In some embodiments, the modulator alters (e.g., increases or decreases) the expression of the gene. In some embodiments, the modulator alters the structure of the gene. In some embodiments, the modulator alters (e.g., increases or decreases) an activity (e.g., an enzymatic activity) of the gene product. In some embodiments, the modulator alters (e.g., increases or decreases) the level (e.g., abundance) of the gene product. In some embodiments, the modulator alters (e.g., increases or decreases) the stability of the gene product. In some embodiments, the modulator alters (e.g., increases or decreases) transduction efficiency of an AAV particle, e.g., as determined by an assay described herein (e.g., an FACS analysis, e.g., as described in Examples 1 or 2).

In some embodiments, the gene or gene product is AAV-R, GPR108, WDR11 or MRE11. In some embodiments, the gene or gene product is AAV-R. In some embodiments, the gene or gene product is GPR108. In some embodiments, the gene or gene product is WDR11. In some embodiments, the gene or gene product is MRE11.

In some embodiments, the method results in a high gene modulating (e.g., gene editing) efficiency (e.g., at least 60%, 65%, 70%, 75%, 80%, 85%, 90%, 95%, or 98%) in a first tissue, with limited impact (e.g., a gene modulating (e.g., gene editing) efficiency of 20%, 15%, 10%, 5%, 2%, or less) in a second tissue. In some embodiments, the method results in a high modulating (e.g., gene editing) efficiency (e.g., at least 60%, 65%, 70%, 75%, 80%, 85%, 90%, 95%, or 98%) in liver, with limited impact (e.g., a gene modulating (e.g., gene editing) efficiency of 20%, 15%, 10%, 5%, 2%, or less) in skeletal muscle, bone marrow, or both.

In some embodiments, the modulator increases transduction efficiency of an AAV particle. In some embodiments, the transduction efficiency is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference level of transduction efficiency. In some embodiments, the modulator increases transduction to at least 10, 20, 30, 40, 50, or 60 viral copies per genome. In some embodiments, the modulator increases transduction to at least 10³, 10⁴, 10⁵, 10⁶, or 10⁴ viral copies per μg DNA.

In some embodiments, the modulator decreases transduction efficiency of an AAV particle. In some embodiments, the transduction efficiency is decreased by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of transduction efficiency. In some embodiments, the modulator decreases transduction to no more than 0, 1, 2, 3, 4, 5, 6, 7, 8, 9, or 10 viral copies per genome. In some embodiments, the modulator decreases transduction to no more than 10², 10³, or 10⁴ viral copies per μg DNA.

In some embodiments, the reference level of transduction efficiency is the level of transduction efficiency in a cell that has not been contacted with the AAV transduction modulator. In some embodiments, the reference level of transduction efficiency is the level of transduction efficiency before the cell is contacted with the AAV transduction modulator.

In some embodiments, the gene or gene product increases the transduction efficiency of an AAV2 particle, optionally wherein the gene or gene product or gene or gene product does not increase, or does not substantially increase, the transduction efficiency of an AAV9 particle. In some embodiments, the gene or gene product is necessary for the transduction of an AAV2 particle, optionally wherein the gene or gene product is not necessary for the transduction of an AAV9 particle. In some embodiments, the gene or gene product is AAV-R. In some embodiments, the gene or gene product is GRP108.

In some embodiments, the gene or gene product is chosen from EXT1, EXT2, NDST1, BCL10, OAF, PDCD10, LMO4, MALT1, ZNF644, CHUK, GLCE, EP300, ARID4B, RAB21, MED13, DHX15, WASHC5, IKBKB, USP24, HSPA14, CXXC1, UBE2A, INTS8, CDC42, NFKB1, SIN3A, USP7, ARF5, CARD10, JAK1, SLC35C2, PAXK1, ZC3H11A, FAM20B, FAM72A, DHX36, INTS12, RPRD1B, or a combination thereof.

In some embodiments, the gene or gene product is associated with endosomal sorting (e.g., RAB21, WASHC5, or USP7). In some embodiments, the gene or gene product is associated with vesicle-mediated transport (e.g., CDC42 or ARF5). In some embodiments, the gene or gene product is associated with NFKB signaling (e.g., BCL10, MALT1, NFKB1, IKBKB, CHUK, or CARD10). In some embodiments, the gene or gene product is associated with heparan sulfate biosynthesis (e.g., EXT1, EXT2, NDST1, GLCE, or FAM20B). In some embodiments, the gene or gene product is associated with H3K9 methylation (e.g., ZNF644). In some embodiments, the gene or gene product is associated with pre-mRNA processing (e.g., DHX15). In some embodiments, the gene or gene product is associated with transcription (e.g., LMO4, EP300, MED13, SIN3, ARID4B, ZC3H11A, CXXC1, RPRD1B, or UBE2A). In some embodiments, the gene or gene product is associated with integrator complex and/or RNA polymerase II function (e.g., INTS8 or INST12). In some embodiments, the gene or gene product is associated with DNA repair and/or AAV genome resolution (e.g., DHX36, ARID4B, UBE2A, or USP24). In some embodiments, the gene or gene product is associated with cell proliferation and/or apoptosis (e.g., PDCD10). In some embodiments, the gene or gene product is associated with spondylocarpotarsal synostosis syndrome (e.g., OAF). In some embodiments, the gene or gene product is associated with protein folding (e.g., HSPA14). In some embodiments, the gene or gene product is associated with helicase activity (e.g., DHX36). In some embodiments, the gene or gene product is associated with fucosylation of Notch and/or transport of GCP-fucose (e.g., SLC35C2).

In some embodiments, the gene or gene product increases the transduction efficiency of an AAV9 particle, optionally wherein the gene or gene product does not increase, or does not substantially increase, the transduction efficiency of an AAV2 particle. In some embodiments, the gene or gene product is necessary for the transduction of an AAV9 particle, optionally wherein the gene or gene product is not necessary for the transduction of an AAV2 particle. In some embodiments, the gene or gene product is AAV-R. In some embodiments, the gene or gene product is GRP108.

In some embodiments, the gene or gene product is chosen from TMPRSS11B, HTT, SNRNP70, BRD7, DMXL1, RAB10, CNGA1, KLHDC3, CHP1, CYP3A5, ELOVL4, PNISR, ZCCHC14, AZGP1, TPST1, SC5D, ELP2, ELP3, IP09, RAB14, WDR7, XRCC4, GDI2, or a combination thereof.

In some embodiments, the gene or gene product is associated with microtubule-mediated transport or vesicle function (e.g., HT). In some embodiments, the gene or gene product is an ion channel (e.g., CNGA1). In some embodiments, the gene or gene product is associated with nuclear protein import (e.g., IP09). In some embodiments, the gene or gene product is associated with O-sulfation of a tyrosine residue within an acidic motif of a polypeptide (e.g., TPST1). In some embodiments, the gene or gene product is associated with protection of viral RNA (e.g., ZCCHC14).

In some embodiments, the gene or gene product is associated with RNA binding by an AAV protein (e.g., PNISR). In some embodiments, the gene or gene product is associated with calcium ion-regulated exocytosis (e.g., CHP1). In some embodiments, the gene or gene product is associated with intracellular trafficking (e.g., RAB10, RAB14, GDI2, WDR7, or DMXL1). In some embodiments, the gene or gene product is a subunit (e.g., a catalytic tRNA acetyltransferase subunit) of an elongator complex, e.g., of an RNA polymerase (e.g., RNA polymerase II), e.g., ELP2 or ELP3. In some embodiments, the gene or gene product binds to chromatin (e.g., KLHDC3). In some embodiments, the gene or gene product is associated with cholesterol biosynthesis (e.g., SC5D).

In some embodiments, the gene or gene product increases the transduction efficiency of AAV2 and AAV9 particles. In some embodiments, the gene or gene product is necessary for the transduction of AAV2 and AAV9 particles. In some embodiments, the gene or gene product is AAV-R. In some embodiments, the gene or gene product is GRP108.

In some embodiments, the gene or gene product is chosen from KIAA0319L, BAMBI, TM9SF2, PHIP, DCLRE1C, VPS35, GPR108, CYREN, ACP2, F8A2, F8A1, F8A3, RBPJ, ATP2C1, ICMT, RABIF, IER3IP1, RBM10, SMG7, GTF2I, ELAVL1, MEPCE, RAB4A, IER3IP1, SAMD1, or a combination thereof.

In some embodiments, the gene or gene product is associated with influenza infection (e.g., ACP2). In some embodiments, the gene or gene product binds to unmethylated CGIs (e.g., SAMD1). In some embodiments, the gene or gene product is associated with intracellular vesicular transport (e.g., RABIF). In some embodiments, the gene or gene product is associated with posttranslational modification of cysteine residues (e.g., C-terminal cysteine residues), e.g., in a target protein (e.g., ICMT). In some embodiments, the gene or gene product binds to a capsid protein of AAV (e.g., KIAA0319L). In some embodiments, the gene or gene product is associated with inhibition of a TLR (e.g., TLR9), e.g., GPR108. In some embodiments, the gene or gene product is associated with endosome trafficking, e.g., of AAV-R (e.g., VPS35). In some embodiments, the gene or gene product is a calcium ATPase pump (e.g., ATP2C1). In some embodiments, the gene or gene product is associated with Notch signaling (e.g., RBPJ). In some embodiments, the gene or gene product is associated with HSPG metabolism (e.g., TM9SF2). In some embodiments, the gene or gene product is associated with inhibition of NHEJ (e.g., CYREN). In some embodiments, the gene or gene product is associated with viral hairpin resolution (e.g., DCLRE1C). In some embodiments, the gene or gene product is associated with Glc transporter translocation, optionally wherein the gene or gene product binds to insulin receptor substrate 1 protein (e.g., PHIP).

In some embodiments, the gene or gene product decreases the transduction efficiency of an AAV2 particle, optionally wherein the gene or gene product, when modulated, does not decrease, or does not substantially decrease, the transduction efficiency of an AAV9 particle. In some embodiments, the gene or gene product inhibits or prevents the transduction of an AAV2 particle, optionally wherein the gene or gene product does not inhibit or prevent the transduction of an AAV9 particle. In some embodiments, the gene or gene product is WDR11. In some embodiments, the gene or gene product is MRE11.

In some embodiments, the gene or gene product is chosen from GREB1L, BIRC2, TYMSOS, MSL3, SIK3, GTF2H5, PIGW, ATRAID, PPP4R1, ZFC3H1, SETDB1, SMCHD1, TMEM123, UHRF1, CLUL1, SMAD5, SETX, STEEP1, DENND6A, RTN4RL2, MTMR9, STRADA, PSIP1, BMPR1A, HIKESHI, AP3M1, MEN1, MYL12B, CREB1, RAB12, SMTNL1, MRGBP, WTAP, SUMO2, TCN1, ALDH3B1, ARL14EP, MYL12A, RHOD, PGAP2, EIF4A1, PIGN, DAXX, HDLBP, GOLGA1, SARNP, UBE2L6, TRIM49, SIK2, SOX4, OR6Q1, OR4D6, UNC93B1, CATSPERZ, PIGT, PYM1, MICOS13, RIN1, DOT1L, TRIM49C, MAPK20, COL8A1, FASN, KDM3B, PIGF, PIGA, PIGY, GPC3, TRAPPC2L, RAB31, ELP3, or a combination thereof.

In some embodiments, the gene or gene product is associated with a retinoic acid receptor activity (e.g., GREB1L). In some embodiments, the gene or gene product is a thymidylate synthetase (e.g., TYMSOS). In some embodiments, the gene or gene product is associated with chromatin remodeling (e.g., SMCHD1). In some embodiments, the gene or gene product is associated with histone H4 acetylation (e.g., MSL3). In some embodiments, the gene or gene product is associated with transcription and/or DNA repair (e.g., GTF2H5). In some embodiments, the gene or gene product is a histone methyltransferase (e.g., SETDB1). In some embodiments, the gene or gene product is associated with exosomal degradation of polyadenylated RNA (e.g., ZFC3H1). In some embodiments, the gene or gene product is associated with histone deacetylase recruitment to a DNA (e.g., UHRF1). In some embodiments, the gene or gene product is associated with oxidative stress-induced DNA double strand break response (e.g., SETX). In some embodiments, the gene or gene product is associated with histone modification (e.g., MEN1). In some embodiments, the gene or gene product is associated with nucleosome/DNA interaction (e.g., MRGBP). In some embodiments, the gene or gene product is associated with GPI biosynthesis (e.g., PIGW, PIGN, PIGT, PIGF, PIGA, or PIGY). In some embodiments, the gene or gene product is associated with maturation of GPI anchors (e.g., PGAP2). In some embodiments, the gene or gene product is associated with vesicle-mediated endocytosis (e.g., DENND6A, GOLGA1, MTMR9, GPC3, PPP4R1, RAB12, RAB31, RHOD, RIN1, TMEM123, or TRAPPC2L). In some embodiments, the gene or gene product is associated with clathrin-coated vesicle-mediated endocytosis (e.g., AP3M1). In some embodiments, the gene or gene product is associated with a BMP receptor kinase (e.g., SMAD5, BMPR1A, or CREB1). In some embodiments, the gene or gene product is associated with transcription (e.g., PSIP1). In some embodiments, the gene or gene product is associated with nuclear import of an HSP70 protein (e.g., HIKESHI). In some embodiments, the gene or gene product is associated with a Ser/Thr kinase (e.g., SIK2 or SIK3). In some embodiments, the gene or gene product is associated with SUMOylation of a protein (e.g., SUMO2, UBE2L6).

In some embodiments, the gene or gene product decreases the transduction efficiency of an AAV9 particle, optionally wherein the gene or gene product does not decrease, or does not substantially decrease, the transduction efficiency of an AAV2 particle. In some embodiments, the gene or gene product inhibits or prevents the transduction of an AAV9 particle, optionally wherein the gene or gene product does not inhibit or prevent the transduction of an AAV2 particle. In some embodiments, the gene or gene product is WDR11. In some embodiments, the gene or gene product is MRE11.

In some embodiments, gene or gene product is chosen from AP2A1, AP2B1, TM9SF4, ACTR5, PTMA, PAPOLA, PDHA1, PDHB, SLC25A19, GAK, AUNIP, FCHO2, ACTB, LIPT1, UCHL5, INO80E, ECHS1, NELFB, EDC4, PRMT1, PDS5B, PELI3, FBXL20, SLC35A1, or a combination thereof.

In some embodiments, the gene or gene product binds to ATP, a chaperone protein, and/or clathrin (e.g., GAK). In some embodiments, the gene or gene product is associated with clathrin-coated vesicle trafficking (e.g., AP2B1 or AP2A1). In some embodiments, the gene or gene product is associated with clathrin-mediated endocytosis (e.g., FCHO2). In some embodiments, the gene or gene product is associated with vesicular trafficking (e.g., ACTB). In some embodiments, the gene or gene product is associated with an innate immune response (e.g., PELI3 or FBXL20). In some embodiments, the gene or gene product is associated with glucose metabolism (e.g., PDHB or PDHA1). In some embodiments, the gene or gene product is associated with DNA resection and/or homologous recombination, e.g., after DNA damage (e.g., AUNIP). In some embodiments, the gene or gene product is associated with DNA repair, e.g., after DNA damage (e.g., UCHL5, INO80E, PDS5B, ACTR5, or PRMT1). In some embodiments, the gene or gene product is associated with transport of CMP-sialic acid from cytosol to a Golgi vesicle (e.g., SLC35A1). In some embodiments, the gene or gene product is associated with localization of polypeptides comprising glycine-rich transmembrane domains, e.g., to the cell surface (e.g., TM9SF4). In some embodiments, the gene or gene product is associated with poly(A) tail synthesis (e.g., PAPOLA). In some embodiments, the gene or gene product is PTMA. In some embodiments, the gene or gene product is associated with elongation of an mRNA by RNA polymerase II (e.g., NELFB). In some embodiments, the gene or gene product is associated with mRNA degradation (e.g., EDC4). In some embodiments, the gene or gene product encodes a mitochondrial lipoyltransferase (e.g., LIPT1). In some embodiments, the gene or gene product is associated with mitochondrial fatty acid beta-oxidation (e.g., ECHS1). In some embodiments, the gene or gene product encodes a mitochondrial thiamine pyrophosphate carrier (e.g., SLC25A19).

In some embodiments, the gene or gene product decreases the transduction efficiency of AAV2 and AAV9 particles. In some embodiments, the gene or gene product inhibits or prevents the transduction of AAV2 and AAV9 particles.

In some embodiments, the gene or gene product is selected from the group consisting of: PITPNB, PITP, FAM91A1, WDR11, AP1G1, AP1M1, AP1S1, AP1S3, HEATR5B, STX16, AP1B1, PIAS1, DENR, ARL1, ZFAT, TBC1D23, LIN37, RALGAPB, B3GNT2, ELOVL1, AP2B1, KIAA2013, PTEN, MCTS1, NBN, HELZ, SLC38A10, FBXL20, TGIF1, KDSR, CPD, CHD7, USP9X, SIMC1, TAF1, VAPA, MTMR6, RAB1B, SLF2, MRE11, VT11A, MBOAT7, PPP6R3, or a combination thereof.

In some embodiments, the gene or gene product is associated with the long-chain FA elongation cycle (e.g., ELOVL1). In some embodiments, the gene or gene product is associated with lipoic acid biosynthesis (e.g., PIAS1). In some embodiments, the gene or gene product is associated with a protein phosphatase catalytic subunit (e.g., PPP6R3). In some embodiments, the gene or gene product is associated with the WDR11 pathway (e.g., WDR11, FAM91A1, AP1G1, AP1S1, AP1M1, AP1S3, or TBC1D23). In some embodiments, the gene or gene product is associated with retrograde transport from the Golgi apparatus to the endoplasmic reticulum, e.g., of PI and/or PC (e.g., PITP). In some embodiments, the gene or gene product is associated with vesicular transport from a late endosome to a trans-Golgi network (e.g., STX16). In some embodiments, the gene or gene product is associated with an activity or recruitment of a golgin, arfaptin, and/or Arf-GEF to a trans-Golgi network (e.g., ARL1). In some embodiments, the gene or gene product encodes a component of a clathrin-dependent vesicle and/or is associated with AP1G1/AP-1-mediated protein trafficking (e.g., HEATR5B).

In some embodiments, the modulator is: (a) a gene editing system targeted to one or more sites within the gene or a regulatory element thereof; (b) a nucleic acid encoding one or more components of the gene editing system; or (c) a combination thereof. In some embodiments, the gene editing system is a CRISPR/Cas system, a zinc finger nuclease system, a TALEN system, and a meganuclease system.

In some embodiments, the gene editing system binds to a target sequence in an early (e.g., the first, second, or third) exon or intron of the gene. In some embodiments, the gene editing system binds a target sequence of the gene, and the target sequence is upstream of exon 4, e.g., in exon 1, exon 2, or exon 3. In some embodiments, the gene editing system binds to a target sequence in a late exon or intron of the gene. In some embodiments, the gene editing system binds a target sequence of the gene, and the target sequence is downstream of a preantepenultimate exon, e.g., is in an antepenultimate exon, a penultimate exon, or a last exon. In some embodiments, the gene editing system is a CRISPR/Cas system comprising a guide RNA (gRNA) molecule comprising a targeting sequence which hybridizes to a target sequence of the gene. In some embodiments, the CRISPR/Cas system is a CRISPR/Cas9 system. In some embodiments, the CRISPR/Cas system is a CRISPR/Cas12a system.

In some embodiments, the modulator is a small interfering RNA (siRNA) or small hairpin (shRNA) specific for the gene, or a nucleic acid encoding the siRNA or shRNA. In some embodiments, the siRNA or shRNA comprises a nucleotide sequence complementary to a nucleotide sequence of an mRNA encoded by the gene. In some embodiments, the modulator is a guide RNA (gRNA) specific for the gene, or a nucleic acid encoding the gRNA. In some embodiments, the gRNA comprises a nucleotide sequence complementary to a nucleotide sequence of the gene.

In some embodiments, the modulator is an antisense oligonucleotide (ASO) specific for the gene, or a nucleic acid encoding the ASO. In some embodiments, the ASO comprises a nucleotide sequence complementary to a nucleotide sequence of an mRNA encoded by the gene.

In some embodiments, the modulator is a small molecule (e.g., a compound described herein, e.g., in FIG. 7). In some embodiments, the small molecule is a protein degrader.

In some embodiments, the modulator is a protein or a peptide, or a nucleic acid encoding the protein or peptide. In some embodiments, the modulator is an antibody molecule (e.g., an scFv or sdAb). In some embodiments, the modulator is a dominant negative binding partner of a protein encoded by the gene, or a nucleic acid encoding the dominant negative binding partner. In some embodiments, the modulator is a dominant negative variant (e.g., catalytically inactive) of a protein encoded by the gene, or a nucleic acid encoding said dominant negative variant.

In some embodiments, the AAV particle comprises an AAV genome. In some embodiments, the AAV particle comprises an AAV-like particle. In some embodiments, the AAV particle comprises a capsid. In some embodiments, the AAV particle has a serotype of AAV1, AAV2, AAV3, AAV4, AAV5, AAV6, AAV7, AAV8, AAV9, AAV10, AAV11, or a combination thereof. In some embodiments, the AAV particle is an AAV2 particle. In some embodiments, the AAV particle is an AAV9 particle.

In some embodiments, the AAV particle has a tropism towards the CNS (e.g., AAV1, AAV2, AAV4, AAV5, AAV8, or AAV9). In some embodiments, the AAV particle has a tropism towards heart (e.g., AAV1, AAV8, or AAV9). In some embodiments, the AAV particle has a tropism towards kidney (e.g., AAV2). In some embodiments, the AAV particle has a tropism towards liver (e.g., AAV7, AAV8, or AAV9). In some embodiments, the AAV particle has a tropism towards lung (e.g., AAV4, AAV5, AAV6, or AAV9). In some embodiments, the AAV particle has a tropism towards pancreas (e.g., AAV8). In some embodiments, the AAV particle has a tropism towards a photoreceptor cell (e.g., AAV2, AAV5, or AAV8). In some embodiments, the AAV particle has a tropism towards retinal pigment epithelium (RPE) (e.g., AAV1, AAV2, AAV4, AAV5, or AAV8). In some embodiments, the AAV particle has a tropism towards skeletal muscle (e.g., AAV1, AAV6, AAV7, AAV8, or AAV9).

In some embodiments, the AAV particle comprises a nucleotide sequence encoding a therapeutic protein, e.g., a protein associated with a disorder described herein. In some embodiments, the AAV particle comprises a nucleotide sequence encoding NGF, APOE2 (e.g., hAPOE2), TERT (e.g., hTERT), MAPT, GAD, AADC, NTN, GDNF, GCase, HTT, SMN, SMN2, SOD1, or C9orf72. In some embodiments, the AAV particle comprises a nucleotide sequence encoding a therapeutic nucleic acid, e.g., a nucleic acid targeting a nucleotide sequence encoding a protein associated with a disorder described herein.

In some embodiments, the modulator is administered intravenously.

In some embodiments, the subject has not received, or is not receiving, a therapy comprising an AAV genome or AAV particle, when the AAV transduction modulator is administered. In some embodiments, the AAV transduction modulator was administered at least 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the subject receives the therapy. In some embodiments, the AAV transduction modulator was administered no more than 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the subject receives the therapy.

In some embodiments, the subject has received, or is receiving, a therapy comprising an AAV genome or AAV particle, when the AAV transduction modulator is administered. In some embodiments, the subject received the therapy at least 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the AAV transduction modulator is administered. In some embodiments, the subject received the therapy no more than 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the AAV transduction modulator is administered.

In some embodiments, the subject has a disorder or a symptom thereof, or is at risk of having a disorder or a symptom thereof. In some embodiments, the disorder is a neurodegenerative disorder. In some embodiments, the neurodegenerative disorder is Alzheimer's disease, Parkinson's disease, Huntington's disease, multiple sclerosis (MS), amyotrophic lateral sclerosis (ALS), or Batten disease. In some embodiments, the disorder is mild Alzheimer's disease, mild-to-moderate Alzheimer's disease, mild-to-severe Alzheimer's disease, early dementia, Parkinson's disease (e.g., idiopathic Parkinson's disease, bilateral idiopathic Parkinson's disease, mid-to-late stage Parkinson's disease), Huntington's disease (e.g., manifest Huntington's disease), Type 1 SMA, presymptomatic SMA, or amyotrophic lateral sclerosis (ALS) (e.g., familial ALS, ALS with SOD1 mutation, or ALS with C9orf72 mutation). In some embodiments, the disorder is an eye disorder. In some embodiments, the eye disorder is blindness, e.g., inherited or non-inherited blindness. In some embodiments, the eye disorder is Leber's congenital amaurosis, age-related macular degeneration, choroideremia, or color blindness.

In some embodiments, the method reduces the toxicity of the therapy, enhances the efficacy of the therapy, or both.

In some embodiments, the method comprises administering to the subject a plurality of AAV transduction modulators or an AAV modulator that modulates a plurality of genes or gene products associated with AAV transduction efficiency.

In another aspect, the disclosure provides a method of treating a disorder, the method comprising administering to a subject in need thereof an effective amount of a therapy comprising an AAV genome or an AAV particle, wherein a gene or gene product associated with AAV transduction efficiency is modulated in the subject, thereby treating the disorder.

In some embodiments, the subject has not received, or is not receiving, the therapy, when the AAV transduction modulator is administered. In some embodiments, the AAV transduction modulator was administered at least 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the subject receives the therapy. In some embodiments, the AAV transduction modulator was administered no more than 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the subject receives the therapy.

In some embodiments, the disorder is a neurodegenerative disorder. In some embodiments, the neurodegenerative disorder is Alzheimer's disease, Parkinson's disease, Huntington's disease, multiple sclerosis (MS), amyotrophic lateral sclerosis (ALS), or Batten disease. In some embodiments, the disorder is mild Alzheimer's disease, mild-to-moderate Alzheimer's disease, mild-to-severe Alzheimer's disease, early dementia, Parkinson's disease (e.g., idiopathic Parkinson's disease, bilateral idiopathic Parkinson's disease, mid-to-late stage Parkinson's disease), Huntington's disease (e.g., manifest Huntington's disease), Type 1 SMA, presymptomatic SMA, or amyotrophic lateral sclerosis (ALS) (e.g., familial ALS, ALS with SOD1 mutation, or ALS with C9orf72 mutation). In some embodiments, the disorder is an eye disorder. In some embodiments, the eye disorder is blindness, e.g., inherited or non-inherited blindness. In some embodiments, the eye disorder is Leber's congenital amaurosis, age-related macular degeneration, choroideremia, or color blindness. In some embodiments, the method reduces the toxicity of the therapy, enhances the efficacy of the therapy, or both.

In some embodiments, the modulator modulates (e.g., increases or decreases) a gene or gene product associated with AAV transduction efficiency. In some embodiments, the gene or gene product is a mammalian (e.g., human) gene or gene product.

In some embodiments, the modulator inhibits the gene or gene product, e.g., a gene or gene product that is associated with a decreased transduction efficiency of an AAV particle. In some embodiments, the modulator activates the gene or gene product, e.g., a gene or gene product that is associated with an increased transduction efficiency of an AAV particle.

In some embodiments, the gene or gene product is associated with an increased transduction efficiency of an AAV particle. In some embodiments, the gene or gene product is associated with an increased transduction efficiency of an AAV particle of a first serotype (e.g., AAV2), an increased transduction efficiency of an AAV particle of a second serotype (e.g., AAV9), or both. In some embodiments, the gene or gene product is AAV-R. In some embodiments, the gene or gene product is GRP108.

In some embodiments, the gene or gene product is associated with a decreased transduction efficiency of an AAV particle. In some embodiments, the gene or gene product is associated with a decreased transduction efficiency of an AAV particle of a first serotype (e.g., AAV2), a decreased transduction efficiency of an AAV particle of a second serotype (e.g., AAV9), or both. In some embodiments, the gene or gene product is WDR11. In some embodiments, the gene or gene product is MRE11.

In some embodiments, the gene or gene product, when modulated, increases the transduction efficiency of an AAV particle. In some embodiments, the gene or gene product, when modulated, increases the transduction efficiency of an AAV particle of a first serotype (e.g., AAV2), the transduction efficiency of an AAV particle of a second serotype (e.g., AAV9), or both.

In some embodiments, the gene or gene product, when modulated, decreases the transduction efficiency of an AAV particle. In some embodiments, the gene or gene product, when modulated, decreases the transduction efficiency of an AAV particle of a first serotype (e.g., AAV2), the transduction efficiency of an AAV particle of a second serotype (e.g., AAV9), or both.

In some embodiments, the gene product is an RNA (e.g., an mRNA). In some embodiments, the gene product is a protein. In some embodiments, the gene or gene product is preferentially expressed in a target tissue (e.g., brain or liver). The following dependent claims describe the AAV transduction modulator based on its mechanism. In some embodiments, the modulator alters (e.g., increases or decreases) the expression of the gene. In some embodiments, the modulator alters the structure of the gene. In some embodiments, the modulator alters (e.g., increases or decreases) an activity (e.g., an enzymatic activity) of the gene product. In some embodiments, the modulator alters (e.g., increases or decreases) the level (e.g., abundance) of the gene product. In some embodiments, the modulator alters (e.g., increases or decreases) the stability of the gene product. In some embodiments, the modulator alters (e.g., increases or decreases) transduction efficiency of an AAV particle, e.g., as determined by an assay described herein (e.g., an FACS analysis, e.g., as described in Examples 1 or 2).

In some embodiments, the gene or gene product is AAV-R, GPR108, WDR11 or MRE11. In some embodiments, the gene or gene product is AAV-R. In some embodiments, the gene or gene product is GPR108. In some embodiments, the gene or gene product is WDR11. In some embodiments, the gene or gene product is MRE11.

In some embodiments, the method results in a high gene modulating (e.g., gene editing) efficiency (e.g., at least 60%, 65%, 70%, 75%, 80%, 85%, 90%, 95%, or 98%) in a first tissue, with limited impact (e.g., a gene modulating (e.g., gene editing) efficiency of 20%, 15%, 10%, 5%, 2%, or less) in a second tissue. In some embodiments, the method results in a high modulating (e.g., gene editing) efficiency (e.g., at least 60%, 65%, 70%, 75%, 80%, 85%, 90%, 95%, or 98%) in liver, with limited impact (e.g., a gene modulating (e.g., gene editing) efficiency of 20%, 15%, 10%, 5%, 2%, or less) in skeletal muscle, bone marrow, or both.

In some embodiments, the modulator is administered intravenously.

In some embodiments, the modulator increases transduction efficiency of an AAV particle. In some embodiments, the transduction efficiency is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference level of transduction efficiency. In some embodiments, the modulator increases transduction to at least 10, 20, 30, 40, 50, or 60 viral copies per genome. In some embodiments, the modulator increases transduction to at least 10³, 10⁴, 10⁵, 10⁶, or 10⁴ viral copies per μg DNA.

In some embodiments, the modulator decreases transduction efficiency of an AAV particle.

In some embodiments, the transduction efficiency is decreased by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of transduction efficiency. In some embodiments, the modulator decreases transduction to no more than 0, 1, 2, 3, 4, 5, 6, 7, 8, 9, or 10 viral copies per genome. In some embodiments, the modulator decreases transduction to no more than 10², 10³, or 10⁴ viral copies per μg DNA.

In some embodiments, the reference level of transduction efficiency is the level of transduction efficiency in a cell that has not been contacted with the AAV transduction modulator. In some embodiments, the reference level of transduction efficiency is the level of transduction efficiency before the cell is contacted with the AAV transduction modulator.

In some embodiments, the gene or gene product increases the transduction efficiency of an AAV2 particle, optionally wherein the gene or gene product or gene or gene product does not increase, or does not substantially increase, the transduction efficiency of an AAV9 particle. In some embodiments, the gene or gene product is necessary for the transduction of an AAV2 particle, optionally wherein the gene or gene product is not necessary for the transduction of an AAV9 particle. In some embodiments, the gene or gene product is AAV-R. In some embodiments, the gene or gene product is GRP108.

In some embodiments, the gene or gene product is chosen from EXT1, EXT2, NDST1, BCL10, OAF, PDCD10, LMO4, MALT1, ZNF644, CHUK, GLCE, EP300, ARID4B, RAB21, MED13, DHX15, WASHC5, IKBKB, USP24, HSPA14, CXXC1, UBE2A, INTS8, CDC42, NFKB1, SIN3A, USP7, ARF5, CARD10, JAK1, SLC35C2, PAXK1, ZC3H11A, FAM20B, FAM72A, DHX36, INTS12, RPRD1B, or a combination thereof.

In some embodiments, the gene or gene product is associated with endosomal sorting (e.g., RAB21, WASHC5, or USP7). In some embodiments, the gene or gene product is associated with vesicle-mediated transport (e.g., CDC42 or ARF5). In some embodiments, the gene or gene product is associated with NFKB signaling (e.g., BCL10, MALT1, NFKB1, IKBKB, CHUK, or CARD10).

In some embodiments, the gene or gene product is associated with heparan sulfate biosynthesis (e.g., EXT1, EXT2, NDST1, GLCE, or FAM20B). In some embodiments, the gene or gene product is associated with H3K9 methylation (e.g., ZNF644). In some embodiments, the gene or gene product is associated with pre-mRNA processing (e.g., DHX15). In some embodiments, the gene or gene product is associated with transcription (e.g., LMO4, EP300, MED13, SIN3, ARID4B, ZC3H11A, CXXC1, RPRD1B, or UBE2A). In some embodiments, the gene or gene product is associated with integrator complex and/or RNA polymerase II function (e.g., INTS8 or INST12). In some embodiments, the gene or gene product is associated with DNA repair and/or AAV genome resolution (e.g., DHX36, ARID4B, UBE2A, or USP24). In some embodiments, the gene or gene product is associated with cell proliferation and/or apoptosis (e.g., PDCD10). In some embodiments, the gene or gene product is associated with spondylocarpotarsal synostosis syndrome (e.g., OAF). In some embodiments, the gene or gene product is associated with protein folding (e.g., HSPA14). In some embodiments, the gene or gene product is associated with helicase activity (e.g., DHX36). In some embodiments, the gene or gene product is associated with fucosylation of Notch and/or transport of GCP-fucose (e.g., SLC35C2).

In some embodiments, the gene or gene product increases the transduction efficiency of an AAV9 particle, optionally wherein the gene or gene product does not increase, or does not substantially increase, the transduction efficiency of an AAV2 particle. In some embodiments, the gene or gene product is necessary for the transduction of an AAV9 particle, optionally wherein the gene or gene product is not necessary for the transduction of an AAV2 particle. In some embodiments, the gene or gene product is AAV-R. In some embodiments, the gene or gene product is GRP108.

In some embodiments, the gene or gene product is chosen from TMPRSS11B, HTT, SNRNP70, BRD7, DMXL1, RAB10, CNGA1, KLHDC3, CHP1, CYP3A5, ELOVL4, PNISR, ZCCHC14, AZGP1, TPST1, SC5D, ELP2, ELP3, IP09, RAB14, WDR7, XRCC4, GDI2, or a combination thereof.

In some embodiments, the gene or gene product is associated with microtubule-mediated transport or vesicle function (e.g., HTT). In some embodiments, the gene or gene product is an ion channel (e.g., CNGA1). In some embodiments, the gene or gene product is associated with nuclear protein import (e.g., IP09). In some embodiments, the gene or gene product is associated with O-sulfation of a tyrosine residue within an acidic motif of a polypeptide (e.g., TPST1). In some embodiments, the gene or gene product is associated with protection of viral RNA (e.g., ZCCHC14).

In some embodiments, the gene or gene product is associated with RNA binding by an AAV protein (e.g., PNISR). In some embodiments, the gene or gene product is associated with calcium ion-regulated exocytosis (e.g., CHP1). In some embodiments, the gene or gene product is associated with intracellular trafficking (e.g., RAB10, RAB14, GDI2, WDR7, or DMXL1). In some embodiments, the gene or gene product is a subunit (e.g., a catalytic tRNA acetyltransferase subunit) of an elongator complex, e.g., of an RNA polymerase (e.g., RNA polymerase II), e.g., ELP2 or ELP3. In some embodiments, the gene or gene product binds to chromatin (e.g., KLHDC3). In some embodiments, the gene or gene product is associated with cholesterol biosynthesis (e.g., SC5D).

In some embodiments, the gene or gene product increases the transduction efficiency of AAV2 and AAV9 particles. In some embodiments, the gene or gene product is necessary for the transduction of AAV2 and AAV9 particles.

In some embodiments, the gene or gene product is chosen from KIAA0319L, BAMBI, TM9SF2, PHIP, DCLRE1C, VPS35, GPR108, CYREN, ACP2, F8A2, F8A1, F8A3, RBPJ, ATP2C1, ICMT, RABIF, IER3IP1, RBM10, SMG7, GTF2I, ELAVL1, MEPCE, RAB4A, IER3IP1, SAMD1, or a combination thereof.

In some embodiments, the gene or gene product is associated with influenza infection (e.g., ACP2). In some embodiments, the gene or gene product binds to unmethylated CGIs (e.g., SAMD1). In some embodiments, the gene or gene product is associated with intracellular vesicular transport (e.g., RABIF). In some embodiments, the gene or gene product is associated with posttranslational modification of cysteine residues (e.g., C-terminal cysteine residues), e.g., in a target protein (e.g., ICMT). In some embodiments, the gene or gene product binds to a capsid protein of AAV (e.g., KIAA0319L). In some embodiments, the gene or gene product is associated with inhibition of a TLR (e.g., TLR9), e.g., GPR108. In some embodiments, the gene or gene product is associated with endosome trafficking, e.g., of AAV-R (e.g., VPS35). In some embodiments, the gene or gene product is a calcium ATPase pump (e.g., ATP2C1). In some embodiments, the gene or gene product is associated with Notch signaling (e.g., RBPJ). In some embodiments, the gene or gene product is associated with HSPG metabolism (e.g., TM9SF2). In some embodiments, the gene or gene product is associated with inhibition of NHEJ (e.g., CYREN). In some embodiments, the gene or gene product is associated with viral hairpin resolution (e.g., DCLRE1C). In some embodiments, the gene or gene product is associated with Glc transporter translocation, optionally wherein the gene or gene product binds to insulin receptor substrate 1 protein (e.g., PHIP).

In some embodiments, the gene or gene product decreases the transduction efficiency of an AAV2 particle, optionally wherein the gene or gene product, when modulated, does not decrease, or does not substantially decrease, the transduction efficiency of an AAV9 particle. In some embodiments, the gene or gene product inhibits or prevents the transduction of an AAV2 particle, optionally wherein the gene or gene product does not inhibit or prevent the transduction of an AAV9 particle. In some embodiments, the gene or gene product is WDR11. In some embodiments, the gene or gene product is MRE11.

In some embodiments, the gene or gene product is chosen from GREB1L, BIRC2, TYMSOS, MSL3, SIK3, GTF2H5, PIGW, ATRAID, PPP4R1, ZFC3H1, SETDB1, SMCHD1, TMEM123, UHRF1, CLUL1, SMAD5, SETX, STEEP1, DENND6A, RTN4RL2, MTMR9, STRADA, PSIP1, BMPR1A, HIKESHI, AP3M1, MEN1, MYL12B, CREB1, RAB12, SMTNL1, MRGBP, WTAP, SUMO2, TCN1, ALDH3B1, ARL14EP, MYL12A, RHOD, PGAP2, EIF4A1, PIGN, DAXX, HDLBP, GOLGA1, SARNP, UBE2L6, TRIM49, SIK2, SOX4, OR6Q1, OR4D6, UNC93B1, CATSPERZ, PIGT, PYM1, MICOS13, RIN1, DOT1L, TRIM49C, MAPK20, COL8A1, FASN, KDM3B, PIGF, PIGA, PIGY, GPC3, TRAPPC2L, RAB31, ELP3, or a combination thereof.

In some embodiments, the gene or gene product is associated with a retinoic acid receptor activity (e.g., GREB1L). In some embodiments, the gene or gene product is a thymidylate synthetase (e.g., TYMSOS). In some embodiments, the gene or gene product is associated with chromatin remodeling (e.g., SMCHD1). In some embodiments, the gene or gene product is associated with histone H4 acetylation (e.g., MSL3). In some embodiments, the gene or gene product is associated with transcription and/or DNA repair (e.g., GTF2H5). In some embodiments, the gene or gene product is a histone methyltransferase (e.g., SETDB1). In some embodiments, the gene or gene product is associated with exosomal degradation of polyadenylated RNA (e.g., ZFC3H1). In some embodiments, the gene or gene product is associated with histone deacetylase recruitment to a DNA (e.g., UHRF1). In some embodiments, the gene or gene product is associated with oxidative stress-induced DNA double strand break response (e.g., SETX). In some embodiments, the gene or gene product is associated with histone modification (e.g., MEN1). In some embodiments, the gene or gene product is associated with nucleosome/DNA interaction (e.g., MRGBP). In some embodiments, the gene or gene product is associated with GPI biosynthesis (e.g., PIGW, PIGN, PIGT, PIGF, PIGA, or PIGY). In some embodiments, the gene or gene product is associated with maturation of GPI anchors (e.g., PGAP2). In some embodiments, the gene or gene product is associated with vesicle-mediated endocytosis (e.g., DENND6A, GOLGA1, MTMR9, GPC3, PPP4R1, RAB12, RAB31, RHOD, RIN1, TMEM123, or TRAPPC2L). In some embodiments, the gene or gene product is associated with clathrin-coated vesicle-mediated endocytosis (e.g., AP3M1). In some embodiments, the gene or gene product is associated with a BMP receptor kinase (e.g., SMAD5, BMPR1A, or CREB1). In some embodiments, the gene or gene product is associated with transcription (e.g., PSIP1). In some embodiments, the gene or gene product is associated with nuclear import of an HSP70 protein (e.g., HIKESHI). In some embodiments, the gene or gene product is associated with a Ser/Thr kinase (e.g., SIK2 or SIK3). In some embodiments, the gene or gene product is associated with SUMOylation of a protein (e.g., SUMO2, UBE2L6).

In some embodiments, the gene or gene product decreases the transduction efficiency of an AAV9 particle, optionally wherein the gene or gene product does not decrease, or does not substantially decrease, the transduction efficiency of an AAV2 particle. In some embodiments, the gene or gene product inhibits or prevents the transduction of an AAV9 particle, optionally wherein the gene or gene product does not inhibit or prevent the transduction of an AAV2 particle. In some embodiments, the gene or gene product is WDR11. In some embodiments, the gene or gene product is MRE11.

In some embodiments, gene or gene product is chosen from AP2A1, AP2B1, TM9SF4, ACTR5, PTMA, PAPOLA, PDHA1, PDHB, SLC25A19, GAK, AUNIP, FCHO2, ACTB, LIPT1, UCHL5, INO80E, ECHS1, NELFB, EDC4, PRMT1, PDS5B, PELI3, FBXL20, SLC35A1, or a combination thereof.

In some embodiments, the gene or gene product binds to ATP, a chaperone protein, and/or clathrin (e.g., GAK). In some embodiments, the gene or gene product is associated with clathrin-coated vesicle trafficking (e.g., AP2B1 or AP2A1). In some embodiments, the gene or gene product is associated with clathrin-mediated endocytosis (e.g., FCHO2). In some embodiments, the gene or gene product is associated with vesicular trafficking (e.g., ACTB). In some embodiments, the gene or gene product is associated with an innate immune response (e.g., PELI3 or FBXL20). In some embodiments, the gene or gene product is associated with glucose metabolism (e.g., PDHB or PDHA1). In some embodiments, the gene or gene product is associated with DNA resection and/or homologous recombination, e.g., after DNA damage (e.g., AUNIP). In some embodiments, the gene or gene product is associated with DNA repair, e.g., after DNA damage (e.g., UCHL5, INO80E, PDS5B, ACTR5, or PRMT1). In some embodiments, the gene or gene product is associated with transport of CMP-sialic acid from cytosol to a Golgi vesicle (e.g., SLC35A1). In some embodiments, the gene or gene product is associated with localization of polypeptides comprising glycine-rich transmembrane domains, e.g., to the cell surface (e.g., TM9SF4). In some embodiments, the gene or gene product is associated with poly(A) tail synthesis (e.g., PAPOLA). In some embodiments, the gene or gene product is PTMA. In some embodiments, the gene or gene product is associated with elongation of an mRNA by RNA polymerase II (e.g., NELFB). In some embodiments, the gene or gene product is associated with mRNA degradation (e.g., EDC4). In some embodiments, the gene or gene product encodes a mitochondrial lipoyltransferase (e.g., LIPT1). In some embodiments, the gene or gene product is associated with mitochondrial fatty acid beta-oxidation (e.g., ECHS1). In some embodiments, the gene or gene product encodes a mitochondrial thiamine pyrophosphate carrier (e.g., SLC25A19).

In some embodiments, the gene or gene product decreases the transduction efficiency of AAV2 and AAV9 particles. In some embodiments, the gene or gene product inhibits or prevents the transduction of AAV2 and AAV9 particles. In some embodiments, the gene or gene product is WDR11. In some embodiments, the gene or gene product is MRE11.

In some embodiments, the gene or gene product is selected from the group consisting of: PITPNB, PITP, FAM91A1, WDR11, AP1G1, APIM1, APIS1, APIS3, HEATR5B, STX16, APIB1, PIAS1, DENR, ARL1, ZFAT, TBC1D23, LIN37, RALGAPB, B3GNT2, ELOVL1, AP2B1, KIAA2013, PTEN, MCTS1, NBN, HELZ, SLC38A10, FBXL20, TGIF1, KDSR, CPD, CHD7, USP9X, SIMC1, TAF11, VAPA, MTMR6, RAB1B, SLF2, MRE11, VT11A, MBOAT7, PPP6R3, or a combination thereof.

In some embodiments, the gene or gene product is associated with the long-chain FA elongation cycle (e.g., ELOVL1). In some embodiments, the gene or gene product is associated with lipoic acid biosynthesis (e.g., PIAS1). In some embodiments, the gene or gene product is associated with a protein phosphatase catalytic subunit (e.g., PPP6R3). In some embodiments, the gene or gene product is associated with the WDR11 pathway (e.g., WDR11, FAM91A1, AP1G1, AP1S1, AP1M1, AP1S3, or TBC1D23). In some embodiments, the gene or gene product is associated with retrograde transport from the Golgi apparatus to the endoplasmic reticulum, e.g., of PI and/or PC (e.g., PITP). In some embodiments, the gene or gene product is associated with vesicular transport from a late endosome to a trans-Golgi network (e.g., STX16). In some embodiments, the gene or gene product is associated with an activity or recruitment of a golgin, arfaptin, and/or Arf-GEF to a trans-Golgi network (e.g., ARL1). In some embodiments, the gene or gene product encodes a component of a clathrin-dependent vesicle and/or is associated with AP1G1/AP-1-mediated protein trafficking (e.g., HEATR5B).

In some embodiments, the modulator is: (a) a gene editing system targeted to one or more sites within the gene or a regulatory element thereof; (b) a nucleic acid encoding one or more components of the gene editing system; or (c) a combination thereof. In some embodiments, the gene editing system is a CRISPR/Cas system, a zinc finger nuclease system, a TALEN system, and a meganuclease system.

In some embodiments, the gene editing system binds to a target sequence in an early (e.g., the first, second, or third) exon or intron of the gene. In some embodiments, the gene editing system binds a target sequence of the gene, and the target sequence is upstream of exon 4, e.g., in exon 1, exon 2, or exon 3. In some embodiments, the gene editing system binds to a target sequence in a late exon or intron of the gene. In some embodiments, the gene editing system binds a target sequence of the gene, and the target sequence is downstream of a preantepenultimate exon, e.g., is in an antepenultimate exon, a penultimate exon, or a last exon. In some embodiments, the gene editing system is a CRISPR/Cas system comprising a guide RNA (gRNA) molecule comprising a targeting sequence which hybridizes to a target sequence of the gene. In some embodiments, the CRISPR/Cas system is a CRISPR/Cas9 system. In some embodiments, the CRISPR/Cas system is a CRISPR/Cas12a system.

In some embodiments, the modulator is a small interfering RNA (siRNA) or small hairpin (shRNA) specific for the gene, or a nucleic acid encoding the siRNA or shRNA. In some embodiments, the siRNA or shRNA comprises a nucleotide sequence complementary to a nucleotide sequence of an mRNA encoded by the gene. In some embodiments, the modulator is a guide RNA (gRNA) specific for the gene, or a nucleic acid encoding the gRNA. In some embodiments, the gRNA comprises a nucleotide sequence complementary to a nucleotide sequence of the gene.

In some embodiments, the modulator is an antisense oligonucleotide (ASO) specific for the gene, or a nucleic acid encoding the ASO. In some embodiments, the ASO comprises a nucleotide sequence complementary to a nucleotide sequence of an mRNA encoded by the gene.

In some embodiments, the modulator is a small molecule (e.g., a compound described herein, e.g., in FIG. 7). In some embodiments, the small molecule is a protein degrader.

In some embodiments, the modulator is a protein or a peptide, or a nucleic acid encoding the protein or peptide. In some embodiments, the modulator is an antibody molecule (e.g., an scFv or sdAb). In some embodiments, the modulator is a dominant negative binding partner of a protein encoded by the gene, or a nucleic acid encoding the dominant negative binding partner. In some embodiments, the modulator is a dominant negative variant (e.g., catalytically inactive) of a protein encoded by the gene, or a nucleic acid encoding said dominant negative variant.

In some embodiments, the AAV particle comprises an AAV genome. In some embodiments, the AAV particle comprises an AAV-like particle. In some embodiments, the AAV particle comprises a capsid. In some embodiments, the AAV particle has a serotype of AAV1, AAV2, AAV3, AAV4, AAV5, AAV6, AAV7, AAV8, AAV9, AAV10, AAV11, or a combination thereof. In some embodiments, the AAV particle is an AAV2 particle. In some embodiments, the AAV particle is an AAV9 particle.

In some embodiments, the AAV particle has a tropism towards the CNS (e.g., AAV1, AAV2, AAV4, AAV5, AAV8, or AAV9). In some embodiments, the AAV particle has a tropism towards heart (e.g., AAV1, AAV8, or AAV9). In some embodiments, the AAV particle has a tropism towards kidney (e.g., AAV2). In some embodiments, the AAV particle has a tropism towards liver (e.g., AAV7, AAV8, or AAV9). In some embodiments, the AAV particle has a tropism towards lung (e.g., AAV4, AAV5, AAV6, or AAV9). In some embodiments, the AAV particle has a tropism towards pancreas (e.g., AAV8). In some embodiments, the AAV particle has a tropism towards a photoreceptor cell (e.g., AAV2, AAV5, or AAV8). In some embodiments, the AAV particle has a tropism towards retinal pigment epithelium (RPE) (e.g., AAV1, AAV2, AAV4, AAV5, or AAV8). In some embodiments, the AAV particle has a tropism towards skeletal muscle (e.g., AAV1, AAV6, AAV7, AAV8, or AAV9).

In some embodiments, the AAV particle comprises a nucleotide sequence encoding a therapeutic protein, e.g., a protein associated with a disorder described herein. In some embodiments, the AAV particle comprises a nucleotide sequence encoding NGF, APOE2 (e.g., hAPOE2), TERT (e.g., hTERT), MAPT, GAD, AADC, NTN, GDNF, GCase, HTT, SMN, SMN2, SOD1, or C9orf72.

In some embodiments, the AAV particle comprises a nucleotide sequence encoding a therapeutic nucleic acid, e.g., a nucleic acid targeting a nucleotide sequence encoding a protein associated with a disorder described herein.

In some embodiments, the method comprises administering to the subject a plurality of AAV transduction modulators or an AAV modulator that modulates a plurality of genes or gene products associated with AAV transduction efficiency.

In another aspect, the disclosure provides a method of treating a disorder, the method comprising administering to a subject in need thereof an effective amount of (a) an AAV transduction modulator and (b) a therapy comprising an AAV genome or an AAV particle, thereby treating the disorder.

In some embodiments, the subject has not received, or is not receiving, the therapy, when the AAV transduction modulator is administered. In some embodiments, the AAV transduction modulator was administered at least 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the subject receives the therapy. In some embodiments, the AAV transduction modulator was administered no more than 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the subject receives the therapy.

In some embodiments, the subject has received, or is receiving, the therapy, when the AAV transduction modulator is administered. In some embodiments, the subject received the therapy at least 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the AAV transduction modulator is administered. In some embodiments, the subject received the therapy no more than 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the AAV transduction modulator is administered.

In some embodiments, the disorder is a neurodegenerative disorder. In some embodiments, the neurodegenerative disorder is Alzheimer's disease, Parkinson's disease, Huntington's disease, multiple sclerosis (MS), amyotrophic lateral sclerosis (ALS), or Batten disease. In some embodiments, the disorder is mild Alzheimer's disease, mild-to-moderate Alzheimer's disease, mild-to-severe Alzheimer's disease, early dementia, Parkinson's disease (e.g., idiopathic Parkinson's disease, bilateral idiopathic Parkinson's disease, mid-to-late stage Parkinson's disease), Huntington's disease (e.g., manifest Huntington's disease), Type 1 SMA, presymptomatic SMA, or amyotrophic lateral sclerosis (ALS) (e.g., familial ALS, ALS with SOD1 mutation, or ALS with C9orf72 mutation). In some embodiments, the disorder is an eye disorder. In some embodiments, the eye disorder is blindness, e.g., inherited or non-inherited blindness. In some embodiments, the eye disorder is Leber's congenital amaurosis, age-related macular degeneration, choroideremia, or color blindness. In some embodiments, the method reduces the toxicity of the therapy, enhances the efficacy of the therapy, or both.

In some embodiments, the modulator modulates (e.g., increases or decreases) a gene or gene product associated with AAV transduction efficiency. In some embodiments, the gene or gene product is a mammalian (e.g., human) gene or gene product.

In some embodiments, the modulator inhibits the gene or gene product, e.g., a gene or gene product that is associated with a decreased transduction efficiency of an AAV particle. In some embodiments, the modulator activates the gene or gene product, e.g., a gene or gene product that is associated with an increased transduction efficiency of an AAV particle.

In some embodiments, the gene or gene product is associated with an increased transduction efficiency of an AAV particle. In some embodiments, the gene or gene product is associated with an increased transduction efficiency of an AAV particle of a first serotype (e.g., AAV2), an increased transduction efficiency of an AAV particle of a second serotype (e.g., AAV9), or both. In some embodiments, the gene or gene product is AAV-R. In some embodiments, the gene or gene product is GPR108.

In some embodiments, the gene or gene product is associated with a decreased transduction efficiency of an AAV particle. In some embodiments, the gene or gene product is associated with a decreased transduction efficiency of an AAV particle of a first serotype (e.g., AAV2), a decreased transduction efficiency of an AAV particle of a second serotype (e.g., AAV9), or both. In some embodiments, the gene or gene product is WDR11. In some embodiments, the gene or gene product is MRE11.

In some embodiments, the gene or gene product, when modulated, increases the transduction efficiency of an AAV particle. In some embodiments, the gene or gene product, when modulated, increases the transduction efficiency of an AAV particle of a first serotype (e.g., AAV2), the transduction efficiency of an AAV particle of a second serotype (e.g., AAV9), or both.

In some embodiments, the gene or gene product, when modulated, decreases the transduction efficiency of an AAV particle. In some embodiments, the gene or gene product, when modulated, decreases the transduction efficiency of an AAV particle of a first serotype (e.g., AAV2), the transduction efficiency of an AAV particle of a second serotype (e.g., AAV9), or both.

In some embodiments, the gene product is an RNA (e.g., an mRNA). In some embodiments, the gene product is a protein. In some embodiments, the gene or gene product is preferentially expressed in a target tissue (e.g., brain or liver). The following dependent claims describe the AAV transduction modulator based on its mechanism. In some embodiments, the modulator alters (e.g., increases or decreases) the expression of the gene. In some embodiments, the modulator alters the structure of the gene. In some embodiments, the modulator alters (e.g., increases or decreases) an activity (e.g., an enzymatic activity) of the gene product. In some embodiments, the modulator alters (e.g., increases or decreases) the level (e.g., abundance) of the gene product. In some embodiments, the modulator alters (e.g., increases or decreases) the stability of the gene product. In some embodiments, the modulator alters (e.g., increases or decreases) transduction efficiency of an AAV particle, e.g., as determined by an assay described herein (e.g., an FACS analysis, e.g., as described in Examples 1 or 2).

In some embodiments, the gene or gene product is AAV-R, GPR108, WDR11 or MRE11. In some embodiments, the gene or gene product is AAV-R. In some embodiments, the gene or gene product is GPR108. In some embodiments, the gene or gene product is WDR11. In some embodiments, the gene or gene product is MRE11.

In some embodiments, the method results in a high gene modulating (e.g., gene editing) efficiency (e.g., at least 60%, 65%, 70%, 75%, 80%, 85%, 90%, 95%, or 98%) in a first tissue, with limited impact (e.g., a gene modulating (e.g., gene editing) efficiency of 20%, 15%, 10%, 5%, 2%, or less) in a second tissue. In some embodiments, the method results in a high modulating (e.g., gene editing) efficiency (e.g., at least 60%, 65%, 70%, 75%, 80%, 85%, 90%, 95%, or 98%) in liver, with limited impact (e.g., a gene modulating (e.g., gene editing) efficiency of 20%, 15%, 10%, 5%, 2%, or less) in skeletal muscle, bone marrow, or both.

In some embodiments, the modulator is administered intravenously.

In some embodiments, the modulator increases transduction efficiency of an AAV particle. In some embodiments, the transduction efficiency is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference level of transduction efficiency. In some embodiments, the modulator increases transduction to at least 10, 20, 30, 40, 50, or 60 viral copies per genome. In some embodiments, the modulator increases transduction to at least 10³, 10⁴, 10⁵, 10⁶, or 10⁴ viral copies per μg DNA.

In some embodiments, the modulator decreases transduction efficiency of an AAV particle. In some embodiments, the transduction efficiency is decreased by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of transduction efficiency. In some embodiments, the modulator decreases transduction to no more than 0, 1, 2, 3, 4, 5, 6, 7, 8, 9, or 10 viral copies per genome. In some embodiments, the modulator decreases transduction to no more than 10², 10³, or 10⁴ viral copies per μg DNA.

In some embodiments, the reference level of transduction efficiency is the level of transduction efficiency in a cell that has not been contacted with the AAV transduction modulator. In some embodiments, the reference level of transduction efficiency is the level of transduction efficiency before the cell is contacted with the AAV transduction modulator.

In some embodiments, the gene or gene product increases the transduction efficiency of an AAV2 particle, optionally wherein the gene or gene product or gene or gene product does not increase, or does not substantially increase, the transduction efficiency of an AAV9 particle. In some embodiments, the gene or gene product is necessary for the transduction of an AAV2 particle, optionally wherein the gene or gene product is not necessary for the transduction of an AAV9 particle. In some embodiments, the gene or gene product is AAV-R. In some embodiments, the gene or gene product is GRP108.

In some embodiments, the gene or gene product is chosen from EXT1, EXT2, NDST1, BCL10, OAF, PDCD10, LMO4, MALT1, ZNF644, CHUK, GLCE, EP300, ARID4B, RAB21, MED13, DHX15, WASHC5, IKBKB, USP24, HSPA14, CXXC1, UBE2A, INTS8, CDC42, NFKB1, SIN3A, USP7, ARF5, CARD10, JAK1, SLC35C2, PAXK1, ZC3H11A, FAM20B, FAM72A, DHX36, INTS12, RPRD1B, or a combination thereof.

In some embodiments, the gene or gene product is associated with endosomal sorting (e.g., RAB21, WASHC5, or USP7). In some embodiments, the gene or gene product is associated with vesicle-mediated transport (e.g., CDC42 or ARF5). In some embodiments, the gene or gene product is associated with NFKB signaling (e.g., BCL10, MALT1, NFKB1, IKBKB, CHUK, or CARD10). In some embodiments, the gene or gene product is associated with heparan sulfate biosynthesis (e.g., EXT1, EXT2, NDST1, GLCE, or FAM20B). In some embodiments, the gene or gene product is associated with H3K9 methylation (e.g., ZNF644). In some embodiments, the gene or gene product is associated with pre-mRNA processing (e.g., DHX15). In some embodiments, the gene or gene product is associated with transcription (e.g., LMO4, EP300, MED13, SIN3, ARID4B, ZC3H11A, CXXC1, RPRD1B, or UBE2A). In some embodiments, the gene or gene product is associated with integrator complex and/or RNA polymerase II function (e.g., INTS8 or INST12). In some embodiments, the gene or gene product is associated with DNA repair and/or AAV genome resolution (e.g., DHX36, ARID4B, UBE2A, or USP24). In some embodiments, the gene or gene product is associated with cell proliferation and/or apoptosis (e.g., PDCD10). In some embodiments, the gene or gene product is associated with spondylocarpotarsal synostosis syndrome (e.g., OAF). In some embodiments, the gene or gene product is associated with protein folding (e.g., HSPA14). In some embodiments, the gene or gene product is associated with helicase activity (e.g., DHX36). In some embodiments, the gene or gene product is associated with fucosylation of Notch and/or transport of GCP-fucose (e.g., SLC35C2).

In some embodiments, the gene or gene product increases the transduction efficiency of an AAV9 particle, optionally wherein the gene or gene product does not increase, or does not substantially increase, the transduction efficiency of an AAV2 particle. In some embodiments, the gene or gene product is necessary for the transduction of an AAV9 particle, optionally wherein the gene or gene product is not necessary for the transduction of an AAV2 particle. In some embodiments, the gene or gene product is AAV-R. In some embodiments, the gene or gene product is GRP108.

In some embodiments, the gene or gene product is chosen from TMPRSS11B, HTT, SNRNP70, BRD7, DMXL1, RAB10, CNGA1, KLHDC3, CHP1, CYP3A5, ELOVL4, PNISR, ZCCHC14, AZGP1, TPST1, SC5D, ELP2, ELP3, IP09, RAB14, WDR7, XRCC4, GDI2, or a combination thereof.

In some embodiments, the gene or gene product is associated with microtubule-mediated transport or vesicle function (e.g., HT). In some embodiments, the gene or gene product is an ion channel (e.g., CNGA1). In some embodiments, the gene or gene product is associated with nuclear protein import (e.g., IP09). In some embodiments, the gene or gene product is associated with O-sulfation of a tyrosine residue within an acidic motif of a polypeptide (e.g., TPST1). In some embodiments, the gene or gene product is associated with protection of viral RNA (e.g., ZCCHC14).

In some embodiments, the gene or gene product is associated with RNA binding by an AAV protein (e.g., PNISR). In some embodiments, the gene or gene product is associated with calcium ion-regulated exocytosis (e.g., CHP1). In some embodiments, the gene or gene product is associated with intracellular trafficking (e.g., RAB10, RAB14, GDI2, WDR7, or DMXL1). In some embodiments, the gene or gene product is a subunit (e.g., a catalytic tRNA acetyltransferase subunit) of an elongator complex, e.g., of an RNA polymerase (e.g., RNA polymerase II), e.g., ELP2 or ELP3. In some embodiments, the gene or gene product binds to chromatin (e.g., KLHDC3). In some embodiments, the gene or gene product is associated with cholesterol biosynthesis (e.g., SC5D).

In some embodiments, the gene or gene product increases the transduction efficiency of AAV2 and AAV9 particles. In some embodiments, the gene or gene product is necessary for the transduction of AAV2 and AAV9 particles. In some embodiments, the gene or gene product is AAV-R. In some embodiments, the gene or gene product is GRP108.

In some embodiments, the gene or gene product is chosen from KIAA0319L, BAMBI, TM9SF2, PHIP, DCLRE1C, VPS35, GPR108, CYREN, ACP2, F8A2, F8A1, F8A3, RBPJ, ATP2C1, ICMT, RABIF, IER3IP1, RBM10, SMG7, GTF2I, ELAVL1, MEPCE, RAB4A, IER3IP1, SAMD1, or a combination thereof.

In some embodiments, the gene or gene product is associated with influenza infection (e.g., ACP2). In some embodiments, the gene or gene product binds to unmethylated CGIs (e.g., SAMD1). In some embodiments, the gene or gene product is associated with intracellular vesicular transport (e.g., RABIF). In some embodiments, the gene or gene product is associated with posttranslational modification of cysteine residues (e.g., C-terminal cysteine residues), e.g., in a target protein (e.g., ICMT). In some embodiments, the gene or gene product binds to a capsid protein of AAV (e.g., KIAA0319L). In some embodiments, the gene or gene product is associated with inhibition of a TLR (e.g., TLR9), e.g., GPR108. In some embodiments, the gene or gene product is associated with endosome trafficking, e.g., of AAV-R (e.g., VPS35). In some embodiments, the gene or gene product is a calcium ATPase pump (e.g., ATP2C1). In some embodiments, the gene or gene product is associated with Notch signaling (e.g., RBPJ). In some embodiments, the gene or gene product is associated with HSPG metabolism (e.g., TM9SF2). In some embodiments, the gene or gene product is associated with inhibition of NHEJ (e.g., CYREN). In some embodiments, the gene or gene product is associated with viral hairpin resolution (e.g., DCLRE1C). In some embodiments, the gene or gene product is associated with Glc transporter translocation, optionally wherein the gene or gene product binds to insulin receptor substrate 1 protein (e.g., PHIP).

In some embodiments, the gene or gene product decreases the transduction efficiency of an AAV2 particle, optionally wherein the gene or gene product, when modulated, does not decrease, or does not substantially decrease, the transduction efficiency of an AAV9 particle. In some embodiments, the gene or gene product inhibits or prevents the transduction of an AAV2 particle, optionally wherein the gene or gene product does not inhibit or prevent the transduction of an AAV9 particle. In some embodiments, the gene or gene product is WDR11. In some embodiments, the gene or gene product is MRE11.

In some embodiments, the gene or gene product is chosen from GREB1L, BIRC2, TYMSOS, MSL3, SIK3, GTF2H5, PIGW, ATRAID, PPP4R1, ZFC3H1, SETDB1, SMCHD1, TMEM123, UHRF1, CLUL1, SMAD5, SETX, STEEP1, DENND6A, RTN4RL2, MTMR9, STRADA, PSIP1, BMPR1A, HIKESHI, AP3M1, MEN1, MYL12B, CREB1, RAB12, SMTNL1, MRGBP, WTAP, SUMO2, TCN1, ALDH3B1, ARL14EP, MYL12A, RHOD, PGAP2, EIF4A1, PIGN, DAXX, HDLBP, GOLGA1, SARNP, UBE2L6, TRIM49, SIK2, SOX4, OR6Q1, OR4D6, UNC93B1, CATSPERZ, PIGT, PYM1, MICOS13, RIN1, DOT1L, TRIM49C, MAPK20, COL8A1, FASN, KDM3B, PIGF, PIGA, PIGY, GPC3, TRAPPC2L, RAB31, ELP3, or a combination thereof.

In some embodiments, the gene or gene product is associated with a retinoic acid receptor activity (e.g., GREB1L). In some embodiments, the gene or gene product is a thymidylate synthetase (e.g., TYMSOS). In some embodiments, the gene or gene product is associated with chromatin remodeling (e.g., SMCHD1). In some embodiments, the gene or gene product is associated with histone H4 acetylation (e.g., MSL3). In some embodiments, the gene or gene product is associated with transcription and/or DNA repair (e.g., GTF2H5). In some embodiments, the gene or gene product is a histone methyltransferase (e.g., SETDB1). In some embodiments, the gene or gene product is associated with exosomal degradation of polyadenylated RNA (e.g., ZFC3H1). In some embodiments, the gene or gene product is associated with histone deacetylase recruitment to a DNA (e.g., UHRF1). In some embodiments, the gene or gene product is associated with oxidative stress-induced DNA double strand break response (e.g., SETX). In some embodiments, the gene or gene product is associated with histone modification (e.g., MEN1). In some embodiments, the gene or gene product is associated with nucleosome/DNA interaction (e.g., MRGBP). In some embodiments, the gene or gene product is associated with GPI biosynthesis (e.g., PIGW, PIGN, PIGT, PIGF, PIGA, or PIGY). In some embodiments, the gene or gene product is associated with maturation of GPI anchors (e.g., PGAP2). In some embodiments, the gene or gene product is associated with vesicle-mediated endocytosis (e.g., DENND6A, GOLGA1, MTMR9, GPC3, PPP4R1, RAB12, RAB31, RHOD, RIN1, TMEM123, or TRAPPC2L). In some embodiments, the gene or gene product is associated with clathrin-coated vesicle-mediated endocytosis (e.g., AP3M1). In some embodiments, the gene or gene product is associated with a BMP receptor kinase (e.g., SMAD5, BMPR1A, or CREB1). In some embodiments, the gene or gene product is associated with transcription (e.g., PSIP1). In some embodiments, the gene or gene product is associated with nuclear import of an HSP70 protein (e.g., HIKESHI). In some embodiments, the gene or gene product is associated with a Ser/Thr kinase (e.g., SIK2 or SIK3). In some embodiments, the gene or gene product is associated with SUMOylation of a protein (e.g., SUMO2, UBE2L6).

In some embodiments, the gene or gene product decreases the transduction efficiency of an AAV9 particle, optionally wherein the gene or gene product does not decrease, or does not substantially decrease, the transduction efficiency of an AAV2 particle. In some embodiments, the gene or gene product inhibits or prevents the transduction of an AAV9 particle, optionally wherein the gene or gene product does not inhibit or prevent the transduction of an AAV2 particle. In some embodiments, the gene or gene product is WDR11. In some embodiments, the gene or gene product is MRE11.

In some embodiments, gene or gene product is chosen from AP2A1, AP2B1, TM9SF4, ACTR5, PTMA, PAPOLA, PDHA1, PDHB, SLC25A19, GAK, AUNIP, FCHO2, ACTB, LIPT1, UCHL5, INO80E, ECHS1, NELFB, EDC4, PRMT1, PDS5B, PELI3, FBXL20, SLC35A1, or a combination thereof.

In some embodiments, the gene or gene product binds to ATP, a chaperone protein, and/or clathrin (e.g., GAK). In some embodiments, the gene or gene product is associated with clathrin-coated vesicle trafficking (e.g., AP2B1 or AP2A1). In some embodiments, the gene or gene product is associated with clathrin-mediated endocytosis (e.g., FCHO2). In some embodiments, the gene or gene product is associated with vesicular trafficking (e.g., ACTB). In some embodiments, the gene or gene product is associated with an innate immune response (e.g., PELI3 or FBXL20). In some embodiments, the gene or gene product is associated with glucose metabolism (e.g., PDHB or PDHA1). In some embodiments, the gene or gene product is associated with DNA resection and/or homologous recombination, e.g., after DNA damage (e.g., AUNIP). In some embodiments, the gene or gene product is associated with DNA repair, e.g., after DNA damage (e.g., UCHL5, INO80E, PDS5B, ACTR5, or PRMT1). In some embodiments, the gene or gene product is associated with transport of CMP-sialic acid from cytosol to a Golgi vesicle (e.g., SLC35A1). In some embodiments, the gene or gene product is associated with localization of polypeptides comprising glycine-rich transmembrane domains, e.g., to the cell surface (e.g., TM9SF4). In some embodiments, the gene or gene product is associated with poly(A) tail synthesis (e.g., PAPOLA). In some embodiments, the gene or gene product is PTMA. In some embodiments, the gene or gene product is associated with elongation of an mRNA by RNA polymerase II (e.g., NELFB). In some embodiments, the gene or gene product is associated with mRNA degradation (e.g., EDC4). In some embodiments, the gene or gene product encodes a mitochondrial lipoyltransferase (e.g., LIPT1). In some embodiments, the gene or gene product is associated with mitochondrial fatty acid beta-oxidation (e.g., ECHS1). In some embodiments, the gene or gene product encodes a mitochondrial thiamine pyrophosphate carrier (e.g., SLC25A19).

In some embodiments, the gene or gene product decreases the transduction efficiency of AAV2 and AAV9 particles. In some embodiments, the gene or gene product inhibits or prevents the transduction of AAV2 and AAV9 particles. In some embodiments, the gene or gene product is WDR11. In some embodiments, the gene or gene product is MRE11.

In some embodiments, the gene or gene product is selected from the group consisting of: PITPNB, PITP, FAM91A1, WDR11, AP1G1, APIM1, APIS1, APIS3, HEATR5B, STX16, APIB1, PIAS1, DENR, ARL1, ZFAT, TBC1D23, LIN37, RALGAPB, B3GNT2, ELOVL1, AP2B1, KIAA2013, PTEN, MCTS1, NBN, HELZ, SLC38A10, FBXL20, TGIF1, KDSR, CPD, CHD7, USP9X, SIMC1, TAF11, VAPA, MTMR6, RAB1B, SLF2, MRE11, VT11A, MBOAT7, PPP6R3, or a combination thereof.

In some embodiments, the gene or gene product is associated with the long-chain FA elongation cycle (e.g., ELOVL1). In some embodiments, the gene or gene product is associated with lipoic acid biosynthesis (e.g., PIAS1). In some embodiments, the gene or gene product is associated with a protein phosphatase catalytic subunit (e.g., PPP6R3). In some embodiments, the gene or gene product is associated with the WDR11 pathway (e.g., WDR11, FAM91A1, AP1G1, AP1S1, AP1M1, AP1S3, or TBC1D23). In some embodiments, the gene or gene product is associated with retrograde transport from the Golgi apparatus to the endoplasmic reticulum, e.g., of PI and/or PC (e.g., PITP). In some embodiments, the gene or gene product is associated with vesicular transport from a late endosome to a trans-Golgi network (e.g., STX16). In some embodiments, the gene or gene product is associated with an activity or recruitment of a golgin, arfaptin, and/or Arf-GEF to a trans-Golgi network (e.g., ARL1). In some embodiments, the gene or gene product encodes a component of a clathrin-dependent vesicle and/or is associated with AP1G1/AP-1-mediated protein trafficking (e.g., HEATR5B).

In some embodiments, the modulator is: (a) a gene editing system targeted to one or more sites within the gene or a regulatory element thereof; (b) a nucleic acid encoding one or more components of the gene editing system; or (c) a combination thereof. In some embodiments, the gene editing system is a CRISPR/Cas system, a zinc finger nuclease system, a TALEN system, and a meganuclease system.

In some embodiments, the gene editing system binds to a target sequence in an early (e.g., the first, second, or third) exon or intron of the gene. In some embodiments, the gene editing system binds a target sequence of the gene, and the target sequence is upstream of exon 4, e.g., in exon 1, exon 2, or exon 3. In some embodiments, the gene editing system binds to a target sequence in a late exon or intron of the gene. In some embodiments, the gene editing system binds a target sequence of the gene, and the target sequence is downstream of a preantepenultimate exon, e.g., is in an antepenultimate exon, a penultimate exon, or a last exon. In some embodiments, the gene editing system is a CRISPR/Cas system comprising a guide RNA (gRNA) molecule comprising a targeting sequence which hybridizes to a target sequence of the gene. In some embodiments, the CRISPR/Cas system is a CRISPR/Cas9 system. In some embodiments, the CRISPR/Cas system is a CRISPR/Cas12a system.

In some embodiments, the modulator is a small interfering RNA (siRNA) or small hairpin (shRNA) specific for the gene, or a nucleic acid encoding the siRNA or shRNA. In some embodiments, the siRNA or shRNA comprises a nucleotide sequence complementary to a nucleotide sequence of an mRNA encoded by the gene. In some embodiments, the modulator is a guide RNA (gRNA) specific for the gene, or a nucleic acid encoding the gRNA. In some embodiments, the gRNA comprises a nucleotide sequence complementary to a nucleotide sequence of the gene.

In some embodiments, the modulator is an antisense oligonucleotide (ASO) specific for the gene, or a nucleic acid encoding the ASO. In some embodiments, the ASO comprises a nucleotide sequence complementary to a nucleotide sequence of an mRNA encoded by the gene.

In some embodiments, the modulator is a small molecule (e.g., a compound described herein, e.g., in FIG. 7). In some embodiments, the small molecule is a protein degrader.

In some embodiments, the modulator is a protein or a peptide, or a nucleic acid encoding the protein or peptide. In some embodiments, the modulator is an antibody molecule (e.g., an scFv or sdAb). In some embodiments, the modulator is a dominant negative binding partner of a protein encoded by the gene, or a nucleic acid encoding the dominant negative binding partner. In some embodiments, the modulator is a dominant negative variant (e.g., catalytically inactive) of a protein encoded by the gene, or a nucleic acid encoding said dominant negative variant.

In some embodiments, the AAV particle comprises an AAV genome. In some embodiments, the AAV particle comprises an AAV-like particle. In some embodiments, the AAV particle comprises a capsid. In some embodiments, the AAV particle has a serotype of AAV1, AAV2, AAV3, AAV4, AAV5, AAV6, AAV7, AAV8, AAV9, AAV10, AAV11, or a combination thereof. In some embodiments, the AAV particle is an AAV2 particle. In some embodiments, the AAV particle is an AAV9 particle.

In some embodiments, the AAV particle has a tropism towards the CNS (e.g., AAV1, AAV2, AAV4, AAV5, AAV8, or AAV9). In some embodiments, the AAV particle has a tropism towards heart (e.g., AAV1, AAV8, or AAV9). In some embodiments, the AAV particle has a tropism towards kidney (e.g., AAV2). In some embodiments, the AAV particle has a tropism towards liver (e.g., AAV7, AAV8, or AAV9). In some embodiments, the AAV particle has a tropism towards lung (e.g., AAV4, AAV5, AAV6, or AAV9). In some embodiments, the AAV particle has a tropism towards pancreas (e.g., AAV8). In some embodiments, the AAV particle has a tropism towards a photoreceptor cell (e.g., AAV2, AAV5, or AAV8). In some embodiments, the AAV particle has a tropism towards retinal pigment epithelium (RPE) (e.g., AAV1, AAV2, AAV4, AAV5, or AAV8). In some embodiments, the AAV particle has a tropism towards skeletal muscle (e.g., AAV1, AAV6, AAV7, AAV8, or AAV9).

In some embodiments, the AAV particle comprises a nucleotide sequence encoding a therapeutic protein, e.g., a protein associated with a disorder described herein. In some embodiments, the AAV particle comprises a nucleotide sequence encoding NGF, APOE2 (e.g., hAPOE2), TERT (e.g., hTERT), MAPT, GAD, AADC, NTN, GDNF, GCase, HTT, SMN, SMN2, SOD1, or C9orf72.

In some embodiments, the AAV particle comprises a nucleotide sequence encoding a therapeutic nucleic acid, e.g., a nucleic acid targeting a nucleotide sequence encoding a protein associated with a disorder described herein.

In some embodiments, the method comprises administering to the subject a plurality of AAV transduction modulators or an AAV modulator that modulates a plurality of genes or gene products associated with AAV transduction efficiency.

In another aspect, the disclosure provides a method of preparing a subject for a therapy comprising an AAV genome or an AAV particle, the method comprising administering to the subject an effective amount of an AAV transduction modulator, thereby preparing the subject for the therapy.

In some embodiments, the subject has not received, or is not receiving, the therapy, when the AAV transduction modulator is administered. In some embodiments, the AAV transduction modulator was administered at least 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the subject receives the therapy. In some embodiments, the AAV transduction modulator was administered no more than 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the subject receives the therapy.

In some embodiments, the subject has a disorder or a symptom thereof, or is at risk of having a disorder or a symptom thereof. In some embodiments, the disorder is a neurodegenerative disorder.

In some embodiments, the neurodegenerative disorder is Alzheimer's disease, Parkinson's disease, Huntington's disease, multiple sclerosis (MS), amyotrophic lateral sclerosis (ALS), or Batten disease. In some embodiments, the disorder is mild Alzheimer's disease, mild-to-moderate Alzheimer's disease, mild-to-severe Alzheimer's disease, early dementia, Parkinson's disease (e.g., idiopathic Parkinson's disease, bilateral idiopathic Parkinson's disease, mid-to-late stage Parkinson's disease), Huntington's disease (e.g., manifest Huntington's disease), Type 1 SMA, presymptomatic SMA, or amyotrophic lateral sclerosis (ALS) (e.g., familial ALS, ALS with SOD1 mutation, or ALS with C9orf72 mutation). In some embodiments, the disorder is an eye disorder. In some embodiments, the eye disorder is blindness, e.g., inherited or non-inherited blindness. In some embodiments, the eye disorder is Leber's congenital amaurosis, age-related macular degeneration, choroideremia, or color blindness. In some embodiments, the method reduces the toxicity of the therapy, enhances the efficacy of the therapy, or both.

In some embodiments, the modulator modulates (e.g., increases or decreases) a gene or gene product associated with AAV transduction efficiency. In some embodiments, the gene or gene product is a mammalian (e.g., human) gene or gene product.

In some embodiments, the modulator inhibits the gene or gene product, e.g., a gene or gene product that is associated with a decreased transduction efficiency of an AAV particle. In some embodiments, the modulator activates the gene or gene product, e.g., a gene or gene product that is associated with an increased transduction efficiency of an AAV particle.

In some embodiments, the gene or gene product is associated with an increased transduction efficiency of an AAV particle. In some embodiments, the gene or gene product is associated with an increased transduction efficiency of an AAV particle of a first serotype (e.g., AAV2), an increased transduction efficiency of an AAV particle of a second serotype (e.g., AAV9), or both. In some embodiments, the gene or gene product is AAV-R. In some embodiments, the gene or gene product is GPR108.

In some embodiments, the gene or gene product is associated with a decreased transduction efficiency of an AAV particle. In some embodiments, the gene or gene product is associated with a decreased transduction efficiency of an AAV particle of a first serotype (e.g., AAV2), a decreased transduction efficiency of an AAV particle of a second serotype (e.g., AAV9), or both. In some embodiments, the gene or gene product is WDR11. In some embodiments, the gene or gene product is MRE11.

In some embodiments, the gene or gene product, when modulated, increases the transduction efficiency of an AAV particle. In some embodiments, the gene or gene product, when modulated, increases the transduction efficiency of an AAV particle of a first serotype (e.g., AAV2), the transduction efficiency of an AAV particle of a second serotype (e.g., AAV9), or both.

In some embodiments, the gene or gene product, when modulated, decreases the transduction efficiency of an AAV particle. In some embodiments, the gene or gene product, when modulated, decreases the transduction efficiency of an AAV particle of a first serotype (e.g., AAV2), the transduction efficiency of an AAV particle of a second serotype (e.g., AAV9), or both.

In some embodiments, the gene product is an RNA (e.g., an mRNA). In some embodiments, the gene product is a protein. In some embodiments, the gene or gene product is preferentially expressed in a target tissue (e.g., brain or liver). The following dependent claims describe the AAV transduction modulator based on its mechanism. In some embodiments, the modulator alters (e.g., increases or decreases) the expression of the gene. In some embodiments, the modulator alters the structure of the gene. In some embodiments, the modulator alters (e.g., increases or decreases) an activity (e.g., an enzymatic activity) of the gene product. In some embodiments, the modulator alters (e.g., increases or decreases) the level (e.g., abundance) of the gene product. In some embodiments, the modulator alters (e.g., increases or decreases) the stability of the gene product. In some embodiments, the modulator alters (e.g., increases or decreases) transduction efficiency of an AAV particle, e.g., as determined by an assay described herein (e.g., an FACS analysis, e.g., as described in Examples 1 or 2).

In some embodiments, the modulator increases transduction efficiency of an AAV particle. In some embodiments, the transduction efficiency is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference level of transduction efficiency. In some embodiments, the modulator increases transduction to at least 10, 20, 30, 40, 50, or 60 viral copies per genome. In some embodiments, the modulator increases transduction to at least 10³, 10⁴, 10⁵, 10⁶, or 10⁷ viral copies per μg DNA.

In some embodiments, the modulator decreases transduction efficiency of an AAV particle. In some embodiments, the transduction efficiency is decreased by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of transduction efficiency. In some embodiments, the modulator decreases transduction to no more than 0, 1, 2, 3, 4, 5, 6, 7, 8, 9, or 10 viral copies per genome. In some embodiments, the modulator decreases transduction to no more than 10², 10³, or 10⁴ viral copies per μg DNA.

In some embodiments, the reference level of transduction efficiency is the level of transduction efficiency in a cell that has not been contacted with the AAV transduction modulator. In some embodiments, the reference level of transduction efficiency is the level of transduction efficiency before the cell is contacted with the AAV transduction modulator.

In some embodiments, the gene or gene product increases the transduction efficiency of an AAV2 particle, optionally wherein the gene or gene product or gene or gene product does not increase, or does not substantially increase, the transduction efficiency of an AAV9 particle. In some embodiments, the gene or gene product is necessary for the transduction of an AAV2 particle, optionally wherein the gene or gene product is not necessary for the transduction of an AAV9 particle. In some embodiments, the gene or gene product is AAV-R. In some embodiments, the gene or gene product is GRP108.

In some embodiments, the gene or gene product is chosen from EXT1, EXT2, NDST1, BCL10, OAF, PDCD10, LMO4, MALT1, ZNF644, CHUK, GLCE, EP300, ARID4B, RAB21, MED13, DHX15, WASHC5, IKBKB, USP24, HSPA14, CXXC1, UBE2A, INTS8, CDC42, NFKB1, SIN3A, USP7, ARF5, CARD10, JAK1, SLC35C2, PAXK1, ZC3H11A, FAM20B, FAM72A, DHX36, INTS12, RPRD1B, or a combination thereof.

In some embodiments, the gene or gene product is associated with endosomal sorting (e.g., RAB21, WASHC5, or USP7). In some embodiments, the gene or gene product is associated with vesicle-mediated transport (e.g., CDC42 or ARF5). In some embodiments, the gene or gene product is associated with NFKB signaling (e.g., BCL10, MALT1, NFKB1, IKBKB, CHUK, or CARD10).

In some embodiments, the gene or gene product is associated with heparan sulfate biosynthesis (e.g., EXT1, EXT2, NDST1, GLCE, or FAM20B). In some embodiments, the gene or gene product is associated with H3K9 methylation (e.g., ZNF644). In some embodiments, the gene or gene product is associated with pre-mRNA processing (e.g., DHX15). In some embodiments, the gene or gene product is associated with transcription (e.g., LMO4, EP300, MED13, SIN3, ARID4B, ZC3H11A, CXXC1, RPRD1B, or UBE2A). In some embodiments, the gene or gene product is associated with integrator complex and/or RNA polymerase II function (e.g., INTS8 or INST12). In some embodiments, the gene or gene product is associated with DNA repair and/or AAV genome resolution (e.g., DHX36, ARID4B, UBE2A, or USP24). In some embodiments, the gene or gene product is associated with cell proliferation and/or apoptosis (e.g., PDCD10). In some embodiments, the gene or gene product is associated with spondylocarpotarsal synostosis syndrome (e.g., OAF). In some embodiments, the gene or gene product is associated with protein folding (e.g., HSPA14). In some embodiments, the gene or gene product is associated with helicase activity (e.g., DHX36). In some embodiments, the gene or gene product is associated with fucosylation of Notch and/or transport of GCP-fucose (e.g., SLC35C2).

In some embodiments, the gene or gene product increases the transduction efficiency of an AAV9 particle, optionally wherein the gene or gene product does not increase, or does not substantially increase, the transduction efficiency of an AAV2 particle. In some embodiments, the gene or gene product is necessary for the transduction of an AAV9 particle, optionally wherein the gene or gene product is not necessary for the transduction of an AAV2 particle. In some embodiments, the gene or gene product is AAV-R. In some embodiments, the gene or gene product is GRP108.

In some embodiments, the gene or gene product is chosen from TMPRSS11B, HTT, SNRNP70, BRD7, DMXL1, RAB10, CNGA1, KLHDC3, CHP1, CYP3A5, ELOVL4, PNISR, ZCCHC14, AZGP1, TPST1, SC5D, ELP2, ELP3, IPO9, RAB14, WDR7, XRCC4, GDI2, or a combination thereof.

In some embodiments, the gene or gene product is associated with microtubule-mediated transport or vesicle function (e.g., HT). In some embodiments, the gene or gene product is an ion channel (e.g., CNGA1). In some embodiments, the gene or gene product is associated with nuclear protein import (e.g., IP09). In some embodiments, the gene or gene product is associated with O-sulfation of a tyrosine residue within an acidic motif of a polypeptide (e.g., TPST1). In some embodiments, the gene or gene product is associated with protection of viral RNA (e.g., ZCCHC14).

In some embodiments, the gene or gene product is associated with RNA binding by an AAV protein (e.g., PNISR). In some embodiments, the gene or gene product is associated with calcium ion-regulated exocytosis (e.g., CHP1). In some embodiments, the gene or gene product is associated with intracellular trafficking (e.g., RAB10, RAB14, GDI2, WDR7, or DMXL1). In some embodiments, the gene or gene product is a subunit (e.g., a catalytic tRNA acetyltransferase subunit) of an elongator complex, e.g., of an RNA polymerase (e.g., RNA polymerase II), e.g., ELP2 or ELP3. In some embodiments, the gene or gene product binds to chromatin (e.g., KLHDC3). In some embodiments, the gene or gene product is associated with cholesterol biosynthesis (e.g., SC5D).

In some embodiments, the gene or gene product increases the transduction efficiency of AAV2 and AAV9 particles. In some embodiments, the gene or gene product is necessary for the transduction of AAV2 and AAV9 particles. In some embodiments, the gene or gene product is AAV-R. In some embodiments, the gene or gene product is GRP108.

In some embodiments, the gene or gene product is chosen from KIAA0319L, BAMBI, TM9SF2, PHIP, DCLRE1C, VPS35, GPR108, CYREN, ACP2, F8A2, F8A1, F8A3, RBPJ, ATP2C1, ICMT, RABIF, IER3IP1, RBM10, SMG7, GTF2I, ELAVL1, MEPCE, RAB4A, IER3IP1, SAMD1, or a combination thereof.

In some embodiments, the gene or gene product is associated with influenza infection (e.g., ACP2). In some embodiments, the gene or gene product binds to unmethylated CGIs (e.g., SAMD1). In some embodiments, the gene or gene product is associated with intracellular vesicular transport (e.g., RABIF). In some embodiments, the gene or gene product is associated with posttranslational modification of cysteine residues (e.g., C-terminal cysteine residues), e.g., in a target protein (e.g., ICMT). In some embodiments, the gene or gene product binds to a capsid protein of AAV (e.g., KIAA0319L). In some embodiments, the gene or gene product is associated with inhibition of a TLR (e.g., TLR9), e.g., GPR108. In some embodiments, the gene or gene product is associated with endosome trafficking, e.g., of AAV-R (e.g., VPS35). In some embodiments, the gene or gene product is a calcium ATPase pump (e.g., ATP2C1). In some embodiments, the gene or gene product is associated with Notch signaling (e.g., RBPJ). In some embodiments, the gene or gene product is associated with HSPG metabolism (e.g., TM9SF2). In some embodiments, the gene or gene product is associated with inhibition of NHEJ (e.g., CYREN). In some embodiments, the gene or gene product is associated with viral hairpin resolution (e.g., DCLRE1C). In some embodiments, the gene or gene product is associated with Glc transporter translocation, optionally wherein the gene or gene product binds to insulin receptor substrate 1 protein (e.g., PHIP).

In some embodiments, the gene or gene product decreases the transduction efficiency of an AAV2 particle, optionally wherein the gene or gene product, when modulated, does not decrease, or does not substantially decrease, the transduction efficiency of an AAV9 particle. In some embodiments, the gene or gene product inhibits or prevents the transduction of an AAV2 particle, optionally wherein the gene or gene product does not inhibit or prevent the transduction of an AAV9 particle. In some embodiments, the gene or gene product is WDR11. In some embodiments, the gene or gene product is MRE11.

In some embodiments, the gene or gene product is chosen from GREB1L, BIRC2, TYMSOS, MSL3, SIK3, GTF2H5, PIGW, ATRAID, PPP4R1, ZFC3H1, SETDB1, SMCHD1, TMEM123, UHRF1, CLUL1, SMAD5, SETX, STEEP1, DENND6A, RTN4RL2, MTMR9, STRADA, PSIP1, BMPR1A, HIKESHI, AP3M1, MEN1, MYL12B, CREB1, RAB12, SMTNL1, MRGBP, WTAP, SUMO2, TCN1, ALDH3B1, ARL14EP, MYL12A, RHOD, PGAP2, EIF4A1, PIGN, DAXX, HDLBP, GOLGA1, SARNP, UBE2L6, TRIM49, SIK2, SOX4, OR6Q1, OR4D6, UNC93B1, CATSPERZ, PIGT, PYM1, MICOS13, RIN1, DOT1L, TRIM49C, MAPK20, COL8A1, FASN, KDM3B, PIGF, PIGA, PIGY, GPC3, TRAPPC2L, RAB31, ELP3, or a combination thereof.

In some embodiments, the gene or gene product is associated with a retinoic acid receptor activity (e.g., GREB1L). In some embodiments, the gene or gene product is a thymidylate synthetase (e.g., TYMSOS). In some embodiments, the gene or gene product is associated with chromatin remodeling (e.g., SMCHD1). In some embodiments, the gene or gene product is associated with histone H4 acetylation (e.g., MSL3). In some embodiments, the gene or gene product is associated with transcription and/or DNA repair (e.g., GTF2H5). In some embodiments, the gene or gene product is a histone methyltransferase (e.g., SETDB1). In some embodiments, the gene or gene product is associated with exosomal degradation of polyadenylated RNA (e.g., ZFC3H1). In some embodiments, the gene or gene product is associated with histone deacetylase recruitment to a DNA (e.g., UHRF1). In some embodiments, the gene or gene product is associated with oxidative stress-induced DNA double strand break response (e.g., SETX). In some embodiments, the gene or gene product is associated with histone modification (e.g., MEN1). In some embodiments, the gene or gene product is associated with nucleosome/DNA interaction (e.g., MRGBP). In some embodiments, the gene or gene product is associated with GPI biosynthesis (e.g., PIGW, PIGN, PIGT, PIGF, PIGA, or PIGY). In some embodiments, the gene or gene product is associated with maturation of GPI anchors (e.g., PGAP2). In some embodiments, the gene or gene product is associated with vesicle-mediated endocytosis (e.g., DENND6A, GOLGA1, MTMR9, GPC3, PPP4R1, RAB12, RAB31, RHOD, RIN1, TMEM123, or TRAPPC2L). In some embodiments, the gene or gene product is associated with clathrin-coated vesicle-mediated endocytosis (e.g., AP3M1). In some embodiments, the gene or gene product is associated with a BMP receptor kinase (e.g., SMAD5, BMPR1A, or CREB1). In some embodiments, the gene or gene product is associated with transcription (e.g., PSIP1). In some embodiments, the gene or gene product is associated with nuclear import of an HSP70 protein (e.g., HIKESHI). In some embodiments, the gene or gene product is associated with a Ser/Thr kinase (e.g., SIK2 or SIK3). In some embodiments, the gene or gene product is associated with SUMOylation of a protein (e.g., SUMO2, UBE2L6).

In some embodiments, the gene or gene product decreases the transduction efficiency of an AAV9 particle, optionally wherein the gene or gene product does not decrease, or does not substantially decrease, the transduction efficiency of an AAV2 particle. In some embodiments, the gene or gene product inhibits or prevents the transduction of an AAV9 particle, optionally wherein the gene or gene product does not inhibit or prevent the transduction of an AAV2 particle. In some embodiments, the gene or gene product is WDR11. In some embodiments, the gene or gene product is MRE11.

In some embodiments, gene or gene product is chosen from AP2A1, AP2B1, TM9SF4, ACTR5, PTMA, PAPOLA, PDHA1, PDHB, SLC25A19, GAK, AUNIP, FCHO2, ACTB, LIPT1, UCHL5, INO80E, ECHS1, NELFB, EDC4, PRMT1, PDS5B, PELI3, FBXL20, SLC35A1, or a combination thereof.

In some embodiments, the gene or gene product binds to ATP, a chaperone protein, and/or clathrin (e.g., GAK). In some embodiments, the gene or gene product is associated with clathrin-coated vesicle trafficking (e.g., AP2B1 or AP2A1). In some embodiments, the gene or gene product is associated with clathrin-mediated endocytosis (e.g., FCHO2). In some embodiments, the gene or gene product is associated with vesicular trafficking (e.g., ACTB). In some embodiments, the gene or gene product is associated with an innate immune response (e.g., PELI3 or FBXL20). In some embodiments, the gene or gene product is associated with glucose metabolism (e.g., PDHB or PDHA1). In some embodiments, the gene or gene product is associated with DNA resection and/or homologous recombination, e.g., after DNA damage (e.g., AUNIP). In some embodiments, the gene or gene product is associated with DNA repair, e.g., after DNA damage (e.g., UCHL5, INO80E, PDS5B, ACTR5, or PRMT1). In some embodiments, the gene or gene product is associated with transport of CMP-sialic acid from cytosol to a Golgi vesicle (e.g., SLC35A1). In some embodiments, the gene or gene product is associated with localization of polypeptides comprising glycine-rich transmembrane domains, e.g., to the cell surface (e.g., TM9SF4). In some embodiments, the gene or gene product is associated with poly(A) tail synthesis (e.g., PAPOLA). In some embodiments, the gene or gene product is PTMA. In some embodiments, the gene or gene product is associated with elongation of an mRNA by RNA polymerase II (e.g., NELFB). In some embodiments, the gene or gene product is associated with mRNA degradation (e.g., EDC4). In some embodiments, the gene or gene product encodes a mitochondrial lipoyltransferase (e.g., LIPT1). In some embodiments, the gene or gene product is associated with mitochondrial fatty acid beta-oxidation (e.g., ECHS1). In some embodiments, the gene or gene product encodes a mitochondrial thiamine pyrophosphate carrier (e.g., SLC25A19).

In some embodiments, the gene or gene product decreases the transduction efficiency of AAV2 and AAV9 particles. In some embodiments, the gene or gene product inhibits or prevents the transduction of AAV2 and AAV9 particles. In some embodiments, the gene or gene product is WDR11. In some embodiments, the gene or gene product is MRE11.

In some embodiments, the gene or gene product is selected from the group consisting of: PITPNB, PITP, FAM91A1, WDR11, AP1G1, APIM1, APIS1, APIS3, HEATR5B, STX16, APIB1, PIAS1, DENR, ARL1, ZFAT, TBC1D23, LIN37, RALGAPB, B3GNT2, ELOVL1, AP2B1, KIAA2013, PTEN, MCTS1, NBN, HELZ, SLC38A10, FBXL20, TGIF1, KDSR, CPD, CHD7, USP9X, SIMC1, TAF11, VAPA, MTMR6, RAB1B, SLF2, MRE11, VTI1A, MBOAT7, PPP6R3, or a combination thereof.

In some embodiments, the gene or gene product is associated with the long-chain FA elongation cycle (e.g., ELOVL1). In some embodiments, the gene or gene product is associated with lipoic acid biosynthesis (e.g., PIAS1). In some embodiments, the gene or gene product is associated with a protein phosphatase catalytic subunit (e.g., PPP6R3). In some embodiments, the gene or gene product is associated with the WDR11 pathway (e.g., WDR11, FAM91A1, AP1G1, AP1S1, AP1M1, AP1S3, or TBC1D23). In some embodiments, the gene or gene product is associated with retrograde transport from the Golgi apparatus to the endoplasmic reticulum, e.g., of PI and/or PC (e.g., PITP). In some embodiments, the gene or gene product is associated with vesicular transport from a late endosome to a trans-Golgi network (e.g., STX16). In some embodiments, the gene or gene product is associated with an activity or recruitment of a golgin, arfaptin, and/or Arf-GEF to a trans-Golgi network (e.g., ARL1). In some embodiments, the gene or gene product encodes a component of a clathrin-dependent vesicle and/or is associated with AP1G1/AP-1-mediated protein trafficking (e.g., HEATR5B).

In some embodiments, the gene or gene product is AAV-R, GPR108, WDR11 or MRE11. In some embodiments, the gene or gene product is AAV-R. In some embodiments, the gene or gene product is GPR108. In some embodiments, the gene or gene product is WDR11. In some embodiments, the gene or gene product is MRE11.

In some embodiments, the method results in a high gene modulating (e.g., gene editing) efficiency (e.g., at least 60%, 65%, 70%, 75%, 80%, 85%, 90%, 95%, or 98%) in a first tissue, with limited impact (e.g., a gene modulating (e.g., gene editing) efficiency of 20%, 15%, 10%, 5%, 2%, or less) in a second tissue. In some embodiments, the method results in a high modulating (e.g., gene editing) efficiency (e.g., at least 60%, 65%, 70%, 75%, 80%, 85%, 90%, 95%, or 98%) in liver, with limited impact (e.g., a gene modulating (e.g., gene editing) efficiency of 20%, 15%, 10%, 5%, 2%, or less) in skeletal muscle, bone marrow, or both.

In some embodiments, the modulator is administered intravenously.

In some embodiments, the modulator is: (a) a gene editing system targeted to one or more sites within the gene or a regulatory element thereof; (b) a nucleic acid encoding one or more components of the gene editing system; or (c) a combination thereof. In some embodiments, the gene editing system is a CRISPR/Cas system, a zinc finger nuclease system, a TALEN system, and a meganuclease system.

In some embodiments, the gene editing system binds to a target sequence in an early (e.g., the first, second, or third) exon or intron of the gene. In some embodiments, the gene editing system binds a target sequence of the gene, and the target sequence is upstream of exon 4, e.g., in exon 1, exon 2, or exon 3. In some embodiments, the gene editing system binds to a target sequence in a late exon or intron of the gene. In some embodiments, the gene editing system binds a target sequence of the gene, and the target sequence is downstream of a preantepenultimate exon, e.g., is in an antepenultimate exon, a penultimate exon, or a last exon. In some embodiments, the gene editing system is a CRISPR/Cas system comprising a guide RNA (gRNA) molecule comprising a targeting sequence which hybridizes to a target sequence of the gene. In some embodiments, the CRISPR/Cas system is a CRISPR/Cas9 system. In some embodiments, the CRISPR/Cas system is a CRISPR/Cas12a system.

In some embodiments, the modulator is a small interfering RNA (siRNA) or small hairpin (shRNA) specific for the gene, or a nucleic acid encoding the siRNA or shRNA. In some embodiments, the siRNA or shRNA comprises a nucleotide sequence complementary to a nucleotide sequence of an mRNA encoded by the gene. In some embodiments, the modulator is a guide RNA (gRNA) specific for the gene, or a nucleic acid encoding the gRNA. In some embodiments, the gRNA comprises a nucleotide sequence complementary to a nucleotide sequence of the gene.

In some embodiments, the modulator is an antisense oligonucleotide (ASO) specific for the gene, or a nucleic acid encoding the ASO. In some embodiments, the ASO comprises a nucleotide sequence complementary to a nucleotide sequence of an mRNA encoded by the gene.

In some embodiments, the modulator is a small molecule (e.g., a compound described herein, e.g., in FIG. 7). In some embodiments, the small molecule is a protein degrader.

In some embodiments, the modulator is a protein or a peptide, or a nucleic acid encoding the protein or peptide. In some embodiments, the modulator is an antibody molecule (e.g., an scFv or sdAb). In some embodiments, the modulator is a dominant negative binding partner of a protein encoded by the gene, or a nucleic acid encoding the dominant negative binding partner. In some embodiments, the modulator is a dominant negative variant (e.g., catalytically inactive) of a protein encoded by the gene, or a nucleic acid encoding said dominant negative variant.

In some embodiments, the AAV particle comprises an AAV genome. In some embodiments, the AAV particle comprises an AAV-like particle. In some embodiments, the AAV particle comprises a capsid. In some embodiments, the AAV particle has a serotype of AAV1, AAV2, AAV3, AAV4, AAV5, AAV6, AAV7, AAV8, AAV9, AAV10, AAV11, or a combination thereof. In some embodiments, the AAV particle is an AAV2 particle. In some embodiments, the AAV particle is an AAV9 particle.

In some embodiments, the AAV particle has a tropism towards the CNS (e.g., AAV1, AAV2, AAV4, AAV5, AAV8, or AAV9). In some embodiments, the AAV particle has a tropism towards heart (e.g., AAV1, AAV8, or AAV9). In some embodiments, the AAV particle has a tropism towards kidney (e.g., AAV2). In some embodiments, the AAV particle has a tropism towards liver (e.g., AAV7, AAV8, or AAV9). In some embodiments, the AAV particle has a tropism towards lung (e.g., AAV4, AAV5, AAV6, or AAV9). In some embodiments, the AAV particle has a tropism towards pancreas (e.g., AAV8). In some embodiments, the AAV particle has a tropism towards a photoreceptor cell (e.g., AAV2, AAV5, or AAV8). In some embodiments, the AAV particle has a tropism towards retinal pigment epithelium (RPE) (e.g., AAV1, AAV2, AAV4, AAV5, or AAV8). In some embodiments, the AAV particle has a tropism towards skeletal muscle (e.g., AAV1, AAV6, AAV7, AAV8, or AAV9).

In some embodiments, the AAV particle comprises a nucleotide sequence encoding a therapeutic protein, e.g., a protein associated with a disorder described herein. In some embodiments, the AAV particle comprises a nucleotide sequence encoding NGF, APOE2 (e.g., hAPOE2), TERT (e.g., hTERT), MAPT, GAD, AADC, NTN, GDNF, GCase, HTT, SMN, SMN2, SOD1, or C9orf72.

In some embodiments, the AAV particle comprises a nucleotide sequence encoding a therapeutic nucleic acid, e.g., a nucleic acid targeting a nucleotide sequence encoding a protein associated with a disorder described herein.

In some embodiments, the method comprises administering to the subject a plurality of AAV transduction modulators or an AAV modulator that modulates a plurality of genes or gene products associated with AAV transduction efficiency.

In another aspect, the disclosure provides a method of reducing the toxicity of a therapy comprising an AAV genome or an AAV particle, the method comprising administering to a subject in need thereof an effective amount of an AAV transduction modulator, thereby reducing the toxicity of the therapy.

In some embodiments, the subject has not received, or is not receiving, the therapy, when the AAV transduction modulator is administered. In some embodiments, the AAV transduction modulator was administered at least 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the subject receives the therapy. In some embodiments, the AAV transduction modulator was administered no more than 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the subject receives the therapy.

In some embodiments, the subject has received, or is receiving, the therapy, when the AAV transduction modulator is administered. In some embodiments, the subject received the therapy at least 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the AAV transduction modulator is administered. In some embodiments, the subject received the therapy no more than 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the AAV transduction modulator is administered.

In some embodiments, the toxicity is reduced by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of toxicity. In some embodiments, the reference level of toxicity is the level of toxicity in a subject that has not been administered to the AAV transduction modulator. In some embodiments, the reference level of toxicity is the level of toxicity before the subject is administered the AAV transduction modulator. In some embodiments, the method reduces the toxicity in dorsal root ganglion (DRG). In some embodiments, the method reduces the toxicity in liver. In some embodiments, the method reduces the toxicity in cardiomyocytes. In some embodiments, the method reduces the toxicity in retinal pigment epithelium (RPE).

In some embodiments, the subject has a disorder or a symptom thereof, or is at risk of having a disorder or a symptom thereof. In some embodiments, the disorder is a neurodegenerative disorder. In some embodiments, the neurodegenerative disorder is Alzheimer's disease, Parkinson's disease, Huntington's disease, multiple sclerosis (MS), amyotrophic lateral sclerosis (ALS), or Batten disease. In some embodiments, the disorder is mild Alzheimer's disease, mild-to-moderate Alzheimer's disease, mild-to-severe Alzheimer's disease, early dementia, Parkinson's disease (e.g., idiopathic Parkinson's disease, bilateral idiopathic Parkinson's disease, mid-to-late stage Parkinson's disease), Huntington's disease (e.g., manifest Huntington's disease), Type 1 SMA, presymptomatic SMA, or amyotrophic lateral sclerosis (ALS) (e.g., familial ALS, ALS with SOD1 mutation, or ALS with C9orf72 mutation). In some embodiments, the disorder is an eye disorder. In some embodiments, the eye disorder is blindness, e.g., inherited or non-inherited blindness. In some embodiments, the eye disorder is Leber's congenital amaurosis, age-related macular degeneration, choroideremia, or color blindness. In some embodiments, the method reduces the toxicity of the therapy, enhances the efficacy of the therapy, or both.

In some embodiments, the modulator modulates (e.g., increases or decreases) a gene or gene product associated with AAV transduction efficiency. In some embodiments, the gene or gene product is a mammalian (e.g., human) gene or gene product.

In some embodiments, the modulator inhibits the gene or gene product, e.g., a gene or gene product that is associated with a decreased transduction efficiency of an AAV particle. In some embodiments, the modulator activates the gene or gene product, e.g., a gene or gene product that is associated with an increased transduction efficiency of an AAV particle.

In some embodiments, the gene or gene product is associated with an increased transduction efficiency of an AAV particle. In some embodiments, the gene or gene product is associated with an increased transduction efficiency of an AAV particle of a first serotype (e.g., AAV2), an increased transduction efficiency of an AAV particle of a second serotype (e.g., AAV9), or both. In some embodiments, the gene or gene product is AAV-R. In some embodiments, the gene or gene product is GPR108.

In some embodiments, the gene or gene product is associated with a decreased transduction efficiency of an AAV particle. In some embodiments, the gene or gene product is associated with a decreased transduction efficiency of an AAV particle of a first serotype (e.g., AAV2), a decreased transduction efficiency of an AAV particle of a second serotype (e.g., AAV9), or both. In some embodiments, the gene or gene product is WDR11. In some embodiments, the gene or gene product is MRE11.

In some embodiments, the gene or gene product, when modulated, increases the transduction efficiency of an AAV particle. In some embodiments, the gene or gene product, when modulated, increases the transduction efficiency of an AAV particle of a first serotype (e.g., AAV2), the transduction efficiency of an AAV particle of a second serotype (e.g., AAV9), or both.

In some embodiments, the gene or gene product, when modulated, decreases the transduction efficiency of an AAV particle. In some embodiments, the gene or gene product, when modulated, decreases the transduction efficiency of an AAV particle of a first serotype (e.g., AAV2), the transduction efficiency of an AAV particle of a second serotype (e.g., AAV9), or both.

In some embodiments, the gene product is an RNA (e.g., an mRNA). In some embodiments, the gene product is a protein. In some embodiments, the gene or gene product is preferentially expressed in a target tissue (e.g., brain or liver). The following dependent claims describe the AAV transduction modulator based on its mechanism. In some embodiments, the modulator alters (e.g., increases or decreases) the expression of the gene. In some embodiments, the modulator alters the structure of the gene. In some embodiments, the modulator alters (e.g., increases or decreases) an activity (e.g., an enzymatic activity) of the gene product. In some embodiments, the modulator alters (e.g., increases or decreases) the level (e.g., abundance) of the gene product. In some embodiments, the modulator alters (e.g., increases or decreases) the stability of the gene product. In some embodiments, the modulator alters (e.g., increases or decreases) transduction efficiency of an AAV particle, e.g., as determined by an assay described herein (e.g., an FACS analysis, e.g., as described in Examples 1 or 2).

In some embodiments, the gene or gene product is AAV-R, GPR108, WDR11 or MRE11. In some embodiments, the gene or gene product is AAV-R. In some embodiments, the gene or gene product is GPR108. In some embodiments, the gene or gene product is WDR11. In some embodiments, the gene or gene product is MRE11.

In some embodiments, the method results in a high gene modulating (e.g., gene editing) efficiency (e.g., at least 60%, 65%, 70%, 75%, 80%, 85%, 90%, 95%, or 98%) in a first tissue, with limited impact (e.g., a gene modulating (e.g., gene editing) efficiency of 20%, 15%, 10%, 5%, 2%, or less) in a second tissue. In some embodiments, the method results in a high modulating (e.g., gene editing) efficiency (e.g., at least 60%, 65%, 70%, 75%, 80%, 85%, 90%, 95%, or 98%) in liver, with limited impact (e.g., a gene modulating (e.g., gene editing) efficiency of 20%, 15%, 10%, 5%, 2%, or less) in skeletal muscle, bone marrow, or both.

In some embodiments, the modulator is administered intravenously.

In some embodiments, the modulator increases transduction efficiency of an AAV particle.

In some embodiments, the transduction efficiency is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference level of transduction efficiency. In some embodiments, the modulator increases transduction to at least 10, 20, 30, 40, 50, or 60 viral copies per genome. In some embodiments, the modulator increases transduction to at least 10³, 10⁴, 10⁵, 10⁶, or 10⁷ viral copies per μg DNA.

In some embodiments, the modulator decreases transduction efficiency of an AAV particle. In some embodiments, the transduction efficiency is decreased by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of transduction efficiency. In some embodiments, the modulator decreases transduction to no more than 0, 1, 2, 3, 4, 5, 6, 7, 8, 9, or 10 viral copies per genome. In some embodiments, the modulator decreases transduction to no more than 10², 10³, or 10⁴ viral copies per μg DNA.

In some embodiments, the reference level of transduction efficiency is the level of transduction efficiency in a cell that has not been contacted with the AAV transduction modulator. In some embodiments, the reference level of transduction efficiency is the level of transduction efficiency before the cell is contacted with the AAV transduction modulator.

In some embodiments, the gene or gene product increases the transduction efficiency of an AAV2 particle, optionally wherein the gene or gene product or gene or gene product does not increase, or does not substantially increase, the transduction efficiency of an AAV9 particle. In some embodiments, the gene or gene product is necessary for the transduction of an AAV2 particle, optionally wherein the gene or gene product is not necessary for the transduction of an AAV9 particle.

In some embodiments, the gene or gene product is AAV-R. In some embodiments, the gene or gene product is GRP108.

In some embodiments, the gene or gene product is chosen from EXT1, EXT2, NDST1, BCL10, OAF, PDCD10, LMO4, MALT1, ZNF644, CHUK, GLCE, EP300, ARID4B, RAB21, MED13, DHX15, WASHC5, IKBKB, USP24, HSPA14, CXXC1, UBE2A, INTS8, CDC42, NFKB1, SIN3A, USP7, ARF5, CARD10, JAK1, SLC35C2, PAXK1, ZC3H11A, FAM20B, FAM72A, DHX36, INTS12, RPRD1B, or a combination thereof.

In some embodiments, the gene or gene product is associated with endosomal sorting (e.g., RAB21, WASHC5, or USP7). In some embodiments, the gene or gene product is associated with vesicle-mediated transport (e.g., CDC42 or ARF5). In some embodiments, the gene or gene product is associated with NFKB signaling (e.g., BCL10, MALT1, NFKB1, IKBKB, CHUK, or CARD10).

In some embodiments, the gene or gene product is associated with heparan sulfate biosynthesis (e.g., EXT1, EXT2, NDST1, GLCE, or FAM20B). In some embodiments, the gene or gene product is associated with H3K9 methylation (e.g., ZNF644). In some embodiments, the gene or gene product is associated with pre-mRNA processing (e.g., DHX15). In some embodiments, the gene or gene product is associated with transcription (e.g., LMO4, EP300, MED13, SIN3, ARID4B, ZC3H11A, CXXC1, RPRD1B, or UBE2A). In some embodiments, the gene or gene product is associated with integrator complex and/or RNA polymerase II function (e.g., INTS8 or INST12). In some embodiments, the gene or gene product is associated with DNA repair and/or AAV genome resolution (e.g., DHX36, ARID4B, UBE2A, or USP24). In some embodiments, the gene or gene product is associated with cell proliferation and/or apoptosis (e.g., PDCD10). In some embodiments, the gene or gene product is associated with spondylocarpotarsal synostosis syndrome (e.g., OAF). In some embodiments, the gene or gene product is associated with protein folding (e.g., HSPA14). In some embodiments, the gene or gene product is associated with helicase activity (e.g., DHX36). In some embodiments, the gene or gene product is associated with fucosylation of Notch and/or transport of GCP-fucose (e.g., SLC35C2).

In some embodiments, the gene or gene product increases the transduction efficiency of an AAV9 particle, optionally wherein the gene or gene product does not increase, or does not substantially increase, the transduction efficiency of an AAV2 particle. In some embodiments, the gene or gene product is necessary for the transduction of an AAV9 particle, optionally wherein the gene or gene product is not necessary for the transduction of an AAV2 particle. In some embodiments, the gene or gene product is AAV-R. In some embodiments, the gene or gene product is GRP108.

In some embodiments, the gene or gene product is chosen from TMPRSS11B, HTT, SNRNP70, BRD7, DMXL1, RAB10, CNGA1, KLHDC3, CHP1, CYP3A5, ELOVL4, PNISR, ZCCHC14, AZGP1, TPST1, SC5D, ELP2, ELP3, IPO9, RAB14, WDR7, XRCC4, GDI2, or a combination thereof.

In some embodiments, the gene or gene product is associated with microtubule-mediated transport or vesicle function (e.g., HT). In some embodiments, the gene or gene product is an ion channel (e.g., CNGA1). In some embodiments, the gene or gene product is associated with nuclear protein import (e.g., IP09). In some embodiments, the gene or gene product is associated with O-sulfation of a tyrosine residue within an acidic motif of a polypeptide (e.g., TPST1). In some embodiments, the gene or gene product is associated with protection of viral RNA (e.g., ZCCHC14). In some embodiments, the gene or gene product is associated with RNA binding by an AAV protein (e.g., PNISR). In some embodiments, the gene or gene product is associated with calcium ion-regulated exocytosis (e.g., CHP1). In some embodiments, the gene or gene product is associated with intracellular trafficking (e.g., RAB10, RAB14, GDI2, WDR7, or DMXL1). In some embodiments, the gene or gene product is a subunit (e.g., a catalytic tRNA acetyltransferase subunit) of an elongator complex, e.g., of an RNA polymerase (e.g., RNA polymerase II), e.g., ELP2 or ELP3. In some embodiments, the gene or gene product binds to chromatin (e.g., KLHDC3). In some embodiments, the gene or gene product is associated with cholesterol biosynthesis (e.g., SC5D).

In some embodiments, the gene or gene product increases the transduction efficiency of AAV2 and AAV9 particles. In some embodiments, the gene or gene product is necessary for the transduction of AAV2 and AAV9 particles. In some embodiments, the gene or gene product is AAV-R. In some embodiments, the gene or gene product is GRP108.

In some embodiments, the gene or gene product is chosen from KIAA0319L, BAMBI, TM9SF2, PHIP, DCLRE1C, VPS35, GPR108, CYREN, ACP2, F8A2, F8A1, F8A3, RBPJ, ATP2C1, ICMT, RABIF, IER3IP1, RBM10, SMG7, GTF2I, ELAVL1, MEPCE, RAB4A, IER3IP1, SAMD1, or a combination thereof.

In some embodiments, the gene or gene product is associated with influenza infection (e.g., ACP2). In some embodiments, the gene or gene product binds to unmethylated CGIs (e.g., SAMD1). In some embodiments, the gene or gene product is associated with intracellular vesicular transport (e.g., RABIF). In some embodiments, the gene or gene product is associated with posttranslational modification of cysteine residues (e.g., C-terminal cysteine residues), e.g., in a target protein (e.g., ICMT). In some embodiments, the gene or gene product binds to a capsid protein of AAV (e.g., KIAA0319L). In some embodiments, the gene or gene product is associated with inhibition of a TLR (e.g., TLR9), e.g., GPR108. In some embodiments, the gene or gene product is associated with endosome trafficking, e.g., of AAV-R (e.g., VPS35). In some embodiments, the gene or gene product is a calcium ATPase pump (e.g., ATP2C1). In some embodiments, the gene or gene product is associated with Notch signaling (e.g., RBPJ). In some embodiments, the gene or gene product is associated with HSPG metabolism (e.g., TM9SF2). In some embodiments, the gene or gene product is associated with inhibition of NHEJ (e.g., CYREN). In some embodiments, the gene or gene product is associated with viral hairpin resolution (e.g., DCLRE1C). In some embodiments, the gene or gene product is associated with Glc transporter translocation, optionally wherein the gene or gene product binds to insulin receptor substrate 1 protein (e.g., PHIP).

In some embodiments, the gene or gene product decreases the transduction efficiency of an AAV2 particle, optionally wherein the gene or gene product, when modulated, does not decrease, or does not substantially decrease, the transduction efficiency of an AAV9 particle. In some embodiments, the gene or gene product inhibits or prevents the transduction of an AAV2 particle, optionally wherein the gene or gene product does not inhibit or prevent the transduction of an AAV9 particle. In some embodiments, the gene or gene product is WDR11. In some embodiments, the gene or gene product is MRE11.

In some embodiments, the gene or gene product is chosen from GREB1L, BIRC2, TYMSOS, MSL3, SIK3, GTF2H5, PIGW, ATRAID, PPP4R1, ZFC3H1, SETDB1, SMCHD1, TMEM123, UHRF1, CLUL1, SMAD5, SETX, STEEP1, DENND6A, RTN4RL2, MTMR9, STRADA, PSIP1, BMPR1A, HIKESHI, AP3M1, MEN1, MYL12B, CREB1, RAB12, SMTNL1, MRGBP, WTAP, SUMO2, TCN1, ALDH3B1, ARL14EP, MYL12A, RHOD, PGAP2, EIF4A1, PIGN, DAXX, HDLBP, GOLGA1, SARNP, UBE2L6, TRIM49, SIK2, SOX4, OR6Q1, OR4D6, UNC93B1, CATSPERZ, PIGT, PYM1, MICOS13, RIN1, DOT1L, TRIM49C, MAPK20, COL8A1, FASN, KDM3B, PIGF, PIGA, PIGY, GPC3, TRAPPC2L, RAB31, ELP3, or a combination thereof.

In some embodiments, the gene or gene product is associated with a retinoic acid receptor activity (e.g., GREB1L). In some embodiments, the gene or gene product is a thymidylate synthetase (e.g., TYMSOS). In some embodiments, the gene or gene product is associated with chromatin remodeling (e.g., SMCHD1). In some embodiments, the gene or gene product is associated with histone H4 acetylation (e.g., MSL3). In some embodiments, the gene or gene product is associated with transcription and/or DNA repair (e.g., GTF2H5). In some embodiments, the gene or gene product is a histone methyltransferase (e.g., SETDB1). In some embodiments, the gene or gene product is associated with exosomal degradation of polyadenylated RNA (e.g., ZFC3H1). In some embodiments, the gene or gene product is associated with histone deacetylase recruitment to a DNA (e.g., UHRF1). In some embodiments, the gene or gene product is associated with oxidative stress-induced DNA double strand break response (e.g., SETX). In some embodiments, the gene or gene product is associated with histone modification (e.g., MEN1). In some embodiments, the gene or gene product is associated with nucleosome/DNA interaction (e.g., MRGBP). In some embodiments, the gene or gene product is associated with GPI biosynthesis (e.g., PIGW, PIGN, PIGT, PIGF, PIGA, or PIGY). In some embodiments, the gene or gene product is associated with maturation of GPI anchors (e.g., PGAP2). In some embodiments, the gene or gene product is associated with vesicle-mediated endocytosis (e.g., DENND6A, GOLGA1, MTMR9, GPC3, PPP4R1, RAB12, RAB31, RHOD, RIN1, TMEM123, or TRAPPC2L). In some embodiments, the gene or gene product is associated with clathrin-coated vesicle-mediated endocytosis (e.g., AP3M1). In some embodiments, the gene or gene product is associated with a BMP receptor kinase (e.g., SMAD5, BMPR1A, or CREB1). In some embodiments, the gene or gene product is associated with transcription (e.g., PSIP1). In some embodiments, the gene or gene product is associated with nuclear import of an HSP70 protein (e.g., HIKESHI). In some embodiments, the gene or gene product is associated with a Ser/Thr kinase (e.g., SIK2 or SIK3). In some embodiments, the gene or gene product is associated with SUMOylation of a protein (e.g., SUMO2, UBE2L6).

In some embodiments, the gene or gene product decreases the transduction efficiency of an AAV9 particle, optionally wherein the gene or gene product does not decrease, or does not substantially decrease, the transduction efficiency of an AAV2 particle. In some embodiments, the gene or gene product inhibits or prevents the transduction of an AAV9 particle, optionally wherein the gene or gene product does not inhibit or prevent the transduction of an AAV2 particle. In some embodiments, the gene or gene product is WDR11. In some embodiments, the gene or gene product is MRE11.

In some embodiments, gene or gene product is chosen from AP2A1, AP2B1, TM9SF4, ACTR5, PTMA, PAPOLA, PDHA1, PDHB, SLC25A19, GAK, AUNIP, FCHO2, ACTB, LIPT1, UCHL5, INO80E, ECHS1, NELFB, EDC4, PRMT1, PDS5B, PELI3, FBXL20, SLC35A1, or a combination thereof.

In some embodiments, the gene or gene product binds to ATP, a chaperone protein, and/or clathrin (e.g., GAK). In some embodiments, the gene or gene product is associated with clathrin-coated vesicle trafficking (e.g., AP2B1 or AP2A1). In some embodiments, the gene or gene product is associated with clathrin-mediated endocytosis (e.g., FCHO2). In some embodiments, the gene or gene product is associated with vesicular trafficking (e.g., ACTB). In some embodiments, the gene or gene product is associated with an innate immune response (e.g., PELI3 or FBXL20). In some embodiments, the gene or gene product is associated with glucose metabolism (e.g., PDHB or PDHA1). In some embodiments, the gene or gene product is associated with DNA resection and/or homologous recombination, e.g., after DNA damage (e.g., AUNIP). In some embodiments, the gene or gene product is associated with DNA repair, e.g., after DNA damage (e.g., UCHL5, INO80E, PDS5B, ACTR5, or PRMT1). In some embodiments, the gene or gene product is associated with transport of CMP-sialic acid from cytosol to a Golgi vesicle (e.g., SLC35A1). In some embodiments, the gene or gene product is associated with localization of polypeptides comprising glycine-rich transmembrane domains, e.g., to the cell surface (e.g., TM9SF4). In some embodiments, the gene or gene product is associated with poly(A) tail synthesis (e.g., PAPOLA). In some embodiments, the gene or gene product is PTMA. In some embodiments, the gene or gene product is associated with elongation of an mRNA by RNA polymerase II (e.g., NELFB). In some embodiments, the gene or gene product is associated with mRNA degradation (e.g., EDC4). In some embodiments, the gene or gene product encodes a mitochondrial lipoyltransferase (e.g., LIPT1). In some embodiments, the gene or gene product is associated with mitochondrial fatty acid beta-oxidation (e.g., ECHS1). In some embodiments, the gene or gene product encodes a mitochondrial thiamine pyrophosphate carrier (e.g., SLC25A19).

In some embodiments, the gene or gene product decreases the transduction efficiency of AAV2 and AAV9 particles. In some embodiments, the gene or gene product inhibits or prevents the transduction of AAV2 and AAV9 particles. In some embodiments, the gene or gene product is WDR11. In some embodiments, the gene or gene product is MRE11.

In some embodiments, the gene or gene product is selected from the group consisting of: PITPNB, PITP, FAM91A1, WDR11, AP1G1, APIM1, APIS1, APIS3, HEATR5B, STX16, APIB1, PIAS1, DENR, ARL1, ZFAT, TBC1D23, LIN37, RALGAPB, B3GNT2, ELOVL1, AP2B1, KIAA2013, PTEN, MCTS1, NBN, HELZ, SLC38A10, FBXL20, TGIF1, KDSR, CPD, CHD7, USP9X, SIMC1, TAF11, VAPA, MTMR6, RAB1B, SLF2, MRE11, VTI1A, MBOAT7, PPP6R3, or a combination thereof.

In some embodiments, the gene or gene product is associated with the long-chain FA elongation cycle (e.g., ELOVL1). In some embodiments, the gene or gene product is associated with lipoic acid biosynthesis (e.g., PIAS1). In some embodiments, the gene or gene product is associated with a protein phosphatase catalytic subunit (e.g., PPP6R3). In some embodiments, the gene or gene product is associated with the WDR11 pathway (e.g., WDR11, FAM91A1, AP1G1, AP1S1, AP1M1, AP1S3, or TBC1D23). In some embodiments, the gene or gene product is associated with retrograde transport from the Golgi apparatus to the endoplasmic reticulum, e.g., of PI and/or PC (e.g., PITP). In some embodiments, the gene or gene product is associated with vesicular transport from a late endosome to a trans-Golgi network (e.g., STX16). In some embodiments, the gene or gene product is associated with an activity or recruitment of a golgin, arfaptin, and/or Arf-GEF to a trans-Golgi network (e.g., ARL1). In some embodiments, the gene or gene product encodes a component of a clathrin-dependent vesicle and/or is associated with AP1G1/AP-1-mediated protein trafficking (e.g., HEATR5B).

In some embodiments, the modulator is: (a) a gene editing system targeted to one or more sites within the gene or a regulatory element thereof; (b) a nucleic acid encoding one or more components of the gene editing system; or (c) a combination thereof. In some embodiments, the gene editing system is a CRISPR/Cas system, a zinc finger nuclease system, a TALEN system, and a meganuclease system.

In some embodiments, the gene editing system binds to a target sequence in an early (e.g., the first, second, or third) exon or intron of the gene. In some embodiments, the gene editing system binds a target sequence of the gene, and the target sequence is upstream of exon 4, e.g., in exon 1, exon 2, or exon 3. In some embodiments, the gene editing system binds to a target sequence in a late exon or intron of the gene. In some embodiments, the gene editing system binds a target sequence of the gene, and the target sequence is downstream of a preantepenultimate exon, e.g., is in an antepenultimate exon, a penultimate exon, or a last exon. In some embodiments, the gene editing system is a CRISPR/Cas system comprising a guide RNA (gRNA) molecule comprising a targeting sequence which hybridizes to a target sequence of the gene. In some embodiments, the CRISPR/Cas system is a CRISPR/Cas9 system. In some embodiments, the CRISPR/Cas system is a CRISPR/Cas12a system.

In some embodiments, the modulator is a small interfering RNA (siRNA) or small hairpin (shRNA) specific for the gene, or a nucleic acid encoding the siRNA or shRNA. In some embodiments, the siRNA or shRNA comprises a nucleotide sequence complementary to a nucleotide sequence of an mRNA encoded by the gene. In some embodiments, the modulator is a guide RNA (gRNA) specific for the gene, or a nucleic acid encoding the gRNA. In some embodiments, the gRNA comprises a nucleotide sequence complementary to a nucleotide sequence of the gene.

In some embodiments, the modulator is an antisense oligonucleotide (ASO) specific for the gene, or a nucleic acid encoding the ASO. In some embodiments, the ASO comprises a nucleotide sequence complementary to a nucleotide sequence of an mRNA encoded by the gene.

In some embodiments, the modulator is a small molecule (e.g., a compound described herein, e.g., in FIG. 7). In some embodiments, the small molecule is a protein degrader.

In some embodiments, the modulator is a protein or a peptide, or a nucleic acid encoding the protein or peptide. In some embodiments, the modulator is an antibody molecule (e.g., an scFv or sdAb). In some embodiments, the modulator is a dominant negative binding partner of a protein encoded by the gene, or a nucleic acid encoding the dominant negative binding partner. In some embodiments, the modulator is a dominant negative variant (e.g., catalytically inactive) of a protein encoded by the gene, or a nucleic acid encoding said dominant negative variant.

In some embodiments, the AAV particle comprises an AAV genome. In some embodiments, the AAV particle comprises an AAV-like particle. In some embodiments, the AAV particle comprises a capsid. In some embodiments, the AAV particle has a serotype of AAV1, AAV2, AAV3, AAV4, AAV5, AAV6, AAV7, AAV8, AAV9, AAV10, AAV11, or a combination thereof. In some embodiments, the AAV particle is an AAV2 particle. In some embodiments, the AAV particle is an AAV9 particle.

In some embodiments, the AAV particle has a tropism towards the CNS (e.g., AAV1, AAV2, AAV4, AAV5, AAV8, or AAV9). In some embodiments, the AAV particle has a tropism towards heart (e.g., AAV1, AAV8, or AAV9). In some embodiments, the AAV particle has a tropism towards kidney (e.g., AAV2). In some embodiments, the AAV particle has a tropism towards liver (e.g., AAV7, AAV8, or AAV9). In some embodiments, the AAV particle has a tropism towards lung (e.g., AAV4, AAV5, AAV6, or AAV9). In some embodiments, the AAV particle has a tropism towards pancreas (e.g., AAV8). In some embodiments, the AAV particle has a tropism towards a photoreceptor cell (e.g., AAV2, AAV5, or AAV8). In some embodiments, the AAV particle has a tropism towards retinal pigment epithelium (RPE) (e.g., AAV1, AAV2, AAV4, AAV5, or AAV8). In some embodiments, the AAV particle has a tropism towards skeletal muscle (e.g., AAV1, AAV6, AAV7, AAV8, or AAV9).

In some embodiments, the AAV particle comprises a nucleotide sequence encoding a therapeutic protein, e.g., a protein associated with a disorder described herein. In some embodiments, the AAV particle comprises a nucleotide sequence encoding NGF, APOE2 (e.g., hAPOE2), TERT (e.g., hTERT), MAPT, GAD, AADC, NTN, GDNF, GCase, HTT, SMN, SMN2, SOD1, or C9orf72. In some embodiments, the AAV particle comprises a nucleotide sequence encoding a therapeutic nucleic acid, e.g., a nucleic acid targeting a nucleotide sequence encoding a protein associated with a disorder described herein.

In some embodiments, the method comprises administering to the subject a plurality of AAV transduction modulators or an AAV modulator that modulates a plurality of genes or gene products associated with AAV transduction efficiency.

In another aspect, the disclosure provides a method of enhancing the efficacy of a therapy comprising an AAV genome or an AAV particle, the method comprising administering to a subject in need thereof an effective amount of an AAV transduction modulator, thereby enhancing the efficacy of the therapy.

In some embodiments, the subject has not received, or is not receiving, the therapy, when the AAV transduction modulator is administered. In some embodiments, the subject has not received, or is not receiving, the therapy, when the AAV transduction modulator is administered. In some embodiments, the AAV transduction modulator was administered at least 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the subject receives the therapy. In some embodiments, the AAV transduction modulator was administered no more than 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the subject receives the therapy.

In some embodiments, the subject has received, or is receiving, the therapy, when the AAV transduction modulator is administered. In some embodiments, the subject received the therapy at least 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the AAV transduction modulator is administered. In some embodiments, the subject received the therapy no more than 1, 2, 3, 4, 5, 6, 7, 8, 9, 10, 11, 12, 13, 14, 15, 16, 17, 18, 19, 20, 21, 22, or 23 hours, 1, 2, 3, 4, 5, or 6 days, 1, 2, 3, or 4 weeks, 1, 2, 3, 4, 5, or 6 months, before the AAV transduction modulator is administered.

In some embodiments, the efficacy is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference. In some embodiments, the reference level of efficacy is the level of efficacy in a subject that has not been administered to the AAV transduction modulator. In some embodiments, the reference level of efficacy is the level of efficacy before the subject receives the AAV transduction modulator.

In some embodiments, the subject has a disorder or a symptom thereof, or is at risk of having a disorder or a symptom thereof. In some embodiments, the disorder is a neurodegenerative disorder.

In some embodiments, the neurodegenerative disorder is Alzheimer's disease, Parkinson's disease, Huntington's disease, multiple sclerosis (MS), amyotrophic lateral sclerosis (ALS), or Batten disease.

In some embodiments, the disorder is mild Alzheimer's disease, mild-to-moderate Alzheimer's disease, mild-to-severe Alzheimer's disease, early dementia, Parkinson's disease (e.g., idiopathic Parkinson's disease, bilateral idiopathic Parkinson's disease, mid-to-late stage Parkinson's disease), Huntington's disease (e.g., manifest Huntington's disease), Type 1 SMA, presymptomatic SMA, or amyotrophic lateral sclerosis (ALS) (e.g., familial ALS, ALS with SOD1 mutation, or ALS with C9orf72 mutation). In some embodiments, the disorder is an eye disorder. In some embodiments, the eye disorder is blindness, e.g., inherited or non-inherited blindness. In some embodiments, the eye disorder is Leber's congenital amaurosis, age-related macular degeneration, choroideremia, or color blindness. In some embodiments, the method reduces the toxicity of the therapy, enhances the efficacy of the therapy, or both.

In some embodiments, the modulator modulates (e.g., increases or decreases) a gene or gene product associated with AAV transduction efficiency. In some embodiments, the gene or gene product is a mammalian (e.g., human) gene or gene product.

In some embodiments, the modulator inhibits the gene or gene product, e.g., a gene or gene product that is associated with a decreased transduction efficiency of an AAV particle. In some embodiments, the modulator activates the gene or gene product, e.g., a gene or gene product that is associated with an increased transduction efficiency of an AAV particle.

In some embodiments, the gene or gene product is associated with an increased transduction efficiency of an AAV particle. In some embodiments, the gene or gene product is associated with an increased transduction efficiency of an AAV particle of a first serotype (e.g., AAV2), an increased transduction efficiency of an AAV particle of a second serotype (e.g., AAV9), or both. In some embodiments, the gene or gene product is AAV-R. In some embodiments, the gene or gene product is GPR108.

In some embodiments, the gene or gene product is associated with a decreased transduction efficiency of an AAV particle. In some embodiments, the gene or gene product is associated with a decreased transduction efficiency of an AAV particle of a first serotype (e.g., AAV2), a decreased transduction efficiency of an AAV particle of a second serotype (e.g., AAV9), or both. In some embodiments, the gene or gene product is WDR11. In some embodiments, the gene or gene product is MRE11.

In some embodiments, the gene or gene product, when modulated, increases the transduction efficiency of an AAV particle. In some embodiments, the gene or gene product, when modulated, increases the transduction efficiency of an AAV particle of a first serotype (e.g., AAV2), the transduction efficiency of an AAV particle of a second serotype (e.g., AAV9), or both.

In some embodiments, the gene or gene product, when modulated, decreases the transduction efficiency of an AAV particle. In some embodiments, the gene or gene product, when modulated, decreases the transduction efficiency of an AAV particle of a first serotype (e.g., AAV2), the transduction efficiency of an AAV particle of a second serotype (e.g., AAV9), or both.

In some embodiments, the gene product is an RNA (e.g., an mRNA). In some embodiments, the gene product is a protein. In some embodiments, the gene or gene product is preferentially expressed in a target tissue (e.g., brain or liver). The following dependent claims describe the AAV transduction modulator based on its mechanism. In some embodiments, the modulator alters (e.g., increases or decreases) the expression of the gene. In some embodiments, the modulator alters the structure of the gene. In some embodiments, the modulator alters (e.g., increases or decreases) an activity (e.g., an enzymatic activity) of the gene product. In some embodiments, the modulator alters (e.g., increases or decreases) the level (e.g., abundance) of the gene product. In some embodiments, the modulator alters (e.g., increases or decreases) the stability of the gene product. In some embodiments, the modulator alters (e.g., increases or decreases) transduction efficiency of an AAV particle, e.g., as determined by an assay described herein (e.g., an FACS analysis, e.g., as described in Examples 1 or 2).

In some embodiments, the gene or gene product is AAV-R, GPR108, WDR11 or MRE11. In some embodiments, the gene or gene product is AAV-R. In some embodiments, the gene or gene product is GPR108. In some embodiments, the gene or gene product is WDR11. In some embodiments, the gene or gene product is MRE11.

In some embodiments, the method results in a high gene modulating (e.g., gene editing) efficiency (e.g., at least 60%, 65%, 70%, 75%, 80%, 85%, 90%, 95%, or 98%) in a first tissue, with limited impact (e.g., a gene modulating (e.g., gene editing) efficiency of 20%, 15%, 10%, 5%, 2%, or less) in a second tissue. In some embodiments, the method results in a high modulating (e.g., gene editing) efficiency (e.g., at least 60%, 65%, 70%, 75%, 80%, 85%, 90%, 95%, or 98%) in liver, with limited impact (e.g., a gene modulating (e.g., gene editing) efficiency of 20%, 15%, 10%, 5%, 2%, or less) in skeletal muscle, bone marrow, or both.

In some embodiments, the modulator is administered intravenously.

In some embodiments, the modulator increases transduction efficiency of an AAV particle. In some embodiments, the transduction efficiency is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference level of transduction efficiency. In some embodiments, the modulator increases transduction to at least 10, 20, 30, 40, 50, or 60 viral copies per genome. In some embodiments, the modulator increases transduction to at least 10³, 10⁴, 10⁵, 10⁶, or 10⁷ viral copies per μg DNA.

In some embodiments, the modulator decreases transduction efficiency of an AAV particle. In some embodiments, the transduction efficiency is decreased by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of transduction efficiency. In some embodiments, the modulator decreases transduction to no more than 0, 1, 2, 3, 4, 5, 6, 7, 8, 9, or 10 viral copies per genome. In some embodiments, the modulator decreases transduction to no more than 10², 10³, or 10⁴ viral copies per μg DNA.

In some embodiments, the reference level of transduction efficiency is the level of transduction efficiency in a cell that has not been contacted with the AAV transduction modulator. In some embodiments, the reference level of transduction efficiency is the level of transduction efficiency before the cell is contacted with the AAV transduction modulator.

In some embodiments, the gene or gene product increases the transduction efficiency of an AAV2 particle, optionally wherein the gene or gene product or gene or gene product does not increase, or does not substantially increase, the transduction efficiency of an AAV9 particle. In some embodiments, the gene or gene product is necessary for the transduction of an AAV2 particle, optionally wherein the gene or gene product is not necessary for the transduction of an AAV9 particle.

In some embodiments, the gene or gene product is AAV-R. In some embodiments, the gene or gene product is GRP108.

In some embodiments, the gene or gene product is chosen from EXT1, EXT2, NDST1, BCL10, OAF, PDCD10, LMO4, MALT1, ZNF644, CHUK, GLCE, EP300, ARID4B, RAB21, MED13, DHX15, WASHC5, IKBKB, USP24, HSPA14, CXXC1, UBE2A, INTS8, CDC42, NFKB1, SIN3A, USP7, ARF5, CARD10, JAK1, SLC35C2, PAXK1, ZC3H11A, FAM20B, FAM72A, DHX36, INTS12, RPRD1B, or a combination thereof.

In some embodiments, the gene or gene product is associated with endosomal sorting (e.g., RAB21, WASHC5, or USP7). In some embodiments, the gene or gene product is associated with vesicle-mediated transport (e.g., CDC42 or ARF5). In some embodiments, the gene or gene product is associated with NFKB signaling (e.g., BCL10, MALT1, NFKB1, IKBKB, CHUK, or CARD10). In some embodiments, the gene or gene product is associated with heparan sulfate biosynthesis (e.g., EXT1, EXT2, NDST1, GLCE, or FAM20B). In some embodiments, the gene or gene product is associated with H3K9 methylation (e.g., ZNF644). In some embodiments, the gene or gene product is associated with pre-mRNA processing (e.g., DHX15). In some embodiments, the gene or gene product is associated with transcription (e.g., LMO4, EP300, MED13, SIN3, ARID4B, ZC3H11A, CXXC1, RPRD1B, or UBE2A). In some embodiments, the gene or gene product is associated with integrator complex and/or RNA polymerase II function (e.g., INTS8 or INST12). In some embodiments, the gene or gene product is associated with DNA repair and/or AAV genome resolution (e.g., DHX36, ARID4B, UBE2A, or USP24). In some embodiments, the gene or gene product is associated with cell proliferation and/or apoptosis (e.g., PDCD10). In some embodiments, the gene or gene product is associated with spondylocarpotarsal synostosis syndrome (e.g., OAF). In some embodiments, the gene or gene product is associated with protein folding (e.g., HSPA14). In some embodiments, the gene or gene product is associated with helicase activity (e.g., DHX36). In some embodiments, the gene or gene product is associated with fucosylation of Notch and/or transport of GCP-fucose (e.g., SLC35C2).

In some embodiments, the gene or gene product increases the transduction efficiency of an AAV9 particle, optionally wherein the gene or gene product does not increase, or does not substantially increase, the transduction efficiency of an AAV2 particle. In some embodiments, the gene or gene product is necessary for the transduction of an AAV9 particle, optionally wherein the gene or gene product is not necessary for the transduction of an AAV2 particle. In some embodiments, the gene or gene product is AAV-R. In some embodiments, the gene or gene product is GRP108.

In some embodiments, the gene or gene product is chosen from TMPRSS11B, HTT, SNRNP70, BRD7, DMXL1, RAB10, CNGA1, KLHDC3, CHP1, CYP3A5, ELOVL4, PNISR, ZCCHC14, AZGP1, TPST1, SC5D, ELP2, ELP3, IP09, RAB14, WDR7, XRCC4, GDI2, or a combination thereof.

In some embodiments, the gene or gene product is associated with microtubule-mediated transport or vesicle function (e.g., HT). In some embodiments, the gene or gene product is an ion channel (e.g., CNGA1). In some embodiments, the gene or gene product is associated with nuclear protein import (e.g., IP09). In some embodiments, the gene or gene product is associated with O-sulfation of a tyrosine residue within an acidic motif of a polypeptide (e.g., TPST1). In some embodiments, the gene or gene product is associated with protection of viral RNA (e.g., ZCCHC14).

In some embodiments, the gene or gene product is associated with RNA binding by an AAV protein (e.g., PNISR). In some embodiments, the gene or gene product is associated with calcium ion-regulated exocytosis (e.g., CHP1). In some embodiments, the gene or gene product is associated with intracellular trafficking (e.g., RAB10, RAB14, GDI2, WDR7, or DMXL1). In some embodiments, the gene or gene product is a subunit (e.g., a catalytic tRNA acetyltransferase subunit) of an elongator complex, e.g., of an RNA polymerase (e.g., RNA polymerase II), e.g., ELP2 or ELP3. In some embodiments, the gene or gene product binds to chromatin (e.g., KLHDC3). In some embodiments, the gene or gene product is associated with cholesterol biosynthesis (e.g., SC5D).

In some embodiments, the gene or gene product increases the transduction efficiency of AAV2 and AAV9 particles. In some embodiments, the gene or gene product is necessary for the transduction of AAV2 and AAV9 particles. In some embodiments, the gene or gene product is AAV-R. In some embodiments, the gene or gene product is GRP108.

In some embodiments, the gene or gene product is chosen from KIAA0319L, BAMBI, TM9SF2, PHIP, DCLRE1C, VPS35, GPR108, CYREN, ACP2, F8A2, F8A1, F8A3, RBPJ, ATP2C1, ICMT, RABIF, IER3IP1, RBM10, SMG7, GTF2I, ELAVL1, MEPCE, RAB4A, IER3IP1, SAMD1, or a combination thereof.

In some embodiments, the gene or gene product is associated with influenza infection (e.g., ACP2). In some embodiments, the gene or gene product binds to unmethylated CGIs (e.g., SAMD1). In some embodiments, the gene or gene product is associated with intracellular vesicular transport (e.g., RABIF). In some embodiments, the gene or gene product is associated with posttranslational modification of cysteine residues (e.g., C-terminal cysteine residues), e.g., in a target protein (e.g., ICMT). In some embodiments, the gene or gene product binds to a capsid protein of AAV (e.g., KIAA0319L). In some embodiments, the gene or gene product is associated with inhibition of a TLR (e.g., TLR9), e.g., GPR108. In some embodiments, the gene or gene product is associated with endosome trafficking, e.g., of AAV-R (e.g., VPS35). In some embodiments, the gene or gene product is a calcium ATPase pump (e.g., ATP2C1). In some embodiments, the gene or gene product is associated with Notch signaling (e.g., RBPJ). In some embodiments, the gene or gene product is associated with HSPG metabolism (e.g., TM9SF2). In some embodiments, the gene or gene product is associated with inhibition of NHEJ (e.g., CYREN). In some embodiments, the gene or gene product is associated with viral hairpin resolution (e.g., DCLRE1C). In some embodiments, the gene or gene product is associated with Glc transporter translocation, optionally wherein the gene or gene product binds to insulin receptor substrate 1 protein (e.g., PHIP).

In some embodiments, the gene or gene product decreases the transduction efficiency of an AAV2 particle, optionally wherein the gene or gene product, when modulated, does not decrease, or does not substantially decrease, the transduction efficiency of an AAV9 particle. In some embodiments, the gene or gene product inhibits or prevents the transduction of an AAV2 particle, optionally wherein the gene or gene product does not inhibit or prevent the transduction of an AAV9 particle. In some embodiments, the gene or gene product is WDR11. In some embodiments, the gene or gene product is MRE11.

In some embodiments, the gene or gene product is chosen from GREB1L, BIRC2, TYMSOS, MSL3, SIK3, GTF2H5, PIGW, ATRAID, PPP4R1, ZFC3H1, SETDB1, SMCHD1, TMEM123, UHRF1, CLUL1, SMAD5, SETX, STEEP1, DENND6A, RTN4RL2, MTMR9, STRADA, PSIP1, BMPR1A, HIKESHI, AP3M1, MEN1, MYL12B, CREB1, RAB12, SMTNL1, MRGBP, WTAP, SUMO2, TCN1, ALDH3B1, ARL14EP, MYL12A, RHOD, PGAP2, EIF4A1, PIGN, DAXX, HDLBP, GOLGA1, SARNP, UBE2L6, TRIM49, SIK2, SOX4, OR6Q1, OR4D6, UNC93B1, CATSPERZ, PIGT, PYM1, MICOS13, RIN1, DOT1L, TRIM49C, MAPK20, COL8A1, FASN, KDM3B, PIGF, PIGA, PIGY, GPC3, TRAPPC2L, RAB31, ELP3, or a combination thereof.

In some embodiments, the gene or gene product is associated with a retinoic acid receptor activity (e.g., GREB1L). In some embodiments, the gene or gene product is a thymidylate synthetase (e.g., TYMSOS). In some embodiments, the gene or gene product is associated with chromatin remodeling (e.g., SMCHD1). In some embodiments, the gene or gene product is associated with histone H4 acetylation (e.g., MSL3). In some embodiments, the gene or gene product is associated with transcription and/or DNA repair (e.g., GTF2H5). In some embodiments, the gene or gene product is a histone methyltransferase (e.g., SETDB1). In some embodiments, the gene or gene product is associated with exosomal degradation of polyadenylated RNA (e.g., ZFC3H1). In some embodiments, the gene or gene product is associated with histone deacetylase recruitment to a DNA (e.g., UHRF1). In some embodiments, the gene or gene product is associated with oxidative stress-induced DNA double strand break response (e.g., SETX). In some embodiments, the gene or gene product is associated with histone modification (e.g., MEN1). In some embodiments, the gene or gene product is associated with nucleosome/DNA interaction (e.g., MRGBP). In some embodiments, the gene or gene product is associated with GPI biosynthesis (e.g., PIGW, PIGN, PIGT, PIGF, PIGA, or PIGY). In some embodiments, the gene or gene product is associated with maturation of GPI anchors (e.g., PGAP2). In some embodiments, the gene or gene product is associated with vesicle-mediated endocytosis (e.g., DENND6A, GOLGA1, MTMR9, GPC3, PPP4R1, RAB12, RAB31, RHOD, RIN1, TMEM123, or TRAPPC2L). In some embodiments, the gene or gene product is associated with clathrin-coated vesicle-mediated endocytosis (e.g., AP3M1). In some embodiments, the gene or gene product is associated with a BMP receptor kinase (e.g., SMAD5, BMPR1A, or CREB1). In some embodiments, the gene or gene product is associated with transcription (e.g., PSIP1). In some embodiments, the gene or gene product is associated with nuclear import of an HSP70 protein (e.g., HIKESHI). In some embodiments, the gene or gene product is associated with a Ser/Thr kinase (e.g., SIK2 or SIK3). In some embodiments, the gene or gene product is associated with SUMOylation of a protein (e.g., SUMO2, UBE2L6).

In some embodiments, the gene or gene product decreases the transduction efficiency of an AAV9 particle, optionally wherein the gene or gene product does not decrease, or does not substantially decrease, the transduction efficiency of an AAV2 particle. In some embodiments, the gene or gene product inhibits or prevents the transduction of an AAV9 particle, optionally wherein the gene or gene product does not inhibit or prevent the transduction of an AAV2 particle. In some embodiments, the gene or gene product is WDR11. In some embodiments, the gene or gene product is MRE11.

In some embodiments, gene or gene product is chosen from AP2A1, AP2B1, TM9SF4, ACTR5, PTMA, PAPOLA, PDHA1, PDHB, SLC25A19, GAK, AUNIP, FCHO2, ACTB, LIPT1, UCHL5, INO80E, ECHS1, NELFB, EDC4, PRMT1, PDS5B, PELI3, FBXL20, SLC35A1, or a combination thereof.

In some embodiments, the gene or gene product binds to ATP, a chaperone protein, and/or clathrin (e.g., GAK). In some embodiments, the gene or gene product is associated with clathrin-coated vesicle trafficking (e.g., AP2B1 or AP2A1). In some embodiments, the gene or gene product is associated with clathrin-mediated endocytosis (e.g., FCHO2). In some embodiments, the gene or gene product is associated with vesicular trafficking (e.g., ACTB). In some embodiments, the gene or gene product is associated with an innate immune response (e.g., PELI3 or FBXL20). In some embodiments, the gene or gene product is associated with glucose metabolism (e.g., PDHB or PDHA1). In some embodiments, the gene or gene product is associated with DNA resection and/or homologous recombination, e.g., after DNA damage (e.g., AUNIP). In some embodiments, the gene or gene product is associated with DNA repair, e.g., after DNA damage (e.g., UCHL5, INO80E, PDS5B, ACTR5, or PRMT1). In some embodiments, the gene or gene product is associated with transport of CMP-sialic acid from cytosol to a Golgi vesicle (e.g., SLC35A1). In some embodiments, the gene or gene product is associated with localization of polypeptides comprising glycine-rich transmembrane domains, e.g., to the cell surface (e.g., TM9SF4). In some embodiments, the gene or gene product is associated with poly(A) tail synthesis (e.g., PAPOLA). In some embodiments, the gene or gene product is PTMA. In some embodiments, the gene or gene product is associated with elongation of an mRNA by RNA polymerase II (e.g., NELFB). In some embodiments, the gene or gene product is associated with mRNA degradation (e.g., EDC4). In some embodiments, the gene or gene product encodes a mitochondrial lipoyltransferase (e.g., LIPT1). In some embodiments, the gene or gene product is associated with mitochondrial fatty acid beta-oxidation (e.g., ECHS1). In some embodiments, the gene or gene product encodes a mitochondrial thiamine pyrophosphate carrier (e.g., SLC25A19).

In some embodiments, the gene or gene product decreases the transduction efficiency of AAV2 and AAV9 particles. In some embodiments, the gene or gene product inhibits or prevents the transduction of AAV2 and AAV9 particles. In some embodiments, the gene or gene product is WDR11. In some embodiments, the gene or gene product is MRE11.

In some embodiments, the gene or gene product is selected from the group consisting of: PITPNB, PITP, FAM91A1, WDR11, AP1G1, APIM1, APIS1, APIS3, HEATR5B, STX16, APIB1, PIAS1, DENR, ARL1, ZFAT, TBC1D23, LIN37, RALGAPB, B3GNT2, ELOVL1, AP2B1, KIAA2013, PTEN, MCTS1, NBN, HELZ, SLC38A10, FBXL20, TGIF1, KDSR, CPD, CHD7, USP9X, SIMC1, TAF11, VAPA, MTMR6, RAB1B, SLF2, MRE11, VT11A, MBOAT7, PPP6R3, or a combination thereof.

In some embodiments, the gene or gene product is associated with the long-chain FA elongation cycle (e.g., ELOVL1). In some embodiments, the gene or gene product is associated with lipoic acid biosynthesis (e.g., PIAS1). In some embodiments, the gene or gene product is associated with a protein phosphatase catalytic subunit (e.g., PPP6R3). In some embodiments, the gene or gene product is associated with the WDR11 pathway (e.g., WDR11, FAM91A1, AP1G1, AP1S1, AP1M1, AP1S3, or TBC1D23). In some embodiments, the gene or gene product is associated with retrograde transport from the Golgi apparatus to the endoplasmic reticulum, e.g., of PI and/or PC (e.g., PITP). In some embodiments, the gene or gene product is associated with vesicular transport from a late endosome to a trans-Golgi network (e.g., STX16). In some embodiments, the gene or gene product is associated with an activity or recruitment of a golgin, arfaptin, and/or Arf-GEF to a trans-Golgi network (e.g., ARL1). In some embodiments, the gene or gene product encodes a component of a clathrin-dependent vesicle and/or is associated with AP1G1/AP-1-mediated protein trafficking (e.g., HEATR5B).

In some embodiments, the modulator is: (a) a gene editing system targeted to one or more sites within the gene or a regulatory element thereof; (b) a nucleic acid encoding one or more components of the gene editing system; or (c) a combination thereof. In some embodiments, the gene editing system is a CRISPR/Cas system, a zinc finger nuclease system, a TALEN system, and a meganuclease system.

In some embodiments, the gene editing system binds to a target sequence in an early (e.g., the first, second, or third) exon or intron of the gene. In some embodiments, the gene editing system binds a target sequence of the gene, and the target sequence is upstream of exon 4, e.g., in exon 1, exon 2, or exon 3. In some embodiments, the gene editing system binds to a target sequence in a late exon or intron of the gene. In some embodiments, the gene editing system binds a target sequence of the gene, and the target sequence is downstream of a preantepenultimate exon, e.g., is in an antepenultimate exon, a penultimate exon, or a last exon. In some embodiments, the gene editing system is a CRISPR/Cas system comprising a guide RNA (gRNA) molecule comprising a targeting sequence which hybridizes to a target sequence of the gene. In some embodiments, the CRISPR/Cas system is a CRISPR/Cas9 system. In some embodiments, the CRISPR/Cas system is a CRISPR/Cas12a system.

In some embodiments, the modulator is a small interfering RNA (siRNA) or small hairpin (shRNA) specific for the gene, or a nucleic acid encoding the siRNA or shRNA. In some embodiments, the siRNA or shRNA comprises a nucleotide sequence complementary to a nucleotide sequence of an mRNA encoded by the gene. In some embodiments, the modulator is a guide RNA (gRNA) specific for the gene, or a nucleic acid encoding the gRNA. In some embodiments, the gRNA comprises a nucleotide sequence complementary to a nucleotide sequence of the gene.

In some embodiments, the modulator is an antisense oligonucleotide (ASO) specific for the gene, or a nucleic acid encoding the ASO. In some embodiments, the ASO comprises a nucleotide sequence complementary to a nucleotide sequence of an mRNA encoded by the gene.

In some embodiments, the modulator is a small molecule (e.g., a compound described herein, e.g., in FIG. 7). In some embodiments, the small molecule is a protein degrader.

In some embodiments, the modulator is a protein or a peptide, or a nucleic acid encoding the protein or peptide. In some embodiments, the modulator is an antibody molecule (e.g., an scFv or sdAb). In some embodiments, the modulator is a dominant negative binding partner of a protein encoded by the gene, or a nucleic acid encoding the dominant negative binding partner. In some embodiments, the modulator is a dominant negative variant (e.g., catalytically inactive) of a protein encoded by the gene, or a nucleic acid encoding said dominant negative variant.

In some embodiments, the AAV particle comprises an AAV genome. In some embodiments, the AAV particle comprises an AAV-like particle. In some embodiments, the AAV particle comprises a capsid. In some embodiments, the AAV particle has a serotype of AAV1, AAV2, AAV3, AAV4, AAV5, AAV6, AAV7, AAV8, AAV9, AAV10, AAV11, or a combination thereof. In some embodiments, the AAV particle is an AAV2 particle. In some embodiments, the AAV particle is an AAV9 particle.

In some embodiments, the AAV particle has a tropism towards the CNS (e.g., AAV1, AAV2, AAV4, AAV5, AAV8, or AAV9). In some embodiments, the AAV particle has a tropism towards heart (e.g., AAV1, AAV8, or AAV9). In some embodiments, the AAV particle has a tropism towards kidney (e.g., AAV2). In some embodiments, the AAV particle has a tropism towards liver (e.g., AAV7, AAV8, or AAV9). In some embodiments, the AAV particle has a tropism towards lung (e.g., AAV4, AAV5, AAV6, or AAV9). In some embodiments, the AAV particle has a tropism towards pancreas (e.g., AAV8). In some embodiments, the AAV particle has a tropism towards a photoreceptor cell (e.g., AAV2, AAV5, or AAV8). In some embodiments, the AAV particle has a tropism towards retinal pigment epithelium (RPE) (e.g., AAV1, AAV2, AAV4, AAV5, or AAV8). In some embodiments, the AAV particle has a tropism towards skeletal muscle (e.g., AAV1, AAV6, AAV7, AAV8, or AAV9).

In some embodiments, the AAV particle comprises a nucleotide sequence encoding a therapeutic protein, e.g., a protein associated with a disorder described herein. In some embodiments, the AAV particle comprises a nucleotide sequence encoding NGF, APOE2 (e.g., hAPOE2), TERT (e.g., hTERT), MAPT, GAD, AADC, NTN, GDNF, GCase, HTT, SMN, SMN2, SOD1, or C9orf72.

In some embodiments, the AAV particle comprises a nucleotide sequence encoding a therapeutic nucleic acid, e.g., a nucleic acid targeting a nucleotide sequence encoding a protein associated with a disorder described herein.

In some embodiments, the method comprises administering to the subject a plurality of AAV transduction modulators or an AAV modulator that modulates a plurality of genes or gene products associated with AAV transduction efficiency.

In an aspect, the disclosure features a method of producing a cell having an increased AAV transduction efficiency, comprising contacting a cell with an AAV transduction modulator, thereby producing the cell.

In some embodiments, the cell is a brain cell, a liver cell, a spinal cord cell, a dorsal root ganglion (DRG) cell, a spleen cell, a lymph node cell, a kidney cell, a lung cell, a heart cell, a muscle cell (e.g., a skeletal muscle cell, femur muscle cell), a diaphragm cell, a bone marrow cell, or a gonad cell. In some embodiments, the cell is a central nervous system (CNS) cell. In some embodiments, the CNS cell is an astrocyte, an oligodendrocyte, a microglial cell, or an ependymal cell. In some embodiments, the cell is a brain cell. In some embodiments, the brain cell is a neuron or glial cell. In some embodiments, the cell is a DRG cell. In some embodiments, the cell is a liver cell. In some embodiments, the liver cell is a hepatocyte, hepatic stellate cell, a Kupffer cell, or a liver sinusoidal endothelial cell.

In some embodiments, the cell is contacted with the AAV transduction modulator in vitro. In some embodiments, the cell is contacted with the AAV transduction modulator ex vivo. In some embodiments, the cell is contacted with the AAV transduction modulator in vivo.

In some embodiments, the cell is obtained from a subject. In some embodiments, the subject has a disorder or a symptom thereof, or is at risk of having a disorder or a symptom thereof. In some embodiments, the disorder is a neurodegenerative disorder. In some embodiments, the neurodegenerative disorder is Alzheimer's disease, Parkinson's disease, Huntington's disease, multiple sclerosis (MS), amyotrophic lateral sclerosis (ALS), or Batten disease. In some embodiments, the disorder is mild Alzheimer's disease, mild-to-moderate Alzheimer's disease, mild-to-severe Alzheimer's disease, early dementia, Parkinson's disease (e.g., idiopathic Parkinson's disease, bilateral idiopathic Parkinson's disease, mid-to-late stage Parkinson's disease), Huntington's disease (e.g., manifest Huntington's disease), Type 1 SMA, presymptomatic SMA, or amyotrophic lateral sclerosis (ALS) (e.g., familial ALS, ALS with SOD1 mutation, or ALS with C9orf72 mutation). In some embodiments, the disorder is an eye disorder. In some embodiments, the eye disorder is blindness, e.g., inherited or non-inherited blindness. In some embodiments, the eye disorder is Leber's congenital amaurosis, age-related macular degeneration, choroideremia, or color blindness. In some embodiments, the method reduces the toxicity of the therapy, enhances the efficacy of the therapy, or both.

In some embodiments, the modulator modulates (e.g., increases or decreases) a gene or gene product associated with AAV transduction efficiency. In some embodiments, the gene or gene product is a mammalian (e.g., human) gene or gene product.

In some embodiments, the modulator inhibits the gene or gene product, e.g., a gene or gene product that is associated with a decreased transduction efficiency of an AAV particle. In some embodiments, the modulator activates the gene or gene product, e.g., a gene or gene product that is associated with an increased transduction efficiency of an AAV particle.

In some embodiments, the gene or gene product is associated with an increased transduction efficiency of an AAV particle. In some embodiments, the gene or gene product is associated with an increased transduction efficiency of an AAV particle of a first serotype (e.g., AAV2), an increased transduction efficiency of an AAV particle of a second serotype (e.g., AAV9), or both. In some embodiments, the gene or gene product is AAV-R. In some embodiments, the gene or gene product is GPR108.

In some embodiments, the gene or gene product is associated with a decreased transduction efficiency of an AAV particle. In some embodiments, the gene or gene product is associated with a decreased transduction efficiency of an AAV particle of a first serotype (e.g., AAV2), a decreased transduction efficiency of an AAV particle of a second serotype (e.g., AAV9), or both. In some embodiments, the gene or gene product is WDR11. In some embodiments, the gene or gene product is MRE11.

In some embodiments, the gene or gene product, when modulated, increases the transduction efficiency of an AAV particle. In some embodiments, the gene or gene product, when modulated, increases the transduction efficiency of an AAV particle of a first serotype (e.g., AAV2), the transduction efficiency of an AAV particle of a second serotype (e.g., AAV9), or both.

In some embodiments, the gene or gene product, when modulated, decreases the transduction efficiency of an AAV particle. In some embodiments, the gene or gene product, when modulated, decreases the transduction efficiency of an AAV particle of a first serotype (e.g., AAV2), the transduction efficiency of an AAV particle of a second serotype (e.g., AAV9), or both.

In some embodiments, the gene product is an RNA (e.g., an mRNA). In some embodiments, the gene product is a protein. In some embodiments, the gene or gene product is preferentially expressed in a target tissue (e.g., brain or liver). The following dependent claims describe the AAV transduction modulator based on its mechanism. In some embodiments, the modulator alters (e.g., increases or decreases) the expression of the gene. In some embodiments, the modulator alters the structure of the gene. In some embodiments, the modulator alters (e.g., increases or decreases) an activity (e.g., an enzymatic activity) of the gene product. In some embodiments, the modulator alters (e.g., increases or decreases) the level (e.g., abundance) of the gene product. In some embodiments, the modulator alters (e.g., increases or decreases) the stability of the gene product. In some embodiments, the modulator alters (e.g., increases or decreases) transduction efficiency of an AAV particle, e.g., as determined by an assay described herein (e.g., an FACS analysis, e.g., as described in Examples 1 or 2).

In some embodiments, the modulator increases transduction efficiency of an AAV particle. In some embodiments, the transduction efficiency is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference level of transduction efficiency. In some embodiments, the modulator increases transduction to at least 10, 20, 30, 40, 50, or 60 viral copies per genome. In some embodiments, the modulator increases transduction to at least 10³, 10⁴, 10⁵, 10⁶, or 10⁷ viral copies per μg DNA.

In some embodiments, the modulator decreases transduction efficiency of an AAV particle. In some embodiments, the transduction efficiency is decreased by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of transduction efficiency. In some embodiments, the modulator decreases transduction to no more than 0, 1, 2, 3, 4, 5, 6, 7, 8, 9, or 10 viral copies per genome. In some embodiments, the modulator decreases transduction to no more than 10², 10³, or 10⁴ viral copies per μg DNA.

In some embodiments, the reference level of transduction efficiency is the level of transduction efficiency in a cell that has not been contacted with the AAV transduction modulator. In some embodiments, the reference level of transduction efficiency is the level of transduction efficiency before the cell is contacted with the AAV transduction modulator.

In some embodiments, the gene or gene product increases the transduction efficiency of an AAV2 particle, optionally wherein the gene or gene product or gene or gene product does not increase, or does not substantially increase, the transduction efficiency of an AAV9 particle. In some embodiments, the gene or gene product is necessary for the transduction of an AAV2 particle, optionally wherein the gene or gene product is not necessary for the transduction of an AAV9 particle. In some embodiments, the gene or gene product is AAV-R. In some embodiments, the gene or gene product is GRP108.

In some embodiments, the gene or gene product is chosen from EXT1, EXT2, NDST1, BCL10, OAF, PDCD10, LMO4, MALT1, ZNF644, CHUK, GLCE, EP300, ARID4B, RAB21, MED13, DHX15, WASHC5, IKBKB, USP24, HSPA14, CXXC1, UBE2A, INTS8, CDC42, NFKB1, SIN3A, USP7, ARF5, CARD10, JAK1, SLC35C2, PAXK1, ZC3H11A, FAM20B, FAM72A, DHX36, INTS12, RPRD1B, or a combination thereof.

In some embodiments, the gene or gene product is associated with endosomal sorting (e.g., RAB21, WASHC5, or USP7). In some embodiments, the gene or gene product is associated with vesicle-mediated transport (e.g., CDC42 or ARF5). In some embodiments, the gene or gene product is associated with NFKB signaling (e.g., BCL10, MALT1, NFKB1, IKBKB, CHUK, or CARD10). In some embodiments, the gene or gene product is associated with heparan sulfate biosynthesis (e.g., EXT1, EXT2, NDST1, GLCE, or FAM20B). In some embodiments, the gene or gene product is associated with H3K9 methylation (e.g., ZNF644). In some embodiments, the gene or gene product is associated with pre-mRNA processing (e.g., DHX15). In some embodiments, the gene or gene product is associated with transcription (e.g., LMO4, EP300, MED13, SIN3, ARID4B, ZC3H11A, CXXC1, RPRD1B, or UBE2A). In some embodiments, the gene or gene product is associated with integrator complex and/or RNA polymerase II function (e.g., INTS8 or INST12). In some embodiments, the gene or gene product is associated with DNA repair and/or AAV genome resolution (e.g., DHX36, ARID4B, UBE2A, or USP24). In some embodiments, the gene or gene product is associated with cell proliferation and/or apoptosis (e.g., PDCD10). In some embodiments, the gene or gene product is associated with spondylocarpotarsal synostosis syndrome (e.g., OAF). In some embodiments, the gene or gene product is associated with protein folding (e.g., HSPA14). In some embodiments, the gene or gene product is associated with helicase activity (e.g., DHX36). In some embodiments, the gene or gene product is associated with fucosylation of Notch and/or transport of GCP-fucose (e.g., SLC35C2).

In some embodiments, the gene or gene product increases the transduction efficiency of an AAV9 particle, optionally wherein the gene or gene product does not increase, or does not substantially increase, the transduction efficiency of an AAV2 particle. In some embodiments, the gene or gene product is necessary for the transduction of an AAV9 particle, optionally wherein the gene or gene product is not necessary for the transduction of an AAV2 particle. In some embodiments, the gene or gene product is AAV-R. In some embodiments, the gene or gene product is GRP108.

In some embodiments, the gene or gene product is chosen from TMPRSS11B, HTT, SNRNP70, BRD7, DMXL1, RAB10, CNGA1, KLHDC3, CHP1, CYP3A5, ELOVL4, PNISR, ZCCHC14, AZGP1, TPST1, SC5D, ELP2, ELP3, IPO9, RAB14, WDR7, XRCC4, GDI2, or a combination thereof.

In some embodiments, the gene or gene product is associated with microtubule-mediated transport or vesicle function (e.g., HT). In some embodiments, the gene or gene product is an ion channel (e.g., CNGA1). In some embodiments, the gene or gene product is associated with nuclear protein import (e.g., IP09). In some embodiments, the gene or gene product is associated with O-sulfation of a tyrosine residue within an acidic motif of a polypeptide (e.g., TPST1). In some embodiments, the gene or gene product is associated with protection of viral RNA (e.g., ZCCHC14). In some embodiments, the gene or gene product is associated with RNA binding by an AAV protein (e.g., PNISR). In some embodiments, the gene or gene product is associated with calcium ion-regulated exocytosis (e.g., CHP1). In some embodiments, the gene or gene product is associated with intracellular trafficking (e.g., RAB10, RAB14, GDI2, WDR7, or DMXL1). In some embodiments, the gene or gene product is a subunit (e.g., a catalytic tRNA acetyltransferase subunit) of an elongator complex, e.g., of an RNA polymerase (e.g., RNA polymerase II), e.g., ELP2 or ELP3. In some embodiments, the gene or gene product binds to chromatin (e.g., KLHDC3). In some embodiments, the gene or gene product is associated with cholesterol biosynthesis (e.g., SC5D).

In some embodiments, the gene or gene product increases the transduction efficiency of AAV2 and AAV9 particles. In some embodiments, the gene or gene product is necessary for the transduction of AAV2 and AAV9 particles. In some embodiments, the gene or gene product is AAV-R. In some embodiments, the gene or gene product is GRP108.

In some embodiments, the gene or gene product is chosen from KIAA0319L, BAMBI, TM9SF2, PHIP, DCLRE1C, VPS35, GPR108, CYREN, ACP2, F8A2, F8A1, F8A3, RBPJ, ATP2C1, ICMT, RABIF, IER3IP1, RBM10, SMG7, GTF2I, ELAVL1, MEPCE, RAB4A, IER3IP1, SAMD1, or a combination thereof.

In some embodiments, the gene or gene product is associated with influenza infection (e.g., ACP2). In some embodiments, the gene or gene product binds to unmethylated CGIs (e.g., SAMD1). In some embodiments, the gene or gene product is associated with intracellular vesicular transport (e.g., RABIF). In some embodiments, the gene or gene product is associated with posttranslational modification of cysteine residues (e.g., C-terminal cysteine residues), e.g., in a target protein (e.g., ICMT). In some embodiments, the gene or gene product binds to a capsid protein of AAV (e.g., KIAA0319L). In some embodiments, the gene or gene product is associated with inhibition of a TLR (e.g., TLR9), e.g., GPR108. In some embodiments, the gene or gene product is associated with endosome trafficking, e.g., of AAV-R (e.g., VPS35). In some embodiments, the gene or gene product is a calcium ATPase pump (e.g., ATP2C1). In some embodiments, the gene or gene product is associated with Notch signaling (e.g., RBPJ). In some embodiments, the gene or gene product is associated with HSPG metabolism (e.g., TM9SF2). In some embodiments, the gene or gene product is associated with inhibition of NHEJ (e.g., CYREN). In some embodiments, the gene or gene product is associated with viral hairpin resolution (e.g., DCLRE1C). In some embodiments, the gene or gene product is associated with Glc transporter translocation, optionally wherein the gene or gene product binds to insulin receptor substrate 1 protein (e.g., PHIP).

In some embodiments, the gene or gene product decreases the transduction efficiency of an AAV2 particle, optionally wherein the gene or gene product, when modulated, does not decrease, or does not substantially decrease, the transduction efficiency of an AAV9 particle. In some embodiments, the gene or gene product inhibits or prevents the transduction of an AAV2 particle, optionally wherein the gene or gene product does not inhibit or prevent the transduction of an AAV9 particle. In some embodiments, the gene or gene product is WDR11. In some embodiments, the gene or gene product is MRE11.

In some embodiments, the gene or gene product is chosen from GREB1L, BIRC2, TYMSOS, MSL3, SIK3, GTF2H5, PIGW, ATRAID, PPP4R1, ZFC3H1, SETDB1, SMCHD1, TMEM123, UHRF1, CLUL1, SMAD5, SETX, STEEP1, DENND6A, RTN4RL2, MTMR9, STRADA, PSIP1, BMPR1A, HIKESHI, AP3M1, MEN1, MYL12B, CREB1, RAB12, SMTNL1, MRGBP, WTAP, SUMO2, TCN1, ALDH3B1, ARL14EP, MYL12A, RHOD, PGAP2, EIF4A1, PIGN, DAXX, HDLBP, GOLGA1, SARNP, UBE2L6, TRIM49, SIK2, SOX4, OR6Q1, OR4D6, UNC93B1, CATSPERZ, PIGT, PYM1, MICOS13, RIN1, DOT1L, TRIM49C, MAPK20, COL8A1, FASN, KDM3B, PIGF, PIGA, PIGY, GPC3, TRAPPC2L, RAB31, ELP3, or a combination thereof.

In some embodiments, the gene or gene product is associated with a retinoic acid receptor activity (e.g., GREB1L). In some embodiments, the gene or gene product is a thymidylate synthetase (e.g., TYMSOS). In some embodiments, the gene or gene product is associated with chromatin remodeling (e.g., SMCHD1). In some embodiments, the gene or gene product is associated with histone H4 acetylation (e.g., MSL3). In some embodiments, the gene or gene product is associated with transcription and/or DNA repair (e.g., GTF2H5). In some embodiments, the gene or gene product is a histone methyltransferase (e.g., SETDB1). In some embodiments, the gene or gene product is associated with exosomal degradation of polyadenylated RNA (e.g., ZFC3H1). In some embodiments, the gene or gene product is associated with histone deacetylase recruitment to a DNA (e.g., UHRF1). In some embodiments, the gene or gene product is associated with oxidative stress-induced DNA double strand break response (e.g., SETX). In some embodiments, the gene or gene product is associated with histone modification (e.g., MEN1). In some embodiments, the gene or gene product is associated with nucleosome/DNA interaction (e.g., MRGBP). In some embodiments, the gene or gene product is associated with GPI biosynthesis (e.g., PIGW, PIGN, PIGT, PIGF, PIGA, or PIGY). In some embodiments, the gene or gene product is associated with maturation of GPI anchors (e.g., PGAP2). In some embodiments, the gene or gene product is associated with vesicle-mediated endocytosis (e.g., DENND6A, GOLGA1, MTMR9, GPC3, PPP4R1, RAB12, RAB31, RHOD, RIN1, TMEM123, or TRAPPC2L). In some embodiments, the gene or gene product is associated with clathrin-coated vesicle-mediated endocytosis (e.g., AP3M1). In some embodiments, the gene or gene product is associated with a BMP receptor kinase (e.g., SMAD5, BMPR1A, or CREB1). In some embodiments, the gene or gene product is associated with transcription (e.g., PSIP1). In some embodiments, the gene or gene product is associated with nuclear import of an HSP70 protein (e.g., HIKESHI). In some embodiments, the gene or gene product is associated with a Ser/Thr kinase (e.g., SIK2 or SIK3). In some embodiments, the gene or gene product is associated with SUMOylation of a protein (e.g., SUMO2, UBE2L6).

In some embodiments, the gene or gene product decreases the transduction efficiency of an AAV9 particle, optionally wherein the gene or gene product does not decrease, or does not substantially decrease, the transduction efficiency of an AAV2 particle. In some embodiments, the gene or gene product inhibits or prevents the transduction of an AAV9 particle, optionally wherein the gene or gene product does not inhibit or prevent the transduction of an AAV2 particle. In some embodiments, the gene or gene product is WDR11. In some embodiments, the gene or gene product is MRE11.

In some embodiments, gene or gene product is chosen from AP2A1, AP2B1, TM9SF4, ACTR5, PTMA, PAPOLA, PDHA1, PDHB, SLC25A19, GAK, AUNIP, FCHO2, ACTB, LIPT1, UCHL5, INO80E, ECHS1, NELFB, EDC4, PRMT1, PDS5B, PELI3, FBXL20, SLC35A1, or a combination thereof.

In some embodiments, the gene or gene product binds to ATP, a chaperone protein, and/or clathrin (e.g., GAK). In some embodiments, the gene or gene product is associated with clathrin-coated vesicle trafficking (e.g., AP2B1 or AP2A1). In some embodiments, the gene or gene product is associated with clathrin-mediated endocytosis (e.g., FCHO2). In some embodiments, the gene or gene product is associated with vesicular trafficking (e.g., ACTB). In some embodiments, the gene or gene product is associated with an innate immune response (e.g., PELI3 or FBXL20). In some embodiments, the gene or gene product is associated with glucose metabolism (e.g., PDHB or PDHA1). In some embodiments, the gene or gene product is associated with DNA resection and/or homologous recombination, e.g., after DNA damage (e.g., AUNIP). In some embodiments, the gene or gene product is associated with DNA repair, e.g., after DNA damage (e.g., UCHL5, INO80E, PDS5B, ACTR5, or PRMT1). In some embodiments, the gene or gene product is associated with transport of CMP-sialic acid from cytosol to a Golgi vesicle (e.g., SLC35A1). In some embodiments, the gene or gene product is associated with localization of polypeptides comprising glycine-rich transmembrane domains, e.g., to the cell surface (e.g., TM9SF4). In some embodiments, the gene or gene product is associated with poly(A) tail synthesis (e.g., PAPOLA). In some embodiments, the gene or gene product is PTMA. In some embodiments, the gene or gene product is associated with elongation of an mRNA by RNA polymerase II (e.g., NELFB). In some embodiments, the gene or gene product is associated with mRNA degradation (e.g., EDC4). In some embodiments, the gene or gene product encodes a mitochondrial lipoyltransferase (e.g., LIPT1). In some embodiments, the gene or gene product is associated with mitochondrial fatty acid beta-oxidation (e.g., ECHS1). In some embodiments, the gene or gene product encodes a mitochondrial thiamine pyrophosphate carrier (e.g., SLC25A19).

In some embodiments, the gene or gene product decreases the transduction efficiency of AAV2 and AAV9 particles. In some embodiments, the gene or gene product inhibits or prevents the transduction of AAV2 and AAV9 particles. In some embodiments, the gene or gene product is WDR11. In some embodiments, the gene or gene product is MRE11.

In some embodiments, the gene or gene product is selected from the group consisting of: PITPNB, PITP, FAM91A1, WDR11, AP1G1, AP1M1, AP1S1, AP1S3, HEATR5B, STX16, AP1B1, PIAS1, DENR, ARL1, ZFAT, TBC1D23, LIN37, RALGAPB, B3GNT2, ELOVL1, AP2B1, KIAA2013, PTEN, MCTS1, NBN, HELZ, SLC38A10, FBXL20, TGIF1, KDSR, CPD, CHD7, USP9X, SIMC1, TAF11, VAPA, MTMR6, RAB1B, SLF2, MRE11, VT11A, MBOAT7, PPP6R3, or a combination thereof.

In some embodiments, the gene or gene product is associated with the long-chain FA elongation cycle (e.g., ELOVL1). In some embodiments, the gene or gene product is associated with lipoic acid biosynthesis (e.g., PIAS1). In some embodiments, the gene or gene product is associated with a protein phosphatase catalytic subunit (e.g., PPP6R3). In some embodiments, the gene or gene product is associated with the WDR11 pathway (e.g., WDR11, FAM91A1, AP1G1, AP1S1, AP1M1, AP1S3, or TBC1D23). In some embodiments, the gene or gene product is associated with retrograde transport from the Golgi apparatus to the endoplasmic reticulum, e.g., of PI and/or PC (e.g., PITP). In some embodiments, the gene or gene product is associated with vesicular transport from a late endosome to a trans-Golgi network (e.g., STX16). In some embodiments, the gene or gene product is associated with an activity or recruitment of a golgin, arfaptin, and/or Arf-GEF to a trans-Golgi network (e.g., ARL1). In some embodiments, the gene or gene product encodes a component of a clathrin-dependent vesicle and/or is associated with AP1G1/AP-1-mediated protein trafficking (e.g., HEATR5B).

In some embodiments, the modulator is: (a) a gene editing system targeted to one or more sites within the gene or a regulatory element thereof; (b) a nucleic acid encoding one or more components of the gene editing system; or (c) a combination thereof. In some embodiments, the gene editing system is a CRISPR/Cas system, a zinc finger nuclease system, a TALEN system, and a meganuclease system.

In some embodiments, the gene editing system binds to a target sequence in an early (e.g., the first, second, or third) exon or intron of the gene. In some embodiments, the gene editing system binds a target sequence of the gene, and the target sequence is upstream of exon 4, e.g., in exon 1, exon 2, or exon 3. In some embodiments, the gene editing system binds to a target sequence in a late exon or intron of the gene. In some embodiments, the gene editing system binds a target sequence of the gene, and the target sequence is downstream of a preantepenultimate exon, e.g., is in an antepenultimate exon, a penultimate exon, or a last exon. In some embodiments, the gene editing system is a CRISPR/Cas system comprising a guide RNA (gRNA) molecule comprising a targeting sequence which hybridizes to a target sequence of the gene. In some embodiments, the CRISPR/Cas system is a CRISPR/Cas9 system. In some embodiments, the CRISPR/Cas system is a CRISPR/Cas12a system.

In some embodiments, the modulator is a small interfering RNA (siRNA) or small hairpin (shRNA) specific for the gene, or a nucleic acid encoding the siRNA or shRNA. In some embodiments, the siRNA or shRNA comprises a nucleotide sequence complementary to a nucleotide sequence of an mRNA encoded by the gene. In some embodiments, the modulator is a guide RNA (gRNA) specific for the gene, or a nucleic acid encoding the gRNA. In some embodiments, the gRNA comprises a nucleotide sequence complementary to a nucleotide sequence of the gene.

In some embodiments, the modulator is an antisense oligonucleotide (ASO) specific for the gene, or a nucleic acid encoding the ASO. In some embodiments, the ASO comprises a nucleotide sequence complementary to a nucleotide sequence of an mRNA encoded by the gene.

In some embodiments, the modulator is a small molecule (e.g., a compound described herein, e.g., in FIG. 7). In some embodiments, the small molecule is a protein degrader.

In some embodiments, the modulator is a protein or a peptide, or a nucleic acid encoding the protein or peptide. In some embodiments, the modulator is an antibody molecule (e.g., an scFv or sdAb). In some embodiments, the modulator is a dominant negative binding partner of a protein encoded by the gene, or a nucleic acid encoding the dominant negative binding partner. In some embodiments, the modulator is a dominant negative variant (e.g., catalytically inactive) of a protein encoded by the gene, or a nucleic acid encoding said dominant negative variant.

In some embodiments, the AAV particle comprises an AAV genome. In some embodiments, the AAV particle comprises an AAV-like particle. In some embodiments, the AAV particle comprises a capsid. In some embodiments, the AAV particle has a serotype of AAV1, AAV2, AAV3, AAV4, AAV5, AAV6, AAV7, AAV8, AAV9, AAV10, AAV11, or a combination thereof. In some embodiments, the AAV particle is an AAV2 particle. In some embodiments, the AAV particle is an AAV9 particle.

In some embodiments, the AAV particle has a tropism towards the CNS (e.g., AAV1, AAV2, AAV4, AAV5, AAV8, or AAV9). In some embodiments, the AAV particle has a tropism towards heart (e.g., AAV1, AAV8, or AAV9). In some embodiments, the AAV particle has a tropism towards kidney (e.g., AAV2). In some embodiments, the AAV particle has a tropism towards liver (e.g., AAV7, AAV8, or AAV9). In some embodiments, the AAV particle has a tropism towards lung (e.g., AAV4, AAV5, AAV6, or AAV9). In some embodiments, the AAV particle has a tropism towards pancreas (e.g., AAV8). In some embodiments, the AAV particle has a tropism towards a photoreceptor cell (e.g., AAV2, AAV5, or AAV8). In some embodiments, the AAV particle has a tropism towards retinal pigment epithelium (RPE) (e.g., AAV1, AAV2, AAV4, AAV5, or AAV8). In some embodiments, the AAV particle has a tropism towards skeletal muscle (e.g., AAV1, AAV6, AAV7, AAV8, or AAV9).

In some embodiments, the AAV particle comprises a nucleotide sequence encoding a therapeutic protein, e.g., a protein associated with a disorder described herein. In some embodiments, the AAV particle comprises a nucleotide sequence encoding NGF, APOE2 (e.g., hAPOE2), TERT (e.g., hTERT), MAPT, GAD, AADC, NTN, GDNF, GCase, HTT, SMN, SMN2, SOD1, or C9orf72.

In some embodiments, the AAV particle comprises a nucleotide sequence encoding a therapeutic nucleic acid, e.g., a nucleic acid targeting a nucleotide sequence encoding a protein associated with a disorder described herein.

In some embodiments, the method comprises contacting the cell with a plurality of AAV transduction modulators or an AAV modulator that modulates a plurality of genes or gene products associated with AAV transduction efficiency.

In another aspect, the disclosure provides a cell produced by a method described herein. In another aspect, the disclosure provides a cell comprising an AAV modulator described herein and an AAV particle (e.g., an AAV particle described herein).

In another aspect, the disclosure provides a pharmaceutical composition comprising an AAV modulator described herein and an AAV particle (e.g., an AAV particle described herein). In another aspect, the disclosure provides a kit comprising an AAV modulator described herein and an AAV particle (e.g., an AAV particle described herein).

In another aspect, the disclosure provides an AAV transduction modulator for use in a method of modulating transduction efficiency of an AAV particle in a cell or a subject, e.g., in accordance with a method described herein.

In another aspect, the disclosure provides an AAV transduction modulator for use in combination with an AAV genome or an AAV particle in a method of treating a disorder in a subject, e.g., in accordance with a method described herein.

In another aspect, the disclosure provides an AAV transduction modulator for use in a method of preparing a subject for a therapy comprising an AAV genome or an AAV particle, e.g., in accordance with a method described herein.

In another aspect, the disclosure provides an AAV transduction modulator for use in a method of reducing the toxicity of a therapy comprising an AAV genome or an AAV particle in a subject, e.g., in accordance with a method described herein.

In another aspect, the disclosure provides an AAV transduction modulator for use in a method of increasing the efficacy of a therapy comprising an AAV genome or an AAV particle in a subject, e.g., in accordance with a method described herein.

In another aspect, the disclosure provides use of an AAV transduction modulator in the manufacture of a medicament for modulating transduction efficiency of an AAV particle in a cell or a subject, e.g., in accordance with a method described herein.

In another aspect, the disclosure provides use of an AAV transduction modulator in the manufacture of a medicament in combination with an AAV genome or an AAV particle for treating a disorder in a subject, e.g., in accordance with a method described herein.

In another aspect, the disclosure provides use of an AAV transduction modulator in the manufacture of a medicament for preparing a subject for a therapy comprising an AAV genome or an AAV particle, e.g., in accordance with a method described herein.

In another aspect, the disclosure provides use of an AAV transduction modulator in the manufacture of a medicament for reducing the toxicity of a therapy comprising an AAV genome or an AAV particle in a subject, e.g., in accordance with a method described herein.

In another aspect, the disclosure provides use of an AAV transduction modulator in the manufacture of a medicament for increasing the efficacy of a therapy comprising an AAV genome or an AAV particle in a subject, e.g., in accordance with a method described herein.

In some embodiments of any of the above aspects, the gene or gene product is AAV-R, GPR108, WDR11 or MRE11. In some embodiments, the gene or gene product is AAV-R. In some embodiments, the gene or gene product is GPR108. In some embodiments, the gene or gene product is WDR11. In some embodiments, the gene or gene product is MRE11.

In some embodiments of any of the above aspects, the method results in a high gene modulating (e.g., gene editing) efficiency (e.g., at least 60%, 65%, 70%, 75%, 80%, 85%, 90%, 95%, or 98%) in a first tissue, with limited impact (e.g., a gene modulating (e.g., gene editing) efficiency of 20%, 15%, 10%, 5%, 2%, or less) in a second tissue. In some embodiments, the method results in a high modulating (e.g., gene editing) efficiency (e.g., at least 60%, 65%, 70%, 75%, 80%, 85%, 90%, 95%, or 98%) in liver, with limited impact (e.g., a gene modulating (e.g., gene editing) efficiency of 20%, 15%, 10%, 5%, 2%, or less) in skeletal muscle, bone marrow, or both.

In some embodiments of any of the above aspects, the modulator is administered intravenously.

### BRIEF DESCRIPTION OF THE DRAWINGS

FIG. 1A is a diagram showing an exemplary pooled screening next-generation sequencing (NGS) workflow for a genome-wide CRISPR/Cas9 screen for modulators of AAV transduction.

FIG. 1B is a diagram showing an overview of the AAV CRISPR screening methodology used to identify genetic modulators of AAV2 or AAV9 transduction in human (Huh-7) and murine (AML-12) cells.

FIGS. 2A-2D are a series of diagrams showing identification of common genes modulating AAV2 and AAV9 transduction. Shown is the result of the whole-genome CRISPR screen in Huh-7 using either AAV2 or AAV9 vectors. The genes significantly impacting the transduction of each serotypes are plotted based upon their RSA scores. The genes identified to significantly affect AAV2 transduction were plotted on the Y axis while the genes identified in the AAV9 screen were plotted on the X axis. The data points highlighted on the upper right quadrant of A and C represent all the genes that were shown to significantly promote (A) or inhibit (C) the transduction of Huh-7 cells by both serotypes. The exact lists of those host factors are indicated in (B) (genes promoting AAV2/AAV9 transduction) and (D) (genes preventing AAV2/AAV9 transduction).

FIG. 3 is a series of graphs showing validation of selected hits across the Huh-7 and SK-N-SH cell lines. AAV-R, GPR108, TM9SF2, WDR11, FAM91A1 and PITPNB genes were individually knocked down by CRISPR/Cas9 editing in Huh-7 cells (upper panels) or SK-N-SH cells (lower panels). After selecting the cells for 21 days, each modified cell line was transduced with either AAV2 or AAV9 and flow cytometry was performed at day 43 or 4 post-transduction, respectively. The MOI/titer of AAV used for transduction targeted ˜20% GFP positive cells for AAV2 and 60% GFP positive cells for AAV9 based upon previous titration curves. The percentage of GFP positive cells was calculated.

FIGS. 4A-4D are a series of diagrams showing genes promiting or preventing AAV9 transduction. Shown is the result of the whole-genome CRISPR screen in Huh-7 using either AAV2 or AAV9 vectors. The genes specifically increasing or inhibiting AAV9 transduction are highlighted in the bottom right quadrant of the graphs (A) and (C), respectively. The exact lists of those host factors are indicated in (B) (genes promoting AAV9 transduction) and (D) (genes preventing AAV9 transduction).

FIGS. 5A-5D are a series of diagrams showing genes promoting or preventing AAV2 transduction. The genes specifically increasing or inhibiting AAV2 transduction are highlighted in the upper left quadrant of the graphs A and C, respectively. The exact lists of those host factors are indicated in B (genes promoting AAV2 transduction) and D (genes preventing AAV2 transduction).

FIGS. 6A-6C are a series of diagrams showing identification of key host factors promoting or inhibiting AAV9 transduction in murine AML12 cells. Shown are a list of all murine genes that were shown to significantly promote (A) or reduce (B) AAV9 transduction. When the first 50 identified murine genes were compared with the results of the human screen with a similar vector, very few common host factors were identified between the two species. (C) The first 50 genetic hits identified in the murine screen were compared with the results obtained from the screen conducted in Huh-7 cells, revealing very limited overlap between the results.

FIG. 7 is a table showing modulators that can be used to increase or decrease an activity of a candidate gene identified in the CRISPR/AAV screen. Shown is a list of pharmacological compounds targeting specific genes identified from the CRISPR screens that will be tested in vitro for their ability to modulate AAV transduction. Three concentrations (1 μM, 100 nM and 10 nM) are tested in Huh-7 cells at first, before assessing their impact on different cell lines and in vivo.

FIGS. 8A-8B are a series of schematics showing experiments for validating the impact of genes identified in the whole-genome CRISPR/Cas9 screen on AAV9 transduction in vivo, using sgRNA-encapsulated nanoparticles.

FIG. 9 is a graph showing validation of murine sgRNA in AML-12 cell line. AAV-R, GPR108, WDR11, and MRE11 genes were individually knocked down by CRISPR/Cas9 editing in AML-12. After selecting the cells for 21 days, each modified cell line was transduced with either AAV2 or AAV9 and fluorescent imaging was performed at day 4 post-transduction. The vg/mL of AAV used for transduction targeted ˜50% GFP positive cells for. The percentage of GFP positive cells was calculated then compared to AAV treated non-gene edited parental cell line. For each of the genes, the result for cells transduced with AAV2 is shown on the left, and the result for cells transduced with AAV9 is shown on the right.

FIG. 10 is a table showing average percent of gene editing efficiency in the liver, skeletal muscle, and bone marrow. The percentage of editing is the average across the animals for the gene of interest corresponding to the sgRNA/DLP used in the group. Data not shown, no gene editing occurred in the AAV-R, GPR108, WDR11, or MRE11 gene in animals not-treated with the corresponding sgRNA/DLP.

FIGS. 11A-11B are a series of graphs showing AAV biodistribution in various tissues. The table displays a summary of the viral copy number analysis in the bone marrow, heart, liver, skeletal muscle, and spinal cord. Graph A. presents the results as viral copy per μg of gDNA, which graph B presents the data as viral copies per diploid genome.

FIGS. 12A-12B are a series of graphs showing mRNA expression of GFP in the liver (A) and muscle (B), as compared to the sgSCR. A single muscle sample was available from DLP1156/sgAAV-R and DLP1156/sgGPR108 groups.

FIGS. 13A-13C are a series of Western blots showing protein expression of AAV-R, MRE11, and GFP. Shown are the results of Western blot analysis of AAV-R (A), MRE11 (B) and GFP (C) in liver samples.

FIG. 14 is a graph showing direct measurement of fluorescence intensity in liver protein extracts. Shown is measurement of fluorescent intensity at 485 nm in liver homogenates and expressed at relative fluorescence units (RFUs).

FIG. 15 is a series of diagrams showing immunohistochemistry staining of GFP in liver tissue. Across the 7 treatment groups within the study, all the livers were examined for GFP expression using IHC. GFP staining is brown, which is very strong in the sgRNA-scramble treated animals (group 2).

FIG. 16 is a series of diagrams showing in situ hybridization to detect GFP antisense (AS) sequences in liver tissue. All animals were processed for GFP antisense sequences in the liver. Positive expression is designated in brown. This process captures the single cell GFP mRNA expression. The data gives clarity to variety of the mRNA expression levels per cell.

FIG. 17 is a series of diagrams showing in situ hybridization to detect GFP sense (S) sequences in liver tissue. All animals were processed for GFP sense sequences in the liver. Positive expression is designated in brown. This process captures the single cell GFP DNA expression. The data gives clarity to variety of the AAV transduction to each individual cell.

FIGS. 18A-18C are a series of graphs showing percentage of GFP-positive signal across a series of molecular localization endpoints.

### DETAILED DESCRIPTION

The present disclosure is based, at least in part, on genome wide CRISPR screenings that identified genes relevant for AAV transduction. AAVs are a platform for gene therapy delivery, utilized across therapeutic areas. Understanding transduction/tropism is important to both efficacy and safety as ectopic expression or transgene overexpression with AAV-based gene therapies can sometimes cause cellular toxicity (e.g., liver, DRG neurons, cardiomyocytes, RPE). Cellular tropism is dependent on expression of AAV receptors and co-receptors, but also on additional pathways controlling, e.g., intracellular trafficking, nuclear translocation of capsids, uncoating and episome formation. Further understanding of these mechanisms can inform the translatability of preclinical studies and provide mitigation strategies to limit or prevent targeting and expression in susceptible or “unwanted” cell type. The study described in the present disclosure aims to understand key cellular processes impacting AAV transduction across serotypes, cell types, and species. Screens utilizing AAV2 and AAV9 in human cells confirmed top hits important for infections, while highlighting genes more specifically impactful to either AAV2 or AAV9 transduction. Based on the identified genes, AAV transduction modulators can be designed, made, and used in AAV-based gene therapies for treating various disorders.

### Definitions

Unless defined otherwise, all technical and scientific terms used herein have the same meaning as commonly understood by one of ordinary skill in the art to which the disclosure pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an antibody, that retains the ability to specifically interact with (e.g., by binding, steric hinderance, stabilizing/destabilizing, spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide brudge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the term “antibody molecule” or “binding domain” refers to a protein, e.g., an immunoglobulin chain or fragment thereof, comprising at least one immunoglobulin variable domain sequence. The term “antibody molecule” or “binding domain” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (x) and lambda (k) light chains refer to the two major antibody light chain isotypes.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Preferred cancers treated by the methods described herein include multiple myeloma, Hodgkin's lymphoma or non-Hodgkin's lymphoma.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

By “a combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. In will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In some embodiments, the additional therapeutic agent is administered at a therapeutic or lower-than therapeutic dose. In certain embodiments, the concentration of the second therapeutic agent that is required to achieve inhibition, e.g., growth inhibition, is lower when the second therapeutic agent is administered in combination with the first therapeutic agent, than when the second therapeutic agent is administered individually. In certain embodiments, the concentration of the first therapeutic agent that is required to achieve inhibition, e.g., growth inhibition, is lower when the first therapeutic agent is administered in combination with the second therapeutic agent than when the first therapeutic agent is administered individually. In certain embodiments, in a combination therapy, the concentration of the second therapeutic agent that is required to achieve inhibition, e.g., growth inhibition, is lower than the therapeutic dose of the second therapeutic agent as a monotherapy, e.g., 10-20%, 20-30%, 30-40%, 40-50%, 50-60%, 60-70%, 70-80%, or 80-90% lower. In certain embodiments, in a combination therapy, the concentration of the first therapeutic agent that is required to achieve inhibition, e.g., growth inhibition, is lower than the therapeutic dose of the first therapeutic agent as a monotherapy, e.g., 10-20%, 20-30%, 30-40%, 40-50%, 50-60%, 60-70%, 70-80%, or 80-90% lower.

The term “inhibition,” “inhibitor,” or “antagonist” includes a reduction in a certain parameter, e.g., an activity, of a given molecule. For example, inhibition of an activity of at least 5%, 10%, 20%, 30%, 40%, 50%, 60%, 70%, 80%, 90%, 95%, 98%, 99%, or more is included by this term. Thus, inhibition need not be 100%.

The term “activation,” “activator,” or “agonist” includes an increase in a certain parameter, e.g., an activity, of a given molecule. For example, increase of an activity of at least 5%, 10%, 20%, 30%, 40%, 50%, 60%, 70%, 80%, 90%, or more, or at least 1, 2, 3, 4, 5, 6, 7, 8, 9, 10-fold, or more, is included by this term.

The term “derived from,” indicates a relationship between a first and a second molecule. It generally refers to structural similarity between the first molecule and a second molecule and does not connotate or include a process or source limitation on a first molecule that is derived from a second molecule.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “expression vector” refers to a vector comprising a recombinant polynucleotide comprising expression control sequences operatively linked to a nucleotide sequence to be expressed.

An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrastemal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the disclosure). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “subject” is intended to include living organisms suitable for an AAV-based gene therapy, e.g., mammals, human.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or“transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a binding partner (e.g., a tumor antigen) protein present in a sample, but which antibody or ligand does not substantially recognize or bind other molecules in the sample.

Ranges: throughout this disclosure, various aspects of the disclosure can be presented in a range format. It should be understood that the description in range format is merely for convenience and brevity and should not be construed as an inflexible limitation on the scope of the disclosure.

Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

A “gene editing system” as the term is used herein, refers to a system, e.g., one or more molecules, that direct and effect an alteration, e.g., a deletion, of one or more nucleic acids at or near a site of genomic DNA targeted by said system. Gene editing systems are known in the art, and are described more fully below.

A “dominant negative” gene product or protein is one that interferes with the function of another gene product or protein. The other gene product affected can be the same or different from the dominant negative protein. Dominant negative gene products can be of many forms, including truncations, full length proteins with point mutations or fragments thereof, or fusions of full-length wild type or mutant proteins or fragments thereof with other proteins. The level of inhibition observed can be very low. For example, it may require a large excess of the dominant negative protein compared to the functional protein or proteins involved in a process in order to see an effect. It may be difficult to see effects under normal biological assay conditions. As used herein, the term “AAV particle” refers to a viral particle derived from or comprising one or more nucleic acid sequences derived from an adeno-associated virus serotype, including without limitation, an AAV-1, AAV-2, AAV-3, AAV-4, AAV-5, AAV-6, AAV-7, AAV-8 or AAV-9 viral vector. AAV particles may have one or more of the AAV wild-type genes deleted in whole or part, e.g., the rep and/or cap genes, while retaining, e.g., functional flanking inverted terminal repeat (“ITR”) sequences. In some embodiments, an AAV particle may be packaged in a protein shell or capsid, e.g., comprising one or more AAV capsid proteins, which may provide a vehicle for delivery of vector nucleic acid to the nucleus of target cells. In some embodiments, an AAV particle comprises one or more AAV ITR sequences (e.g., AAV2 ITR sequences). In some embodiments, an AAV particle comprises one or more AAV ITR sequences (e.g., AAV2 ITR sequences) but does not contain any additional viral nucleic acid sequence. In some embodiments, the AAV particle components (e.g., ITRs) are derived from a different serotype virus than the rAAV capsid (for example, the AAV particle may comprise ITRs derived from AAV2 and the AAV particle may be packaged into an AAV9 capsid). Embodiments of these viral particle constructs are provided, e.g., in WO/2019/094253 (PCT/US2018/058744), which is incorporated herein by reference in its entirety.

Exemplary Genes and Gene Products Associated with AAV Transduction Efficiency

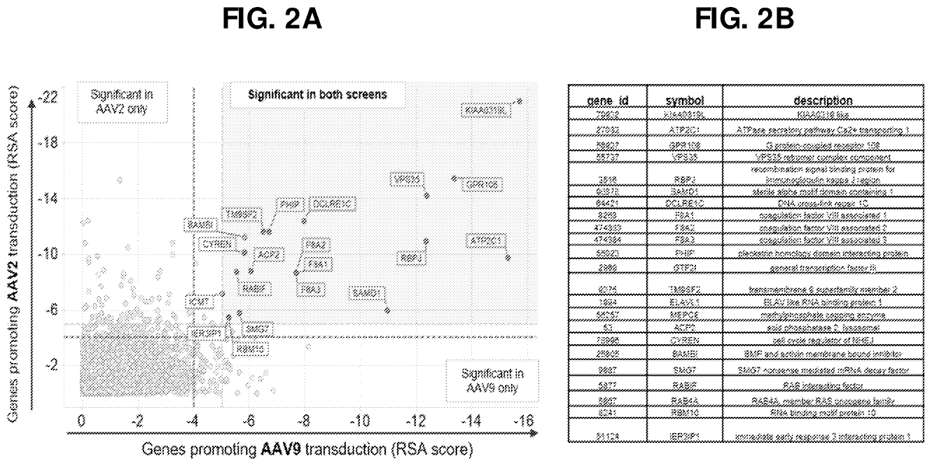

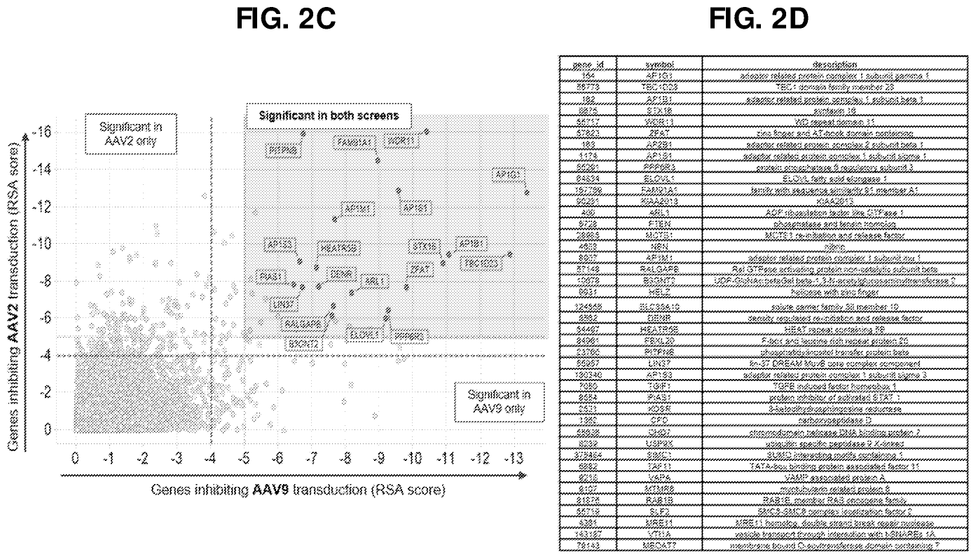

The present disclosure provides genes and gene products associated with AAV transduction efficiency. AAV modulators as described herein can be designed, made, and used based on the discovery of these genes and gene products. In some instances, an AAV modulator as described herein modulates (e.g., increases or decreases) an activity and/or level of a gene or gene product associated with AAV transduction efficiency (e.g., as described herein). In some instances, an exemplary gene or gene product associated with AAV transduction efficiency are described in any of FIGS. 2A-6B.

In some embodiments, the gene or gene product is AAV-R, GPR108, WDR11, or MRE11. In some embodiments, the gene or gene product is AAV-R. In some embodiments, the gene or gene product is GPR108. In some embodiments, the gene or gene product is WDR11. In some embodiments, the gene or gene product is MRE11.

Exemplary Genes and Gene Products that Specifically Promote AAV2 Transduction

In some embodiments, the gene or gene product increases the transduction efficiency of an AAV2 particle. In certain embodiments, the gene or gene product or gene or gene product does not increase, or does not substantially increase, the transduction efficiency of an AAV9 particle. In some embodiments, the gene or gene product is necessary for the transduction of an AAV2 particle. In certain embodiments, the gene or gene product is not necessary for the transduction of an AAV9 particle.

Exemplary genes or gene products include, but are not limited to, EXT1, EXT2, NDST1, BCL10, OAF, PDCD10, LMO4, MALT1, ZNF644, CHUK, GLCE, EP300, ARID4B, RAB21, MED13, DHX15, WASHC5, IKBKB, USP24, HSPA14, CXXC1, UBE2A, INTS8, CDC42, NFKB1, SIN3A, USP7, ARF5, CARD10, JAK1, SLC35C2, PAXK1, ZC3H11A, FAM20B, FAM72A, DHX36, INTS12, and RPRD1B.

In some embodiments, the gene or gene product is associated with endosomal sorting (e.g., RAB21, WASHC5, or USP7). In some embodiments, the gene or gene product is associated with vesicle-mediated transport (e.g., CDC42 or ARF5). In some embodiments, the gene or gene product is associated with NFKB signaling (e.g., BCL10, MALT1, NFKB1, IKBKB, CHUK, or CARD10). In some embodiments, the gene or gene product is associated with heparan sulfate biosynthesis (e.g., EXT1, EXT2, NDST1, GLCE, or FAM20B). In some embodiments, the gene or gene product is associated with H3K9 methylation (e.g., ZNF644). In some embodiments, the gene or gene product is associated with pre-mRNA processing (e.g., DHX15). In some embodiments, the gene or gene product is associated with transcription (e.g., LMO4, EP300, MED13, SIN3, ARID4B, ZC3H11A, CXXC1, RPRD1B, or UBE2A). In some embodiments, the gene or gene product is associated with integrator complex and/or RNA polymerase II function (e.g., INTS8 or INST12). In some embodiments, the gene or gene product is associated with DNA repair and/or AAV genome resolution (e.g., DHX36, ARID4B, UBE2A, or USP24). In some embodiments, the gene or gene product is associated with cell proliferation and/or apoptosis (e.g., PDCD10). In some embodiments, the gene or gene product is associated with spondylocarpotarsal synostosis syndrome (e.g., OAF). In some embodiments, the gene or gene product is associated with protein folding (e.g., HSPA14). In some embodiments, the gene or gene product is associated with helicase activity (e.g., DHX36). In some embodiments, the gene or gene product is associated with fucosylation of Notch and/or transport of GCP-fucose (e.g., SLC35C2).

Exemplary Genes and Gene Products that Specifically Promote AAV9 Transduction

In some embodiments, the gene or gene product increases the transduction efficiency of an AAV9 particle. In certain embodiments, the gene or gene product does not increase, or does not substantially increase, the transduction efficiency of an AAV2 particle. In some embodiments, the gene or gene product is necessary for the transduction of an AAV9 particle. In certain embodiments, the gene or gene product is not necessary for the transduction of an AAV2 particle.

Exemplary genes and gene products include, but are not limited to, TMPRSS11B, HTT, SNRNP70, BRD7, DMXL1, RAB10, CNGA1, KLHDC3, CHP1, CYP3A5, ELOVL4, PNISR, ZCCHC14, AZGP1, TPST1, SC5D, ELP2, ELP3, IPO9, RAB14, WDR7, XRCC4, and GDI2.

In some embodiments, the gene or gene product is associated with microtubule-mediated transport or vesicle function (e.g., HT). In some embodiments, the gene or gene product is an ion channel (e.g., CNGA1). In some embodiments, the gene or gene product is associated with nuclear protein import (e.g., IPO9). In some embodiments, the gene or gene product is associated with O-sulfation of a tyrosine residue within an acidic motif of a polypeptide (e.g., TPST1). In some embodiments, the gene or gene product is associated with protection of viral RNA (e.g., ZCCHC14). In some embodiments, the gene or gene product is associated with RNA binding by an AAV protein (e.g., PNISR). In some embodiments, the gene or gene product is associated with calcium ion-regulated exocytosis (e.g., CHP1). In some embodiments, the gene or gene product is associated with intracellular trafficking (e.g., RAB10, RAB14, GDI2, WDR7, or DMXL1). In some embodiments, the gene or gene product is a subunit (e.g., a catalytic tRNA acetyltransferase subunit) of an elongator complex, e.g., of an RNA polymerase (e.g., RNA polymerase II), e.g., ELP2 or ELP3. In some embodiments, the gene or gene product binds to chromatin (e.g., KLHDC3). In some embodiments, the gene or gene product is associated with cholesterol biosynthesis (e.g., SC5D).

Exemplary Genes and Gene Products that Promote AAV2 and AAV9 Transduction

In some embodiments, the gene or gene product increases the transduction efficiency of AAV2 and AAV9 particles. In some embodiments, the gene or gene product is necessary for the transduction of AAV2 and AAV9 particles.

Exemplary genes and gene products include, but are not limited to, KIAA0319L, BAMBI, TM9SF2, PHIP, DCLRE1C, VPS35, GPR108, CYREN, ACP2, F8A2, F8A1, F8A3, RBPJ, ATP2C1, ICMT, RABIF, IER3IP1, RBM10, SMG7, GTF2I, ELAVL1, MEPCE, RAB4A, IER3IP1, and SAMD1.

In some embodiments, the gene or gene product is associated with influenza infection (e.g., ACP2). In some embodiments, the gene or gene product binds to unmethylated CGIs (e.g., SAMD1). In some embodiments, the gene or gene product is associated with intracellular vesicular transport (e.g., RABIF). In some embodiments, the gene or gene product is associated with posttranslational modification of cysteine residues (e.g., C-terminal cysteine residues), e.g., in a target protein (e.g., ICMT). In some embodiments, the gene or gene product binds to a capsid protein of AAV (e.g., KIAA0319L). In some embodiments, the gene or gene product is associated with inhibition of a TLR (e.g., TLR9), e.g., GPR108. In some embodiments, the gene or gene product is associated with endosome trafficking, e.g., of AAV-R (e.g., VPS35). In some embodiments, the gene or gene product is a calcium ATPase pump (e.g., ATP2C1). In some embodiments, the gene or gene product is associated with Notch signaling (e.g., RBPJ). In some embodiments, the gene or gene product is associated with HSPG metabolism (e.g., TM9SF2). In some embodiments, the gene or gene product is associated with inhibition of NHEJ (e.g., CYREN). In some embodiments, the gene or gene product is associated with viral hairpin resolution (e.g., DCLRE1C). In some embodiments, the gene or gene product is associated with Glc transporter translocation. In certain embodiments, the gene or gene product binds to insulin receptor substrate 1 protein (e.g., PHIP).

Exemplary Genes and Gene Products that Specifically Inhibit AAV2 Transduction

In some embodiments, the gene or gene product decreases the transduction efficiency of an AAV2 particle. In certain embodiments, the gene or gene product, when modulated, does not decrease, or does not substantially decrease, the transduction efficiency of an AAV9 particle. In some embodiments, the gene or gene product inhibits or prevents the transduction of an AAV2 particle. In certain embodiments, the gene or gene product does not inhibit or prevent the transduction of an AAV9 particle.

Exemplary genes and gene products include, but are not limited to, GREB1L, BIRC2, TYMSOS, MSL3, SIK3, GTF2H5, PIGW, ATRAID, PPP4R1, ZFC3H1, SETDB1, SMCHD1, TMEM123, UHRF1, CLUL1, SMAD5, SETX, STEEP1, PSIP1, DENND6A, RTN4RL2, MTMR9, STRADA, PSIP1, BMPR1A, HIKESHI, AP3M1, MEN1, MYL12B, CREB1, RAB12, SMTNL1, MRGBP, WTAP, SUMO2, TCN1, ALDH3B1, ARL14EP, MYL12A, RHOD, PGAP2, EIF4A1, PIGN, DAXX, HDLBP, GOLGA1, SARNP, UBE2L6, TRIM49, SIK2, SOX4, OR6Q1, OR4D6, UNC93B1, CATSPERZ, PIGT, PYM1, MICOS13, RIN1, DOT1L, TRIM49C, MAPK20, COL8A1, FASN, KDM3B, and ELP3.

In some embodiments, the gene or gene product is associated with a retinoic acid receptor activity (e.g., GREB1L).

In some embodiments, the gene or gene product is a thymidylate synthetase (e.g., TYMSOS).

In some embodiments, the gene or gene product is associated with chromatin remodeling (e.g., SMCHD1). In some embodiments, the gene or gene product is associated with histone H4 acetylation (e.g., MSL3). In some embodiments, the gene or gene product is associated with transcription and/or DNA repair (e.g., GTF2H5). In some embodiments, the gene or gene product is a histone methyltransferase (e.g., SETDB1). In some embodiments, the gene or gene product is associated with exosomal degradation of polyadenylated RNA (e.g., ZFC3H1). In some embodiments, the gene or gene product is associated with histone deacetylase recruitment to a DNA (e.g., UHRF1). In some embodiments, the gene or gene product is associated with oxidative stress-induced DNA double strand break response (e.g., SETX). In some embodiments, the gene or gene product is associated with histone modification (e.g., MEN1). In some embodiments, the gene or gene product is associated with nucleosome/DNA interaction (e.g., MRGBP). In some embodiments, the gene or gene product is associated with GPI biosynthesis (e.g., PIGW, PIGN, PIGT, PIGF, PIGA, or PIGY). In some embodiments, the gene or gene product is associated with maturation of GPI anchors (e.g., PGAP2). In some embodiments, the gene or gene product is associated with vesicle-mediated endocytosis (e.g., DENND6A, GOLGA1, MTMR9, GPC3, PPP4R1, RAB12, RAB31, RHOD, RIN1, TMEM123, or TRAPPC2L). In some embodiments, the gene or gene product is associated with clathrin-coated vesicle-mediated endocytosis (e.g., AP3M1). In some embodiments, the gene or gene product is associated with a BMP receptor kinase (e.g., SMAD5, BMPR1A, or CREB1). In some embodiments, the gene or gene product is associated with transcription (e.g., PSIP1). In some embodiments, the gene or gene product is associated with nuclear import of an HSP70 protein (e.g., HIKESHI). In some embodiments, the gene or gene product is associated with a Ser/Thr kinase (e.g., SIK2 or SIK3). In some embodiments, the gene or gene product is associated with SUMOylation of a protein (e.g., SUMO2, UBE2L6).

Exemplary Genes and Gene Products that Specifically Inhibit AAV9 Transduction

In some embodiments, the gene or gene product decreases the transduction efficiency of an AAV9 particle. In certain embodiments, the gene or gene product does not decrease, or does not substantially decrease, the transduction efficiency of an AAV2 particle. In some embodiments, the gene or gene product inhibits or prevents the transduction of an AAV9 particle. In certain embodiments, the gene or gene product does not inhibit or prevent the transduction of an AAV2 particle.

Exemplary genes and gene products include, but are not limited to, AP2A1, AP2B1, TM9SF4, ACTR5, PTMA, PAPOLA, PDHA1, PDHB, SLC25A19, GAK, AUNIP, FCHO2, ACTB, LIPT1, UCHL5, INO80E, ECHS1, NELFB, EDC4, PRMT1, PDS5B, PELI3, FBXL20, and SLC35A1.

In some embodiments, the gene or gene product binds to ATP, a chaperone protein, and/or clathrin (e.g., GAK). In some embodiments, the gene or gene product is associated with clathrin-coated vesicle trafficking (e.g., AP2B1 or AP2A1). In some embodiments, the gene or gene product is associated with clathrin-mediated endocytosis (e.g., FCHO2). In some embodiments, the gene or gene product is associated with vesicular trafficking (e.g., ACTB). In some embodiments, the gene or gene product is associated with an innate immune response (e.g., PELI3 or FBXL20). In some embodiments, the gene or gene product is associated with glucose metabolism (e.g., PDHB or PDHA1). In some embodiments, the gene or gene product is associated with DNA resection and/or homologous recombination, e.g., after DNA damage (e.g., AUNIP). In some embodiments, the gene or gene product is associated with DNA repair, e.g., after DNA damage (e.g., UCHL5, INO80E, PDS5B, ACTR5, or PRMT1). In some embodiments, the gene or gene product is associated with transport of CMP-sialic acid from cytosol to a Golgi vesicle (e.g., SLC35A1). In some embodiments, the gene or gene product is associated with localization of polypeptides comprising glycine-rich transmembrane domains, e.g., to the cell surface (e.g., TM9SF4). In some embodiments, the gene or gene product is associated with poly(A) tail synthesis (e.g., PAPOLA). In some embodiments, the gene or gene product is PTMA. In some embodiments, the gene or gene product is associated with elongation of an mRNA by RNA polymerase II (e.g., NELFB). In some embodiments, the gene or gene product is associated with mRNA degradation (e.g., EDC4). In some embodiments, the gene or gene product encodes a mitochondrial lipoyltransferase (e.g., LIPT1). In some embodiments, the gene or gene product is associated with mitochondrial fatty acid beta-oxidation (e.g., ECHS1). In some embodiments, the gene or gene product encodes a mitochondrial thiamine pyrophosphate carrier (e.g., SLC25A19).

Exemplary Genes and Gene Products that Specifically Inhibit AAV2 and AAV9 Transduction

In some embodiments, the gene or gene product decreases the transduction efficiency of AAV2 and AAV9 particles. In some embodiments, the gene or gene product inhibits or prevents the transduction of AAV2 and AAV9 particles.

Exemplary genes and gene products include, but are not limited to, PITPNB, PITP, FAM91A1, WDR11, AP1G1, AP1M1, AP1S1, AP1S3, HEATR5B, STX16, AP1B1, PIAS1, DENR, ARL1, ZFAT, TBC1D23, LIN37, RALGAPB, B3GNT2, ELOVL1, AP2B1, KIAA2013, PTEN, MCTS1, NBN, HELZ, SLC38A10, FBXL20, TGIF1, KDSR, CPD, CHD7, USP9X, SIMC1, TAF11, VAPA, MTMR6, RAB1B, SLF2, MRE11, VTI1A, MBOAT7, and PPP6R3.

In some embodiments, the gene or gene product is associated with the long-chain FA elongation cycle (e.g., ELOVL1). In some embodiments, the gene or gene product is associated with lipoic acid biosynthesis (e.g., PIAS1). In some embodiments, the gene or gene product is associated with a protein phosphatase catalytic subunit (e.g., PPP6R3). In some embodiments, the gene or gene product is associated with the WDR11 pathway (e.g., WDR11, FAM91A1, AP1G1, AP1S1, AP1M1, AP1S3, or TBC1D23). In some embodiments, the gene or gene product is associated with retrograde transport from the Golgi apparatus to the endoplasmic reticulum, e.g., of PI and/or PC (e.g., PITP). In some embodiments, the gene or gene product is associated with vesicular transport from a late endosome to a trans-Golgi network (e.g., STX16). In some embodiments, the gene or gene product is associated with an activity or recruitment of a golgin, arfaptin, and/or Arf-GEF to a trans-Golgi network (e.g., ARL1). In some embodiments, the gene or gene product encodes a component of a clathrin-dependent vesicle and/or is associated with AP1G1/AP-1-mediated protein trafficking (e.g., HEATR5B).

As used herein, the term “EXT1,” “exostosin glycosyltransferase 1” refers to the gene EXT1 and the gene product encoded by the EXT1 gene. It is also known as “EXT,” “LGS,” “TTV,” “LGCR,” or “TRPS2.” In the human genome, EXT1 is located on chromosome 8. Exemplary human EXT1 transcript sequences include, but are not limited to, NCBI Reference Sequence: NM_000127, GI: 1777425437. Exemplary human EXT1 protein sequences include, but are not limited to, NCBI Reference Sequences: NP_000118.2 GI: 46370066 and Q16394.2 GI: 20141422. Without wishing to be bound by theory, it is believed that in some embodiments, EXT1 is involved in heparan sulfate biosynthesis.

As used herein, the term “EXT2,” “exostosin glycosyltransferase 2” refers to the gene EXT2 and the gene product encoded by the EXT2 gene. It is also known as “SOTV,” or “SSMS.” In the human genome, EXT2 is located on chromosome 11. Exemplary human EXT2 transcript sequences include, but are not limited to, NCBI Reference Sequences: NM_207122.2 GI: 1779821707, NM_000401.3 GI: 296010872, NM_001178083.3 GI: 1889698717, NM_001389628.1 GI: 1953526484, NM_001389630.1 GI: 1953526464, XM_024448383.1 GI: 1370458892, and XM_011519950.1 GI: 767965526. Exemplary human EXT2 protein sequences include, but are not limited to, NCBI Reference Sequences: NP_997005.1 GI: 46370069, NP_000392.3 GI: 296010873, NP_001171554.1 GI: 296010875, NP_001376557.1 GI: 1953526485, NP_001376559.1 GI: 1953526465, XP_011518252.1 GI: 767965527, and XP_024304151.1 GI: 1370458893. Without wishing to be bound by theory, it is believed that in some embodiments, EXT2 is involved in heparan sulfate biosynthesis.

As used herein, the term “NDST1,” “N-deacetylase and N-sulfotransferase 1” refers to the gene NDST1 and the gene product encoded by the NDST1 gene. It is also known as “HSST,” “MRT46,” or “NST1.” In the human genome, NDST1 is located on chromosome 5. Exemplary human NDST1 transcript sequences include, but are not limited to, NCBI Reference Sequences: NM_001543.5 GI: 1519314701 and NM_001301063.2 GI: 1676319301. Exemplary human NDST1 protein sequences include, but are not limited to, NCBI Reference Sequences: NP_001534.1 GI: 4505351 and NP_001287992.1 GI: 666637969. Without wishing to be bound by theory, it is believed that in some embodiments, NDST1 is involved in heparan sulfate biosynthesis.

As used herein, the term “BCL10,” “BCL10 immune signaling adaptor” refers to the gene BCL10 and the gene product encoded by the BCL10 gene. It is also known as “CARMEN,” “CIPER,” “IMD37”, “c-E10”, or “mE10.” In the human genome, BCL10 is located on chromosome 1. Exemplary human BCL10 transcript sequences include, but are not limited to, NCBI Reference Sequences: NM_003921.5 GI: 1388742734, NM_001320715.2 GI: 1676440509, XM_011542398.2 GI: 1034563099, XM_011542397.3 GI: 1370455114, and XM_011542399.2 GI: 1034563100. Exemplary human BCL10 protein sequences include, but are not limited to, NCBI Reference Sequences: NP_003912.1 GI: 4502379, NP_001307644.1 GI: 1002639417, XP_011540699.1 GI: 767906661, XP_011540700.1 GI: 767906663, and XP_011540701.1 GI: 767906666. Without wishing to be bound by theory, it is believed that in some embodiments, BCL10 is involved in NFKB signaling.

As used herein, the term “OAF,” “out at first homolog” refers to the gene OAF and the gene product encoded by the OAF gene. It is also known as “NS5ATP13TP2.” In the human genome, OAF is located on chromosome 11. Exemplary human OAF transcript sequences include, but are not limited to, NCBI Reference Sequences: NM_178507.4. Exemplary human OAF protein sequences include, but are not limited to, NCBI Reference Sequences: NP_848602.1.

As used herein, the term “PDCD10,” “programmed cell death 10” refers to the gene PDCD10 and the gene product encoded by the PDCD10 gene. It is also known as “CCM3,” or “TFAR15.” In the human genome, PDCD10 is located on chromosome 3. Exemplary human PDCD10 transcript sequences include, but are not limited to, NCBI Reference Sequences: NM_007217.4 GI: 1520687835, NM_145859.2 GI: 1890280404, NM_145860.2 GI: 1890262549, XM_005247086.5 GI: 1370483092, XM_005247087.5 GI: 1370483093, XM_005247088.4 GI: 1370483087, XM_006713485.4 GI: 1370483091, XM_011512368.3 GI: 1370483088, XM_011512369.3 GI: 1370483089, XM_017005644.2 GI: 1370483090, XM_017005645.2 GI: 1370483096, XM_024453329.1 GI: 1370483094, XM_024453330.1 GI: 1370483097 and XM_024453331.1 GI: 1370483099. Exemplary human PDCD10 protein sequences include, but are not limited to, NCBI Reference Sequences: NP_009148.2 GI: 20127517, NP_665858.1 GI: 22538792, NP_665859.1 GI: 22538794, XP_005247143.1 GI: 530373856, XP_005247144.1 GI: 530373858, XP_005247145.1 GI: 530373860, XP_006713548.1 GI: 578807020, XP_011510670.1 GI: 767925584, XP_011510671.1 GI: 767925586, XP_016861133.1 GI: 1034630983, XP_016861134.1 GI: 1034630989, XP_024309097.1 GI: 1370483095, XP_024309098.1 GI: 1370483098, and XP_024309099.1 GI: 1370483100. Without wishing to be bound by theory, it is believed that in some embodiments, PDCD10 is involved in cell proliferation and/or apoptosis.

As used herein, the term “LMO4,” “LIM domain only 4” refers to the gene LMO4 and the gene product encoded by the LMO4 gene. It is also known as “LIM-Only 4 Protein,” “LMO-4,” or “LIM Domain Transcription Factor LMO4.” In the human genome, LMO4 is located on chromosome 1. Exemplary human LMO4 transcript sequences include, but are not limited to, NCBI Reference Sequences: NM_006769.4 GI: 1519245993 and NM_001369491.1 GI: 1610577010. Exemplary human LMO4 protein sequences include, but are not limited to, NCBI Reference Sequences: NP_006760.1 GI: 5803072 and NP_001356420.1 GI: 1610577011. Without wishing to be bound by theory, it is believed that in some embodiments, LMO4 is associated with transcription.

As used herein, the term “MALT1,” “MALT1 paracaspase” refers to the gene MALT1 and the gene product encoded by the MALT1 gene. It is also known as “IMD12,” “MLT1,” “MLT”, or “PCASP1.” In the human genome, MALT1 is located on chromosome 18. Exemplary human MALT1 transcript sequences include, but are not limited to, NCBI Reference Sequences: NM_006785.4 GI: 1393270561, NM_173844.3 GI: 1675049238, XM_011525794.1 GI: 767998143, XR_001753134.1 GI: 1034603353, XR_001753135.1 GI: 1034603354, and XR_001753136.1 GI: 1034603355. Exemplary human MALT1 protein sequences include, but are not limited to, NCBI Reference Sequences: NP_006776.1 GI: 5803078, NP_776216.1 GI: 27886566, and XP_011524096.1 GI: 767998144. Without wishing to be bound by theory, it is believed that in some embodiments, MALT1 is involved in NFKB signaling.

As used herein, the term “ZNF644,” “zinc finger protein 644” refers to the gene ZNF644 and the gene product encoded by the ZNF644 gene. It is also known as “BM-005,” “MYP21,” “ZEP-2”, or “NatF.” In the human genome, ZNF644 is located on chromosome 1. Exemplary human ZNF644 transcript sequences include, but are not limited to, NCBI Reference Sequences: NM_201269.3 GI: 1519312808, NM_016620.4 GI: 1890327857, NM_032186.5 GI: 1890272283, XM_005271257.5 GI: 1370454817, XM_005271260.5 GI: 1370454818, XM_011542258.3 GI: 1370454816, XM_011542259.3 GI: 1370454819, XM_011542260.3 GI: 1370454820, XM_011542261.3 GI: 1370454822, XM_017002487.2 GI: 1370454821, XM_017002488.2 GI: 1370454823, XM_017002489.2 GI: 1370454824, XM_017002490.2 GI: 1370454825, XM_017002491.2 GI: 1370454826, XM_017002492.2 GI: 1370454827, XM_017002493.2 GI: 1370454828, and XM_017002494.1 GI: 1034562409. Exemplary human ZNF644 protein sequences include, but are not limited to, NCBI Reference Sequences: NP_958357.1 GI: 41152093, NP_057704.2 GI: 41152095, NP_115562.3 GI: 41152091, XP_005271314.1 GI: 530363442, XP_005271317.1 GI: 530363448, XP_011540560.1 GI: 767906309, XP_011540561.1 GI: 767906312, XP_011540562.1 GI: 767906314, XP_011540563.1 GI: 767906317, XP_016857976.1 GI: 1034562395, XP_016857977.1 GI: 1034562398, XP_016857978.1 GI: 1034562400, XP_016857979.1 GI: 1034562402, XP_016857980.1 GI: 1034562404, XP_016857981.1 GI: 1034562406, XP_016857982.1 GI: 1034562408, and XP_016857983.1 GI: 1034562410. Without wishing to be bound by theory, it is believed that in some embodiments, ZNF644 is involved in H3K9 methylation.

As used herein, the term “CHUK,” “component of inhibitor of nuclear factor kappa B kinase complex” refers to the gene CHUK and the gene product encoded by the CHUK gene. It is also known as “BPS2,” “IKBKA,” “IKK-alpha,” “IKK1,” “IKKA,” “NFKBIKA”, or “TCF16.” In the human genome, CHUK is located on chromosome 10. Exemplary human CHUK transcript sequences include, but are not limited to, NCBI Reference Sequences: NM_001278.5 GI: 1519245267, NM_001320928.2 GI: 1890265705, XM_017015611.1 GI: 1034566253, XM_017015612.1 GI: 1034566256, XM_017015613.1 GI: 1034566258, XR_001747010.1 GI: 1034566255, and XR_001747011.1 GI: 1034566260. Exemplary human CHUK protein sequences include, but are not limited to, NCBI Reference Sequences: NP_001269.3 GI: 62241001, NP_001307857.1 GI: 1004170674, XP_016871100.1 GI: 1034566254, XP_016871101.1 GI: 1034566257, and XP_016871102.1 GI: 1034566259. Without wishing to be bound by theory, it is believed that in some embodiments, CHUK is involved NFKB signaling.

As used herein, the term “GLCE,” “glucuronic acid epimerase” refers to the gene GLCE and the gene product encoded by the GLCE gene. It is also known as “HSEPI.” In the human genome, GLCE is located on chromosome 15. Exemplary human GLCE transcript sequences include, but are not limited to, NCBI Reference Sequences: NM_015554.3 GI: 1519244225 NM_001324091.2 GI: 1890343420, NM_001324092.2 GI: 1675178323, NM_001324093.2 GI: 1676317756, NM_001324094.2 GI: 1675094819, XM_005254298.3 GI: 1034590279, XM_017022073.1 GI: 1034590277, and XM_017022074.2 GI: 1370466481. Exemplary human GLCE protein sequences include, but are not limited to, NCBI Reference Sequences: NP_056369.1 GI: 51317380, NP_001311020.1 GI: 1022943262, NP_001311021.1 GI: 1022943153, NP_001311022.1 GI: 1022943120, NP_001311023.1 GI: 1022943129, XP_005254355.1 GI: 530405672, XP_016877562.1 GI: 1034590278, and XP_016877563.1 GI: 1034590281. Without wishing to be bound by theory, it is believed that in some embodiments, GLCE is involved in heparan sulfate biosynthesis.

As used herein, the term “EP300,” “E1A binding protein p300” refers to the gene EP300 and the gene product encoded by the EP300 gene. It is also known as “KAT3B,” “MKHK2,” “RSTS2”, or “p300.” In the human genome, EP300 is located on chromosome 22. Exemplary human EP300 transcript sequences include, but are not limited to, NCBI Reference Sequences: NM_001429.4 GI: 1519315586 and NM_001362843.2 GI: 1675014866. Exemplary human EP300 protein sequences include, but are not limited to, NCBI Reference Sequences: NP_001420.2 GI: 50345997 and NP_001349772.1 GI: 1384865987. Without wishing to be bound by theory, it is believed that in some embodiments, EP300 is involved in transcription.

As used herein, the term “ARID4B,” “AT-rich interaction domain 4B refers to the gene ARID4B and the gene product encoded by the ARID4B gene. It is also known as “BCAA,” “BRCAA1,” “RBBP1L1,” “RBP1L1,” or “SAP180.” In the human genome, ARID4B is located on chromosome 1. Exemplary human ARID4B transcript sequences include, but are not limited to, NCBI Reference Sequences: NM_016374.6 GI: 1519245583, NM_031371.4 GI: 1675088876, NM_001206794.2 GI: 1890255187, NR_135074.2 GI: 1700447956, XM_006711781.3 GI: 1370453448, XM_011544212.3 GI: 1370453447, XM_017001472.1 GI: 1034559171, XM_024447626.1 GI: 1370453449, XM_024447628.1 GI: 1370453454, XR_001737226.2 GI: 1370453451, XR_001737227.2 GI: 1370453452, and XR_002956802.1 GI: 1370453453. Exemplary human ARID4B protein sequences include, but are not limited to, NCBI Reference Sequences: NP_057458.4 GI: 22035677, NP_112739.2 GI: 22035679, NP_001193723.1 GI: 332164768, XP_006711844.1 GI: 578802247, XP_011542514.1 GI: 767912487, XP_016856961.1 GI: 1034559172, XP_024303394.1 GI: 1370453450, and XP_024303396.1 GI: 1370453455. Without wishing to be bound by theory, it is believed that in some embodiments, ARID4B is involved in DNA repair and/or AAV genome resolution.

As used herein, the term “RAB21,” “RAB21, member RAS oncogene family” refers to the gene RAB21 and the gene product encoded by the RAB21 gene. In the human genome, RAB21 is located on chromosome 12. Exemplary human RAB21 transcript sequences include, but are not limited to, NCBI Reference Sequences: NM_014999.4. Exemplary human RAB21 protein sequences include, but are not limited to, NCBI Reference Sequences: NP_055814.1. Without wishing to be bound by theory, it is believed that in some embodiments, RAB21 is involved in endosomal sorting.

As used herein, the term “MED13,” “mediator complex subunit 13 refers to the gene MED13 and the gene product encoded by the MED13 gene. It is also known as “ARC250,” “DRIP250,” “HSPC221,” “MRD61,” “THRAP1,” or “TRAP240.” In the human genome, MED13 is located on chromosome 17. Exemplary human MED13 transcript sequences include, but are not limited to, NCBI Reference Sequences: NM_005121.3 GI: 1519316353, XM_011525551.2 GI: 1034602548, XM_011525552.2 GI: 1034602549, and XM_011525553.3 GI: 1370472821. Exemplary human MED13 protein sequences include, but are not limited to, NCBI Reference Sequences: NP_005112.2 GI: 102468717, XP_011523853.1 GI: 767996720, XP_011523854.1 GI: 767996722, and XP_011523855.1 GI: 767996724. Without wishing to be bound by theory, it is believed that in some embodiments, MED13 is involved in transcription.

As used herein, the term “DHX15,” “DEAH-box helicase 15” refers to the gene DHX15 and the gene product encoded by the DHX15 gene. It is also known as “DBP1,” “DDX15,” “PRP43,” “PRPF43,” or “PrPp43p.” In the human genome, DHX15 is located on chromosome 4. Exemplary human DHX15 transcript sequences include, but are not limited to, NCBI Reference Sequences: NM_001358.3 GI: 1519245693 and XR_001741152.2 GI: 1370486348. Exemplary human DHX15 protein sequences include, but are not limited to, NCBI Reference Sequences: NP_001349.2. Without wishing to be bound by theory, it is believed that in some embodiments, DHX15 is involved in pre-mRNA processing.

As used herein, the term “WASHC5,” “WASH complex subunit 5” refers to the gene WASHC5 and the gene product encoded by the WASHC5 gene. It is also known as “KIAA0196,” “RTSC,” “RTSC1,” or “SPG8.” In the human genome, WASHC5 is located on chromosome 8. Exemplary human WASHC5 transcript sequences include, but are not limited to, NCBI Reference Sequences: NM_014846.4 GI: 1519314648, NM_001330609.2 GI: 1889621155, XM_011517409.1 GI: 767953896, and XM_017014113.2 GI: 1370513296. Exemplary human WASHC5 protein sequences include, but are not limited to, NCBI Reference Sequences: NP_055661.3 GI: 120952851, NP_001317538.1 GI: 1060604623, XP_011515711.1 GI: 767953897, and XP_016869602.1 GI: 1034662311. Without wishing to be bound by theory, it is believed that in some embodiments, WASHC5 is involved in endosomal sorting.

As used herein, the term “IKBKB,” “inhibitor of nuclear factor kappa B kinase subunit beta refers to the gene IKBKB and the gene product encoded by the IKBKB gene. It is also known as “IKK-beta,” “IKK2,” “IKKB,” “IMD15,” “IMD15A,” “IMD15B,” or “NFKBIKB.” In the human genome, IKBKB is located on chromosome 8. Exemplary human IKBKB transcript sequences include, but are not limited to, NCBI Reference Sequences: NM_001556.3 GI: 1409838258, NM_001190720.3 GI: 1831772119, NM_001242778.2 GI: 1677531062, NR_033818.2 GI: 1700447925, NR_033819.2 GI: 1701971073, NR_040009.2 GI: 1701216173, XM_005273490.3 GI: 1370512308, XM_005273491.5 GI: 1370512309, XM_005273492.4 GI: 1370512310, XM_005273493.4 GI: 1370512311, XM_005273494.3 GI: 1370512312, XM_005273495.2 GI: 1034660193, XM_005273496.4 GI: 1370512313, XM_005273498.4 GI: 1370512314, XM_011544517.2 GI: 1034660185, XM_011544518.2 GI: 1034660186, XM_011544519.2 GI: 1034660188, XM_011544520.2 GI: 1034660189, XM_011544521.2 GI: 1034660195, XM_011544522.2 GI: 1034660200, XM_017013396.1 GI: 1034660198, XR_949402.3 GI: 1370512316, and XR_001745530.2 GI: 1370512315. Exemplary human IKBKB protein sequences include, but are not limited to, NCBI Reference Sequences: NP_001547.1 GI: 41352712, NP_001177649.2 GI: 1831772120, NP_001229707.1 GI: 338753358, XP_005273547.1 GI: 530387747, XP_005273548.1 GI: 530387749, XP_005273549.1 GI: 530387751, XP_005273550.1 GI: 530387753, XP_005273551.1 GI: 530387755, XP_005273552.1 GI: 530387757, XP_005273553.1 GI: 530387759,XP_005273555.1 GI: 530387763, XP_011542819.1 GI: 767950854, XP_011542820.1 GI: 767950856, XP_011542821.1 GI: 767950859, XP_011542822.1 GI: 767950861, XP_011542823.1 GI: 767950865, XP_011542824.1 GI: 767950867, and XP_016868885.1 GI: 1034660199. Without wishing to be bound by theory, it is believed that in some embodiments, IKBKB is involved in NFKB signaling.

As used herein, the term “USP24,” “ubiquitin specific peptidase 24” refers to the gene USP24 and the gene product encoded by the USP24 gene. In the human genome, USP24 is located on chromosome 1. Exemplary human USP24 transcript sequences include, but are not limited to, NCBI Reference Sequences: NM_015306.3 GI: 1519244982, XM_005270690.3 GI: 1034557146, XM_017000831.1 GI: 1034557137, XM_017000832.1 GI: 1034557139, XM_017000833.1 GI: 1034557142, XM_017000834.1 GI: 1034557144, XM_017000835.1 GI: 1034557149, XM_017000836.1 GI: 1034557151, XM_017000837.1 GI: 1034557154, XR_001737080.1 GI: 1034557141, XR_001737081.1 GI: 1034557147, XR_001737082.1 GI: 1034557148, and XR_001737083.1 GI: 1034557153. Exemplary human USP24 protein sequences include, but are not limited to, NCBI Reference Sequences: NP_056121.2 GI: 260064009, XP_005270747.1 GI: 530362260, XP_016856320.1 GI: 1034557138, XP_016856321.1 GI: 1034557140, XP_016856322.1 GI: 1034557143, XP_016856323.1 GI: 1034557145, XP_016856324.1 GI: 1034557150 and XP_016856325.1 GI: 1034557152 Without wishing to be bound by theory, it is believed that in some embodiments, USP24 is involved in DNA repair and/or AAV genome resolution.

As used herein, the term “HSPA14,” “heat shock protein family A (Hsp70) member 14” refers to the gene HSPA14 and the gene product encoded by the HSPA14 gene. It is also known as “HSP70-4” or “HSP70L1.” In the human genome, HSPA14 is located on chromosome 10. Exemplary human HSPA14 transcript sequences include, but are not limited to, NCBI Reference Sequences: NM_016299.4. Exemplary human HSPA14 protein sequences include, but are not limited to, NCBI Reference Sequences: NP_057383.2. Without wishing to be bound by theory, it is believed that in some embodiments, HSPA14 is involved in protein folding.

As used herein, the term “CXXC1,” “CXXC finger protein 1” refers to the gene CXXC1 and the gene product encoded by the CXXC1 gene. It is also known as “2410002I16Rik,” “5830420C16Rik,” “CFP1,” “CGBP,” “HsT2645,” “PCCX1,” “PHF18,” “SPP1,” “ZCGPC1,” or “hCGBP.” In the human genome, CXXC1 is located on chromosome 6. Exemplary human CXXC1 transcript sequences include, but are not limited to, NCBI Reference Sequences: NM_014593.4 GI: 1887789654, NM_001101654.2 GI: 1887789695, XM_011525940.2 GI: 1034603914, XM_011525941.2 GI: 1034603917, XM_017025718.2 GI: 1370473435, and XM_017025719.1 GI: 1034603918. Exemplary human CXXC1 protein sequences include, but are not limited to, NCBI Reference Sequences: NP_055408.2 GI: 156142180, NP_001095124.1 GI: 156142182, XP_011524242.1 GI: 767998613, XP_011524243.1 GI: 767998615, XP_016881207.1 GI: 1034603916 and XP_016881208.1 GI: 1034603919. Without wishing to be bound by theory, it is believed that in some embodiments, CXXC1 is involved in transcription.

As used herein, the term “UBE2A,” “ubiquitin conjugating enzyme E2 A” refers to the gene UBE2A and the gene product encoded by the UBE2A gene. It is also known as “HHR6A,” “MRXS30,” “MRXSN,” “RAD6A,” or “UBC2.” In the human genome, UBE2A is located on chromosome X. Exemplary human UBE2A transcript sequences include, but are not limited to, NCBI Reference Sequences: NM_003336.4 GI: 1519245248, NM_181762.3 GI: 167631938, and NM_001282161.2 GI: 1675115840. Exemplary human UBE2A protein sequences include, but are not limited to, NCBI Reference Sequences: NP_003327.2 GI: 32967280, NP_861427.1 GI: 32967276, and NP_001269090.1 GI: 531990811. Without wishing to be bound by theory, it is believed that in some embodiments, UBE2A is involved in DNA repair and/or AAV genome resolution.

As used herein, the term “INTS8,” “integrator complex subunit 8” refers to the gene INTS8 and the gene product encoded by the INTS8 gene. It is also known as “C8orf52,” “INT8,” or “NEDCHS.” In the human genome, INTS8 is located on chromosome 8. Exemplary human INTS8 transcript sequences include, but are not limited to, NCBI Reference Sequences: NM_017864.4 GI: 1653962538, NR_073444.2 GI: 1890394573, NR_073445.2 GI: 1890526262, XM_017013616.1 GI: 1034660853, XM_017013617.1 GI: 1034660855, XM_017013618.1 GI: 1034660857, and XM_017013619.1 GI: 1034660859. Exemplary human INTS8 protein sequences include, but are not limited to, NCBI Reference Sequences: NP_060334.2 GI: 70608109, XP_016869105.1 GI: 1034660854, XP_016869106.1 GI: 1034660856, XP_016869107.1 GI: 1034660858, and XP_016869108.1 GI: 1034660860. Without wishing to be bound by theory, it is believed that in some embodiments, INTS8 is involved in integrator complex and/or RNA polymerase II function.

As used herein, the term “CDC42,” “cell division cycle 42” refers to the gene CDC42 and the gene product encoded by the CDC42 gene. It is also known as “CDC42Hs,” “G25K,” or “TKS.” In the human genome, CDC42 is located on chromosome 1. Exemplary human CDC42 transcript sequences include, but are not limited to, NCBI Reference Sequences: NM_001791.4 GI: 1530739946, NM_044472.3 GI: 1558440769, and NM_001039802.2 GI: 1675018072. Exemplary human CDC42 protein sequences include, but are not limited to, NCBI Reference Sequences: NP_001782.1 GI: 4757952, NP_426359.1 GI: 16357472, and NP_001034891.1 GI: 89903012. Without wishing to be bound by theory, it is believed that in some embodiments, CDC42 is involved in vesicle-mediated transport.

As used herein, the term “NFKB1,” “nuclear factor kappa B subunit 1” refers to the gene NFKB1 and the gene product encoded by the NFKB1 gene. It is also known as “CVID12,” “EBP-1,” “KBF1,” “NF-kB,” “NF-kB,” “NF-kappa-B1,” “NF-kappaB,” “NF-kappabeta,” “NFKB-p105,” “NFKB-p50,” or “NFkappaB.” In the human genome, NFKB1 is located on chromosome 4. Exemplary human NFKB1 transcript sequences include, but are not limited to, NCBI Reference Sequences: NM_003998.4 GI: 1519314697, NM_001165412.2 GI: 1674985997, NM_001319226.2 GI: 1675096365, NM_001382625.1 GI: 1843419864, NM_001382626.1 GI: 1843419786, NM_001382627.1 GI: 1843419885, NM_001382628.1 GI: 1843419817, XM_024454068.1 GI: 1370486991, and XM_024454069.1 GI: 1370486993. Exemplary human NFKB1 protein sequences include, but are not limited to, NCBI Reference Sequences: NP_003989.2 GI: 34577122, NP_001158884.1 GI: 259155302, NP_001306155.1 GI: 984880773, NP_001369554.1 GI: 1843419865, NP_001369555.1 GI: 1843419787, NP_001369556.1 GI: 1843419886, NP_001369557.1 GI: 1843419818, XP_024309836.1 GI: 1370486992 and XP_024309837.1 GI: 1370486994. Without wishing to be bound by theory, it is believed that in some embodiments, NFKB1 is involved in NFKB signaling.

As used herein, the term “SIN3A,” “SIN3 transcription regulator family member A” refers to the gene SIN3A and the gene product encoded by the SIN3A gene. It is also known as “WITKOS.” In the human genome, SIN3A is located on chromosome 15. Exemplary human SIN3A transcript sequences include, but are not limited to, NCBI Reference Sequences: NM_001145358.2 GI: 1653961902, NM_015477.3 GI: 1676439946, NM_001145357.2 GI: 1675172641, XM_006720465.3 GI: 1034590258, XM_006720466.3 GI: 1034590257, XM_006720467.3 GI: 1034590259 and XM_024449896.1 GI: 1370466468. Exemplary human SIN3A protein sequences include, but are not limited to, NCBI Reference Sequences: NP_001138830.1 GI: 223941785, NP_056292.1 GI: 23397666, NP_001138829.1 GI: 223941782, XP_006720528.1 GI: 578826806, XP_006720529.1 GI: 578826808, XP_006720530.1 GI: 578826810, and XP_024305664.1 GI: 1370466469.

As used herein, the term “USP7,” “ubiquitin specific peptidase 7” refers to the gene USP7 and the gene product encoded by the USP7 gene. It is also known as “HAFOUS,” “HAUSP,” or “TEFL.” In the human genome, USP7 is located on chromosome 16. Exemplary human USP7 transcript sequences include, but are not limited to, NCBI Reference Sequences: NM_003470.3 GI: 1519246443, NM_001286457.2 GI: 1676318454, NM_001286458.2 GI: 1676319373, NM_001321858.2 GI: 1890275316, NR_135826.2 GI: 1889767395, XM_017023652.1 GI: 1034595922, and XM_017023653.2 GI: 1370469174. Exemplary human USP7 protein sequences include, but are not limited to, NCBI Reference Sequences: NP_003461.2 GI: 150378533, NP_001273386.2 GI: 1676318455, NP_001273387.1 GI: 557129022, NP_001308787.1 GI: 1013061510, XP_016879141.1 GI: 1034595923, and XP_016879142.1 GI: 1034595925. Without wishing to be bound by theory, it is believed that in some embodiments, USP7 is involved in endosomal sorting.

As used herein, the term “ARF5,” “ADP ribosylation factor 5” refers to the gene ARF5 and the gene product encoded by the ARF5 gene. In the human genome, ARF5 is located on chromosome 7. Exemplary human ARF5 transcript sequences include, but are not limited to, NCBI Reference Sequences: NM_001662.4. Exemplary human ARF5 protein sequences include, but are not limited to, NCBI Reference Sequences: NP_001653.1. Without wishing to be bound by theory, it is believed that in some embodiments, ARF5 is involved in vesicle-mediated transport.

As used herein, the term “CARD10,” “caspase recruitment domain family member 10” refers to the gene CARD10 and the gene product encoded by the CARD10 gene. It is also known as “BIMP1,” “CARMA3.” In the human genome, CARD10 is located on chromosome 22. Exemplary human CARD10 transcript sequences include, but are not limited to, NCBI Reference Sequences: NM_014550.4. Exemplary human CARD10 protein sequences include, but are not limited to, NCBI Reference Sequences: NP_055365.2. Without wishing to be bound by theory, it is believed that in some embodiments, CARD10 is involved in NFKB signaling.

As used herein, the term “JAK1,” “Janus kinase 1” refers to the gene JAK1 and the gene product encoded by the JAK1 gene. It is also known as “AIIDE,” “JAK1A,” “JAK1B,” or “JTK3.” In the human genome, JAK1 is located on chromosome 1. Exemplary human JAK1 transcript sequences include, but are not limited to, NCBI Reference Sequences: NM_002227.4 GI: 1519311468, NM_001320923.2 GI: 1890333623, NM_001321852.2 GI: 1675178724, NM_001321853.2 GI: 1676439693, NM_001321854.2 GI: 1676319264, NM_001321855.2 GI: 1676324969, NM_001321856.2 GI: 1890266248, and NM_001321857.2 GI: 1676318223. Exemplary human JAK1 protein sequences include, but are not limited to, NCBI Reference Sequences: NP_002218.2 GI: 102469034, NP_001307852.1 GI: 1004007314, NP_001308781.1 GI: 1013071061, NP_001308782.1 GI: 1013070983, NP_001308783.1 GI: 1013071038, NP_001308784.1 GI: 1013071063, NP_001308785.1 GI: 1013070973, and NP_001308786.1 GI: 1013398012.

As used herein, the term “SLC35C2,” “solute carrier family 35 member C2” refers to the gene SLC35C2 and the gene product encoded by the SLC35C2 gene. It is also known as “BA39402.1,” “C20orf5,” “CGI-15” or “OVCOV1.” In the human genome, SLC35C2 is located on chromosome 20. Exemplary human SLC35C2 transcript sequences include, but are not limited to, NCBI Reference Sequences: NM_015945.12 GI: 1519244453, NM_173073.4 GI: 1675112536, NM_173179.4 GI: 1675178534, NM_001281457.2 GI: 1676318312, NM_001281458.2 GI: 1890336228, NM_001281459.2 GI: 1675178824, NM_001281460.2 GI: 1675138001, XM_011528831.1 GI: 768017221, XM_011528832.1 GI: 768017224, XM_011528833.1 GI: 768017227, XM_011528834.2 GI: 1370480506, XM_011528835.2 GI: 1370480507, XM_011528836.1 GI: 768017239, XM_011528837.1 GI: 768017241, XM_011528838.2 GI: 1370480513, XM_017027861.1 GI: 1034625164, XM_017027862.2 GI: 1370480509, XR_936541.2 GI: 1370480508, XR_936543.2 GI: 1370480511, XR_001754283.2 GI: 1370480510, and XR_001754284.2 GI: 1370480512. Exemplary human SLC35C2 protein sequences include, but are not limited to, NCBI Reference Sequences: NP_057029.8 GI: 21314776, NP_775096.1 GI: 27881496, NP_775271.1 GI: 27881499, NP_001268386.1 GI: 527317354, NP_001268387.1 GI: 527317356, NP_001268388.1 GI: 527317358, NP_001268389.1 GI: 527317360, XP_011527133.1 GI: 768017222, XP_011527134.1 GI: 768017225, XP_011527135.1 GI: 768017228, XP_011527136.1 GI: 768017231, XP_011527137.1 GI: 768017234, XP_011527138.1 GI: 768017240, XP_011527139.1 GI: 768017242, XP_016883350.1 GI: 1034625165, and XP_016883351.1 GI: 1034625167. Without wishing to be bound by theory, it is believed that in some embodiments, SLC35C2 is involved in fucosylation of Notch and/or transport of GCP-fucose.

As used herein, the term “ZC3H11A,” “zinc finger CCCH-type containing 11A” refers to the gene ZC3H11A and the gene product encoded by the ZC3H11A gene. It is also known as “ZC3HDC11A.” In the human genome, ZC3H11A is located on chromosome 1. Exemplary human ZC3H11A transcript sequences include, but are not limited to, NCBI Reference Sequences: NM_001376342.1 GI: 1775678349, NM_014827.6 GI: 1779859786, NM_001319238.2 GI: 1779859753, NM_001319239.2 GI: 1779859778, NM_001350261.2 GI: 1779859788, NM_001350262.2 GI: 1779859763, NM_001350263.2 GI: 1779859768, NM_001350264.2 GI: 1779859773, NM_001350265.2 GI: 1779859749, NM_001350266.2 GI: 1779859745, NM_001376334.1 GI: 1775678608, NM_001376335.1 GI: 1775678477, NM_001376336.1 GI: 1775678469, NM_001376337.1 GI: 1775678421, NM_001376338.1 GI: 1775678502, NM_001376339.1 GI: 1775678302, NM_001376340.1 GI: 1775678444, NM_001376341.1 GI: 1775678436, NM_001376343.1 GI: 1775678399, NM_001376344.1 GI: 1775678285, NM_001376345.1 GI: 1775678281, NM_001376346.1 GI: 1775678291, NM_001376347.1 GI: 1775678293, NM_001376348.1 GI: 1775678538, NM_001376349.1 GI: 1775678550, NM_001376350.1 GI: 1775678383, NM_001376351.1 GI: 1775678407, NM_001376352.1 GI: 1775678579, NM_001376353.1 GI: 1775678528, NM_001376354.1 GI: 1775678569, NM_001376355.1 GI: 1775678512, NM_001376356.1 GI: 1775678571, NM_001376357.1 GI: 1775678471, NM_001376358.1 GI: 1775678430, NM_001376359.1 GI: 1775678332, NM_001376360.1 GI: 1775678376, NM_001376361.1 GI: 1775678354, NM_001376362.1 GI: 1775678536, NM_001376363.1 GI: 1775678320, NM_001376364.1 GI: 1775678440, NM_001376365.1 GI: 1775678504, NM_001376366.1 GI: 1775986593, NM_001376367.1 GI: 1775678367, XM_011510201.1 GI: 767911031 and XM_017002959.2 GI: 1370455504. Exemplary human ZC3H11A protein sequences include, but are not limited to, NCBI Reference Sequences: NP_001363271.1 GI: 1775678350, NP_055642.3 GI: 114842410, NP_001306167.1 GI: 984880743, NP_001306168.1 GI: 984880681, NP_001337190.1 GI: 1176461019, NP_001337191.1 GI: 1177678354, NP_001337192.1 GI: 1176461092, NP_001337193.1 GI: 1176461025, NP_001337194.1 GI: 1176461029, NP_001337195.1 GI: 1176461125, NP_001363263.1 GI: 1775678609, NP_001363264.1 GI: 1775678478, NP_001363265.1 GI: 1775678470, NP_001363266.1 GI: 1775678422, NP_001363267.1 GI: 1775678503, NP_001363268.1 GI: 1775678303, NP_001363269.1 GI: 1775678445, NP_001363270.1 GI: 1775678437, NP_001363272.1 GI: 1775678400, NP_001363273.1 GI: 1775678286, NP_001363274.1 GI: 1775678282, NP_001363275.1 GI: 1775678292, NP_001363276.1 GI: 1775678294, NP_001363277.1 GI: 1775678539, NP_001363278.1 GI: 1775678551, NP_001363279.1 GI: 1775678384, NP_001363280.1 GI: 1775678408, NP_001363281.1 GI: 1775678580, NP_001363282.1 GI: 1775678529, NP_001363283.1 GI: 1775678570, NP_001363284.1 GI: 1775678513, NP_001363285.1 GI: 1775678572, NP_001363286.1 GI: 1775678472, NP_001363287.1 GI: 1775678431, NP_001363288.1 GI: 1775678333, NP_001363289.1 GI: 1775678377, NP_001363290.1 GI: 1775678355, NP_001363291.1 GI: 1775678537, NP_001363292.1 GI: 1775678321, NP_001363293.1 GI: 1775678441, NP_001363294.1 GI: 1775678505, NP_001363295.1 GI: 1775986594, NP_001363296.1 GI: 1775678368, XP_011508503.1 GI: 767911032, and XP_016858448.1 GI: 1034563751. Without wishing to be bound by theory, it is believed that in some embodiments, ZC3H11A is involved in transcription.

As used herein, the term “FAM20B,” “FAM20B glycosaminoglycan xylosylkinase” refers to the gene FAM20B and the gene product encoded by the FAM20B gene. It is also known as “gxk1.” In the human genome, FAM20B is located on chromosome 1. Exemplary human FAM20B transcript sequences include, but are not limited to, NCBI Reference Sequences: NM_014864.4 GI: 1519315768, NM_001324310.2 GI: 1890271721, NM_001324311.2 GI: 1890327865, XM_017003001.2 GI: 1370455540, XM_017003002.1 GI: 1034563865, and XR_002958229.1 GI: 1370455541. Exemplary human FAM20B protein sequences include, but are not limited to, NCBI Reference Sequences: NP_055679.1 GI: 7662150, NP_001311239.1 GI: 1024249351, NP_001311240.1 GI: 1024249342, XP_016858490.1 GI: 1034563864, and XP_016858491.1 GI: 1034563866. Without wishing to be bound by theory, it is believed that in some embodiments, FAM20B is involved in heparan sulfate biosynthesis.

As used herein, the term “FAM72A,” “family with sequence similarity 72 member A” refers to the gene FAM72A and the gene product encoded by the FAM72A gene. It is also known as “LMPIP,” “Ugene,” or “p17.” In the human genome, FAM72A is located on chromosome 1. Exemplary human FAM72A transcript sequences include, but are not limited to, NCBI Reference Sequences: NM_001123168.3 GI: 1869284272, NM_001317901.2 GI: 1869284204, NM_001385240.1 GI: 1869284171, NM_001385241.1 GI: 1869284215, NM_001385242.1 GI: 1869284244, NM_001385243.1 GI: 1869284213, NM_001385244.1 GI: 1869284177, NM_001385245.1 GI: 1869284183, NM_001385248.1 GI: 1869284193, NM_001385249.1 GI: 1869284262, NM_001385251.1 GI: 1869284200, NR_134239.2 GI: 1869284280, XM_011509966.3 GI: 1370454438, and XM_024449545.1 GI: 1370454433. Exemplary human FAM72A protein sequences include, but are not limited to, NCBI Reference Sequences: NP_001116640.1 GI: 178057345, NP_001304830.1 GI: 961525476, NP_001372169.1 GI: 1869284172, NP_001372170.1 GI: 1869284216, NP_001372171.1 GI: 1869284245, NP_001372172.1 GI: 1869284214, NP_001372173.1 GI: 1869284178, NP_001372174.1 GI: 1869284184, NP_001372177.1 GI: 1869284194, NP_001372178.1 GI: 1869284263, NP_001372180.1 GI: 1869284201, XP_011508268.1 GI: 767910388 and XP_024305313.1 GI: 1370454434.

As used herein, the term “DHX36,” “DEAH-box helicase 36” refers to the gene DHX36 and the gene product encoded by the DHX36 gene. It is also known as “DDX36,” “G4R1,” “MLEL1”, or “RHAU.” In the human genome, DHX36 is located on chromosome 3. Exemplary human DHX36 transcript sequences include, but are not limited to, NCBI Reference Sequences: NM_020865.3 GI: 1519241676 and NM_001114397.2 GI: 1890274553. Exemplary human DHX36 protein sequences include, but are not limited to, NCBI Reference Sequences: NP_065916.2 GI: 167830433 and NP_001107869.1 GI: 167830436. Without wishing to be bound by theory, it is believed that in some embodiments, DHX36 is involved in helicase activity, DNA repair and/or AAV genome resolution.

As used herein, the term “INTS12,” “integrator complex subunit 12” refers to the gene INTS12 and the gene product encoded by the INTS12 gene. It is also known as “INT12,” “PHF22,” or “SBBI22.” In the human genome, INTS12 is located on chromosome 4. Exemplary human INTS12 transcript sequences include, but are not limited to, NCBI Reference Sequences: NM_020395.4 GI: 1519313827, NM_001142471.2 GI: 1890284121, XM_005263148.5 GI: 1370487345, XM_011532143.2 GI: 1034640720, XM_011532145.2 GI: 1034640721, XR_001741293.1 GI: 1034640723, and XR_001741294.1 GI: 1034640724. Exemplary human INTS12 protein sequences include, but are not limited to, NCBI Reference Sequences: NP_065128.2 GI: 21361851, NP_001135943.1 GI: 215599585, XP_005263205.1 GI: 530378004, XP_011530445.1 GI: 767932391, and XP_011530447.1 GI: 767932395.

As used herein, the term “RPRD1B,” “regulation of nuclear pre-mRNA domain containing 1B” refers to the gene RPRD1B and the gene product encoded by the RPRD1B gene. It is also known as “C20orf77,” “CREPT,” “K-H,” “Kub5-Hera,” “NET60,” or “dJ1057B20.2.” In the human genome, RPRD1B is located on chromosome 20. Exemplary human RPRD1B transcript sequences include, but are not limited to, NCBI Reference Sequences: NM_021215.4 GI: 1653960413, XM_005260477.4 GI: 1370480788, XM_005260480.5 GI: 1370480789, XM_011528954.3 GI: 1370480787, XM_017027990.2 GI: 1370480785, XM_017027991.1 GI: 1034625581, XR_936597.3 GI: 1370480786, and XR_001754361.1 GI: 1034625583. Exemplary human RPRD1B protein sequences include, but are not limited to, NCBI Reference Sequences: NP_067038.1 GI: 11034845, XP_005260534.1 GI: 530418240, XP_005260537.1 GI: 530418246, XP_011527256.1 GI: 768017833, XP_016883479.1 GI: 1034625577, and XP_016883480.1 GI: 1034625582. Without wishing to be bound by theory, it is believed that in some embodiments, RPRD1B is involved in transcription.

As used herein, the term “TMPRSS11B,” “transmembrane serine protease 11B” refers to the gene TMPRSS11B and the gene product encoded by the TMPRSS11B gene. It is also known as “HATL5.” In the human genome, TMPRSS11B is located on chromosome 4. Exemplary human TMPRSS11B transcript sequences include, but are not limited to, NCBI Reference Sequences: NM_182502.3 GI: 187761336, XM_011531608.2 GI: 1034638401, and XM_011531609.1 GI: 767931074. Exemplary human TMPRSS11B protein sequences include, but are not limited to, NCBI Reference Sequences: NP_872308.2 GI: 187761337, XP_011529910.1 GI: 767931073, and XP_011529911.1 GI: 767931075.

As used herein, the term “HTT,” “huntingtin” refers to the gene HTT and the gene product encoded by the HTT gene. It is also known as “HD,” “IT15,” or “LOMARS.” In the human genome, HTT is located on chromosome 4. Exemplary human HTT transcript sequences include, but are not limited to, NCBI Reference Sequences: NM_001388492.1 GI: 1933851640 and NM_002111.8 GI: 1034301421. Exemplary human HTT protein sequences include, but are not limited to, NCBI Reference Sequences: NP_001375421.1 GI: 1933851641 and NP_002102.4 GI: 90903231. Without wishing to be bound by theory, it is believed that in some embodiments, HTT is involved in microtubule-mediated transport or vesicle function.

As used herein, the term “SNRNP70,” “small nuclear ribonucleoprotein U1 subunit 70” refers to the gene SNRNP70 and the gene product encoded by the SNRNP70 gene. It is also known as “RNPU1Z,” “RPU1,” “SNRP70,” “Snp1,” “U1-70K,” “U170K,” “U1AP,” or “U1 RNP.” In the human genome, SNRNP70 is located on chromosome 19. Exemplary human SNRNP70 transcript sequences include, but are not limited to, NCBI Reference Sequences: M_003089.6 GI: 1519242493, NM_001301069.2 GI: 1675105714, XM_011527240.2 GI: 1034609072, and XM_011527241.2 GI: 1034609074. Exemplary human SNRNP70 protein sequences include, but are not limited to, NCBI Reference Sequences: NP_003080.2 GI: 29568103, NP_001287998.1 GI: 666637959, XP_011525542.2 GI: 1034609073, and XP_011525543.1 GI: 768010688.

As used herein, the term “BRD7,” “bromodomain containing 7” refers to the gene BRD7 and the gene product encoded by the BRD7 gene. It is also known as “BP75,” CELTIX1,” “NAG4,” or “SMARCI1.” In the human genome, BRD7 is located on chromosome 16. Exemplary human BRD7 transcript sequences include, but are not limited to, NCBI Reference Sequences: NM_013263.5 GI: 1677529864, NM_001173984.3 GI: 1677501396, XM_011523046.3 GI: 1370468336, XM_011523047.2 GI: 1034594417, XM_011523048.2 GI: 1034594418, XM_011523049.2 GI: 1034594419, XM_011523050.3 GI: 1370468340, XM_017023179.1 GI: 1034594420, XM_017023180.1 GI: 1034594422, XM_017023181.1 GI: 1034594428, XR_933289.3 GI: 1370468337, XR_933290.3 GI: 1370468338, XR_933291.3 GI: 1370468341, and XR_001751896.2 GI: 1370468339. Exemplary human BRD7 protein sequences include, but are not limited to, NCBI Reference Sequences: NP_037395.2 GI: 41350212, NP_001167455.1 GI: 291219913, XP_011521348.1 GI: 767989828, XP_011521349.1 GI: 767989830, XP_011521350.1 GI: 767989832, XP_011521351.1 GI: 767989834, XP_011521352.1 GI: 767989838, XP_016878668.1 GI: 1034594421, XP_016878669.1 GI: 1034594423, and XP_016878670.1 GI: 1034594429.

As used herein, the term “DMXL1,” “Dmx like 1” refers to the gene DMXL1 and the gene product encoded by the DMXL1 gene. In the human genome, DMXL1 is located on chromosome 5. Exemplary human DMXL1 transcript sequences include, but are not limited to, NCBI Reference Sequences: NM_001290321.3 GI: 1839439017, NM_005509.6 GI: 1677556815, NM_001349239.2 GI: 1677530983, NM_001349240.2 GI: 1917203644, NM_001387933.1 GI: 1917203286, NM_001387934.1 GI: 1917203548, NM_001387937.1 GI: 1917203690, NM_001387938.1 GI: 1917203706, NR_170867.1 GI: 1917204838, NR_170868.1 GI: 1917204849, NR_170869.1 GI: 1917204840, XM_005271909.4 GI: 1034643906, XM_005271910.5 GI: 1370488580, XM_005271912.2 GI: 1034643913, XM_011543212.2 GI: 1034643905, XM_011543213.2 GI: 1034643907, XM_011543214.2 GI: 1034643926, XM_011543215.2 GI: 1034643927, XM_017009143.1 GI: 1034643914, XM_017009145.1 GI: 1034643918, XM_017009144.1 GI: 1034643916, XM_017009146.1 GI: 1034643920, XM_017009147.1 GI: 1034643922, XM_017009148.1 GI: 1034643924, XR_948239.2 GI: 1034643928, and XR_001742026.2 GI: 1370488581. Exemplary human DMXL1 protein sequences include, but are not limited to, NCBI Reference Sequences: NP_001277250.1 GI: 594140514, NP_005500.4 GI: 148528998, NP_001336168.1 GI: 1154067688, NP_001336169.1 GI: 1154067690, NP_001374862.1 GI: 1917203287, NP_001374863.1 GI: 1917203549, NP_001374866.1 GI: 1917203691, NP_001374867.1 GI: 1917203707, XP_005271966.1 GI: 530379741, XP_005271967.1 GI: 530379743, XP_005271969.1 GI: 530379747, XP_011541514.1 GI: 767935464, XP_011541515.1 GI: 767935467, XP_011541516.1 GI: 767935470, XP_011541517.1 GI: 767935472, XP_016864632.1 GI: 1034643915, XP_016864634.1 GI: 1034643919, XP_016864633.1 GI: 1034643917, XP_016864635.1 GI: 1034643921 XP_016864637.1 GI: 1034643925. Without wishing to be bound by theory, it is believed that in some embodiments, DMXL1 is involved in intracellular trafficking.

As used herein, the term “RAB10,” “RAB10, member RAS oncogene family” refers to the gene RAB10 and the gene product encoded by the RAB10 gene. In the human genome, RAB10 is located on chromosome 2. Exemplary human RAB10 transcript sequences include, but are not limited to, NCBI Reference Sequences: NM_016131.5 GI: 1519315187 and XM_024452565.1 GI: 1370476585. Exemplary human RAB10 protein sequences include, but are not limited to, NCBI Reference Sequences: NP_057215.3 GI: 256222019 and XP_024308333.1 GI: 1370476586. Without wishing to be bound by theory, it is believed that in some embodiments, RAB10 is involved in intracellular trafficking.

As used herein, the term “CNGA1,” “cyclic nucleotide gated channel subunit alpha 1” refers to the gene CNGA1 and the gene product encoded by the CNGA1 gene. It is also known as “CNCG,” “CNCG1,” “CNG-1,” “CNG1,” “RCNC1,” “RCNCa,” “RCNCalpha,” or “RP49.” In the human genome, CNGA1 is located on chromosome 4. Exemplary human CNGA1 transcript sequences include, but are not limited to, NCBI Reference Sequences: NM_001379270.1 GI: 1825751503, NM_000087.5 GI: 1825723785, NM_001142564.2 GI: 1825761141, XM_005248049.4 GI: 1034638335, and XM_011513623.2 GI: 1034638339. Exemplary human CNGA1 protein sequences include, but are not limited to, NCBI Reference Sequences: NP_001366199.1 GI: 1825751504, NP_000078.3 GI: 1825723786, NP_001136036.2 GI: 1825761142, XP_005248106.2 GI: 1034638336, and XP_011511925.1 GI: 767930182.

As used herein, the term “KLHDC3,” “kelch domain containing 3” refers to the gene KLHDC3 and the gene product encoded by the KLHDC3 gene. It is also known as “PEAS,” “dJ20C7.3.” In the human genome, KLHDC3 is located on chromosome 6. Exemplary human KLHDC3 transcript sequences include, but are not limited to, NCBI Reference Sequences: NM_057161.4 GI: 1519244620, XM_024446320.1 GI: 1370507304, and NR_040101.2 GI: 1701111634. Exemplary human KLHDC3 protein sequences include, but are not limited to, NCBI Reference Sequences: NP_476502.1 GI: 16945972 and XP_024302088.1 GI: 1370507305.

As used herein, the term “CHP1,” “calcineurin like EF-hand protein 1” refers to the gene CHP1 and the gene product encoded by the CHP1 gene. It is also known as “CHP,” “SLC9A1BP,” “SPAX9,” “Sid470p,” “p22,” or “p24.” In the human genome, CHP1 is located on chromosome 15. Exemplary human CHP1 transcript sequences include, but are not limited to, NCBI Reference Sequences: NM_007236.5 GI: 1519242780 and XM_017021879.2 GI: 1370466153. Exemplary human CHP1 protein sequences include, but are not limited to, NCBI Reference Sequences: NP_009167.1 GI: 6005731 and XP_016877368.1 GI: 1034589580. Without wishing to be bound by theory, it is believed that in some embodiments, CHP1 is involved in calcium ion-regulated exocytosis.

As used herein, the term “CYP3A5,” “cytochrome P450 family 3 subfamily A member 5” refers to the gene CYP3A5 and the gene product encoded by the CYP3A5 gene. It is also known as “CP35,” “CYPIIIA5,” “P450PCN3,” or “PCN3.” In the human genome, CYP3A5 is located on chromosome 7. Exemplary human CYP3A5 transcript sequences include, but are not limited to, NCBI Reference Sequences: NM_000777.5 GI: 1464296603, NM_001190484.3 GI: 1676319063, NM_001291829.2 GI: 1676318553, NM_001291830.2 GI: 1676439747, and NR_033807.3 GI: 1700447993. Exemplary human CYP3A5 protein sequences include, but are not limited to, NCBI Reference Sequences: NP_000768.1 GI: 4503231, NP_001177413.1 GI: 306518609, NP_001278758.1 GI: 628601849, and NP_001278759.1 GI: 628601851.

As used herein, the term “ELOVL4,” “ELOVL fatty acid elongase 4” refers to the gene ELOVL4 and the gene product encoded by the ELOVL4 gene. It is also known as “ADMD,” “CT118,” “ISQMR,” “SCA34,” “STGD2,” or “STGD3.” In the human genome, ELOVL4 is located on chromosome 6. Exemplary human ELOVL4 transcript sequences include, but are not limited to, NCBI Reference Sequence: NM_022726.4. Exemplary human ELOVL4 protein sequences include, but are not limited to, NCBI Reference Sequence: NP_073563.1.

As used herein, the term “PNISR,” “PNN interacting serine and arginine rich protein” refers to the gene PNISR and the gene product encoded by the PNISR gene. It is also known as “C6orf111,” “HSPC306,” “SFRS18,” “SRrp130,” or “bA98I9.2.” In the human genome, PNISR is located on chromosome 6. Exemplary human PNISR transcript sequences include, but are not limited to, NCBI Reference Sequences: NM_032870.4 GI: 1519312939, NM_015491.3 GI: 1890261252, NM_001322405.2 GI: 1675097003, NM_001322406.2 GI: 1674985875, NM_001322408.2 GI: 1675132982, NM_001322410.2 GI: 1889564404, NM_001322412.2 GI: 1889462223, NM_001322413.2 GI: 1889518186, NM_001322414.2 GI: 1676317653, NM_001322415.2 GI: 1675144735, NM_001322416.2 GI: 1890335651, NM_001322417.2 GI: 1676318041, NM_001322418.2 GI: 1676319692, NM_001322419.2 GI: 1676346511, NR_136326.2 GI: 1700447845, XM_005266912.4 GI: 1370507860, XM_017010710.2 GI: 1370507864, XM_017010711.1 GI: 1034649769, XM_017010712.1 GI: 1034649771, XM_017010713.1 GI: 1034649773, XM_024446397.1 GI: 1370507862, and XM_024446398.1 GI: 1370507865. Exemplary human PNISR protein sequences include, but are not limited to, NCBI Reference Sequences: NP_116259.2 GI: 154146193, NP_056306.1 GI: 154146223, NP_001309334.1 GI: 1016841103, NP_001309335.1 GI: 1016841173, NP_001309337.1 GI: 1016841126, NP_001309339.1 GI: 1016841152, NP_001309341.1 GI: 1016841051, NP_001309342.1 GI: 1016841188, NP_001309343.1 GI: 1016841074, NP_001309344.1 GI: 1016841045, NP_001309345.1 GI: 1016841080, NP_001309346.1 GI: 1016840992, NP_001309347.1 GI: 1016840968, NP_001309348.1 GI: 1016841122, XP_005266969.3 GI: 1370507861, XP_016866199.1 GI: 1034649768, XP_016866200.1 GI: 1034649770, XP_016866201.1 GI: 1034649772, XP_016866202.1 GI: 1034649774, XP_024302165.1 GI: 1370507863, and XP_024302166.1 GI: 1370507866. Without wishing to be bound by theory, it is believed that in some embodiments, PNISR is involved in RNA binding by an AAV protein.

As used herein, the term “ZCCHC14,” “zinc finger CCHC-type containing 14” refers to the gene ZCCHC14 and the gene product encoded by the ZCCHC14 gene. It is also known as “BDG-29” or “BDG29.” In the human genome, ZCCHC14 is located on chromosome 16. Exemplary human ZCCHC14 transcript sequences include, but are not limited to, NCBI Reference Sequences: NM_015144.3 GI: 1704602913, XM_005255858.3 GI: 767989629, XM_011522964.2 GI: 1034594071, XM_017023082.2 GI: 1370468161, and XR_243401.3 GI: 767989631. Exemplary human ZCCHC14 protein sequences include, but are not limited to, NCBI Reference Sequences: NP_055959.2 GI: 1704602914, XP_005255915.2 GI: 767989630, XP_011521266.1 GI: 767989635, and XP_016878571.1 GI: 1034594070. Without wishing to be bound by theory, it is believed that in some embodiments, ZCCHC14 is involved in protection of viral RNA.

As used herein, the term “AZGP1,” “alpha-2-glycoprotein 1, zinc-binding” refers to the gene AZGP1 and the gene product encoded by the AZGP1 gene. It is also known as “ZA2G,” “ZAG.” In the human genome, AZGP1 is located on chromosome 7. Exemplary human AZGP1 transcript sequences include, but are not limited to, NCBI Reference Sequences: NM_001185.4. Exemplary human AZGP1 protein sequences include, but are not limited to, NCBI Reference Sequences: NP_001176.1.

As used herein, the term “TPST1,” “tyrosylprotein sulfotransferase 1” refers to the gene TPST1 and the gene product encoded by the TPST1 gene. It is also known as “TANGO13A.” In the human genome, TPST1 is located on chromosome 7. Exemplary human TPST1 transcript sequences include, but are not limited to, NCBI Reference Sequences: NM_003596.4 GI: 1519314127, XM_005250642.3 GI: 1370511155, XM_011516634.3 GI: 1370511158, XM_011516635.3 GI: 1370511159, XM_017012724.1 GI: 1034657193, XM_017012726.2 GI: 1370511156, and XM_017012727.2 GI: 1370511157. Exemplary human TPST1 protein sequences include, but are not limited to, NCBI Reference Sequences: NP_003587.1 GI: 4507665, XP_005250699.1 GI: 530386524, XP_011514936.1 GI: 767948891, XP_011514937.1 GI: 767948893, XP_016868213.1 GI: 1034657194, XP_016868215.1 GI: 1034657198, and XP_016868216.1 GI: 1034657200. Without wishing to be bound by theory, it is believed that in some embodiments, TPST1 is involved O-sulfation of a tyrosine residue within an acidic motif of a polypeptide.

As used herein, the term “SC5D,” “sterol-C5-desaturase” refers to the gene SC5D and the gene product encoded by the SC5D gene. It is also known as “ERG3,” “S5DES,” or “SC5DL.” In the human genome, SC5D is located on chromosome 11. Exemplary human SC5D transcript sequences include, but are not limited to, NCBI Reference Sequences: NM_006918.5 GI: 1519245754 and NM_001024956.3 GI: 1890248668. Exemplary human SC5D protein sequences include, but are not limited to, NCBI Reference Sequences: NP_008849.2 GI: 68160941 and NP_001020127.1 GI: 68160945. Without wishing to be bound by theory, it is believed that in some embodiments, SC5D is involved in cholesterol biosynthesis.

As used herein, the term “ELP2,” “elongator acetyltransferase complex subunit 2” refers to the gene ELP2 and the gene product encoded by the ELP2 gene. It is also known as “MRT58,” “SHINC-2,” “STATIP1,” or “StIP.” In the human genome, ELP2 is located on chromosome 18. Exemplary human ELP2 transcript sequences include, but are not limited to, NCBI Reference Sequences: NM_018255.4 GI: 1519244511, NM_001242875.3 GI: 1676319536, NM_001242876.3 GI: 1676440759, NM_001242877.3 GI: 1676320024, NM_001242878.3 GI: 1675144742, NM_001242879.3 GI: 1676324826, NM_001324465.2 GI: 1675005228, NM_001324466.2 GI: 1675068870, NM_001324467.2 GI: 1676317767, NM_001324468.2 GI: 1676318610, NR_040110.3 GI: 1701969756, NR_136897.2 GI: 1700660398, NR_136898.2 GI: 1700660347, NR_137173.2 GI: 1701108951, and XR_430081.2 GI: 1034604246. Exemplary human ELP2 protein sequences include, but are not limited to, NCBI Reference Sequences: NP_060725.1 GI: 8922735, NP_001229804.1 GI: 338968885, NP_001229805.1 GI: 338968888, NP_001229806.1 GI: 338968890, NP_001229807.1 GI: 338968892, NP_001229808.1 GI: 338968894, NP_001311394.1 GI: 1026191057, NP_001311395.1 GI: 1026191045, NP_001311396.1 GI: 1026191048, and NP_001311397.1 GI: 1026191061.

As used herein, the term “ELP3,” “elongator acetyltransferase complex subunit 3” refers to the gene ELP3 and the gene product encoded by the ELP3 gene. It is also known as “KAT9.” In the human genome, ELP3 is located on chromosome 8. Exemplary human ELP3 transcript sequences include, but are not limited to, NCBI Reference Sequences: NM_018091.6 GI: 1519245905, NM_001284220.2 GI: 1675178308, NM_001284222.2 GI: 1676440523, NM_001284224.2 GI: 1676441760, NM_001284225.2 GI: 1890333780, NM_001284226.2 GI: 1890334018, XM_006716354.3 GI: 1370512533, XM_017013604.2 GI: 1370512528, XM_024447184.1 GI: 1370512529, and XM_024447185.1 GI: 1370512531. Exemplary human ELP3 protein sequences include, but are not limited to, NCBI Reference Sequences: NP_060561.3 GI: 23510283, NP_001271151.1 GI: 545746373, NP_001271153.1 GI: 545746266, NP_001271154.1 GI: 545746350, NP_001271155.1 GI: 545746360, XP_006716417.1 GI: 578815360, XP_016869093.1 GI: 1034660802, XP_024302952.1 GI: 1370512530, XP_024302952.1 GI: 1370512530, and NP_001271149.1 GI: 545746389.

As used herein, the term “IPO9,” “importin 9” refers to the gene IP09 and the gene product encoded by the IP09 gene. It is also known as “Imp9.” In the human genome, IP09 is located on chromosome 1. Exemplary human IP09 transcript sequences include, but are not limited to, NCBI Reference Sequence: NM_018085.5. Exemplary human IP09 protein sequences include, but are not limited to, NCBI Reference Sequence: NP_060555.2. Without wishing to be bound by theory, it is believed that in some embodiments, IP09 is involved nuclear protein import.

As used herein, the term “RAB14,” “RAB14, member RAS oncogene family” refers to the gene RAB14 and the gene product encoded by the RAB14 gene. It is also known as “FBP” or “RAB-14.” In the human genome, RAB14 is located on chromosome 9. Exemplary human RAB14 transcript sequences include, but are not limited to, NCBI Reference Sequence: NM_016322.4. Exemplary human RAB14 protein sequences include, but are not limited to, NCBI Reference Sequence: NP_057406.2. Without wishing to be bound by theory, it is believed that in some embodiments, RAB14 is involved in intracellular trafficking.

As used herein, the term “WDR7,” “WD repeat domain 7” refers to the gene WDR7 and the gene product encoded by the WDR7 gene. It is also known as “TRAG.” In the human genome, WDR7 is located on chromosome 18. Exemplary human WDR7 transcript sequences include, but are not limited to, NCBI Reference Sequences: NM_015285.3 GI: 1840394179, NM_052834.3 GI: 1840394160, NM_001382485.1 GI: 1840394202, NM_001382487.1 GI: 1840394220, XM_011525888.2 GI: 1370473340, XM_017025683.1 GI: 1034603793, XR_001753170.2 GI: 1370473341, and XR_001753171.2 GI: 1370473342. Exemplary human WDR7 protein sequences include, but are not limited to, NCBI Reference Sequences: NP_056100.2 GI: 73747877, NP_443066.2 GI: 73747879, NP_001369414.1 GI: 1840394203, NP_001369416.1 GI: 1840394221, XP_011524190.1 GI: 767998466, and XP_016881172.1 GI: 1034603794. Without wishing to be bound by theory, it is believed that in some embodiments, WDR7 is involved in intracellular trafficking.

As used herein, the term “XRCC4,” “X-ray repair cross complementing 4” refers to the gene XRCC4 and the gene product encoded by the XRCC4 gene. It is also known as “SSMED.” In the human genome, XRCC4 is located on chromosome 5. Exemplary human XRCC4 transcript sequences include, but are not limited to, NCBI Reference Sequences: NM_003401.5 GI: 1677538315, NM_022406.5 GI: 1890342175, NM_022550.4 GI: 1890275318, NM_001318012.3 GI: 1890335896, NM_001318013.2 GI: 1677530491, XM_011543626.1 GI: 767936485, XM_017009827.2 GI: 1370505598, XM_017009828.2 GI: 1370505599, and XM_017009829.2 GI: 1370505615. Exemplary human XRCC4 protein sequences include, but are not limited to, NCBI Reference Sequences: NP_003392.1 GI: 4507945, NP_071801.1 GI: 12408647, NP_072044.1 GI: 12408649, NP_001304941.1 GI: 966751406, NP_001304942.1 GI: 966751408, XP_011541928.1 GI: 767936486, XP_016865316.1 GI: 1034646058, XP_016865317.1 GI: 1034646060, and XP_016865318.1 GI: 1034646062.

As used herein, the term “GDI2,” “GDP dissociation inhibitor 2” refers to the gene GDI2 and the gene product encoded by the GDI2 gene. It is also known as “HEL-S-46e,” or “RABGDIB.” In the human genome, GDI2 is located on chromosome 10. Exemplary human GDI2 transcript sequences include, but are not limited to, NCBI Reference Sequences: NM_001494.4 GI: 1519243133, NM_001115156.2 GI: 1890249601, and XM_017016071.2 GI: 1370456869. Exemplary human GDI2 protein sequences include, but are not limited to, NCBI Reference Sequences: NP_001485.2 GI: 6598323, NP_001108628.1 GI: 169646441, and XP_016871560.1 GI: 1034567732. Without wishing to be bound by theory, it is believed that in some embodiments, GDI2 is involved in intracellular trafficking.

As used herein, the term “KIAA0319L,” “KIAA0319 like” refers to the gene KIAA0319L and the gene product encoded by the KIAA0319L gene. It is also known as “AAVR,” or “AAVRL.” In the human genome, KIAA0319L is located on chromosome 1. Exemplary human KIAA0319L transcript sequences include, but are not limited to, NCBI Reference Sequences: NM_024874.5 GI: 1519316171, XM_006710907.1 GI: 578799788, XM_006710909.2 GI: 1370454596, XM_006710910.1 GI: 578799794, XM_011542179.2 GI: 1034561954, XM_011542180.1 GI: 767906108, XM_017002366.1 GI: 1034561946, XM_017002367.1 GI: 1034561948, XM_017002368.1 GI: 1034561950, XM_017002369.2 GI: 1370454595, XM_017002370.1 GI: 1034561957, XM_017002371.1 GI: 1034561959, XM_017002373.2 GI: 1370454599, XM_017002372.2 GI: 1370454597, XM_017002374.1 GI: 1034561971, XM_017002377.1 GI: 1034561977, XM_017002378.1 GI: 1034561979, XR_246296.1 GI: 530363371, XR_946763.2 GI: 1034561956, XR_001737420.1 GI: 1034561955, XR_001737421.1 GI: 1034561963, XR_001737423.1 GI: 1034561967, XR_001737424.1 GI: 1034561968, XR_001737425.1 GI: 1034561969, XR_001737426.1 GI: 1034561970, and XR_002957627.1 GI: 1370454598. Exemplary human KIAA0319L protein sequences include, but are not limited to, NCBI Reference Sequences: NP_079150.3 GI: 33359221, XP_006710970.1 GI: 578799789, XP_006710972.1 GI: 578799793, XP_006710973.1 GI: 578799795, XP_011540481.1 GI: 767906106, XP_011540482.1 GI: 767906109, XP_016857855.1 GI: 1034561947, XP_016857856.1 GI: 1034561949, XP_016857857.1 GI: 1034561951, XP_016857858.1 GI: 1034561953, XP_016857859.1 GI: 1034561958, XP_016857860.1 GI: 1034561960, XP_016857862.1 GI: 1034561966, XP_016857861.1 GI: 1034561962, XP_016857863.1 GI: 1034561972, XP_016857866.1 GI: 1034561978, and XP_016857867.1 GI: 1034561980. In some instances, KIAA0319L may be an ortholog of a murine AU040320 gene.

As used herein, the term “BAMBI,” “BMP and activin membrane bound inhibitor” refers to the gene BAMBI and the gene product encoded by the BAMBI gene. In the human genome, BAMBI is located on chromosome 10. Exemplary human BAMBI transcript sequences include, but are not limited to, NCBI Reference Sequence: NM_012342.3. Exemplary human BAMBI protein sequences include, but are not limited to, NCBI Reference Sequence: NP_036474.1.

As used herein, the term “TM9SF2,” “transmembrane 9 superfamily member 2” refers to the gene TM9SF2 and the gene product encoded by the TM9SF2 gene. It is also known as “P76.” In the human genome, TM9SF2 is located on chromosome 13. Exemplary human TM9SF2 transcript sequences include, but are not limited to, NCBI Reference Sequence: NM_004800.3. Exemplary human TM9SF2 protein sequences include, but are not limited to, NCBI Reference Sequence: NP_004791.1. Without wishing to be bound by theory, it is believed that in some embodiments, TM9SF2 is involved in HSPG metabolism.

As used herein, the term “PHIP,” “pleckstrin homology domain interacting protein” refers to the gene PHIP and the gene product encoded by the PHIP gene. It is also known as “BRWD2,” “CHUJANS,” “DCAF14,” “DIDOD,” “WDR11,” or “ndrp.” In the human genome, PHIP is located on chromosome 6. Exemplary human PHIP transcript sequences include, but are not limited to, NCBI Reference Sequences: NM_017934.7 GI: 1519244214, XM_005248729.5 GI: 1370508281, XM_011535918.3 GI: 1370508282, XM_011535919.1 GI: 767943079, XM_017010989.2 GI: 1370508283, and XM_017010990.2 GI: 1370508284. Exemplary human PHIP protein sequences include, but are not limited to, NCBI Reference Sequences: NP_060404.4 GI: 1081684143, XP_005248786.1 GI: 530383162, XP_011534220.1 GI: 767943077, XP_011534221.1 GI: 767943080, XP_016866478.1 GI: 1034650679, and XP_016866479.1 GI: 1034650681.

As used herein, the term “DCLRE1C,” “DNA cross-link repair 1C” refers to the gene DCLRE1C and the gene product encoded by the DCLRE1C gene. It is also known as “A-SCID,” “DCLREC1C,” “RS-SCID,” “SCIDA,” or “SNM1C.” In the human genome, DCLRE1C is located on chromosome 10. Exemplary human DCLRE1C transcript sequences include, but are not limited to, NCBI Reference Sequences: NM_001033855.3 GI: 1519313702, NM_022487.4 GI: 1676439808, NM_001033857.3 GI: 1675014622, NM_001033858.3 GI: 1674985937, NM_001289076.2 GI: 1675088889, NM_001289077.2 GI: 1676440107, NM_001289078.2 GI: 1675178651, NM_001289079.2 GI: 1676318328, NM_001350965.2 GI: 1675178179, NM_001350966.2 GI: 1675070869, NM_001350967.2 GI: 1675178647, NR_110297.2 GI: 1701108655, NR_146960.1 GI: 1187443743, NR_146961.2 GI: 1676453019, NR_146962.1 GI: 1187443745, XM_006717491.4 GI: 1370457643, XM_011519616.1 GI: 767960405, XM_011519617.1 GI: 767960407, XM_011519619.1 GI: 767960412, XM_011519620.3 GI: 1370457648, XM_011519621.2 GI: 1370457651, XM_017016557.1 GI: 1034569251, XM_017016558.1 GI: 1034569254, XM_024448134.1 GI: 1370457644, XM_024448135.1 GI: 1370457646, XR_930515.2 GI: 1370457650, XR_001747185.2 GI: 1370457649, and XR_001747187.1 GI: 1034569264. Exemplary human DCLRE1C protein sequences include, but are not limited to, NCBI Reference Sequences: NP_001029027.1 GI: 76496497, NP_071932.2 GI: 76496495, NP_001029029.1 GI: 76496499, NP_001029030.1 GI: 76496501, NP_001276005.1 GI: 574276598, NP_001276006.1 GI: 574276608, NP_001276007.1 GI: 574276692, NP_001276008.1 GI: 574276944, NP_001337894.1 GI: 1187443721, NP_001337895.1 GI: 1187443723, NP_001337896.1 GI: 1187443725, XP_006717554.1 GI: 578818534, XP_011517918.1 GI: 767960406, XP_011517919.1 GI: 767960408, XP_011517921.1 GI: 767960413, XP_011517922.1 GI: 767960415, XP_011517923.1 GI: 767960420, XP_016872046.1 GI: 1034569252, XP_016872047.1 GI: 1034569255, XP_024303902.1 GI: 1370457645, and XP_024303903.1 GI: 1370457647. Without wishing to be bound by theory, it is believed that in some embodiments, DCLRE1C is involved in viral hairpin resolution.

As used herein, the term “VPS35,” “VPS35 retromer complex component” refers to the gene VPS35 and the gene product encoded by the VPS35 gene. It is also known as “MEM3,” or “PARK17.” In the human genome, VPS35 is located on chromosome 16. Exemplary human VPS35 transcript sequences include, but are not limited to, NCBI Reference Sequences: NM_018206.6 GI: 1653961133, XM_005256045.3 GI: 1034595269, and XM_011523227.3 GI: 1370468835. Exemplary human VPS35 protein sequences include, but are not limited to, NCBI Reference Sequences: NP_060676.2 GI: 17999541, XP_005256102.1 GI: 530424105, and XP_011521529.1 GI: 767990260. Without wishing to be bound by theory, it is believed that in some embodiments, VPS35 is involved in endosome trafficking.

As used herein, the term “GPR108,” “G protein-coupled receptor 108” refers to the gene GPR108 and the gene product encoded by the GPR108 gene. It is also known as “LUSTR2.” In the human genome, GPR108 is located on chromosome 19. Exemplary human GPR108 transcript sequences include, but are not limited to, NCBI Reference Sequences: NM_001080452.2 GI: 2031835807, NM_020171.2 GI: 1388872084, NM_001394714.1 GI: 2031835746, NM_001394715.1 GI: 2031835788, NM_001394716.1 GI: 2031835801, NM_001394717.1 GI: 2031835748, NM_001394718.1 GI: 2031835760, NM_001394719.1 GI: 2031835799, NM_001394720.1 GI: 2031835809, NM_001394721.1 GI: 2031835737, NM_001394722.1 GI: 2031835784, NM_001394723.1 GI: 2031835805, NM_001394724.1 GI: 2031835795, NM_001394725.1 GI: 2031835735, NM_001394726.1 GI: 2032005899, NM_001394727.1 GI: 2031835765, NM_001394728.1 GI: 2031835780, and XM_024451618.1 GI: 1370475541. Exemplary human GPR108 protein sequences include, but are not limited to, NCBI Reference Sequences: NP_001073921.1 GI: 122937301, NP_064556.1 GI: 130489591, NP_001381643.1 GI: 2031835747, NP_001381644.1 GI: 2031835789, NP_001381645.1 GI: 2031835802, NP_001381646.1, NP_001381647.1 GI: 2031835761 GI: 2031835749, NP_001381648.1 GI: 2031835800, NP_001381649.1 GI: 2031835810, NP_001381650.1 GI: 2031835738, NP_001381651.1 GI: 2031835785, NP_001381652.1 GI: 2031835806, NP_001381653.1 GI: 2031835796, NP_001381654.1 GI: 2031835736, NP_001381655.1 GI: 2032005900, NP_001381656.1 GI: 2031835766, NP_001381657.1 GI: 2031835781, and XP_024307386.1 GI: 1370475542. Without wishing to be bound by theory, it is believed that in some embodiments, GPR108 is involved in with inhibition of a TLR.

As used herein, the term “CYREN,” “cell cycle regulator of NHEJ” refers to the gene CYREN and the gene product encoded by the CYREN gene. It is also known as “C7orf49,” “MRI,” or “MRI-2.” In the human genome, CYREN is located on chromosome 7. Exemplary human CYREN transcript sequences include, but are not limited to, NCBI Reference Sequences: NM_024033.4 GI: 1832254209, NM_001243749.2 GI: 1889526520, NM_001243751.2 GI: 1890266942, NM_001243752.2 GI: 1889544905, NM_001243753.2 GI: 1890284058, NM_001243754.2 GI: 1890343331, NM_001243755.2 GI: 1889625831, NM_001305629.2 GI: 1889579912, NM_001305630.2 GI: 1890343751, NM_001363329.2 GI: 1890258885, NM_001363330.2 GI: 1890243018, XM_017012591.2 GI: 1370510948, XM_017012592.2 GI: 1370510949, XM_017012593.2 GI: 1370510950, XM_017012594.2 GI: 1370510951, XM_017012595.1 GI: 1034656830, and XM_024446930.1 GI: 1370510952. Exemplary human CYREN protein sequences include, but are not limited to, NCBI Reference Sequences: NP_076938.2 GI: 208022703, NP_001230678.1 GI: 344217761, NP_001230680.1 GI: 344217765, NP_001230681.1 GI: 344217767, NP_001230682.1 GI: 344217769, NP_001230683.1 GI: 344217771, NP_001230684.1 GI: 344217706, NP_001292558.1 GI: 779175606, NP_001292559.1 GI: 779174613, NP_001350258.1 GI: 1390015657, NP_001350259.1 GI: 1389992039, XP_016868080.1 GI: 1034656823, XP_016868081.1 GI: 1034656825, XP_016868082.1 GI: 1034656827, XP_016868083.1 GI: 1034656829, XP_016868084.1 GI: 1034656831, and XP_024302698.1 GI: 1370510953. Without wishing to be bound by theory, it is believed that in some embodiments, CYREN is involved in inhibition of NHEJ.

As used herein, the term “ACP2,” “acid phosphatase 2, lysosomal” refers to the gene ACP2 and the gene product encoded by the ACP2 gene. It is also known as “LAP.” In the human genome, ACP2 is located on chromosome 11. Exemplary human ACP2 transcript sequences include, but are not limited to, NCBI Reference Sequences: NM_001610.4 GI: 1519243523, NM_001302489.2 GI: 1890270791, NM_001302490.2 GI: 1890261409, NM_001302491.2 GI: 1890284431, NM_001302492.2 GI: 1890248306, NM_001357016.2 GI: 1890283997, and XR_001747908.2 GI: 1370459645. Exemplary human ACP2 protein sequences include, but are not limited to, NCBI Reference Sequences: NP_001601.1 GI: 4557010, NP_001289418.1 GI: 699964207, NP_001289419.1 GI: 699964215, NP_001289420.1 GI: 699964217, NP_001289421.1 GI: 699964219, and NP_001343945.1 GI: 1257317299. Without wishing to be bound by theory, it is believed that in some embodiments, ACP2 is associated with influenza infection.

As used herein, the term “F8A2,” “coagulation factor VIII associated 2” refers to the gene F8A2 and the gene product encoded by the F8A2 gene. It is also known as “HAP40.” In the human genome, F8A2 is located on chromosome X. Exemplary human F8A2 transcript sequences include, but are not limited to, NCBI Reference Sequence: NM_001007523.2. Exemplary human F8A2 protein sequences include, but are not limited to, NCBI Reference Sequence: NP_001007524.1.

As used herein, the term “F8A1,” “coagulation factor VIII associated 1” refers to the gene F8A1 and the gene product encoded by the F8A1 gene. It is also known as “DXS522E,” “F8A,” or “HAP40.” In the human genome, F8A1 is located on chromosome X. Exemplary human F8A1 transcript sequences include, but are not limited to, NCBI Reference Sequence: NM_012151.4. Exemplary human F8A1 protein sequences include, but are not limited to, NCBI Reference Sequence: NP_036283.2.

As used herein, the term “F8A3,” “coagulation factor VIII associated 3” refers to the gene F8A3 and the gene product encoded by the F8A3 gene. In the human genome, F8A3 is located on chromosome X. Exemplary human F8A3 transcript sequences include, but are not limited to, NCBI Reference Sequence: NM_001007524.2. Exemplary human F8A3 protein sequences include, but are not limited to, NCBI Reference Sequence: NP_001007525.1.

As used herein, the term “RBPJ,” “recombination signal binding protein for immunoglobulin kappa J region” refers to the gene RBPJ and the gene product encoded by the RBPJ gene. It is also known as “AOS3,” “CBF1,” “CBF-1,” “IGKJRB,” “IGKJRB1,” “KBF2,” “RBP-J,” “RBP-JK,” “RBP-J kappa,” “RBPJK,” “RBPSUH,” “suh”, or “csl.” In the human genome, RBPJ is located on chromosome 4. Exemplary human RBPJ transcript sequences include, but are not limited to, NCBI Reference Sequences: NM_015874.6 GI: 1864263252, NM_005349.4 GI: 1827625711, NM_203283.5 GI: 1898807824, NM_203284.3 GI: 1827625792, NM_001363577.2 GI: 1677500017, NM_001374400.1 GI: 1751363247, NM_001374401.1 GI: 1751363264, NM_001374402.1 GI: 1751363226, NM_001374403.1 GI: 1751363295, NM_001379406.1 GI: 1827625795, NM_001379407.1 GI: 1827625793, NM_001379408.1 GI: 1827625801, NM_001379409.1 GI: 1827625712, XM_011513840.3 GI: 1370486896, XM_017008170.2 GI: 1370486889, XM_017008171.2 GI: 1370486890 and XM_017008174.2 GI: 1370486894. Exemplary human RBPJ protein sequences include, but are not limited to, NCBI Reference Sequences: NP_056958.3 GI: 42560229, NP_005340.2 GI: 42560227, NP_976028.2 GI: 1898807825, NP_976029.1 GI: 42560223, NP_001350506.1 GI: 1391723665, NP_001361329.1 GI: 1751363248, NP_001361330.1 GI: 1751363265, NP_001361331.1 GI: 1751363227, NP_001361332.1 GI: 1751363296, NP_001366335.1 GI: 1827625796, NP_001366336.1 GI: 1827625794, NP_001366337.1 GI: 1827625802, NP_001366338.1 GI: 1827625713, XP_011512142.1 GI: 767929782, XP_016863659.1 GI: 1034639775, XP_016863660.1 GI: 1034639777, and XP_016863663.1 GI: 1034639783. Without wishing to be bound by theory, it is believed that in some embodiments, RBPJ is involved in Notch signaling.

As used herein, the term “ATP2C1,” “ATPase secretory pathway Ca2+ transporting 1” refers to the gene ATP2C1 and the gene product encoded by the ATP2C1 gene. It is also known as “ATP2C1A,” “BCPM,” “HHD”, “PMR1”, “SPCA1” or “hSPCA1.” In the human genome, ATP2C1 is located on chromosome 3. Exemplary human ATP2C1 transcript sequences include, but are not limited to, NCBI Reference Sequences: NM_001378687.1 GI: 1818371420, NM_014382.5 GI: 1890268947, NM_001001485.3 GI: 1890270720, NM_001001486.2 GI: 1811242813, NM_001001487.2 GI: 1811242792, NM_001199179.3 GI: 1890333373, NM_001199180.2 GI: 1677530482, NM_001199181.3 GI: 1811125338, NM_001199182.2 GI: 1677537706, NM_001199183.2 GI: 1677537664, NM_001199184.3 GI: 1890334324, NM_001199185.2 GI: 1677530151, NM_001378511.1 GI: 1811242745, NM_001378512.1 GI: 1811242741, NM_001378513.1 GI: 1811242788, NM_001378514.1 GI: 1811242778, and XM_011512686.2 GI: 1034632572. Exemplary human ATP2C1 protein sequences include, but are not limited to, NCBI Reference Sequences: NP_001365616.1 GI: 1818371421, NP_055197.2 GI: 48762685, NP_001001485.1 GI: 48762687, NP_001001486.1 GI: 48762689, NP_001001487.1 GI: 48762691, NP_001186108.1 GI: 312836763, NP_001186109.1 GI: 312836765, NP_001186110.1 GI: 312836767, NP_001186111.1 GI: 312836769, NP_001186112.1 GI: 312836771, NP_001186113.1 GI: 312836773, NP_001186114.1 GI: 312836775, NP_001365440.1 GI: 1811242746, NP_001365441.1 GI: 1811242742, NP_001365442.1 GI: 1811242789, NP_001365443.1 GI: 1811242779 and XP_011510988.1 GI: 767926392.

As used herein, the term “ICMT,” “isoprenylcysteine carboxyl methyltransferase” refers to the gene ICMT and the gene product encoded by the ICMT gene. It is also known as “HSTE14”, “MST098”, “MSTP098”, “PCCMT”, “PCMT,” or “PPMT.” In the human genome, ICMT is located on chromosome 1. Exemplary human ICMT transcript sequences include, but are not limited to, NCBI Reference Sequences: NM_012405.4 GI: 1519313708 and XM_011541140.2 GI: 1034557253. Exemplary human ICMT protein sequences include, but are not limited to, NCBI Reference Sequences: NP_036537.1 GI: 6912430 and XP_011539442.1 GI: 767903485. Without wishing to be bound by theory, it is believed that in some embodiments, ICMT is involved posttranslational modification of cysteine residues (e.g., C-terminal cysteine residues), e.g., in a target protein.

As used herein, the term “RABIF,” “RAB interacting factor” refers to the gene RABIF and the gene product encoded by the RABIF gene. It is also known as “MSS4,” “RASGFR3,” or “RASGRF3.” In the human genome, RABIF is located on chromosome 1. Exemplary human RABIF transcript sequences include, but are not limited to, NCBI Reference Sequences: NM_002871.5. Exemplary human RABIF protein sequences include, but are not limited to, NCBI Reference Sequences: NP_002862.2. Without wishing to be bound by theory, it is believed that in some embodiments, RABIF is involved in intracellular vesicular transport.

As used herein, the term “IER3IP1,” “immediate early response 3 interacting protein 1” refers to the gene IER3IP1 and the gene product encoded by the IER3IP1 gene. It is also known as “HSPC039,” “MEDS,” or “PR02309.” In the human genome, IER3IP1 is located on chromosome 18. Exemplary human IER3IP1 transcript sequences include, but are not limited to, NCBI Reference Sequences: NM_016097.5. Exemplary human IER3IP1 protein sequences include, but are not limited to, NCBI Reference Sequences: NP_057181.1.

As used herein, the term “RBM10,” “RNA binding motif protein 10” refers to the gene RBM10 and the gene product encoded by the RBM10 gene. It is also known as “DXS8237E”, “GPATC9”, “GPATCH9”, “S1-1”, “TARPS,” or “ZRANB5.” In the human genome, RBM10 is located on chromosome X. Exemplary human RBM10 transcript sequences include, but are not limited to, NCBI Reference Sequences: NM_005676.5 GI: 1519245581, NM_152856.3 GI: 1676319259, NM_001204466.2 GI: 1674985860,NM_001204467.2 GI: 1676318417,NM_001204468.2 GI: 1890332849,XM_005272677.4 GI: 1034675405,M_005272678.4 GI: 1034675406,XM_005272679.4 GI: 1034675407,XM_005272679.4 GI: 1034675407, XM_017029884.2 GI: 1370525041,XM_024452457.1 GI: 1370525025,XM_024452458.1 GI: 1370525027,XM_024452459.1 GI: 1370525029, XM_024452460.1 GI: 1370525031,XM_024452461.1 GI: 1370525033, XM_024452462.1 GI: 1370525035,XM_024452463.1 GI: 1370525037, XM_024452464.1 GI: 1370525039, and XM_024452465.1 GI: 1370525042. Exemplary human RBM10 protein sequences include, but are not limited to, NCBI Reference Sequences: NP_005667.2 GI: 20127479,NP_690595.1 GI: 23111018, NP_001191395.1 GI: 325120982,NP_001191396.1 GI: 325120984,NP_001191397.1 GI: 325120986,XP_005272734.1 GI: 530421598,XP_005272735.1 GI: 530421600,XP_005272736.1 GI: 530421602,XP_016885374.1 GI: 1034675411,XP_016885373.1 GI: 1034675409,XP_024308225.1 GI: 1370525026,XP_024308226.1 GI: 1370525028, XP_024308227.1 GI: 1370525030,XP_024308228.1 GI: 1370525032,XP_024308229.1 GI: 1370525034,XP_024308230.1 GI: 1370525036,XP_024308231.1 GI: 1370525038,XP_024308232.1 GI: 1370525040, and XP_024308233.1 GI: 1370525043.

As used herein, the term “SMG7,” “SMG7 nonsense mediated mRNA decay factor” refers to the gene SMG7 and the gene product encoded by the SMG7 gene. It is also known as “Clorf16,” “EST1C,” or “SGA56M.” In the human genome, SMG7 is located on chromosome 1. Exemplary human SMG7 transcript sequences include, but are not limited to, NCBI Reference Sequences: NM_001375584.1 GI: 1770726525,NM_173156.3 GI: 1677538354,NM_201568.3 GI: 1677498966,NM_201569.3 GI: 1677499314,NM_001174061.2 GI: 1677499774,NM_001331007.2 GI: 1677538543,NM_001350219.2 GI: 1677539466,NM_001350220.2 GI: 1677501685,NM_001350221.2 GI: 1677530910,NM_001375585.1 GI: 1770726527, NM_001394133.1 GI: 2021392911,NM_001394134.1 GI: 2021392906,NM_001394135.1 GI: 2021392924,NM_001394136.1 GI: 2021392870,NM_001394137.1 GI: 2021392885,NM_001394138.1 GI: 2021392909,NM_001394139.1 GI: 2021392893,M_001394140.1 GI: 2021392877,NM_001394141.1 GI: 2021392879,NM_001394142.1 GI: 2021392915, NM_001394143.1 GI:2021392902, NM_001394144.1 GI: 2021392891,NM_001394145.1 GI: 2021392900, NM_001394146.1 GI: 2021392875,NM_001394147.1 GI: 2021392881,XM_005245653.5 GI: 1370455510,XM_011510205.3 GI: 1370455506,XM_011510206.3 GI: 1370455508,XM_011510207.3 GI: 1370455509,XM_017002969.1 GI: 1034563782, and XM_017002970.1 GI: 1034563784. Exemplary human SMG7 protein sequences include, but are not limited to, NCBI Reference Sequences: NP_001362513.1 GI: 1770726526,NP_775179.1 GI: 42475558, NP_963862.1 GI: 42476070,NP_963863.2 GI: 291327487, NP_001167532.1 GI: 291327485, NP_001317936.1 GI: 1061899765,NP_001337148.1 GI: 1175460267,NP_001337149.1 GI: 1175460293,NP_001337150.1 GI: 1175460328,NP_001362514.1 GI: 1770726528,NP_001381062.1 GI: 2021392912, NP_001381063.1 GI: 2021392907, NP_001381064.1 GI: 2021392925,NP_001381065.1 GI: 2021392871,NP_001381066.1 GI: 2021392886,NP_001381067.1 GI: 2021392910,NP_001381068.1 GI: 2021392894,NP_001381069.1 GI: 2021392878,NP_001381070.1 GI: 2021392880,NP_001381071.1 GI: 2021392916, NP_001381072.1 GI: 2021392903,NP_001381073.1 GI: 2021392892, NP_001381074.1 GI: 2021392901, NP_001381075.1 GI: 2021392876, NP_001381076.1 GI: 2021392882,XP_005245710.1 GI: 530365723,XP_011508507.1 GI: 767911044, XP_011508508.1 GI: 767911049,XP_011508509.1 GI: 767911052, XP_016858458.1 GI: 1034563783, and XP_016858459.1 GI: 1034563785.

As used herein, the term “GTF2I,” “general transcription factor IIi” refers to the gene GTF2I and the gene product encoded by the GTF2I gene. It is also known as “BAP135”, “BTKAP1”, “DIWS”, “GTFII-I”, “IB291”, “SPIN”, “TFII-I”, “WBS,” or “WBSCR6.” In the human genome, GTF2I is located on chromosome 7. Exemplary human GTF2I transcript sequences include, but are not limited to, NCBI Reference Sequences: NM_032999.4 GI: 1677538443, NM_001518.5 GI: 1677531336, NM_033000.4 GI: 1677473409, NM_033001.4 GI: 1677537301, NM_001163636.3 GI: 1677498311 and NM_001280800.2 GI: 1890273846. Exemplary human GTF2I protein sequences include, but are not limited to, NCBI Reference Sequences: NP_127492.1 GI: 14670350, NP_001509.3 GI: 169881252, NP_127493.1 GI: 14670352, NP_127494.1 GI: 14670354, NP_001157108.1 GI: 254692934, and NP_001267729.1 GI: 526253077.

As used herein, the term “ELAVL1,” “ELAV like RNA binding protein 1” refers to the gene ELAVL1 and the gene product encoded by the ELAVL1 gene. It is also known as “ELAV1, “HUR”, “Hua,” or “MelG.” In the human genome, ELAVL1 is located on chromosome 19. Exemplary human ELAVL1 transcript sequences include, but are not limited to, NCBI Reference Sequences: NM_001419.3 GI: 1519245119, XR_001753627.2 GI: 1370474630, and XR_001753628.2 GI: 1370474631. Exemplary human ELAVL1 protein sequences include, but are not limited to, NCBI Reference Sequence: NP_001410.2.

As used herein, the term “MEPCE,” “methylphosphate capping enzyme” refers to the gene MEPCE and the gene product encoded by the MEPCE gene. It is also known as “BCDIN3.” In the human genome, MEPCE is located on chromosome 7. Exemplary human MEPCE transcript sequences include, but are not limited to, NCBI Reference Sequences: NM_019606.6 GI: 1519312573, NM_001194990.2 GI: 1676441753, NM_001194991.2 GI: 1675076437, NM_001194992.2 GI: 1676440219, and NM_001363486.2 GI: 1675176086. Exemplary human MEPCE protein sequences include, but are not limited to, NCBI Reference Sequences: NP_062552.2 GI: 47271406, NP_001181919.1 GI: 303521310, NP_001181920.1 GI: 303521337, NP_001181921.1 GI: 303521460, and NP_001350415.1 GI: 1390249168.

As used herein, the term “RAB4A,” “RAB4A, member RAS oncogene family” refers to the gene RAB4A and the gene product encoded by the RAB4A gene. It is also known as “HRES-1”, “HRES-1/RAB4”, “HRES1” or “RAB4.” In the human genome, RAB4A is located on chromosome 1. Exemplary human RAB4A transcript sequences include, but are not limited to, NCBI Reference Sequences: NM_004578.4 GI: 1653960951, NM_001271998.2 GI: 1890242165, and NR_073545.2 GI: 1890398232. Exemplary human RAB4A protein sequences include, but are not limited to, NCBI Reference Sequences: NP_004569.2 GI: 19923260 and NP_001258927.1 GI: 433660831.

As used herein, the term “IER3IP1,” “immediate early response 3 interacting protein 1” refers to the gene IER3IP1 and the gene product encoded by the IER3IP1 gene. It is also known as “HSPC039,” “MEDS,” or “PR02309.” In the human genome, IER3IP1 is located on chromosome 18. Exemplary human IER3IP1 transcript sequences include, but are not limited to, NCBI Reference Sequence: NM_016097.5. Exemplary human IER3IP1 protein sequences include, but are not limited to, NCBI Reference Sequence: NP_057181.1.

As used herein, the term “SAMD1,” “sterile alpha motif domain containing 1” refers to the gene SAMD1 and the gene product encoded by the SAMD1 gene. In the human genome, SAMD1 is located on chromosome 19. Exemplary human SAMD1 transcript sequences include, but are not limited to, NCBI Reference Sequence NM_138352.3. Exemplary human SAMD1 protein sequences include, but are not limited to, NCBI Reference Sequence: NP_612361.1.

As used herein, the term “AP2A1,” “adaptor related protein complex 2 subunit alpha 1” refers to the gene AP2A1 and the gene product encoded by the AP2A1 gene. It is also known as “ADTAA,” “AP2-ALPHA,” or “CLAPA1.” In the human genome, AP2A1 is located on chromosome 19. Exemplary human AP2A1 transcript sequences include, but are not limited to, NCBI Reference Sequences: NM_130787.3 GI: 1677530496, NM_014203.3 GI: 1677556772. XM_011526556.2 GI: 1034606499, and XM_011526557.3 GI: 1370474558. Exemplary human AP2A1 protein sequences include, but are not limited to, NCBI Reference Sequences: NP_570603.2 GI: 19913416, NP_055018.2 GI: 19913414, XP_011524858.1 GI: 768007010, and XP_011524859.1 GI: 768007014. Without wishing to be bound by theory, it is believed that in some embodiments, AP2A1 is involved in clathrin-coated vesicle trafficking.

As used herein, the term “AP2B1,” “adaptor related protein complex 2 subunit beta 1” refers to the gene AP2B1 and the gene product encoded by the AP2B1 gene. It is also known as “ADTB2”, “AP105B”, “AP2-BETA,” or “CLAPB1.” In the human genome, AP2B1 is located on chromosome 17. Exemplary human AP2B1 transcript sequences include, but are not limited to, NCBI Reference Sequences: NM_001030006.2 GI: 1519245384, NM_001282.3 GI: 1676317279, XM_005257937.4 GI: 1370470335, XM_005257938.3 GI: 1370470336, XM_005257941.3 GI: 1370470338, XM_011524448.2 GI: 1370470331, XM_011524449.3 GI: 1370470332, XM_011524450.2 GI: 1370470333, XM_011524451.2 GI: 1370470334, XM_011524452.1 GI: 767993865, XM_011524453.1 GI: 767993867, XM_011524454.1 GI: 767993869, XM_011524455.2 GI: 1034598536, XM_017024284.2 GI: 1370470337, XM_017024285.1 GI: 1034598534, XM_017024286.1 GI: 1034598537, and XM_017024287.2 GI: 1370470339. Exemplary human AP2B1 protein sequences include, but are not limited to, NCBI Reference Sequences: NP_001025177.1 GI: 71773106, NP_001273.1 GI: 4557469, XP_005257994.1 GI: 530410945, XP_005257995.1 GI: 530410947, XP_005257998.1 GI: 530410953, XP_011522750.1 GI: 767993857, XP_011522751.1 GI: 767993859, XP_011522752.1 GI: 767993861, XP_011522753.1 GI: 767993863, XP_011522754.1 GI: 767993866, XP_011522755.1 GI: 767993868, XP_011522756.1 GI: 767993870, XP_011522757.1 GI: 767993872, XP_016879773.1 GI: 1034598533, XP_016879774.1 GI: 1034598535, XP_016879775.1 GI: 1034598538, and XP_016879776.1 GI: 1034598540. Without wishing to be bound by theory, it is believed that in some embodiments, AP2B1 is involved in clathrin-coated vesicle trafficking.

As used herein, the term “TM9SF4,” “transmembrane 9 superfamily member 4” refers to the gene TM9SF4 and the gene product encoded by the TM9SF4 gene. It is also known as “dJ836N17.2.” In the human genome, TM9SF4 is located on chromosome 20. Exemplary human TM9SF4 transcript sequences include, but are not limited to, NCBI Reference Sequences: NM_014742.4 GI: 1653960410, NM_001363731.2 GI: 1890270685, XM_017028154.1 GI: 1034626121, XM_017028155.1 GI: 1034626123, XM_017028156.1 GI: 1034626125, XM_017028157.1 GI: 1034626127, XM_017028158.1 GI: 1034626129, XM_017028159.1 GI: 1034626131, XM_017028160.1 GI: 1034626133, and XM_017028161.1 GI: 1034626135. Exemplary human TM9SF4 protein sequences include, but are not limited to, NCBI Reference Sequences: NP_055557.2 GI: 164519076, NP_001350660.1 GI: 1393169939, XP_016883643.1 GI: 1034626122, XP_016883644.1 GI: 1034626124, XP_016883645.1 GI: 1034626126, XP_016883646.1 GI: 1034626128, XP_016883647.1 GI: 1034626130, XP_016883648.1 GI: 1034626132, XP_016883649.1 GI: 1034626134, and XP_016883650.1 GI: 1034626136. Without wishing to be bound by theory, it is believed that in some embodiments, TM9SF4 is involved in localization of polypeptides comprising glycine-rich transmembrane domains, e.g., to the cell surface.

As used herein, the term “ACTR5,” “actin related protein 5” refers to the gene ACTR5 and the gene product encoded by the ACTR5 gene. It is also known as “Arp5,” or “INO80M.” In the human genome, ACTR5 is located on chromosome 20. Exemplary human ACTR5transcript sequences include, but are not limited to, NCBI Reference Sequence: NM_024855.4. Exemplary human ACTR5 protein sequences include, but are not limited to, NCBI Reference Sequence NP_079131.3. Without wishing to be bound by theory, it is believed that in some embodiments, ACTR5 is involved in with DNA repair, e.g., after DNA damage.

As used herein, the term “PTMA,” “prothymosin alpha” refers to the gene PTMA and the gene product encoded by the PTMA gene. It is also known as “TMSA.” In the human genome, PTMA is located on chromosome 2. Exemplary human PTMA transcript sequences include, but are not limited to, NCBI Reference Sequences: NM_002823.5 GI: 1843978475 and NM_001099285.2 GI: 1843978432. Exemplary human PTMA protein sequences include, but are not limited to, NCBI Reference Sequences: NP_002814.3 GI: 151101404 and NP_001092755.1 GI: 151101407.

As used herein, the term “PAPOLA,” “poly(A) polymerase alpha” refers to the gene PAPOLA and the gene product encoded by the PAPOLA gene. It is also known as “PAP,” or “PAP-alpha.” In the human genome, PAPOLA is located on chromosome 14. Exemplary human PAPOLA transcript sequences include, but are not limited to, NCBI Reference Sequences: NM_032632.5 GI: 1919270907, NM_001252006.1 GI: 354681990, NM_001252007.1 GI: 354681992, NM_001293627.1 GI: 651166637, NM_001293628.2 GI: 1889599212, NM_001293632.3 GI: 1890275058, NM_001363662.3 GI: 1890341920, NM_001363664.3 GI: 1890266986, NM_001363665.3 GI: 1890257812, and NM_001363666.3 GI: 1889638692. Exemplary human PAPOLA protein sequences include, but are not limited to, NCBI Reference Sequences: NP_116021.2 GI: 32490557, NP_001238935.1 GI: 354681991, NP_001238936.1 GI: 354681993, NP_001280556.1 GI: 651166638, NP_001280557.1 GI: 651166631, NP_001280561.1 GI: 651166787, NP_001350591.1 GI: 1393428056, NP_001350593.1 GI: 1393169738, NP_001350594.1 GI: 1393169572, and NP_001350595.1 GI: 1393169776. Without wishing to be bound by theory, it is believed that in some embodiments, PAPOLA is involved in poly(A) tail synthesis.

As used herein, the term “PDHA1,” “pyruvate dehydrogenase E1 subunit alpha 1” refers to the gene PDHA1 and the gene product encoded by the PDHA1 gene. It is also known as “PDHA, “PDHAD”, “PDHCE1A,” or “PHE1A.” In the human genome, PDHA1 is located on chromosome X. Exemplary human PDHA1 transcript sequences include, but are not limited to, NCBI Reference Sequences: NM_000284.4 GI: 1653961155, NM_001173454.2 GI: 1843979930, NM_001173455.2 GI: 1843979931, NM_001173456.2 GI: 1843979929, and XM_017029574.2 GI: 1370517093. Exemplary human PDHA1 protein sequences include, but are not limited to, NCBI Reference Sequences: NP_000275.1 GI: 4505685, NP_001166925.1 GI: 291084742, NP_001166926.1 GI: 291084744, NP_001166927.1 GI: 291084757, and XP_016885063.1 GI: 1034674482. Without wishing to be bound by theory, it is believed that in some embodiments, PDHA1 is involved in glucose metabolism.

As used herein, the term “PDHB,” “pyruvate dehydrogenase E1 subunit beta” refers to the gene PDHB and the gene product encoded by the PDHB gene. It is also known as “PDHBD, “PDHE1-B”, “PDHE1B,” or “PHE1B.” In the human genome, PDHB is located on chromosome 3. Exemplary human PDHB transcript sequences include, but are not limited to, NCBI Reference Sequences: NM_000925.4 GI: 1519242530, NM_001173468.2 GI: 1889486831, NM_001315536.2 GI: 1890267224, and NR_033384.2 GI: 1890527347. Exemplary human PDHB protein sequences include, but are not limited to, NCBI Reference Sequences: NP_000916.2 GI: 156564403, NP_001166939.1 GI: 291084858, and NP_001302465.1 GI: 937576204. Without wishing to be bound by theory, it is believed that in some embodiments, PDHB is involved in glucose metabolism.

As used herein, the term “SLC25A19,” “solute carrier family 25 member 19” refers to the gene SLC25A19 and the gene product encoded by the SLC25A19 gene. It is also known as “DNC”, “MCPHA”, “MUP1”, “THMD3”, “THMD4,” or “TPC.” In the human genome, SLC25A19 is located on chromosome 17. Exemplary human SLC25A19 transcript sequences include, but are not limited to, NCBI Reference Sequences: NM_001126121.2 GI: 1519243709, NM_021734.5 GI: 1890265749, NM_001126122.2 GI: 1890263339, XM_005257559.4 GI: 1370471798, XM_005257560.2 GI: 1034600730, XM_005257561.4 GI: 1370471800, XM_005257562.2 GI: 1034600729, XM_006722007.2 GI: 1034600732, XM_017024926.2 GI: 1370471802, XM_017024927.2 GI: 1370471803, and XM_017024928.2 GI: 1370471804. Exemplary human SLC25A19 protein sequences include, but are not limited to, NCBI Reference Sequences: NP_001119593.1 GI: 186928858, NP_068380.3 GI: 186928856, NP_001119594.1 GI: 186928860, XP_005257616.1 GI: 530412630, P_005257617.1 GI: 530412632, XP_005257618.1 GI: 530412634, XP_005257619.1 GI: 530412636, XP_006722070.1 GI: 578831220, XP_016880415.1 GI: 1034600734, XP_016880416.1 GI: 1034600736, and XP_016880417.1 GI: 1034600738.

As used herein, the term “GAK,” “cyclin G associated kinase” refers to the gene GAK and the gene product encoded by the GAK gene. It is also known as “DNAJ26,” or “DNAJC26.” In the human genome, GAK is located on chromosome 4. Exemplary human GAK transcript sequences include, but are not limited to, NCBI Reference Sequences: NM_005255.4 GI: 1519315059, NM_001318134.2 GI: 1675141630, XM_005272268.2 GI: 1034639251, XM_005272270.2 GI: 1034639259, XM_011513425.2 GI: 1034639247, XM_011513426.2 GI: 1034639248, XM_011513427.2 GI: 1034639249, XM_011513428.2 GI: 1034639250, XM_011513429.2 GI: 1034639252, XM_011513430.1 GI: 767929048, XM_011513431.2 GI: 1034639255, XM_011513432.2 GI: 1034639256, XM_011513434.2 GI: 1034639260, XM_017007991.1 GI: 1034639253, XM_017007992.1 GI: 1034639257, XM_017007993.1 GI: 1034639261, XM_017007994.1 GI: 1034639263, and XM_017007995.1 GI: 1034639265. Exemplary human GAK protein sequences include, but are not limited to, NCBI Reference Sequences: NP_005246.2 GI: 157384971, NP_001305063.1 GI: 970414541, XP_005272325.1 GI: 530427333, XP_005272327.1 GI: 530427337, XP_011511727.1 GI: 767929039, XP_011511728.1 GI: 767929041, XP_011511729.1 GI: 767929043, XP_011511730.1 GI: 767929045, XP_011511731.1 GI: 767929047 XP_011511732.1 GI: 767929049, XP_011511733.1 GI: 767929051, XP_011511734.1 GI: 767929053, XP_011511736.1 GI: 767929057, XP_016863480.1 GI: 1034639254, XP_016863481.1 GI: 1034639258, XP_016863482.1 GI: 1034639262, XP_016863483.1 GI: 1034639264, and XP_016863484.1 GI: 1034639266.

As used herein, the term “AUNIP,” “aurora kinase A and ninein interacting protein” refers to the gene AUNIP and the gene product encoded by the AUNIP gene. It is also known as “AIBP,” or “Clorf135.” In the human genome, AUNIP is located on chromosome 1. Exemplary human AUNIP transcript sequences include, but are not limited to, NCBI Reference Sequences: NM_024037.3 GI: 1677539366 and NM_001287490.2 GI: 1890269921. Exemplary human AUNIP protein sequences include, but are not limited to, NCBI Reference Sequences: NP_076942.1 GI: 13128990 and NP_001274419.1 GI: 566559859. Without wishing to be bound by theory, it is believed that in some embodiments, AUNIP is involved in with DNA resection and/or homologous recombination, e.g., after DNA damage.

As used herein, the term “FCHO2,” “FCH and mu domain containing endocytic adaptor 2” refers to the gene FCHO2 and the gene product encoded by the FCHO2 gene. In the human genome, FCHO2 is located on chromosome 5. Exemplary human FCHO2 transcript sequences include, but are not limited to, NCBI Reference Sequences: NM_138782.3 GI: 1519312139, NM_001146032.2 GI: 1676317056, XM_017009016.2 GI: 1370488431, XM_017009017.2 GI: 1370488432, XM_017009018.2 GI: 1370488433, XM_017009019.2 GI: 1370488434, XM_017009020.2 GI: 1370488436, XM_017009021.2 GI: 1370488437, XM_017009022.2 GI: 1370488438, XM_017009023.2 GI: 1370488439, and XR_001741993.2 GI: 1370488435. Exemplary human FCHO2 protein sequences include, but are not limited to, NCBI Reference Sequences: NP_620137.2 GI: 226371723, NP_001139504.1 GI: 226371725, XP_016864505.1 GI: 1034643509, XP_016864506.1 GI: 1034643511, XP_016864507.1 GI: 1034643513, XP_016864508.1 GI: 1034643515, XP_016864509.1 GI: 1034643518, XP_016864510.1 GI: 1034643520, XP_016864511.1 GI: 1034643522, and XP_016864512.1 GI: 1034643524. Without wishing to be bound by theory, it is believed that in some embodiments, FCHO2 is involved in clathrin-mediated endocytosis.

As used herein, the term “ACTB,” “actin beta” refers to the gene ACTB and the gene product encoded by the ACTB gene. It is also known as “BRWS1,” or “PS1TP5BP1.” In the human genome, ACTB is located on chromosome 7. Exemplary human ACTB transcript sequences include, but are not limited to, NCBI Reference Sequence: NM_001101.5. Exemplary human ACTB protein sequences include, but are not limited to, NCBI Reference Sequence: NP_001092.1. Without wishing to be bound by theory, it is believed that in some embodiments, ACTB is involved in vesicular trafficking.

As used herein, the term “LIPT1,” “lipoyltransferase 1” refers to the gene LIPT1 and the gene product encoded by the LIPT1 gene. In the human genome, LIPT1 is located on chromosome 2. Exemplary human LIPT1 transcript sequences include, but are not limited to, NCBI Reference Sequences: NM_145199.3 GI: 1519314998, NM_015929.4 GI: 1675128718, NM_145197.3 GI: 1675116628, NM_145198.3 GI: 1676318578, NM_001204830.2 GI: 1890346109, NR_037935.2 GI: 1890350262, and NR_037936.2 GI: 1700660402. Exemplary human LIPT1 protein sequences include, but are not limited to, NCBI Reference Sequences: NP_660200.1 GI: 21729884, NP_057013.1 GI: 7706252, NP_660198.1 GI: 21729880, NP_660199.1 GI: 21729882, and NP_001191759.1 GI: 325651943.

As used herein, the term “UCHL5,” “ubiquitin C-terminal hydrolase L5” refers to the gene UCHL5 and the gene product encoded by the UCHL5 gene. It is also known as “CGI-70”, “INO80R”, “UCH-L5,” or “UCH37.” In the human genome, UCHL5 is located on chromosome 1. Exemplary human UCHL5 transcript sequences include, but are not limited to, NCBI Reference Sequences: NM_001199261.3 GI: 1677498713, NM_015984.5 GI: 1677499918, NM_001199262.3 GI: 1677499101, NM_001199263.3 GI: 1677500535, NM_001350840.2 GI: 1677530706, NM_001350841.2 GI: 1677500705, M_001350842.2 GI: 1677501845, NM_001350843.2 GI: 1677537781, NM_001350844.2 GI: 1677538347, NM_001350845.2 GI: 1677498797, NM_001350846.2 GI: 1677499458, NM_001350847.2 GI: 1677496678, NM_001350848.2 GI: 1677530178, NM_001350849.2 GI: 1677500311, NM_001350850.2 GI: 1677478356, NM_001350851.2 GI: 1677556738, NM_001350852.2 GI: 1677501892, NR_037607.2 GI: 1677539654, NR_146930.2 GI: 1700447760, NR_146931.2 GI: 1700660389, XM_005245246.5 GI: 1370453397, XM_006711367.4 GI: 1370453390, XM_006711369.3 GI: 1370453394, XM_006711370.4 GI: 1370453402, XM_006711371.3 GI: 1034559064, XM_011509604.2 GI: 1370453391, XM_011509607.2 GI: 1034559050, XM_011509608.3 GI: 1370453403, XM_017001431.2 GI: 1370453395, XM_017001430.2 GI: 1370453392, XM_017001433.2 GI: 1370453400, XM_017001432.2 GI: 1370453396, XM_017001436.2 GI: 1370453404, XM_017001438.1 GI: 1034559062, XM_017001439.1 GI: 1034559065, XM_017001443.1 GI: 1034559074, XR_921823.3 GI: 1370453398, XR_001737214.2 GI: 1370453393, XR_001737217.2 GI: 1370453399, XR_001737218.2 GI: 1370453401, XR_001737219.2 GI: 1370453405, XR_002956782.1 GI: 1370453406, and XR_002956783.1 GI: 1370453407. Exemplary human UCHL5 protein sequences include, but are not limited to, NCBI Reference Sequences: NP_001186190.1 GI: 312922359, NP_057068.1 GI: 7706753, NP_001186191.1 GI: 312922361, NP_001186192.1 GI: 312922364, NP_001337769.1 GI: 1186128475, NP_001337770.1 GI: 1186128449, NP_001337771.1 GI: 1186517924, NP_001337772.1 GI: 1186517903, NP_001337773.1 GI: 1186128426, NP_001337774.1 GI: 1186128416, NP_001337775.1 GI: 1186128509, NP_001337776.1 GI: 1186128513, NP_001337777.1 GI: 1186128412, NP_001337778.1 GI: 1186128457, P_001337779.1 GI: 1186128465, P_001337780.1 GI: 1186517889, NP_001337781.1 GI: 1186128523, XP_005245303.1 GI: 530364883, XP_006711430.1 GI: 578801056, XP_006711432.1 GI: 578801060, XP_006711433.3 GI: 1034559047, XP_006711434.1 GI: 578801068, XP_011507906.1 GI: 767909463, XP_011507909.1 GI: 767909476, XP_011507910.1 GI: 767909480, XP_016856920.1 GI: 1034559037, XP_016856919.1 GI: 1034559031, XP_016856922.1 GI: 1034559044, XP_016856921.1 GI: 1034559039, XP_016856925.1 GI: 1034559058, XP_016856927.1 GI: 1034559063, XP_016856928.1 GI: 1034559066, and XP_016856932.1 GI: 1034559075. Without wishing to be bound by theory, it is believed that in some embodiments, UCHL5 is involved in DNA repair, e.g., after DNA damage.

As used herein, the term “INO80E,” “INO80 complex subunit E” refers to the gene INO80E and the gene product encoded by the INO80E gene. It is also known as “CCDC95.” In the human genome, INO80E is located on chromosome 16. Exemplary human INO80E transcript sequences include, but are not limited to, NCBI Reference Sequences: NM_173618.3 GI: 1519243905, NM_001304562.2 GI: 1675067975, NM_001304563.2 GI: 1676317488, NR_130786.2 GI: 1700447788, NR_130787.2 GI: 1701970477, NR_134855.2 GI: 1700447928, XM_011545809.3 GI: 1370468313, XM_011545811.3 GI: 1370468315, XM_011545812.3 GI: 1370468316, XM_017023169.2 GI: 1370468306, XM_024450236.1 GI: 1370468307, XM_024450237.1 GI: 1370468309, XM_024450238.1 GI: 1370468311, XR_001751894.2 GI: 1370468318, XR_002957798.1 GI: 1370468314, and XR_002957799.1 GI: 1370468317. Exemplary human INO80E protein sequences include, but are not limited to, NCBI Reference Sequences: NP_775889.1 GI: 27734727, NP_001291491.1 GI: 752507708, NP_001291492.1 GI: 752293507, XP_011544111.1 GI: 767988460, XP_011544113.1 GI: 767988465, XP_011544114.1 GI: 767988468, XP_016878658.1 GI: 1034594377, XP_024306004.1 GI: 1370468308, XP_024306005.1 GI: 1370468310, and XP_024306006.1 GI: 1370468312. Without wishing to be bound by theory, it is believed that in some embodiments, INO80E is involved in DNA repair, e.g., after DNA damage.

As used herein, the term “ECHS1,” “enoyl-CoA hydratase, short chain 1” refers to the gene ECHS1 and the gene product encoded by the ECHS1 gene. It is also known as “ECHS1D” or “SCEH.” In the human genome, ECHS1 is located on chromosome 10. Exemplary human ECHS1 transcript sequences include, but are not limited to, NCBI Reference Sequences: NM_004092.4 GI: 1519313412 and XR_002956965.1 GI: 1370456580. Exemplary human ECHS1 protein sequences include, but are not limited to, NCBI Reference Sequence: NP_004083.3 GI: 194097323. Without wishing to be bound by theory, it is believed that in some embodiments, ECHS1 is involved in mitochondrial fatty acid beta-oxidation.

As used herein, the term “NELFB,” “negative elongation factor complex member B” refers to the gene NELFB and the gene product encoded by the NELFB gene. It is also known as “COBRA1,” or “NELF-B.” In the human genome, NELFB is located on chromosome 9. Exemplary human NELFB transcript sequences include, but are not limited to, NCBI Reference Sequence: NM_015456.5 GI: 1519314791. Exemplary human NELFB protein sequences include, but are not limited to, NCBI Reference Sequence: NP_056271.3 GI: 862669351. Without wishing to be bound by theory, it is believed that in some embodiments, NELFB is involved in elongation of an mRNA by RNA polymerase II.

As used herein, the term “EDC4,” “enhancer of mRNA decapping 4” refers to the gene EDC4 and the gene product encoded by the EDC4 gene. It is also known as “GE1”, “Ge-1”, “HEDL5”, “HEDLS”, “RCD-8,” or “RCD8.” In the human genome, EDC4 is located on chromosome 16. Exemplary human EDC4 transcript sequences include, but are not limited to, NCBI Reference Sequence: NM_014329.5 GI: 1519311623. Exemplary human EDC4 protein sequences include, but are not limited to, NCBI Reference Sequence: QTW92174.1 GI: 2026653161. Without wishing to be bound by theory, it is believed that in some embodiments, EDC4 is involved in mRNA degradation.

As used herein, the term “PRMT1,” “protein arginine methyltransferase 1” refers to the gene PRMT1 and the gene product encoded by the PRMT1 gene. It is also known as “ANM1”, “HCP1”, “HRMTIL2,” or “IR1B4.” In the human genome, PRMT1 is located on chromosome 19. Exemplary human PRMT1 transcript sequences include, but are not limited to, NCBI Reference Sequences: NM_001536.6 GI: 1843978399, NM_198318.5 GI: 1843978383, NM_001207042.3 GI: 1843978501, NR_033397.5 GI: 1843978545, XM_005258842.1 GI: 530416390, XM_017026734.1 GI: 1034607688, XM_017026735.1 GI: 1034607690, and XM_017026736.1 GI: 1034607692. Exemplary human PRMT1 protein sequences include, but are not limited to, NCBI Reference Sequences: NP_001527.3 GI: 154759421, NP_938074.2 GI: 151301219, NP_001193971.1 GI: 333360913, XP_005258899.1 GI: 530416391, XP_016882223.1 GI: 1034607689, XP_016882224.1 GI: 1034607691, and XP_016882225.1 GI: 1034607693. Without wishing to be bound by theory, it is believed that in some embodiments, PRMT1 is involved in DNA repair, e.g., after DNA damage.

As used herein, the term “PDS5B,” “PDS5 cohesin associated factor B” refers to the gene PDS5B and the gene product encoded by the PDS5B gene. It is also known as “APRIN,” “AS3,” or “CG008.” In the human genome, PDS5B is located on chromosome 13. Exemplary human PDS5B transcript sequences include, but are not limited to, NCBI Reference Sequences: NM_015032.4 GI: 1519312474, XM_005266298.4 GI: 1034584014, XM_011534999.2 GI: 1034584017, XM_011535000.2 GI: 1034584018, XM_011535002.3 GI: 1370463706, XM_017020448.1 GI: 1034584015, XM_017020449.1 GI: 1034584019, XM_017020450.1 GI: 1034584021, XM_017020451.1 GI: 1034584023, XM_017020452.2 GI: 1370463705, and XM_017020453.1 GI: 1034584027. Exemplary human PDS5B protein sequences include, but are not limited to, NCBI Reference Sequences: NP_055847.1 GI: 7657269, XP_005266355.1 GI: 530402180, XP_011533301.1 GI: 767977463, XP_011533302.1 GI: 767977465, XP_011533304.1 GI: 767977470, XP_016875937.1 GI: 1034584016, XP_016875938.1 GI: 1034584020, XP_016875939.1 GI: 1034584022, XP_016875940.1 GI: 1034584024, XP_016875941.1 GI: 1034584026, and P_016875942.1 GI: 1034584028. Without wishing to be bound by theory, it is believed that in some embodiments, PDS5B is involved in DNA repair, e.g., after DNA damage.

As used herein, the term “PELI3,” “pellino E3 ubiquitin protein ligase family member 3” refers to the gene PELI3 and the gene product encoded by the PELI3 gene. In the human genome, PELI3 is located on chromosome 11. Exemplary human PELI3 transcript sequences include, but are not limited to, NCBI Reference Sequences: NM_145065.3 GI: 1519314877, NM_001098510.2 GI: 1890264363, NM_001243135.2 GI: 1890284473, NM_001243136.2 GI: 1889672634, XM_011544884.2 GI: 1034572805, and XM_017017465.1 GI: 1034572806. Exemplary human PELI3 protein sequences include, but are not limited to, NCBI Reference Sequences NP_659502.2 GI: 148612798, NP_001091980.1 GI: 148612833, NP_001230064.1 GI: 341823687, NP_001230065.1 GI: 341823689, XP_011543186.1 GI: 767967798, and XP_016872954.1 GI: 1034572807. Without wishing to be bound by theory, it is believed that in some embodiments, PELI3 is involved in innate immune response.

As used herein, the term “SLC35A1,” “solute carrier family 35 member A1” refers to the gene SLC35A1 and the gene product encoded by the SLC35A1 gene. It is also known as “CDG2F”, “CMPST”, “CST,” or “hCST.” In the human genome, SLC35A1 is located on chromosome 6. Exemplary human SLC35A1 transcript sequences include, but are not limited to, NCBI Reference Sequences: NM_006416.5 GI: 1519246076 and NM_001168398.2 GI: 1890335541. Exemplary human SLC35A1 protein sequences include, but are not limited to, NCBI Reference Sequences: NP_006407.1 GI: 5453621 and NP_001161870.1 GI: 270288773. Without wishing to be bound by theory, it is believed that in some embodiments, SLC35A1 is involved in transport of CMP-sialic acid from cytosol to a Golgi vesicle.

As used herein, the term “GREB1L,” “GREB1 like retinoic acid receptor coactivator” refers to the gene GREB1L and the gene product encoded by the GREB1L gene. It is also known as “C18orR5”, “DFNA80”, “KIAA1772,” or “RHDA3.” In the human genome, GREB1L is located on chromosome 18. Exemplary human GREB1L transcript sequences include, but are not limited to, NCBI Reference Sequences: NM_001142966.3 GI: 1890341525, XM_006722547.3 GI: 1370473848, XM_011526179.3 GI: 1370473849, XM_017025988.1 GI: 1034604738, XM_017025989.1 GI: 1034604740, XM_017025990.1 GI: 1034604742, XM_017025991.1 GI: 1034604744, XM_017025992.1 GI: 1034604747, XM_017025993.1 GI: 1034604749, XM_017025994.1 GI: 1034604752, XM_017025995.1 GI: 1034604754, and XM_017025996.1 GI: 1034604756. Exemplary human GREB1L protein sequences include, but are not limited to, NCBI Reference Sequences: NP_001136438.1 GI: 219282747, XP_006722610.1 GI: 578832707, XP_011524481.1 GI: 767999262, XP_016881477.1 GI: 1034604739, XP_016881478.1 GI: 1034604741, XP_016881479.1 GI: 1034604743, XP_016881480.1 GI: 1034604745, XP_016881481.1 GI: 1034604748, XP_016881482.1 GI: 1034604750, XP_016881483.1 GI: 1034604753, XP_016881484.1 GI: 1034604755, and XP_016881485.1 GI: 1034604757. Without wishing to be bound by theory, it is believed that in some embodiments, GREB1L is involved in retinoic acid receptor activity.

As used herein, the term “BIRC2,” “baculoviral IAP repeat containing 2” refers to the gene BIRC2 and the gene product encoded by the BIRC2 gene. It is also known as “API1”, “HIAP2”, “Hiap-2”, “MIHB”, “RNF48”, “c-IAP1” or “cIAP1.” In the human genome, BIRC2 is located on chromosome 11. Exemplary human BIRC2 transcript sequences include, but are not limited to, NCBI Reference Sequences: NM_001166.5 GI: 1919270910, NM_001256163.1 GI: 390608636, and NM_001256166.2 GI: 1677500091. Exemplary human BIRC2 protein sequences include, but are not limited to, NCBI Reference Sequences: NP_001157.1 GI: 4502141, NP_001243092.1 GI: 390608637, and NP_001243095.1 GI: 390608639.

As used herein, the term “IYMSOS,” “TYMS opposite strand RNA” refers to the gene TYMSOS and the gene product encoded by the TYMSOS gene. It is also known as “C18orf56.” In the human genome, TYMSOS is located on chromosome 18. Exemplary human TYMSOS transcript sequences include, but are not limited to, NCBI Reference Sequence: NR_171001.1 GI: 1928917883. Exemplary human TYMSOS protein sequences include, but are not limited to, NCBI Reference Sequence: Q8TAI1.2 GI: 294862427.

As used herein, the term “PPP6R3,” “protein phosphatase 6 regulatory subunit 3” refers to the gene PPP6R3 and the gene product encoded by the PPP6R3 gene. It is also known as “C11orf23”, “PP6R3”, “SAP190”, “SAPL”, “SAPLa,” or “SAPS3.” In the human genome, PPP6R3 is located on chromosome 11. Exemplary human PPP6R3 transcript sequences include, but are not limited to, NCBI Reference Sequences: NM_001164161.2 GI: 1677500144, NM_018312.5 GI: 1677499417, NM_001164160.2 GI: 1677538413, NM_001164160.2 GI: 1677538413, NM_001164162.2 GI: 1677499229, NM_001164163.2 GI: 1677479624, NM_001164164.2 GI: 1677498594, NM_001352347.2 GI: 1677531896, NM_001352348.2 GI: 1677499052, NM_001352350.2 GI: 1677501263, NM_001352351.2 GI: 1677500439, NM_001352352.2 GI: 1677529940, NM_001352353.2 GI: 1677502275, NM_001352354.2 GI: 1677501535, NM_001352355.2 GI: 1677501395, NM_001352356.2 GI: 1677498748, NM_001352357.2 GI: 1677538115, NM_001352358.2 GI: 1677499332, NM_001352359.2 GI: 1677498278, NM_001352360.2 GI: 1677501444, NM_001352361.2 GI: 1677501992, NM_001352362.2 GI: 1677500794, NM_001352363.2 GI: 1677530125, NM_001352364.2 GI: 1677500385, NM_001352365.2 GI: 1677474270, NM_001352366.2 GI: 1677531035, NM_001352368.2 GI: 1677501519, NM_001352369.2 GI: 1677531693, NM_001352370.2 GI: 1677530717, NM_001352371.2 GI: 1677500667, NM_001352372.2 GI: 1677500520, NM_001352373.2 GI: 1677477342, NM_001352374.2 GI: 1677499588, NM_001352375.2 GI: 1677531068, NM_001352376.2 GI: 1677500178, NM_001352377.2 GI: 1677531348, NM_001352378.2 GI: 1677529770, NM_001352379.2 GI: 1677500360, NM_001352380.2 GI: 1677500029, NR_147965.2 GI: 1677539176, NR_147966.2 GI: 1700660455, NR_147967.2 GI: 1701961850, NR_147968.2 GI: 1701961858,NR_147969.2 GI: 1700447875, NR_147970.2 GI: 1677539248, NR_147971.2 GI: 1701216205, XM_006718608.3 GI: 1034574378, XM_006718612.3 GI: 1034574382, XM_006718614.2 GI: 767968449, XM_006718621.2 GI: 767968459, XM_006718622.3 GI: 1034574414, XM_006718623.3 GI: 1034574415, XM_006718624.2 GI: 767968466, XM_006718627.3 GI: 1034574432, XM_011545137.2 GI: 1034574376, XM_011545138.2 GI: 1034574377, XM_011545139.1 GI: 767968441, XM_011545141.1 GI: 767968447, XM_017017969.1 GI: 1034574396, XM_017017974.1 GI: 1034574407, XM_017017977.2 GI: 1370459710, XM_024448602.1 GI: 1370459708, XM_024448601.1 GI: 1370459706, and XM_024448603.1 GI: 1370459711. Exemplary human PPP6R3 protein sequences include, but are not limited to, NCBI Reference Sequences: NP_001157633.1 GI: 255918194, NP_060782.2 GI: 13489083,NP_001157632.1 GI: 255918192,NP_001157634.1 GI: 255918196,NP_001157635.1 GI: 255918198,NP_001157636.1 GI: 255918200, NP_001339276.1 GI: 1204934633, NP_001339277.1 GI: 1204934703, NP_001339279.1 GI: 1204934576,NP_001339280.1 GI: 1204934568, NP_001339281.1 GI: 1204934610,NP_001339282.1 GI: 1204934572,NP_001339283.1 GI: 1204934600, NP_001339284.1 GI: 1204934565, NP_001339285.1 GI: 1204934628,NP_001339286.1 GI: 1204934655, NP_001339287.1 GI: 1204934635, NP_001339288.1 GI: 1204934691, NP_001339289.1 GI: 1204934701, NP_001339290.1 GI: 1204934686, NP_001339291.1 GI: 1204934688, NP_001339292.1 GI: 1204934683,NP_001339293.1 GI: 1204934602, NP_001339294.1 GI: 1204934648, NP_001339295.1 GI: 1204934710, NP_001339297.1 GI: 1204934641, NP_001339298.1 GI: 1204934590, NP_001339299.1 GI: 1204934661, NP_001339300.1 GI: 1204934608, NP_001339301.1 GI: 1204934592, NP_001339302.1 GI: 1204934606, NP_001339303.1 GI: 1204934584, NP_001339304.1 GI: 1204934699, NP_001339305.1 GI: 1204934582, NP_001339306.1 GI: 1204934657, NP_001339307.1 GI: 1204934679, NP_001339308.1 GI: 1204934586, NP_001339309.1 GI: 1204934708, XP_006718671.1 GI: 578821551, XP_006718675.1 GI: 578821559, XP_006718677.1 GI: 578821563, XP_006718684.1 GI: 578821577,XP_006718685.1 GI: 578821579, XP_006718686.1 GI: 578821581, XP_006718687.1 GI: 578821583, XP_006718690.1 GI: 578821589, XP_011543439.1 GI: 767968437, XP_011543440.1 GI: 767968439, XP_011543441.1 GI: 767968442, XP_011543443.1 GI: 767968448, XP_016873458.1 GI: 1034574397, XP_016873463.1 GI: 1034574408, XP_016873466.1 GI: 1034574417, XP_024304370.1 GI: 1370459709, XP_024304369.1 GI: 1370459707, and XP_024304371.1 GI: 1370459712

As used herein, the term “PITPNB,” “phosphatidylinositol transfer protein beta” refers to the gene PITPNB and the gene product encoded by the PITPNB gene. It is also known as “PI-TP-beta,” “PtdInsTP,” or “VIB1B.” In the human genome, PITPNB is located on chromosome 22. Exemplary human PITPNB transcript sequences include, but are not limited to, NCBI Reference Sequences: NM_012399.5 GI: 1653961049, NM_001284277.2 GI: 1889692876, NM_001284278.2 GI: 1889510257, XM_011530052.2 GI: 1034628647, XM_017028707.1 GI: 1034628648, XR_001755190.1 GI: 1034628650, and XR_002958679.1 GI: 1370482098. Exemplary human PITPNB protein sequences include, but are not limited to, NCBI Reference Sequences: NP_036531.1 GI: 6912594, NP_001271206.1 GI: 546232151, NP_001271207.1 GI: 546232153, XP_011528354.1 GI: 768024217, and XP_016884196.1 GI: 1034628649.

As used herein, the term “TGIF1,” “TGFB induced factor homeobox 1” refers to the gene TGIF1 and the gene product encoded by the TGIF1 gene. It is also known as “HPE4,” or “TGIF.” In the human genome, TGIF1 is located on chromosome 18. Exemplary human TGIF1 transcript sequences include, but are not limited to, NCBI Reference Sequences: NM_003244.4 GI: 1751363255, NM_170695.5 GI: 1751363270, NM_173207.4 GI: 1751363242, NM_173208.3 GI: 1751363245, NM_173209.3 GI: 1751363290, NM_173210.4 GI: 1751363225, NM_173211.2 GI: 1751363230, NM_174886.3 GI: 1751363249, NM_001278682.2 GI: 1751363246, NM_001278684.2 GI: 1751363267, NM_001278686.3 GI: 1889658060, NM_001374396.1 GI: 1751363258, NM_001374397.1 GI: 1751363276, and XM_017025958.1 GI: 1034604653. Exemplary human TGIF1 protein sequences include, but are not limited to, NCBI Reference Sequences: NP_003235.1 GI: 4507473, NP_733796.3 GI: 1751363271, NP_775299.1 GI: 28178845, NP_775300.1 GI: 28178849, NP_775301.1 GI: 28178851, NP_775302.1 GI: 28178853, NP_775303.1 GI: 28178855, NP_777480.1 GI: 28178857, NP_001265611.1 GI: 522838237, NP_001265613.1 GI: 522838242, NP_001265615.1 GI: 522838246, NP_001361325.1 GI: 1751363259, NP_001361326.1 GI: 1751363277, and XP_016881447.1 GI: 1034604654.

As used herein, the term “LIN37,” “lin-37 DREAM MuvB core complex component” refers to the gene bLIN37 and the gene product encoded by the LIN37 gene. It is also known as “F25965,” “ZK418.4,” or “lin-37.” In the human genome, LIN37 is located on chromosome 19. Exemplary human LIN37 transcript sequences include, but are not limited to, NCBI Reference Sequences: NM_019104.3 GI: 1519244030, NM_001369780.1 GI: 1621332311, and NR_163146.1 GI: 1621332351. Exemplary human LIN37 protein sequences include, but are not limited to, NCBI Reference Sequences: NP_061977.1 GI: 28144916 and NP_001356709.1 GI: 1621332312.

As used herein, the term “TAF11,” “TATA-box binding protein associated factor 11” refers to the gene TAF11 and the gene product encoded by the TAF11 gene. It is also known as “MGC:15243,” “PRO2134”, “TAF2I,” or “TAFII28.” In the human genome, TAF11 is located on chromosome 6. Exemplary human TAF11 transcript sequences include, but are not limited to, NCBI Reference Sequences: NM_005643.4 GI: 1653961404, NM_001270488.1 GI: 394953993, and XM_011514827.2 GI: 1034651423. Exemplary human TAF11 protein sequences include, but are not limited to, NCBI Reference Sequences: NP_005634.1 GI: 5032151, NP_001257417.1 GI: 394953994, and XP_011513129.1 GI: 767940559.

As used herein, the term “ZFAT,” “zinc finger and AT-hook domain containing” refers to the gene ZFAT and the gene product encoded by the ZFAT gene. It is also known as “AITD3,” “ZFAT1,” or “ZNF406.” In the human genome, ZFAT is located on chromosome 8. Exemplary human ZFAT transcript sequences include, but are not limited to, NCBI Reference Sequences: NP_065914.2 GI: 46487911, NP_001025110.2 GI: 292658775, NP_001161055.1 GI: 262399392, NP_001167628.1 GI: 292658778, NP_001167629.1 GI: 292658780, NP_001276323.1 GI: 574957236, XP_011515505.1 GI: 767953382, XP_011515506.1 GI: 767953384, XP_011515508.1 GI: 767953388, and XP_016869205.1 GI: 1034661127. Exemplary human ZFAT protein sequences include, but are not limited to, NCBI Reference Sequences: NP_065914.2 GI: 46487911, NP_001025110.2 GI: 292658775, NP_001161055.1 GI: 262399392, NP_001167628.1 GI: 292658778, NP_001167629.1 GI: 292658780, NP_001276323.1 GI: 574957236, XP_011515505.1 GI: 767953382, XP_011515506.1 GI: 767953384, XP_011515508.1 GI: 767953388, and XP_016869205.1 GI: 1034661127.

As used herein, the term “FAM91A1,” “family with sequence similarity 91 member A1” refers to the gene FAM91A1 and the gene product encoded by the FAM91A1 gene. In the human genome, FAM91A1 is located on chromosome 8. Exemplary human FAM91A1 transcript sequences include, but are not limited to, NCBI Reference Sequences: NM_144963.4 GI: 1519315093, NM_001317917.2 GI: 1676316959, NM_001317918.1 GI: 961658071, and XR_001745485.2 GI: 1370511941. Exemplary human FAM91A1 protein sequences include, but are not limited to, NCBI Reference Sequences: NP_659400.3 GI: 1519315094, NP_001304846.2 GI: 1676316960, and NP_001304847.1 GI: 961658072. Without wishing to be bound by theory, it is believed that in some embodiments, FAM91A1 is involved in WDR11 pathway.

As used herein, the term “WDR11,” “WD repeat domain 11” refers to the gene WDR11 and the gene product encoded by the WDR11 gene. It is also known as “BRWD2”, “DR11”, “HH14”, “SRI1,” or “WDR15.” In the human genome, WDR11 is located on chromosome 10. Exemplary human WDR11 transcript sequences include, but are not limited to, NCBI Reference Sequences: NM_018117.12 GI: 1519312651, M_005269963.2 GI: 1034568729, XM_017016397.1 GI: 1034568726, XM_017016398.1 GI: 1034568731, XM_017016399.1 GI: 1034568735, XM_017016400.2 GI: 1370457396, XM_024448075.1 GI: 1370457397, XR_428707.3 GI: 1370457393, XR_001747136.2 GI: 1370457394, and XR_001747137.2 GI: 1370457395. Exemplary human WDR11 protein sequences include, but are not limited to, NCBI Reference Sequences: NP_060587.8 GI: 13324688, XP_005270020.1 GI: 530393964, XP_016871886.1 GI: 1034568727, XP_016871887.1 GI: 1034568732, XP_016871888.1 GI: 1034568736, XP_016871889.1 GI: 1034568738, and XP_024303843.1 GI: 1370457398. Without wishing to be bound by theory, it is believed that in some embodiments, WDR11 is involved in WDR11 pathway.

As used herein, the term “ELP3,” “elongator acetyltransferase complex subunit 3” refers to the gene ELP3 and the gene product encoded by the ELP3 gene. It is also known as “KAT9.” In the human genome, ELP3 is located on chromosome 8. Exemplary human ELP3 transcript sequences include, but are not limited to, NCBI Reference Sequences: NM_018091.6 GI: 1519245905, NM_001284220.2 GI: 1675178308, NM_001284222.2 GI: 1676440523, NM_001284224.2 GI: 1676441760, NM_001284225.2 GI: 1890333780, NM001284226.2 GI: 1890334018, XM_006716354.3 GI: 1370512533, XM_017013604.2 GI: 1370512528, XM_024447184.1 GI: 1370512529, and XM_024447185.1 GI: 1370512531. Exemplary human ELP3 protein sequences include, but are not limited to, NCBI Reference Sequences: NP_060561.3 GI: 23510283, NP_001271149.1 GI: 545746389, NP_001271151.1 GI: 545746373, NP_001271153.1 GI: 545746266, NP_001271154.1 GI: 545746350, NP_001271155.1 GI: 545746360, XP_006716417.1 GI: 578815360, XP_016869093.1 GI: 1034660802, XP_024302952.1 GI: 1370512530, and XP_024302953.1 GI: 1370512532.

As used herein, the term “KDM3B,” “lysine demethylase 3B” refers to the gene KDM3B and the gene product encoded by the KDM3B gene. It is also known as “5qNCA, C5orf7, DIJOS, JMJD1B,” or “NET22.” In the human genome, KDM3B is located on chromosome 5. Exemplary human KDM3B transcript sequences include, but are not limited to, NCBI Reference Sequences: NM_016604.4 GI: 1519312226, XM_005272018.4 GI: 1034645276, XM_011543488.2 GI: 1034645274, XM_011543489.2 GI: 1034645275, XM_017009584.1 GI: 1034645277, and XM_024446115.1 GI: 1370496960. Exemplary human KDM3B protein sequences include, but are not limited to, NCBI Reference Sequences: NP_057688.3 GI: 1519312227, XP_005272075.1 GI: 530380015, XP_011541790.1 GI: 767936123, XP_011541791.1 GI: 767936125, XP_016865073.1 GI: 1034645278, and XP_024301883.1 GI: 1370496961.

As used herein, the term “UNC93B1,” “unc-93 homolog B1, TLR signaling regulator” refers to the gene UNC93B1 and the gene product encoded by the UNC93B1 gene. It is also known as “IIAE1”, “UNC93”, “UNC93B,” or “Unc-93B1.” In the human genome, UNC93B1 is located on chromosome 11. Exemplary human UNC93B1 transcript sequences include, but are not limited to, NCBI Reference Sequences: NM_030930.4 GI: 1519243978, XM_011545290.1 GI: 767968839, XP_011543592.1 GI: 767968840, and XM_011545291.2 GI: 1034575685. Exemplary human UNC93B1 protein sequences include, but are not limited to, NCBI Reference Sequences: NP_112192.2 GI: 45580709, and XP_011543593.1 GI: 767968842.

As used herein, the term “TBC1D23” “TBC1 domain family member 23” refers to the gene TBC1D23 and the gene product encoded by the TBC1D23 gene. It is also known as “NS4ATP1,” or “PCH11.” In the human genome, TBC1D23 is located on chromosome 3. Exemplary human TBC1D23 transcript sequences include, but are not limited to, NCBI Reference Sequences: NM_001199198.3 GI: 1519245870, NM_018309.5 GI: 1674985908, XM_011512974.3 GI: 1370484657, and XM_017006841.2 GI: 1370484658. Exemplary human TBC1D23 protein sequences include, but are not limited to, NCBI Reference Sequences: NP_001186127.1 GI: 312837064, NP_060779.2 GI: 217035118, XP_011511276.1 GI: 767927131, and XP_016862330.1 GI: 1034634492. Without wishing to be bound by theory, it is believed that in some embodiments, TBC1D23 is involved in WDR11 pathway.

As used herein, the term “CATSPERZ,” “catsper channel auxiliary subunit zeta” refers to the gene CATSPERZ and the gene product encoded by the CATSPERZ gene. It is also known as “C11orf20,” or “TEX40.” In the human genome, CATSPERZ is located on chromosome 11. Exemplary human CATSPERZ transcript sequences include, but are not limited to, NCBI Reference Sequence: NM_001039496.2 GI: 1519245208. Exemplary human CATSPERZ protein sequences include, but are not limited to, NCBI Reference Sequence: NP_001034585.1 GI: 148839350.

As used herein, the term “HIKESHI,” “heat shock protein nuclear import factor hikeshi” refers to the gene HIKESHI and the gene product encoded by the HIKESHI gene. It is also known as “C11orf73”, “HLD13”, “HSPC138”, “HSPC179”, “L7RN6,” or “OPI10.” In the human genome, HIKESHI is located on chromosome 11. Exemplary human HIKESHI transcript sequences include, but are not limited to, NCBI Reference Sequences: NM_016401.4 GI: 1519315830, NM_001322404.2 GI: 1676318183, NM_001322407.2 GI: 1676316874, NM_001322409.2 GI: 1676317764, NR_024597.2 GI: 1676452109, NR_024598.2 GI: 1676452898, NR_136324.2 GI: 1701945878, XM_017017914.2 GI: 1370459624, XM_017017915.1 GI: 1034574206, XR_949963.3 GI: 1370459625, and XR_001747904.2 GI: 1370459623. Exemplary human HIKESHI protein sequences include, but are not limited to, NCBI Reference Sequences: NP_057485.2 GI: 21361535, NP_001309333.1 GI: 1016841140, NP_001309336.1 GI: 1016841148, NP_001309338.1 GI: 1016841090, XP_016873403.1 GI: 1034574204, and XP_016873404.1 GI: 1034574207. Without wishing to be bound by theory, it is believed that in some embodiments, HIKESHI is involved in nuclear import of an HSP70 protein.

As used herein, the term “TMEM123,” “transmembrane protein 123” refers to the gene TMEM123 and the gene product encoded by the TMEM123 gene. It is also known as “KCT3,” “PORIMIN,” or “PORMIN.” In the human genome, TMEM123 is located on chromosome 11. Exemplary human TMEM123 transcript sequences include, but are not limited to, NCBI Reference Sequence: NM_052932.3 GI: 1519242071. Exemplary human TMEM123 protein sequences include, but are not limited to, NCBI Reference Sequences: CCQ43808.1 GI: 444733223. Without wishing to be bound by theory, it is believed that in some embodiments, TMEM123 is involved in vesicle-mediated endocytosis.

As used herein, the term “HELZ,” “helicase with zinc finger” refers to the gene HELZ and the gene product encoded by the HELZ gene. It is also known as “DHRC,” “DRHC,” or “HUMORF5.” In the human genome, HELZ is located on chromosome 17. Exemplary human HELZ transcript sequences include, but are not limited to, NCBI Reference Sequences: NM_014877.4 GI: 1519313142, NM_001330447.2 GI: 1676316917, XM_005257888.5 GI: 1370472804, XM_006722214.4 GI: 1370472803, XM_006722215.3 GI: 1034602512, XM_006722216.3 GI: 1034602515, XM_011525544.2 GI: 1034602509, XM_017025477.2 GI: 1370472805, XM_017025478.1 GI: 1034602516, XR_001752712.2 GI: 1370472806, XR_001752713.2 GI: 1370472807, and XR_001752714.2 GI: 1370472808. Exemplary human HELZ protein sequences include, but are not limited to, NCBI Reference Sequences: NP_055692.3 GI: 1519313143, NP001317376.2 GI: 1676316918, XP_005257945.1 GI: 530413304, XP_006722277.1 GI: 578831745, XP_006722278.1 GI: 578831750, XP_006722279.1 GI: 578831752, XP_011523846.1 GI: 767996699, XP_016880966.1 GI: 1034602514, and XP_016880967.1 GI: 1034602517.

As used herein, the term “SARNP,” “SAP domain containing ribonucleoprotein” refers to the gene SARNP and the gene product encoded by the SARNP gene. It is also known as “CIP29”, “HCC1”, “HSPC316,” or “THO1.” In the human genome, SARNP is located on chromosome 12. Exemplary human SARNP transcript sequences include, but are not limited to, NCBI Reference Sequences: NM_033082.4 GI: 1519314114, NR_026722.2 GI: 1890350977, and NR_026723.2 GI: 1890608286. Exemplary human SARNP protein sequences include, but are not limited to, NCBI Reference Sequence: NP_149073.1 GI: 32129199.

As used herein, the term “PSIP1,” “PC4 and SFRS1 interacting protein 1” refers to the gene PSIP1 and the gene product encoded by the PSIP1 gene. It is also known as “DFS70”, “LEDGF”, “PAIP”, “PSIP2”, “p52,” or “p75.” In the human genome, PSIP1 is located on chromosome 9. Exemplary human PSIP1 transcript sequences include, but are not limited to, NCBI Reference Sequences: NM_033222.5 GI: 1519241968, NM_021144.4 GI: 1890342066, NM_001128217.3 GI: 1890258155, NM_001317898.3 GI: 1890272938, NM_001317900.3 GI: 1890270743, XM_024447399.1 GI: 1370513697, XM_024447400.1 GI: 1370513699, XM_024447401.1 GI: 1370513701, and XM_024447402.1 GI: 1370513703. Exemplary human PSIP1 protein sequences include, but are not limited to, NCBI Reference Sequences: NP_150091.2 GI: 19923653, NP_066967.3 GI: 190014586, NP_001121689.1 GI: 190014588, NP_001304827.1 GI: 961513278, NP_001304829.1 GI: 961526013, XP_024303167.1 GI: 1370513698, XP_024303168.1 GI: 1370513700, XP_024303169.1 GI: 1370513702, and XP_024303170.1 GI: 1370513704. Without wishing to be bound by theory, it is believed that in some embodiments, PSIP1 is involved in transcription.

As used herein, the term “WTAP,” “VT1 associated protein” refers to the gene WTAP and the gene product encoded by the WTAP gene. It is also known as “Mum2.” In the human genome, WTAP is located on chromosome 6. Exemplary human WTAP transcript sequences include, but are not limited to, NCBI Reference Sequences: NM_001270531.2 GI: 1519245524, NM_004906.5 GI: 1675034286, NM_152857.3 GI: 1890334336, NM_152858.3 GI: 1890341477, NM_001270532.2 GI: 1890258879, NM_001270533.2 GI: 1675176106, XM_017011514.2 GI: 1370508971, XM_017011515.1 GI: 1034652352, XM_017011516.2 GI: 1370508974, and XM_024446597.1 GI: 1370508972. Exemplary human WTAP protein sequences include, but are not limited to, NCBI Reference Sequences: NP_001257460.1 GI: 395455090, NP_004897.2 GI: 21361159, NP_690596.1 GI: 23199974, NP_690597.1 GI: 23199976, NP_001257461.1 GI: 395455092, NP_001257462.1 GI: 395455094, XP_016867003.1 GI: 1034652351, XP_016867004.1 GI: 1034652353, XP_016867005.1 GI: 1034652355, and XP_024302365.1 GI: 1370508973.

As used herein, the term “MTMR9,” “myotubularin related protein 9” refers to the gene MTMR9 and the gene product encoded by the MTMR9 gene. It is also known as “C8orf9,” “LIP-STYX,”or “MTMR8.” In the human genome, MTMR9 is located on chromosome 8. Exemplary human MTMR9 transcript sequences include, but are not limited to, NCBI Reference Sequences: NM_015458.4 GI: 1519314712, XM_011543830.3 GI: 1370512772, XM_011543831.2 GI: 1034661267, and XM_017013753.2 GI: 1370512773. Exemplary human MTMR9 protein sequences include, but are not limited to, NCBI Reference Sequences: NP_056273.2 GI: 19923424, XP_011542132.1 GI: 767950327, XP_011542133.1 GI: 767950329, and XP_016869242.1 GI: 1034661269. Without wishing to be bound by theory, it is believed that in some embodiments, MTMR9 is involved vesicle-mediated endocytosis.

As used herein, the term “MBOAT7,” “membrane bound O-acyltransferase domain containing 7” refers to the gene MBOAT7 and the gene product encoded by the MBOAT7 gene. It is also known as “BB1”, “LENG4”, “LPIAT”, “LPLAT”, “LRC4”,” MBOA7”, “MRT57”, “OACT7,” or “hMBOA-7.” In the human genome, MBOAT7 is located on chromosome 19. Exemplary human MBOAT7 transcript sequences include, but are not limited to, NCBI Reference Sequences: NM_024298.5 GI: 1519244886, NM_001146056.3 GI: 1675144256, NM_001146082.3 GI: 1675158206, NM_001146083.3 GI: 1890265709, XM_011527299.3 GI: 1370475930, XM_011527300.2 GI: 1034609460, and XM_017027296.2 GI: 1370475931. Exemplary human MBOAT7 protein sequences include, but are not limited to, NCBI Reference Sequences: NP_077274.3 GI: 225703079, NP_001139528.1 GI: 225703081, NP_001139554.1 GI: 225703037, NP_001139555.1 GI: 225703033, XP_011525601.1 GI: 768011018, XP_011525602.1 GI: 768011022, and XP_016882785.1 GI: 103460946. Without wishing to be bound by theory, it is believed that in some embodiments, MBOAT7 is involved in with vesicle-mediated endocytosis.

As used herein, the term “VT11A,” “vesicle transport through interaction with t-SNAREs 1A” refers to the gene VT11A and the gene product encoded by the VT11A gene. It is also known as “MMDS3”, “MVti1”, “VIMIRP2,” or “Vti1-rp2.” In the human genome, VT11A is located on chromosome 10. Exemplary human VT11A transcript sequences include, but are not limited to, NCBI Reference Sequences: NM_145206.4 GI: 1813788147, NM_001318203.2 GI: 1890334186, NM_001318205.2 GI: 1889518129, NM_001365710.2 GI: 1677501521, NM_001365711.1 GI: 1475409205, NM_001365712.1 GI: 1475409081, NM_001365713.1 GI: 1475408996, NM_001365714.1 GI: 1475408890, NR_134521.2 GI: 1890386498, NR_134522.2 GI: 1890394772, and NR_134523.1 GI: 970598273. Exemplary human VTI1A protein sequences include, but are not limited to, NCBI Reference Sequences: NP_660207.2 GI: 113374156, NP_001305132.1 GI: 970598264, NP_001305134.1 GI: 970598269, NP_001352639.1 GI: 1475409158, NP_001352640.1 GI: 1475409206, NP_001352641.1 GI: 1475409082, NP_001352642.1 GI: 1475408997, and NP_001352643.1 GI: 1475408891.

As used herein, the term “MRE11” “MRE11 homolog, double strand break repair nuclease” refers to the gene MRE11 and the gene product encoded by the MRE11 gene. It is also known as “ATLD”, “HNGS1”, “MRE11A,” or “MRE11B.” In the human genome, MRE11 is located on chromosome 11. Exemplary human MRE11 transcript sequences include, but are not limited to, NCBI Reference Sequences: NM_005591.4 GI: 1777425444, NM_005590.4 GI: 1677500399, NM_001330347.2 GI: 1677537463, XM_005274008.3 GI: 1034573789, XM_006718842.3 GI: 1034573786, XM_011542837.2 GI: 1034573785, XM_017017772.1 GI: 1034573782, and XR_947828.2 GI: 1034573784. Exemplary human MRE11 protein sequences include, but are not limited to, NCBI Reference Sequences: NP_005582.1 GI: 5031923, NP_005581.2 GI: 24234690, NP_001317276.1 GI: 1057866489, XP_005274065.1 GI: 530396808, XP_006718905.1 GI: 578822201, XP_011541139.1 GI: 767970186, and XP_016873261.1 GI: 1034573783.

As used herein, the term “SLF2,” “SMC5-SMC6 complex localization factor 2” refers to the gene SLF2 and the gene product encoded by the SLF2 gene. It is also known as “C10orf6,” or “FAM178A.” In the human genome, SLF2 is located on chromosome 10. Exemplary human SLF2 transcript sequences include, but are not limited to, NCBI Reference Sequences: NM_018121.4 GI: 1519473713, NM_001136123.2 GI: 1674995332, NM_001243770.2 GI: 1676317210, XM_005269965.3 GI: 1370457399, XM_011539944.3 GI: 1370457400, and XR_001747138.1 GI: 1034568742. Exemplary human SLF2 protein sequences include, but are not limited to, NCBI Reference Sequences: NP_060591.3 GI: 150456436, NP_001129595.1 GI: 209915547, NP_001230699.1 GI: 344313179, XP_005270022.1 GI: 530393969, and XP_011538246.1 GI: 767963250.

As used herein, the term “MSL3,” “MSL complex subunit 3” refers to the gene MSL3 and the gene product encoded by the MSL3 gene. It is also known as “MRSXBA”, “MRXS36”, “MRXSBA,” or “MSL3L1.” In the human genome, MSL3 is located on chromosome X. Exemplary human MSL3 transcript sequences include, but are not limited to, NCBI Reference Sequences: NM_078629.4 GI: 1653961367, NM_006800.4 GI: 1677501465, NM_078628.2 GI: 1890342633, NM_001193270.2 GI: 532164697, and NM_001282174.1 GI: 532164698. Exemplary human MSL3 protein sequences include, but are not limited to, NCBI Reference Sequences: NP_523353.2 GI: 212275945, NP_006791.2 GI: 17975761, NP_523352.1 GI: 17975755, NP_001180199.1 GI: 300796021, and NP_001269103.1 GI: 532164699. Without wishing to be bound by theory, it is believed that in some embodiments, MSL3 is involved in histone H4 acetylation.

As used herein, the term “SIK3,” “SIK family kinase 3” refers to the gene SIK3 and the gene product encoded by the SIK3 gene. It is also known as “QSK,” “SEMDK,” or “SIK-3.” In the human genome, SIK3 is located on chromosome 11. Exemplary human SIK3 transcript sequences include, but are not limited to, NCBI Reference Sequences: NM_001366686.3 GI: 1912229914, NM_025164.6 GI: 1677530722,NM_001281748.3 GI: 1677530962,NM_001281749.3 GI: 1677530530, NM_001366687.1 GI: 1491609407, XM_005271482.4 GI: 1034572701,XM_005271484.3 GI: 767969906, XM_005271485.3 GI: 1034572711, XM_011542722.1 GI: 767969904,XM_011542723.2 GI: 1034572704,XM_011542724.2 GI: 1034572707, XM_011542725.2 GI: 1034572710, M_011542726.1 GI: 767969915,XM_017017424.1 GI: 1034572702, XM_017017425.1 GI: 1034572705,XM_017017426.1 GI: 1034572708, XM_017017427.1 GI: 1034572712, and XR_001747816.1 GI: 1034572714. Exemplary human SIK3 protein sequences include, but are not limited to, NCBI Reference Sequences: NP_001353615.1 GI: 1491609460, NP_079440.3 GI: 528524618, NP_001268677.1 GI: 528524620, NP_001268678.1 GI: 528524622, NP_001353616.1 GI: 1491609408, XP_005271539.2 GI: 767969903, XP_005271541.2 GI: 767969907, XP_005271542.1 GI: 530397953, XP_011541024.1 GI: 767969905,XP_011541025.1 GI: 767969909,XP_011541026.1 GI: 767969911, XP_011541027.1 GI: 767969913,XP_011541028.1 GI: 767969916,XP_016872913.1 GI: 1034572703, XP_016872914.1 GI: 1034572706, XP_016872915.1 GI: 1034572709, and XP_016872916.1 GI: 1034572713.

As used herein, the term “GTF2H5,” “general transcription factor IIH subunit 5” refers to the gene GTF2H5 and the gene product encoded by the GTF2H5 gene. It is also known as “C6orf175”, “TFB5”, “TFIIH”, “TGF2H5”, “TTD”, “TTD-A”, “TTD3”, “TTDA,” or “bA120J8.2.” In the human genome, GTF2H5 is located on chromosome 6. Exemplary human GTF2H5 transcript sequences include, but are not limited to, NCBI Reference Sequences: NM_207118.3 GI: 1732746154 and M_017010862.1 GI: 1034650264. Exemplary human GTF2H5 protein sequences include, but are not limited to, NCBI Reference Sequences: NP_997001.1 GI: 46359855 and XP_016866351.1 GI: 1034650265. Without wishing to be bound by theory, it is believed that in some embodiments, GTF2H5 is involved in transcription and/or DNA repair.

As used herein, the term “PIGW,” “phosphatidylinositol glycan anchor biosynthesis class W” refers to the gene PIGW and the gene product encoded by the PIGW gene. It is also known as “Gwt1,” or “HPMRS5.” In the human genome, PIGW is located on chromosome 17. Exemplary human PIGW transcript sequences include, but are not limited to, NCBI Reference Sequences: NM_001346754.2 GI: 1519473471 NM_178517.5 GI: 1890340690, and NM_001346755.2 GI: 1890263021. Exemplary human PIGW protein sequences include, but are not limited to, NCBI Reference Sequences: NP_001333683.1 GI: 1092878785, NP_848612.2 GI: 75750492, and NP_001333684.1 GI: 1092878806. Without wishing to be bound by theory, it is believed that in some embodiments, PIGW is involved in GPI biosynthesis.

As used herein, the term “ATRAID,” “all-trans retinoic acid induced differentiation factor” refers to the gene ATRAID and the gene product encoded by the ATRAID gene. It is also known as “APR-3”, “APR-3”, “APR3”, “C2orf28”, “HSPC013” “PRO240,” or “p18” In the human genome, ATRAID is located on chromosome 2. Exemplary human ATRAID transcript sequences include, but are not limited to, NCBI Reference Sequences: NM_001170795.4 GI: 1955885176 and NM_016085.5 GI: 1706971014. Exemplary human ATRAID protein sequences include, but are not limited to, NCBI Reference Sequences: NP_001164266.1 GI: 282847380 and NP_057169.2 GI: 18105012.

As used herein, the term “PPP4R1,” “protein phosphatase 4 regulatory subunit 1” refers to the gene PPP4R1 and the gene product encoded by the PPP4R1 gene. It is also known as “MEG1,” “PP4(Rmeg),” or “PP4R1.” In the human genome, PPP4R1 is located on chromosome 18. Exemplary human PPP4R1 transcript sequences include, but are not limited to, NCBI Reference Sequences: NM_001042388.3 GI: 1677538272, NM_005134.4 GI: 1677502103, NM_001382562.1 GI: 1841839464, NR_052003.2 GI: 1701969358, NR_168403.1 GI: 1841851492, NR_168404.1 GI: 1841851494, XM_011525775.3 GI: 1370473986, XM_011525776.1 GI: 767997972, XM_011525779.3 GI: 1370473988, XM_017026105.2 GI: 1370473987, XM_017026107.1 GI: 1034605108, and XR_001753307.2 GI: 1370473989. Exemplary human PPP4R1 protein sequences include, but are not limited to, NCBI Reference Sequences: NP_001035847.1 GI: 108936953, NP_005125.1 GI: 4826934, NP_001369491.1 GI: 1841839465, XP_011524077.1 GI: 767997971, XP_011524078.1 GI: 767997973, XP_011524081.1 GI: 767997979, XP_016881594.1 GI: 1034605104, and XP_016881596.1 GI: 1034605109. Without wishing to be bound by theory, it is believed that in some embodiments, PPP4R1 is involved in vesicle-mediated endocytosis.

As used herein, the term “ZFC3H1,” “zinc finger C3H1-type containing” refers to the gene ZFC3H1 and the gene product encoded by the ZFC3H1 gene. It is also known as “CCDC131,” “CSRC2,” or “PSRC2.” In the human genome, ZFC3H1 is located on chromosome 12. Exemplary human ZFC3H1 transcript sequences include, but are not limited to, NCBI Reference Sequence: NM_144982.5 GI: 1647818743. Exemplary human ZFC3H1 protein sequences include, but are not limited to, NCBI Reference Sequence: AAH73843.1 GI: 49256579. Without wishing to be bound by theory, it is believed that in some embodiments, ZFC3H1 is involved in exosomal degradation of polyadenylated RNA.

As used herein, the term “SETDB1,” “SET domain bifurcated histone lysine methyltransferase 1” refers to the gene SETDB1 and the gene product encoded by the SETDB1 gene. It is also known as “ESET”, “H3-K9-HMTase4”, “KG1T”,” KMT1E,” or “TDRD21.” In the human genome, SETDB1 is located on chromosome 1. Exemplary human SETDB1 transcript sequences include, but are not limited to, NCBI Reference Sequences: NM_001366418.1 GI: 1485835039, NM_012432.4 GI: 1677499399, NM_001145415.2 GI: 1677498643, NM_001243491.2 GI: 1677500297,NM_001366417.1 GI: 1485835069, NM_001393958.1 GI: 2017363552, NM_001393959.1 GI: 2017363496, NM_001393960.1 GI: 2017363556, NM_001393961.1 GI: 2017363545, NM_001393964.1 GI: 2017363494, NM_001393965.1 GI: 2017363528, NM_001393966.1 GI: 2017363523, NM_001393967.1 GI: 2017363558, NR_172060.1 GI: 2017363584 and NM_001393968.1 GI: 2017363503. Exemplary human SETDB1 protein sequences include, but are not limited to, NCBI Reference Sequences: NP_001353347.1 GI: 1485835040, NP_036564.3 GI: 224177469, NP_001138887.1 GI: 224177467, NP_001230420.1 GI: 343488525, NP_001353346.1 GI: 1485835070, NP_001380887.1 GI: 2017363553, NP_001380888.1 GI: 2017363497, NP_001380889.1 GI: 2017363557, NP_001380890.1 GI: 2017363546, NP_001380893.1 GI: 2017363495, NP_001380894.1 GI: 2017363529, NP_001380895.1 GI: 2017363524, NP_001380896.1 GI: 2017363559, and NP_001380897.1 GI: 2017363504.

As used herein, the term “SMCHD1,” “structural maintenance of chromosomes flexible hinge domain containing 1” refers to the gene SMCHD1 and the gene product encoded by the SMCHD1 gene. It is also known as “BAMS,” or “FSHD2.” In the human genome, SMCHD1 is located on chromosome 18. Exemplary human SMCHD1 transcript sequences include, but are not limited to, NCBI Reference Sequences: NM_015295.3 GI: 1779421293, XM_011525642.1 GI: 767997642, XM_011525643.2 GI: 1034603797, XM_017025684.1 GI: 1034603799, XR_935055.2 GI: 1370473343, XR_001753172.1 GI: 1034603795, XR_001753173.1 GI: 1034603796, XR_001753174.1 GI: 1034603801, XR_001753175.1 GI: 1034603802, XR_001753176.1 GI: 1034603803, XR_001753177.1 GI: 1034603804, XR_001753178.1 GI: 1034603805, and XR_001753179.1 GI: 1034603806. Exemplary human SMCHD1 protein sequences include, but are not limited to, NCBI Reference Sequences: NP_056110.2 GI: 148839305, XP_011523944.1 GI: 767997643, XP_011523945.1 GI: 767997645, and XP_016881173.1 GI: 1034603800. Without wishing to be bound by theory, it is believed that in some embodiments, SMCHD1 is involved in chromatin remodeling.

As used herein, the term “UHRF1,” “ubiquitin like with PHD and ring finger domains 1” refers to the gene UHRF1 and the gene product encoded by the UHRF1 gene. It is also known as “ICBP90”, “Np95”, “RNF106”, “TDRD22”,” hNP95”,” hUHRF1,” or “huNp95.” In the human genome, UHRF1 is located on chromosome 19. Exemplary human UHRF1 transcript sequences include, but are not limited to, NCBI Reference Sequences: NM_001290051.2 GI: 1676317451, NM_001290050.2 GI: 1890277394, NM_013282.5 GI: 1889605047, NM_001048201.3 GI: 1519473788, NM_001290052.2 GI: 1890251165, and XM_011527942.2 GI: 1034607616. Exemplary human UHRF1 protein sequences include, but are not limited to, NCBI Reference Sequences: NP_001041666.1 GI: 115430235, NP_037414.3 GI: 115430233, NP_001276979.1 GI: 586798166, NP_001276980.1 GI: 586798168, NP_001276981.1 GI: 586798170, and XP_011526244.1 GI: 768002066. Without wishing to be bound by theory, it is believed that in some embodiments, UHRF1 is involved in histone deacetylase recruitment to a DNA.

As used herein, the term “CLUL1,” “clusterin like 1” refers to the gene CLUL1 and the gene product encoded by the CLUL1 gene. It is also known as “RA337M.” In the human genome, CLUL1 is located on chromosome 18. Exemplary human CLUL1 transcript sequences include, but are not limited to, NCBI Reference Sequences: NM_001393344.1 GI: 1973482983, NM_014410.6 GI: 1973485589,NM_199167.2 GI: 1973465642, NM_001289036.3 GI: 1973494233,NM_001318522.2 GI: 1973498085, NM_001375492.2 GI: 1973488188,NM_001393345.1 GI: 1973468292, NM_001393346.1 GI: 1973497468,NM_001393347.1 GI: 1973497115, NM_001393348.1 GI: 1973492845, XM_005258103.3 GI: 1034603878, XM_011525648.3 GI: 1370473419, XM_011525649.1 GI: 767997658, XM_011525651.2 GI: 1034603880, XM_011525653.2 GI: 1034603881, and XM_017025707.1 GI: 1034603876. Exemplary human CLUL1 protein sequences include, but are not limited to, NCBI Reference Sequences: NP_001380273.1 GI: 1973482984, NP_055225.1 GI: 7657490, NP_954636.1 GI: 40316926, NP_001275965.2 GI: 1973494234, NP_001305451.1 GI: 972776953, NP_001362421.1 GI: 1771391537, NP_001380274.1 GI: 1973468293, NP_001380275.1 GI: 1973497469, NP_001380276.1 GI: 1973497116, NP_001380277.1 GI: 1973492846, XP_005258160.1 GI: 530424843, XP_011523950.1 GI: 767997657, XP_011523951.1 GI: 767997659, XP_011523953.1 GI: 767997665, XP_011523955.1 GI: 767997670, and XP_016881196.1 GI: 1034603877.

As used herein, the term “SMAD5,” “SMAD family member 5” refers to the gene SMAD5 and the gene product encoded by the SMAD5 gene. It is also known as “DWFC,” “JV5-1,” or “MADH5.” In the human genome, SMAD5 is located on chromosome 5. Exemplary human SMAD5 transcript sequences include, but are not limited to, NCBI Reference Sequences: NM_005903.7 GI: 1519244960, NM_001001419.3 GI: 1676317592, NM_001001420.3 GI: 1890269408, XM_017009470.2 GI: 1370492840, XM_024446046.1 GI: 1370492829, and XM_024446047.1 GI: 1370492847. Exemplary human SMAD5 protein sequences include, but are not limited to, NCBI Reference Sequences: NP_005894.3 GI: 47778925, NP_001001419.1 GI: 47778929, NP_001001420.1 GI: 47778931, XP_016864959.1 GI: 1034644951, XP_024301814.1 GI: 1370492830, and XP_024301815.1 GI: 1370492848.

As used herein, the term “SETX,” “senataxin” refers to the gene SETX and the gene product encoded by the SETX gene. It is also known as “ALS4”, “AOA2”, “SCAN2”, “SCAR1”,” Sen1,” or “bA479K20.2.” In the human genome, SETX is located on chromosome 9. Exemplary human SETX transcript sequences include, but are not limited to, NCBI Reference Sequences: NM_015046.7 GI: 1519311427, NM_001351527.2 GI: 1890342979, NM_001351528.2 GI: 1675015427, XM_005272172.3 GI: 1370514082, XM_005272173.3 GI: 1370514083, XM_011518404.3 GI: 1370514084, XM_011518405.3 GI: 1370514085, XM_011518406.2 GI: 1034664509, XM_011518407.1 GI: 767956715, XM_011518408.3 GI: 1370514086, XM_017014496.1 GI: 1034664513, XR_929739.2 GI: 1034664511, and XR_001746251.1 GI: 1034664512. Exemplary human SETX protein sequences include, but are not limited to, NCBI Reference Sequences: NP_055861.3 GI: 113722133, NP_001338456.1 GI: 1191017961, NP_001338457.1 GI: 1191017963, XP_005272229.1 GI: 530427120, XP_005272230.1 GI: 530427122, XP_011516706.1 GI: 767956710, XP_011516707.1 GI: 767956712, XP_011516708.1 GI: 767956714, XP_011516709.1 GI: 767956716, XP_011516710.1 GI: 767956718, and XP_016869985.1 GI: 1034664514. Without wishing to be bound by theory, it is believed that in some embodiments, SETX is involved in oxidative stress-induced DNA double strand break response.

As used herein, the term “STEEP1,” “STING1 ER exit protein 1” refers to the gene STEEP1 and the gene product encoded by the STEEP1 gene. It is also known as “CXorf56”, “MRX107”, “STEEP,” or “XLID107.” In the human genome, STEEP1 is located on chromosome X. Exemplary human STEEP1 transcript sequences include, but are not limited to, NCBI Reference Sequences: NM_022101.4 GI: 1677500855 NM_001170569.1 GI: 283046675, and NM_001170570.2 GI: 1677500166. Exemplary human STEEP1 protein sequences include, but are not limited to, NCBI Reference Sequences: NP_071384.1 GI: 11545813, NP_001164040.1 GI: 283046676, and NP_001164041.1 GI: 283046682.

As used herein, the term “DENND6A,” “DENN domain containing 6A” refers to the gene DENND6A and the gene product encoded by the DENND6A gene. It is also known as “AFI1A,” or “FAM116A.” In the human genome, DENND6A is located on chromosome 3. Exemplary human DENND6A transcript sequences include, but are not limited to, NCBI Reference Sequences: NM_152678.3 GI: 1519243545, XM_006713019.3 GI: 1034631729, XM_006713020.2 GI: 1034631730, XM_017005864.1 GI: 1034631732, XR_245100.1 GI: 530372045, and XR_001740057.2 GI: 1370483428. Exemplary human DENND6A protein sequences include, but are not limited to, NCBI Reference Sequences: NP_689891.1 GI: 32698777, XP_006713082.1 GI: 578805767, XP_006713083.1 GI: 578805769, and XP_016861353.1 GI: 1034631733. Without wishing to be bound by theory, it is believed that in some embodiments, DENND6A is involved in vesicle-mediated endocytosis.

As used herein, the term “RTN4RL2,” “reticulon 4 receptor like 2” refers to the gene RTN4RL2 and the gene product encoded by the RTN4RL2gene. It is also known as “NGRH1,” or “NgR2.” In the human genome, RTN4RL2 is located on chromosome 11. Exemplary human RTN4RL2 transcript sequences include, but are not limited to, NCBI Reference Sequence: NM_178570.3 GI: 1519242372. Exemplary human RTN4RL2 protein sequences include, but are not limited to, NCBI Reference Sequence: ABI23432.1 GI: 112818153.

As used herein, the term “RAB1B,” “RAB1B, member RAS oncogene family” refers to the gene RAB1B and the gene product encoded by the RAB1B gene. In the human genome, RAB1B is located on chromosome 11. Exemplary human RAB1B transcript sequences include, but are not limited to, NCBI Reference Sequences: NM_030981.3 GI: 1519244442 and XM_017018378.1 GI: 1034575704. Exemplary human RAB1B protein sequences include, but are not limited to, NCBI Reference Sequences: NP_112243.1 GI: 13569962 and XP_016873867.1 GI: 1034575705.

As used herein, the term “MTMR6,” “myotubularin related protein 6” refers to the gene MTMR6 and the gene product encoded by the MTMR6 gene. In the human genome, MTMR6 is located on chromosome 13. Exemplary human MTMR6 transcript sequences include, but are not limited to, NCBI Reference Sequences: NM_004685.5 GI: 1869284250, NM_001385230.1 GI: 1869284187, NM_001385231.1 GI: 1869284168, NM_001385232.1 GI: 1869284205, NM_001385233.1 GI: 1869284256, NM_001385234.1 GI: 1869284175, NM_001385235.1 GI: 1869284198, NM_001385236.1 GI: 1869284189, NM_001385237.1 GI: 1869284236, NM_001385238.1 GI: 1869284219, NR_169592.1 GI: 1869284284, XM_011535307.1 GI: 767978249, and XM_017020846.1 GI: 1034585226. Exemplary human MTMR6 protein sequences include, but are not limited to, NCBI Reference Sequences: NP_004676.3 GI: 134142348, NP_001372159.1 GI: 1869284188, NP_001372160.1 GI: 1869284169, NP_001372161.1 GI: 1869284206, NP_001372162.1 GI: 1869284257, NP_001372163.1 GI: 1869284176, NP_001372164.1 GI: 1869284199, NP_001372165.1 GI: 1869284190, P_001372166.1 GI: 1869284237, NP_001372167.1 GI: 1869284220, XP_011533609.1 GI: 767978250, and XP_016876335.1 GI: 1034585227.

As used herein, the term “VAPA,” “VAMP associated protein A” refers to the gene VAPA and the gene product encoded by the VAPA gene. It is also known as “VAMP-A”, “VAP-33”, “VAP-A”, “VAP33,” or “hVAP-33.” In the human genome, VAPA is located on chromosome 18. Exemplary human VAPA transcript sequences include, but are not limited to, NCBI Reference Sequences: NM_194434.3 GI: 1677501098, NM_003574.6 GI: 1677498770, XM_011525769.3 GI: 1370473950, M_017026079.1 GI: 1034605012, and XM_024451287.1 GI: 1370473951. Exemplary human VAPA protein sequences include, but are not limited to, NCBI Reference Sequences: NP_919415.2 GI: 94721252, NP_003565.4 GI: 94721250, XP_011524071.1 GI: 767997951, XP_016881568.1 GI: 1034605013, and XP_024307055.1 GI: 1370473952.

As used herein, the term “MAP3K20,” “mitogen-activated protein kinase kinase kinase 20” refers to the gene MAP3K20 and the gene product encoded by the MAP3K20 gene. It is also known as “AZK”, “CNM6”, “MLK7”, “MLT”, “MLTK”, “MLTKalpha”, “MLTKbeta”, “MRK”, “SFMMP”, “ZAK”, “mlklak,” or “pk.” In the human genome, MAP3K20 is located on chromosome 2. Exemplary human MAP3K20 transcript sequences include, but are not limited to, NCBI Reference Sequences: NM_016653.3 GI: 1519314972, NM_133646.3 GI: 1676319300, XM_005246640.2 GI: 1034614634, XM_017004323.1 GI: 1034614635, and XM_017004324.1 GI: 1034614637. Exemplary human MAP3K20 protein sequences include, but are not limited to, NCBI Reference Sequences: NP_000685.1 GI: 4502043, NP_001025181.1 GI: 71773324, NP_001154945.1 GI: 238814371, NP_001276987.1 GI: 586946392, and NP_001276988.1 GI: 586946394.

As used herein, the term “COL8A1,” “collagen type VIII alpha 1 chain” refers to the gene COL8A1 and the gene product encoded by the COL8A1 gene. It is also known as “C3orf7.” In the human genome, COL8A1 is located on chromosome 3. Exemplary human COL8A1 transcript sequences include, but are not limited to, NCBI Reference Sequences: NM_020351.4 GI: 1519243384 and NM_001850.5 GI: 1674986814. Exemplary human COL8A1 protein sequences include, but are not limited to, NCBI Reference Sequences: NP_065084.2 GI: 32895368 and NP_001841.2 GI: 17738302.

As used herein, the term “FASN,” “fatty acid synthase” refers to the gene FASN and the gene product encoded by the FASN gene. It is also known as “FAS,” “OA-519,” or “DR27X1.” In the human genome, FASN is located on chromosome 17. Exemplary human FASN transcript sequences include, but are not limited to, NCBI Reference Sequences: NM_004104.5 GI: 1519312524 XM_011523538.2 GI: 1034598774. Exemplary human FASN protein sequences include, but are not limited to, NCBI Reference Sequences: NP_004095.4 GI: 41872631 and XP_011521840.1 GI: 767997328.

As used herein, the term “STRADA,” “STE20 related adaptor alpha” refers to the gene STRADA and the gene product encoded by the STRADA gene. It is also known as “LYK5”, “NY-BR-96”, “PMSE”, “STRAD”, “STRAD alpha,” or “Stlk.” In the human genome, STRADA is located on chromosome 17. Exemplary human STRADA transcript sequences include, but are not limited to, NCBI Reference Sequences: NM_001003787.4 GI: 1519315350, NM_153335.6 GI: 1394533274, NM_001003786.3 GI: 1394533285, NM_001003788.3 GI: 1394533255, NM_001165969.2 GI: 1394533288, NM_001165970.2 GI: 1394533185, NM_001363786.1 GI: 1394533200, NM_001363787.1 GI: 1394533235, NM_001363788.1 GI: 1394533259, NM_001363789.1 GI: 1394533183, NM_001363790.1 GI: 1394533247, NM_001363791.1 GI: 1394533294, NR_156741.2 GI: 1701971339, XM_005257799.3 GI: 1370472599, XM_005257801.5 GI: 1370472602, XM_005257803.5 GI: 1370472604, XM_011525466.3 GI: 1370472596, XM_011525467.3 GI: 1370472598, XM_017025314.2 GI: 1370472603, and XR_243688.3 GI: 1370472606. Exemplary human STRADA protein sequences include, but are not limited to, NCBI Reference Sequences: NP_001003787.1 GI: 51242955, NP_699166.2 GI: 31982873, NP_001003786.1 GI: 51242960, NP_001003788.1 GI: 51242957, NP_001159441.1 GI: 260166664, NP_001159442.1 GI: 260166666, NP_001350715.1 GI: 1394533201, NP_001350716.1 GI: 1394533236, NP_001350717.1 GI: 1394533260, NP_001350718.1 GI: 1394533184, NP_001350719.1 GI: 1394533248, NP_001350720.1 GI: 1394533295, XP_005257856.1 GI: 530413119, XP_005257858.1 GI: 530413123, XP_005257860.1 GI: 530413127, XP_011523768.1 GI: 767996459, XP_011523769.1 GI: 767996461, and XP_016880803.1 GI: 1034602015.

As used herein, the term “DAXX,” “death domain associated protein” refers to the gene DAXX and the gene product encoded by the DAXX gene. It is also known as “BING2”, “DAP6”, “EAP1,” or “SMIM40.” In the human genome, DAXX is located on chromosome 6. Exemplary human DAXX transcript sequences include, but are not limited to, NCBI Reference Sequences: NM_001141969.2 GI: 1519242651, NM_001350.5 GI: 1674986418, M_001141970.2 GI: 1676319834, NM_001254717.2 GI: 1890275313, and XM_005248860.3 GI: 767939291. Exemplary human DAXX protein sequences include, but are not limited to, NCBI Reference Sequences: NP_001135441.1 GI: 215422388, NP_001341.1 GI: 4503257, NP_001135442.1 GI: 215422366, NP_001241646.1 GI: 359843219, and XP_005248917.1 GI: 530381485.

As used herein, the term “PSIP1,” “PC4 and SFRS1 interacting protein 1” refers to the gene PSIP1 and the gene product encoded by the PSIP1 gene. It is also known as “DFS70”, “LEDGF”, “PAIP”, “PSIP2”, “p52,” or “p57.” In the human genome, PSIP1 is located on chromosome 9. Exemplary human PSIP1 transcript sequences include, but are not limited to, NCBI Reference Sequences: NM_033222.5 GI: 1519241968, NM_021144.4 GI: 1890342066, NM_001128217.3 GI: 1890258155, NM_001317898.3 GI: 1890272938, NM_001317900.3 GI: 1890270743, XM_024447399.1 GI: 1370513697, XM_024447400.1 GI: 1370513699, XM_024447401.1 GI: 1370513701, and XM_024447402.1 GI: 1370513703. Exemplary human PSIP1 protein sequences include, but are not limited to, NCBI Reference Sequences: NP_150091.2 GI: 19923653, NP_066967.3 GI: 190014586, NP_001121689.1 GI: 190014588, NP_001304827.1 GI: 961513278, NP_001304829.1 GI: 961526013, XP_024303167.1 GI: 1370513698, XP_024303168.1 GI: 1370513700, XP_024303169.1 GI: 1370513702, and XP_024303170.1 GI: 1370513704. Without wishing to be bound by theory, it is believed that in some embodiments, PSIP1 is involved in transcription.

As used herein, the term “BMPR1A” “bone morphogenetic protein receptor type 1A” refers to the gene BMPR1A and the gene product encoded by the BMPR1A gene. It is also known as “0q23del”, “ACVRLK3”, “ALK3”, “CD292,” or “SKR5.” In the human genome, BMPR1A is located on chromosome 10. Exemplary human BMPR1A transcript sequences include, but are not limited to, NCBI Reference Sequences: NM_004329.3 GI: 1779521761, XM_011540103.2 GI: 1034569315, and XM_011540104.2 GI: 1034569316. Exemplary human BMPR1A protein sequences include, but are not limited to, NCBI Reference Sequences: NP_004320.2 GI: 41349437, XP_011538405.1 GI: 767963636, and XP_011538406.1 GI: 767963638.

As used herein, the term “AP3M1,” “adaptor related protein complex 3 subunit mu 1” refers to the gene AP3M1 and the gene product encoded by the AP3M1 gene. In the human genome, AP3M1 is located on chromosome 10. Exemplary human AP3M1 transcript sequences include, but are not limited to, NCBI Reference Sequences: NM_012095.6 GI: 1677500843, NM_207012.4 GI: 1677498261, NM_001320263.2 GI: 1677499660, NM_001320264.2 GI: 1677499665, NM_001320265.2 GI: 1677530660, NR_135191.2 GI: 1700447798, XM_024447939.1 GI: 1370456871, XR_001747091.2 GI: 1370456873, and XR_001747092.2 GI: 1370456874. Exemplary human AP3M1 protein sequences include, but are not limited to, NCBI Reference Sequences: NP_036227.1 GI: 6912240, NP_996895.1 GI: 46370095, NP_001307192.1 GI: 998429222, NP_001307193.1 GI: 998429224, NP_001307194.1 GI: 998429226, and XP_024303707.1 GI: 1370456872. Without wishing to be bound by theory, it is believed that in some embodiments, AP3M1 is involved in clathrin-coated vesicle-mediated endocytosis.

As used herein, the term “MEN1,” “menin 1” refers to the gene MEN1 and the gene product encoded by the MEN1 gene. It is also known as “MEAL,” or “SCG2.” In the human genome, MEN1 is located on chromosome 11. Exemplary human MEN1 transcript sequences include, but are not limited to, NCBI Reference Sequences: NM_001370259.2 GI: 1779948666, NM_000244.3 GI: 210031708, NM_130799.2 GI: 210031700, NM_130800.2 GI: 210031718, NM_130801.2 GI: 210031723, NM_130802.2 GI: 210031715, NM_130803.2 GI: 210031734, NM_130804.2 GI: 210031781, NM_001370251.1 GI: 1631970671, NM_001370260.1 GI: 1631970690, NM_001370261.1 GI: 1631970718, NM_001370262.1 GI: 1631970736, NM_001370263.1 GI: 1631970714, XM_011545040.1 GI: 767968201, XM_011545041.2 GI: 1034573763, XM_017017765.1 GI: 1034573755, XM_017017766.1 GI: 1034573757, XM_017017767.2 GI: 1370459430, and XM_017017768.1 GI: 1034573761. Exemplary human MEN1 protein sequences include, but are not limited to, NCBI Reference Sequences: NP_001357188.2 GI: 1779948667, NP_000235.2 GI: 18860839, NP_570711.1 GI: 18860847, NP_570712.1 GI: 18860849, NP_570713.1 GI: 18860851, NP_570714.1 GI: 18860853, NP_570715.1 GI: 18860855, NP_570716.1 GI: 18860857, NP_001357180.1 GI: 1631970672, NP_001357189.1 GI: 1631970691, NP_001357190.1 GI: 1631970719, NP_001357191.1 GI: 1631970737, NP_001357192.1 GI: 1631970715, XP_011543342.1 GI: 767968202, XP_011543343.1 GI: 767968204, XP_016873254.1 GI: 1034573756, XP_016873255.1 GI: 1034573758, XP_016873256.1 GI: 1034573760, and XP_016873257.1 GI: 1034573762. Without wishing to be bound by theory, it is believed that in some embodiments, MEN1 is involved in histone modification.

As used herein, the term “MYL12B,” “myosin light chain 12B” refers to the gene MYL12B and the gene product encoded by the MYL12B gene. It is also known as “MLC-B,” or “MRLC2.” In the human genome, MYL12B is located on chromosome 18. Exemplary human MYL12B transcript sequences include, but are not limited to, NCBI Reference Sequences: NM_033546.4 GI: 1813765003, NM_001144944.1 GI: 222144323, and NM_001144945.1 GI: 222144325. Exemplary human MYL12B protein sequences include, but are not limited to, NCBI Reference Sequences: NP_291024.1 GI: 15809016, NP_001138416.1 GI: 222144324, and NP_001138417.1 GI: 222144326.

As used herein, the term “CREB1,” “cAMP responsive element binding protein 1” refers to the gene CREB1 and the gene product encoded by the CREB1 gene. It is also known as “CREB,” or “CREB-1.” In the human genome, CREB1 is located on chromosome 2. Exemplary human CREB1 transcript sequences include, but are not limited to, NCBI Reference Sequences: NM_004379.5 GI: 1519313108, NM_134442.5 GI: 1676316951, NM_001320793.2 GI: 1675162569, NM_001371426.1 GI: 1701944685, NM_001371427.1 GI: 1701944572, NM_001371428.1 GI: 1701944586, NR_135473.2 GI: 1701216066, NR_163946.1 GI: 1701973920, NR_163947.1 GI: 1701973928, XM_011510646.3 GI: 1370476775, XM_011510648.3 GI: 1370476779, XM_011510650.3 GI: 1370476780, XM_017003399.2 GI: 1370476781, XM_017003401.2 GI: 1370476786, XR_241290.2 GI: 1370476783, XR_241292.2 GI: 1370476784, XR_001738636.2 GI: 1370476782, and XR_001738637.2 GI: 1370476785. Exemplary human CREB1 protein sequences include, but are not limited to, NCBI Reference Sequences: NP_004370.1 GI: 4758054, NP_604391.1 GI: 19745184, NP_001307722.1 GI: 1003088836, NP_001358355.1 GI: 1701944686, NP_001358356.1 GI: 1701944573, NP_001358357.1 GI: 1701944587, XP_011508948.1 GI: 767916785, XP_011508950.1 GI: 767916789, XP_011508952.1 GI: 767916793, XP_016858888.1 GI: 1034611771, and XP_016858890.1 GI: 1034611778.

As used herein, the term “SIMC1,” “SUMO interacting motifs containing 1” refers to the gene SIMC1 and the gene product encoded by the SIMC1 gene. It is also known as “C5orf25”, “OOMA1,” or “PLEIAD.” In the human genome, SIMC1 is located on chromosome 5. Exemplary human SIMC1 transcript sequences include, but are not limited to, NCBI Reference Sequences: NM_001308195.2 GI: 1519246537, NM_198567.6 GI: 1890275999, NM_001308196.2 GI: 1890343156, NM_001308200.2 GI: 1890342839, NR_131772.2 GI: 1700447694, XM_011534553.2 GI: 1034644902, XM_011534554.2 GI: 1034644903, XM_011534556.2 GI: 1034644906, XM_017009454.1 GI: 1034644904, XM_017009455.1 GI: 1034644907, and XM_017009456.1 GI: 1034644909. Exemplary human SIMC1 protein sequences include, but are not limited to, NCBI Reference Sequences: NP_001295124.1 GI: 815891034, NP_940969.3 GI: 196259796, NP_001295125.1 GI: 815890967, NP_001295129.1 GI: 815890965, XP_011532855.1 GI: 767938380, XP_011532856.1 GI: 767938382, XP_011532858.1 GI: 767938386, XP_016864943.1 GI: 1034644905, XP_016864944.1 GI: 1034644908, and XP_016864945.1 GI: 1034644910.

As used herein, the term “USP9X,” “ubiquitin specific peptidase 9 X-linked” refers to the gene USP9X and the gene product encoded by the USP9X gene. It is also known as “DFFRX”, “FAF”, “FAM”, “MRX99”, “MRXS99F,” or “XLID99.” In the human genome, USP9X is located on chromosome X. Exemplary human USP9X transcript sequences include, but are not limited to, NCBI Reference Sequences: NM_001039591.3 GI: 1519244693, NM_001039590.3 GI: 1676317946, XM_005272675.4 GI: 1034675403, and XM_005272676.4 GI: 1034675404. Exemplary human USP9X protein sequences include, but are not limited to, NCBI Reference Sequences: NP_001034680.2 GI: 145309311, NP_001034679.2 GI: 145309309, XP_005272732.1 GI: 530421594, and XP_005272733.1 GI: 530421596.

As used herein, the term “RAB12,” “RAB12, member RAS oncogene family” refers to the gene RAB12 and the gene product encoded by the RAB12 gene. In the human genome, RAB12 is located on chromosome 18. Exemplary human RAB12 transcript sequences include, but are not limited to, NCBI Reference Sequences: NM_001025300.3 GI: 1929903198, XM_006722300.3 GI: 1034603581, XR_001753165.1 GI: 1034603579, and XR_001753166.1 GI: 1034603580. Exemplary human RAB12 protein sequences include, but are not limited to, NCBI Reference Sequences: NP_001020471.3 GI: 1929903199 and XP_006722363.1 GI: 578832076. Without wishing to be bound by theory, it is believed that in some embodiments, RAB12 is involved in vesicle-mediated endocytosis.

As used herein, the term “SMTNL1,” “smoothelin like 1” refers to the gene SMTNL1 and the gene product encoded by the SMTNL1 gene. It is also known as “CHASM.” In the human genome, SMTNL1 is located on chromosome 11. Exemplary human SMTNL1 transcript sequences include, but are not limited to, NCBI Reference Sequence: NM_001105565.3 GI: 1862757442. Exemplary human SMTNL1 protein sequences include, but are not limited to, NCBI Reference Sequences: NP_001099035.2 GI: 303304956.

As used herein, the term “MRGBP,” “MRG domain binding protein” refers to the gene MRGBP and the gene product encoded by the MRGBP gene. It is also known as “C20orf20”, “Eaf7”, “MRG15BP,” or “URCC4.” In the human genome, MRGBP is located on chromosome 20. Exemplary human MRGBP transcript sequences include, but are not limited to, NCBI Reference Sequences: NM_018270.6 GI: 1519316164 and NR_136405.2 GI: 1701216204. Exemplary human MRGBP protein sequences include, but are not limited to, NCBI Reference Sequence: NP_060740.1 GI: 8922764. Without wishing to be bound by theory, it is believed that in some embodiments, MRGBP is involved in nucleosome/DNA interaction.

As used herein, the term “SUMO2,” “small ubiquitin like modifier 2” refers to the gene SUMO2 and the gene product encoded by the SUMO2 gene. It is also known as “HSMT3”, “SMT3B”, “SMT3H2”, “SUMO3,” or “Smt3A.” In the human genome, SUMO2 is located on chromosome 17. Exemplary human SUMO2 transcript sequences include, but are not limited to, NCBI Reference Sequences: NM_006937.4 GI: 1519242452 and NM_001005849.2 GI: 1676316991.

Exemplary human SUMO2 protein sequences include, but are not limited to, NCBI Reference Sequences: NP_008868.3 GI: 54792069 and NP_001005849.1 GI: 54792071. Without wishing to be bound by theory, it is believed that in some embodiments, SUMO2 is involved in SUMOylation of a protein.

As used herein, the term “PIGN,” “phosphatidylinositol glycan anchor biosynthesis class N” refers to the gene PIGN and the gene product encoded by the PIGN gene. It is also known as “MCAHS”, “MCAHS1”, “MCD4”, “MDC4,” or “PIG-N.” In the human genome, PIGN is located on chromosome 18. Exemplary human PIGN transcript sequences include, but are not limited to, NCBI Reference Sequences: NM_176787.5 GI: 1519314493, NM_012327.6 GI: 1676319953, XM_011525889.1 GI: 767998468, XM_011525890.1 GI: 767998470, XM_011525891.1 GI: 767998472, XM_011525892.1 GI: 767998475, XM_011525893.1 GI: 767998477, XM_011525894.1 GI: 767998479, XM_011525895.1 GI: 767998481, XM_011525896.1 GI: 767998483, XM_011525898.1 GI: 767998487, XM_017025685.1 GI: 1034603807, and XM_017025686.1 GI: 1034603809. Exemplary human PIGN protein sequences include, but are not limited to, NCBI Reference Sequences: NP_789744.1 GI: 29029537, NP_036459.1 GI: 6912500, XP_011524191.1 GI: 767998469, XP_011524192.1 GI: 767998471, XP_011524193.1 GI: 767998473, XP_011524194.1 GI: 767998476, XP_011524195.1 GI: 767998478, XP_011524196.1 GI: 767998480, XP_011524197.1 GI: 767998482, XP_011524198.1 GI: 767998484, XP_011524200.1 GI: 767998488, XP_016881174.1 GI: 1034603808, and XP_016881175.1 GI: 1034603810. Without wishing to be bound by theory, it is believed that in some embodiments, PIGN is involved in GPI biosynthesis.

As used herein, the term “AP1G1,” “adaptor related protein complex 1 subunit gamma 1” refers to the gene AP1G1 and the gene product encoded by the AP1G1 gene. It is also known as “ADTG,” “CLAPG1,” or “USRISD.” In the human genome, AP1G1 is located on chromosome ______. Exemplary human AP1G1 transcript sequences include, but are not limited to, NCBI Reference Sequences: NM_001128.6 GI: 1677538236 and NM_001030007.2 GI: 1677501274. Exemplary human AP1G1 protein sequences include, but are not limited to, NCBI Reference Sequences: NP_001119.3 GI: 71772942 and NP_001025178.1 GI: 71773010. Without wishing to be bound by theory, it is believed that in some embodiments, AP1G1 is involved in WDR1I pathway.

As used herein, the term “AP1S1,” “adaptor related protein complex 1 subunit sigma 1” refers to the gene AP1S1 and the gene product encoded by the AP1S1 gene. It is also known as “AP19”, “CLAPS1”, “EKV3”, “MEDNIK,” or “SIGMA1A.” In the human genome, AP1S1 is located on chromosome 7. Exemplary human AP1S1 transcript sequences include, but are not limited to, NCBI Reference Sequence: NM_001283.5 GI: 1519312734. Exemplary human AP1S1 protein sequences include, but are not limited to, NCBI Reference Sequence: NP_001274.1 GI: 4557471. Without wishing to be bound by theory, it is believed that in some embodiments, AP1S1 is involved in WDR11 pathway.

As used herein, the term “APIM1,” “adaptor related protein complex 1 subunit mu 1” refers to the gene AP1M1 and the gene product encoded by the AP1M1 gene. It is also known as “AP47”, “CLAPM2”, “CLTNM”, “MU-1A,” or “mulA.” In the human genome, AP1M1 is located on chromosome 19. Exemplary human AP1M1 transcript sequences include, but are not limited to, NCBI Reference Sequences: NM_032493.4 GI: 1677479768 and NM_001130524.2 GI: 1677502062. Exemplary human APIM1 protein sequences include, but are not limited to, NCBI Reference Sequences: NP_115882.1 GI: 14210504 and NP_001123996.1 GI: 194473724. Without wishing to be bound by theory, it is believed that in some embodiments, APIM1 is involved WDR11 pathway.

As used herein, the term “APIS3,” “adaptor related protein complex 1 subunit sigma 3” refers to the gene AP1S3 and the gene product encoded by the AP1S3 gene. It is also known as “PSORS15,” or “sigmalC.” In the human genome, AP1S3 is located on chromosome 2. Exemplary human AP1S3 transcript sequences include, but are not limited to, NCBI Reference Sequences: NM_001039569.2 GI: 1519312447, NR_110905.2 GI: 1701971836, NR_110906.2 GI: 1700660570, and XM_011510600.3 GI: 1370476705. Exemplary human AP1S3 protein sequences include, but are not limited to, NCBI Reference Sequences: NP_001034658.1 GI: 88703051 and XP_011508902.1 GI: 767916648. Without wishing to be bound by theory, it is believed that in some embodiments, APIS3 is involved in WDR11 pathway.

As used herein, the term “ELOVL1,” “ELOVL fatty acid elongase 1” refers to the gene ELOVL1 and the gene product encoded by the ELOVL1 gene. It is also known as “CGI-88,” “IKSH,” or “Ssc1.” In the human genome, ELOVL1 is located on chromosome 1. Exemplary human ELOVL1 transcript sequences include, but are not limited to, NCBI Reference Sequences: NM_022821.4 GI: 1519244245, NM_001256399.2 GI: 1889454319, NM_001256401.2 GI: 1890250128, NM_001256402.2 GI: 1675112624, and NR_046117.2 GI: 1701153270. Exemplary human ELOVL1 protein sequences include, but are not limited to, NCBI Reference Sequences: NP_073732.1 GI: 13489093, NP_001243328.1 GI: 373938448, NP_001243330.1 GI: 373938450, and NP_001243331.1 GI: 373938452. Without wishing to be bound by theory, it is believed that in some embodiments, ELOVL1 is involved in long-chain FA elongation cycle.

As used herein, the term “ARL14EP,” “ADP ribosylation factor like GTPase 14 effector protein” refers to the gene ARL14EP and the gene product encoded by the ARL14EP gene. It is also known as “ARF7EP,” “C1l orf46,” or “dJ299F11.1.” In the human genome, ARL14EP is located on chromosome 11. Exemplary human ARL14EP transcript sequences include, but are not limited to, NCBI Reference Sequences: NM_152316.3 GI: 1519315889 and XM_005252792.4 GI: 1370458767. Exemplary human ARL14EP protein sequences include, but are not limited to, NCBI Reference Sequences: NP_689529.1 GI: 22748693 and XP_005252849.1 GI: 530395070.

As used herein, the term “TRIM49,” “tripartite motif containing 49” refers to the gene TRIM49 and the gene product encoded by the TRIM49 gene. It is also known as “RNF18,” “TRIM49A,” or “TRIM49L2.” In the human genome, TRIM49 is located on chromosome 11.

Exemplary human TRIM49 transcript sequences include, but are not limited to, NCBI Reference Sequences: NM_020358.2 GI: 35493773, XM_017018027.2 GI: 1370459806, and XM_024448617.1 GI: 1370459804. Exemplary human TRIM49 protein sequences include, but are not limited to, NCBI Reference Sequences: NP_065091.1 GI: 9966829, XP_016873516.1 GI: 1034574613, and XP_024304385.1 GI: 1370459805.

As used herein, the term “DOT1L,” “DOT1 like histone lysine methyltransferase” refers to the gene DOT1L and the gene product encoded by the DOT1L gene. It is also known as “DOT1,” or “KMT4.” In the human genome, DOT1L is located on chromosome 19. Exemplary human DOT1L transcript sequences include, but are not limited to, NCBI Reference Sequences: NM_032482.3 GI: 1519244207, XM_005259659.3 GI: 1034609733, XM_005259660.3 GI: 1034609734, XM_011528359.2 GI: 1034609735, XM_011528360.1 GI: 768004399, XM_011528361.2 GI: 1034609738, and XM_017027366.1 GI: 1034609736. Exemplary human DOT1L protein sequences include, but are not limited to, NCBI Reference Sequences: NP_115871.1 GI: 22094135, XP_005259716.1 GI: 530425459, XP_005259717.1 GI: 530425461, XP_011526661.1 GI: 768004396, XP_011526662.1 GI: 768004400, XP_011526663.1 GI: 768004407, and XP_016882855.1 GI: 1034609737.

As used herein, the term “CHD7,” “chromodomain helicase DNA binding protein 7” refers to the gene CHD7 and the gene product encoded by the CHD7 gene. It is also known as “CRG”, “HH5”, “IS3,” or “KAL5.” In the human genome, CHD7 is located on chromosome 8. Exemplary human CHD7 transcript sequences include, but are not limited to, NCBI Reference Sequences: NM_017780.4 GI: 1519245546, NM_001316690.1 GI: 940517010, XM_011517553.2 GI: 1034660840, XM_011517554.3 GI: 1370512560, XM_011517555.2 GI: 1034660844, XM_011517560.2 GI: 1034660847, XM_017013612.1 GI: 1034660842, and XM_017013613.1 GI: 1034660845. Exemplary human CHD7 protein sequences include, but are not limited to, NCBI Reference Sequences: NP_060250.2 GI: 54112403, NP_001303619.1 GI: 940517011, XP_011515855.1 GI: 767951833, XP_011515856.1 GI: 767951835, XP_011515857.1 GI: 767951837, XP_011515862.1 GI: 767951847, XP_016869101.1 GI: 1034660843, and XP_016869102.1 GI: 1034660846.

As used herein, the term “PYM1,” “PYM homolog 1, exon junction complex associated factor” refers to the gene PYM1 and the gene product encoded by the PYM1 gene. It is also known as “PYM,” or “WIBG.” In the human genome, PYM1 is located on chromosome 12. Exemplary human PYM1 transcript sequences include, but are not limited to, NCBI Reference Sequences: NM_032345.3 GI: 1519313934 and NM_001143853.1 GI: 219803945. Exemplary human PYM1 protein sequences include, but are not limited to, NCBI Reference Sequences: NP_115721.1 GI: 14150139 and NP_001137325.1 GI: 219803946.

As used herein, the term “STX16,” “syntaxin 16” refers to the gene STX16 and the gene product encoded by the STX16 gene. It is also known as “SYN16.” In the human genome, STX16 is located on chromosome 20. Exemplary human STX16 transcript sequences include, but are not limited to, NCBI Reference Sequences: NM_001001433.3 GI: 1519242278, NM_003763.6 GI: 1676317536, NM_001134772.3 GI: 1676319200, NM_001134773.3 GI: 1676319858, NM_001204868.2 GI: 1676317907, NR_037941.2 GI: 1700660526, NR_037942.2 GI: 1708260805, and NR_037943.2 GI: 1708260695. Exemplary human STX16 protein sequences include, but are not limited to, NCBI Reference Sequences: NP_116264.2 GI: 27734755, NP_001171835.1 GI: 296531375, NP_001357138.1 GI: 1628814870, and NP_001357137.1 GI: 1628814739. Without wishing to be bound by theory, it is believed that in some embodiments, STX16 is involved in vesicular transport from a late endosome to a trans-Golgi network

As used herein, the term “TCN1,” “transcobalamin 1” refers to the gene TCN1 and the gene product encoded by the TCN1 gene. It is also known as “HC,” “TC-1”, “TC1,” or “TCI.” In the human genome, TCN1 is located on chromosome 11. Exemplary human TCN1 transcript sequences include, but are not limited to, NCBI Reference Sequence: NM_001062.4 GI: 1519314932. Exemplary human TCN1 protein sequences include, but are not limited to, NCBI Reference Sequences: NP_001053.2 GI: 21071008.

As used herein, the term “ALDH3B1,” “aldehyde dehydrogenase 3 family member B1” refers to the gene ALDH3B1 and the gene product encoded by the ALDH3B1 gene. It is also known as “ALDH4,” or “ALDH7.” In the human genome, ALDH3B1 is located on chromosome 11. Exemplary human ALDH3B1 transcript sequences include, but are not limited to, NCBI Reference Sequences: NM_000694.4 GI: 1519312307, NM_001030010.3 GI: 1676319839, NM_001161473.3 GI: 1674986225, NM_001290058.2 GI: 1675053273, and NM_001290059.2 GI: 1676319223. Exemplary human ALDH3B1 protein sequences include, but are not limited to, NCBI Reference Sequences: NP_000685.1 GI: 4502043, NP_001025181.1 GI: 71773324, NP_001154945.1 GI: 238814371, NP_001276987.1 GI: 586946392, and NP_001276988.1 GI: 586946394.

As used herein, the term “FOXK1,” “forkhead box Ki” refers to the gene FOXK1 and the gene product encoded by the FOXK1 gene. It is also known as “FOXK1L.” In the human genome, CXXC1 is located on chromosome 7. Exemplary human FOXK1 transcript sequences include, but are not limited to, NCBI Reference Sequence: M_004514.4 GI: 1519313569. Exemplary human FOXK1 protein sequences include, but are not limited to, NCBI Reference Sequence NP_004505.2 GI: 31563338.

As used herein, the term “MYL12A,” “myosin light chain 12A” refers to the gene MYL12A and the gene product encoded by the MYL12A gene. It is also known as “HEL-S-24”, “MLC-2B”, “MLCB”, “MRCL3”, “MRLC3,” or “MYL2B.” In the human genome, MYL12A is located on chromosome 18. Exemplary human MYL12A transcript sequences include, but are not limited to, NCBI Reference Sequences: NM_006471.4 GI: 1813787182, NM_001303047.2 GI: 1889563402, NM_001303048.1 GI: 740087160, and NM_001303049.2 GI: 1889620061. Exemplary human MYL12A protein sequences include, but are not limited to, NCBI Reference Sequences: NP_006462.1 GI: 5453740, NP_001289976.1 GI: 740087125, NP_001289977.1 GI: 740087161, and NP_001289978.1 GI: 740087210.

As used herein, the term “RHOD,” “ras homolog family member D” refers to the gene RHOD and the gene product encoded by the RHOD gene. It is also known as “ARHD”, “RHOHP1, “RHOM,” or “Rho.” In the human genome, RHOD is located on chromosome 11. Exemplary human RHOD transcript sequences include, but are not limited to, NCBI Reference Sequences: NM_014578.4 GI: 1519316345 and NM_001300886.2 GI: 1676318297. Exemplary human RHOD protein sequences include, but are not limited to, NCBI Reference Sequences: NP_055393.1 GI: 7656900 and NP_001287815.1 GI: 665505975. Without wishing to be bound by theory, it is believed that in some embodiments, RHOD is involved in vesicle-mediated endocytosis.

As used herein, the term “PGAP2,” “post-GPI attachment to proteins 2” refers to the gene PGAP2 and the gene product encoded by the PGAP2 gene. It is also known as “CWH43-N”, “FRAG1”, “HPMRS3”, “MRT17” or “MRT21.” In the human genome, PGAP2 is located on chromosome 11. Exemplary human PGAP2 transcript sequences include, but are not limited to, NCBI Reference Sequences: NM_014489.4 GI: 1653960654, NM_001145438.2 GI: 371940849, NM_001256235.1 GI: 371940850, NM_001256236.1 GI: 371940852, NM_001256237.1 GI: 371940854, NM_001256238.1 GI: 371940856, NM_001256239.2 GI: 1677499573, NM_001256240.2 GI: 1676318184, NM_001283038.1 GI: 545688906, NM_001283039.1 GI: 545688909, NM_001283040.1 GI: 545688911, NM_001346397.2 GI: 1675003151, NM_001346398.2 GI: 1675172629,NM_001346399.2 GI: 1676317496, NM_001346400.2 GI: 1676325159,NM_001346401.2 GI: 1675109331, NM_001346402.2 GI: 1676347923, NM_001346403.1 GI: 1077181875, NM_001346404.1 GI: 1077158368, NM_001346405.1 GI: 1077160070, NR_027016.3 GI: 1708260686, NR_027017.4 GI: 1701948247, NR_027018.2 GI: 372266099, NR_045923.2 GI: 1714614892, NR_045925.2 GI: 1701108991, NR_045926.2 GI: 1701969752, NR_045927.2 GI: 1714608444, NR_045929.2 GI: 1708260827, NR_104270.2 GI: 1714612117, NR_104271.2 GI: 1701109875, NR_104272.2 GI: 1708352870, NR_144427.2 GI: 1676453093, NR_144428.2 GI: 1700447854, NR_144429.2 GI: 1676452803, NR_144430.2 GI: 1676443505, XM_006718181.3 GI: 1034573064, XM_006718185.2 GI: 767965661, XM_006718186.2 GI: 1034573074, XM_006718190.3 GI: 1034573080, XM_006718191.3 GI: 1034573081, XM_006718193.2 GI: 767965670, XM_011519990.2 GI: 1034573054, XM_011519991.2 GI: 1034573056, XM_011519992.1 GI: 767965633, XM_011519994.2 GI: 1034573060, XM_011519996.1 GI: 767965641, XM_011519997.1 GI: 767965643, XM_011519998.2 GI: 1034573063, XM_011519999.1 GI: 767965647, XM_011520002.1 GI: 767965653, XM_011520003.2 GI: 1034573065, XM_011520004.2 GI: 1034573066, XM_011520006.2 GI: 1034573082, XM_011520007.2 GI: 1034573083, XM_017017559.1 GI: 1034573076, XM_017017558.1 GI: 1034573071, XM_017017560.1 GI: 1034573078, XM_024448442.1 GI: 1370459140, XM_024448444.1 GI: 1370459144, XM_024448443.1 GI: 1370459142, XM_024448446.1 GI: 1370459148, and XM_024448445.1 GI: 1370459146. Exemplary human PGAP2 protein sequences include, but are not limited to, NCBI Reference Sequences: NP_055304.1 GI: 7657102, NP_001138910.1 GI: 224177563, NP_001243164.1 GI: 371940851, NP_001243165.1 GI: 371940853,NP_001243166.1 GI: 371940855, NP_001243167.1 GI: 371940857,NP_001243168.1 GI: 371940860, NP_001243169.1 GI: 371940862, NP_001269967.1 GI: 545688907,NP_001269968.1 GI: 545688910, NP_001269969.1 GI: 545688912, NP_001333326.1 GI: 1077185865, NP_001333327.1 GI: 1077206158,NP_001333328.1 GI: 1077171324, NP_001333329.1 GI: 1077172999, NP_001333330.1 GI: 1077171645, NP_001333331.1 GI: 1077204729, NP_001333332.1 GI: 1077181876, NP_001333333.1 GI: 1077158369, NP_001333334.1 GI: 1077160071, XP_006718244.1 GI: 578820421, XP_006718248.1 GI: 578820429, XP_006718249.1 GI: 578820431, XP_006718253.1 GI: 578820439, XP_006718254.1 GI: 578820441, XP_006718256.1 GI: 578820445, XP_011518292.2 GI: 1034573055, XP_011518293.2 GI: 1034573057, XP_011518294.1 GI: 767965634, XP_011518296.2 GI: 1034573061, XP_011518298.1 GI: 767965642, XP_011518299.1 GI: 767965644, XP_011518300.1 GI: 767965646,XP_011518301.1 GI: 767965648, XP_011518304.1 GI: 767965654,XP_011518305.1 GI: 767965657, XP_011518306.1 GI: 767965659, XP_011518308.1 GI: 767965667, XP_011518309.1 GI: 767965669, XP_016873048.1 GI: 1034573077, XP_016873047.1 GI: 1034573072, XP_016873049.1 GI: 1034573079, XP_024304210.1 GI: 1370459141, XP_024304212.1 GI: 1370459145, XP_024304211.1 GI: 1370459143, XP_024304214.1 GI: 1370459149, and XP_024304213.1 GI: 1370459147. Without wishing to be bound by theory, it is believed that in some embodiments, PGAP2 is involved in maturation of GPI anchors.

As used herein, the term “EIF4A1,” “eukaryotic translation initiation factor 4A1” refers to the gene EIF4A1 and the gene product encoded by the EIF4A1 gene. It is also known as “DDX2A”, “EIF-4A”, “EIF4A”, “eIF-4A-I,” or “eIF4A-I.” In the human genome, EIF4A1 is located on chromosome 17. Exemplary human EIF4A1 transcript sequences include, but are not limited to, NCBI Reference Sequences: NM_001416.4 GI: 1519242938 and NM_001204510.2 GI: 1674994772. Exemplary human EIF4A1 protein sequences include, but are not limited to, NCBI Reference Sequences: P_001407.1 GI: 4503529 and NP_001191439.1 GI: 325197164.

As used herein, the term “HDLBP,” “high density lipoprotein binding protein” refers to the gene HDLBP and the gene product encoded by the HDLBP gene. It is also known as “HBP,” “PR02900,” or “VGL.” In the human genome, HDLBP is located on chromosome 2. Exemplary human HDLBP transcript sequences include, but are not limited to, NCBI Reference Sequences: NM_005336.6 GI: 1914185842, NM_203346.6 GI: 1890323569, NM_001243900.3 GI: 1890332853, NM_001320965.3 GI: 1890284507, NM_001320966.3 GI: 1889510161, M_001320967.3 GI: 1889422387, XM_005247002.4 GI: 1370477463, XM_006712475.4 GI: 1370477459XM_006712475.4 GI: 1370477459, XM_011511058.3 GI: 1370477460, XM_011511060.3 GI: 1370477462, XM_017003940.2 GI: 1370477461, XM_017003941.2 GI: 1370477464, XM_017003942.1 GI: 1034613482, XM_024452832.1 GI: 1370477455, and XM_024452833.1 GI: 1370477457. Exemplary human HDLBP protein sequences include, but are not limited to, NCBI Reference Sequences: NP_005327.1 GI: 4885409, NP_976221.1 GI: 42716280, NP_001230829.1 GI: 345199283, NP_001307894.1 GI: 1004170707, NP_001307895.1 GI: 1004170709, NP_001307896.1 GI: 1004170711, XP_005247059.2 GI: 578804201, XP_006712538.1 GI: 578804197, XP_011509360.1 GI: 767917817, XP_011509362.1 GI: 767917822, XP_016859429.1 GI: 1034613476, XP_016859430.1 GI: 1034613481, XP_016859431.1 GI: 1034613483, XP_024308600.1 GI: 1370477456, and XP_024308601.1 GI: 1370477458.

As used herein, the term “GOLGA1,” “golgin A1” refers to the gene GOLGA1 and the gene product encoded by the GOLGA1 gene. It is also known as “golgin-97.” In the human genome, GOLGA1 is located on chromosome 9. Exemplary human GOLGA1 transcript sequences include, but are not limited to, NCBI Reference Sequences: NM_002077.4 GI: 1653961230, XM_005251929.4 GI: 1370514315, XM_006717062.4 GI: 1370514314, XM_006717063.4 GI: 1370514316, and XR_929766.1 GI: 767957102. Exemplary human GOLGA1 protein sequences include, but are not limited to, NCBI Reference Sequences: NP_002068.2 GI: 1653961231, XP_005251986.1 GI: 530390993, XP_006717125.1 GI: 578817349, and XP_006717126.1 GI: 578817351. Without wishing to be bound by theory, it is believed that in some embodiments, GOLGA1 is involved in vesicle-mediated endocytosis.

As used herein, the term “UBE2L6,” “ubiquitin conjugating enzyme E2 L6refers to the gene UBE2L6 and the gene product encoded by the UBE2L6 gene. It is also known as “RIG-B,” or “UBCH8.” In the human genome, UBE2L6 is located on chromosome 11. Exemplary human UBE2L6 transcript sequences include, but are not limited to, NCBI Reference Sequences: NM_004223.5 GI: 1519245691, and NM_198183.3 GI: 1890284508. Exemplary human UBE2L6 protein sequences include, but are not limited to, NCBI Reference Sequences: P_004214.1 GI: 4759282 and NP_937826.1 GI: 38157986.

As used herein, the term “SIK2,” “salt inducible kinase 2” refers to the gene SIK2 and the gene product encoded by the SIK2 gene. It is also known as “LOH11CR1I”, “QIK”, “SIK-2,” or “SNFILK2.” In the human genome, SIK2 is located on chromosome 11. Exemplary human SIK2 transcript sequences include, but are not limited to, NCBI Reference Sequences: NM_015191.3 GI: 1519316251, XM_017017417.1 GI: 1034572660, and XM_017017418.1 GI: 1034572662. Exemplary human SIK2 protein sequences include, but are not limited to, NCBI Reference Sequences: NP_056006.1 GI: 38569460, XP_016872906.1 GI: 1034572661, and XP_016872907.1 GI: 1034572663.

As used herein, the term “SOX4,” “SRY-box transcription factor 4” refers to the gene SOX4 and the gene product encoded by the SOX4 gene. It is also known as “CSS10,” or “EVI16.” In the human genome, SOX4 is located on chromosome 6. Exemplary human SOX4 transcript sequences include, but are not limited to, NCBI Reference Sequence: NM_003107.3 GI: 1653961080. Exemplary human SOX4 protein sequences include, but are not limited to, NCBI Reference Sequence: AAH72668.1 GI: 49257399.

As used herein, the term “OR6Q1,” “olfactory receptor family 6 subfamily Q member 1” refers to the gene OR6Q1 and the gene product encoded by the OR6Q1 gene. It is also known as “OR11-226.” In the human genome, OR6Q1 is located on chromosome 11. Exemplary human OR6Q1 transcript sequences include, but are not limited to, NCBI Reference Sequence: NM_001005186.2. Exemplary human OR6Q1 protein sequences include, but are not limited to, NCBI Reference Sequences: NP_001005186.2.

As used herein, the term “OR4D6,” “olfactory receptor family 4 subfamily D member 6” refers to the gene OR4D6 and the gene product encoded by the OR4D6 gene. It is also known as “OR11-250.” In the human genome, OR4D6 is located on chromosome 11. Exemplary human OR4D6 transcript sequences include, but are not limited to, NCBI Reference Sequence: NM_001004708.1 GI: 52317244. Exemplary human OR4D6 protein sequences include, but are not limited to, NCBI Reference Sequence: NP_001004708.1 GI: 52317245.

As used herein, the term “PIGT,” “phosphatidylinositol glycan anchor biosynthesis class T” refers to the gene PIGT and the gene product encoded by the PIGT gene. It is also known as “CGI-06”, “MCAHS3”, “NDAP,” or “PNH2.” In the human genome, PIGT is located on chromosome 20. Exemplary human PIGT transcript sequences include, but are not limited to, NCBI Reference Sequences: NM_015937.6 GI: 1519244523, NM_001184728.3 GI: 1674986191, NM_001184729.3 GI: 1675063910, NM_001184730.3 GI: 1675004817, NR_047691.2 GI: 1701108635, NR_047692.2 GI: 1701946125, NR_047693.2 GI: 1701970906, NR_047694.2 GI: 1701304417, and NR_047695.2 GI: 1701969862. Exemplary human PIGT protein sequences include, but are not limited to, NCBI Reference Sequences: NP_057021.2 GI: 23397653, NP_001171657.1 GI: 296080710, NP_001171658.1 GI: 296080712, and NP_001171659.1 GI: 296080714.

As used herein, the term “MICOS13,” “mitochondrial contact site and cristae organizing system subunit 13” refers to the gene MICOS13 and the gene product encoded by the MICOS13 gene. It is also known as “C19orf70”, “MIC12”, “MIC13”, “P117,” or “QIL1.” In the human genome, MICOS13 is located on chromosome 19. Exemplary human MICOS13 transcript sequences include, but are not limited to, NCBI Reference Sequences: NM_205767.3 GI: 1804891996, NM_001308240.2 GI: 1889557604, NM_001365761.2 GI: 1889423811, and XM_011527675.2 GI: 1034606077. Exemplary human MICOS13 protein sequences include, but are not limited to, NCBI Reference Sequences: NP_991330.1 GI: 45387955, NP_001295169.1 GI: 815891001, NP_001352690.1 GI: 1475409107, and XP_011525977.1 GI: 768000783.

As used herein, the term “RIN1,” “Ras and Rab interactor 1” refers to the gene RIN1 and the gene product encoded by the RIN1 gene. In the human genome, RIN1 is located on chromosome 1. Exemplary human RIN1 transcript sequences include, but are not limited to, NCBI Reference Sequences: NM_004292.3 GI: 1519314357, NM_001363559.2 GI: 1676318736, NM_001363560.2 GI: 1674986268, and XM_017018587.1 GI: 1034576387. Exemplary human RIN1 protein sequences include, but are not limited to, NCBI Reference Sequences: NP_004283.2 GI: 68989256, NP_001350488.1 GI: 1391723708, NP_001350489.1 GI: 1391723722, and XP_016874076.1 GI: 1034576388. Without wishing to be bound by theory, it is believed that in some embodiments, RIN1 is involved in vesicle-mediated endocytosis.

As used herein, the term “TRIM49C,” “tripartite motif containing 49C” refers to the gene TRIM49C and the gene product encoded by the TRIM49C gene. It is also known as “TRIM49L2.” In the human genome, TRIM49C is located on chromosome 11. Exemplary human TRIM49C transcript sequences include, but are not limited to, NCBI Reference Sequences: NM_001195234.1 GI: 305377071, XM_017018126.1 GI: 1034574928, and XM_024448656.1 GI: 1370459937. Exemplary human TRIM49C protein sequences include, but are not limited to, NCBI Reference Sequences: NP_001182163.1 GI: 305377072, XP_016873615.1 GI: 1034574929, and XP_024304424.1 GI: 1370459938.

As used herein, the term “HEATR5B,” “HEAT repeat containing 5B” refers to the gene HEATR5B and the gene product encoded by the HEATR5B gene. It is also known as “p200,” or “p200a.” In the human genome, HEATR5B is located on chromosome 2. Exemplary human HEATR5B transcript sequences include, but are not limited to, NCBI Reference Sequences: NM_019024.3 GI: 1519311867, XM_006712034.2 GI: 767914750, XM_006712035.4 GI: 1370478015, XM_011532934.3 GI: 1370478016, XM_011532935.3 GI: 1370478019, XM_017004378.1 GI: 1034614775, XM_017004379.2 GI: 1370478017, XM_017004380.2 GI: 1370478018, XR_001738786.1 GI: 1034614778, XR_001738787.1 GI: 1034614783, XR_001738788.1 GI: 1034614784, XR_001738789.1 GI: 1034614786, and XR_001738790.1 GI: 1034614787. Exemplary human HEATR5B protein sequences include, but are not limited to, NCBI Reference Sequences: NP_061897.1 GI: 55749742, XP_006712097.1 GI: 578802958, XP_006712098.1 GI: 578802960, XP_011531236.1 GI: 767914753, XP_011531237.1 GI: 767914755, XP_016859867.1 GI: 1034614776, XP_016859868.1 GI: 1034614780, and XP_016859869.1 GI: 1034614782. Without wishing to be bound by theory, it is believed that in some embodiments, HEATR5B is involved AP1G1/AP-1-mediated protein trafficking.

As used herein, the term “CPD,” “carboxypeptidase D” refers to the gene CPD and the gene product encoded by the CPD gene. It is also known as “GP180.” In the human genome, CPD is located on chromosome 17. Exemplary human CPD transcript sequences include, but are not limited to, NCBI Reference Sequences: NM_001304.5 GI: 1519312638 and NM_001199775.1 GI: 315138989. Exemplary human CPD protein sequences include, but are not limited to, NCBI Reference Sequences: NP_001295.2 GI: 22202611 and NP_001186704.1 GI: 315138990.

As used herein, the term “ARL1,” “ADP ribosylation factor like GTPase 1” refers to the gene ARL1 and the gene product encoded by the ARL1 gene. It is also known as “ARFL1.” In the human genome, ARL1 is located on chromosome 12. Exemplary human ARL1 transcript sequences include, but are not limited to, NCBI Reference Sequences: NM_001177.6 GI: 1519243442 and NM_001301068.1 GI: 666638014. Exemplary human ARL1 protein sequences include, but are not limited to, NCBI Reference Sequences: NP_001168.1 GI: 4502227 and NP_001287997.1 GI: 666638015. Without wishing to be bound by theory, it is believed that in some embodiments, ARL1 is involved in an activity or recruitment of a golgin, arfaptin, and/or Arf-GEF to a trans-Golgi network.

As used herein, the term “AP1B1,” “adaptor related protein complex 1 subunit beta 1” refers to the gene AP1B1 and the gene product encoded by the AP1B1 gene. It is also known as “ADTB1”, “AP105A”, “BAM22”, “CLAPB2,” or “KIDAR.” In the human genome, AP1B1 is located on chromosome 22. Exemplary human AP1B1 transcript sequences include, but are not limited to, NCBI Reference Sequences: NM_001127.4 GI: 1811715246, NM_145730.3 GI: 1890346350, NM_001166019.2 GI: 1677499085, NM_001378562.1 GI: 1811715267, NM_001378563.1 GI: 1811715216, NM_001378564.1 GI: 1811715174, NM_001378565.1 GI: 1811715286, and NM_001378566.1 GI: 1811715277. Exemplary human AP1B1 protein sequences include, but are not limited to, NCBI Reference Sequences: NP_001118.3 GI: 260436862, NP_663782.2 GI: 260436860, NP_001159491.1 GI: 260436864, NP_001365491.1 GI: 1811715268, NP_001365492.1 GI: 1811715217, NP_001365493.1 GI: 1811715175, NP_001365494.1 GI: 1811715287, and NP_001365495.1 GI: 1811715278.

As used herein, the term “AP1B1,” “adaptor related protein complex 1 subunit beta 1” refers to the gene AP1B1 and the gene product encoded by the AP1B1 gene. It is also known as “ADTB1”, “AP105A”, “BAM22”, “CLAPB2,” or “KIDAR.” In the human genome, AP1B1 is located on chromosome 22 Sequences: NM_001127.4 GI: 1811715246, NM_145730.3 GI: 1890346350, NM_001166019.2 GI: 1677499085, NM_001378562.1 GI: 1811715267, NM_001378563.1 GI: 1811715216, NM_001378564.1 GI: 1811715174, NM_001378565.1 GI: 1811715286, and NM_001378566.1 GI: 181171527. Exemplary human AP1B1 protein sequences include, but are not limited to, NCBI Reference Sequences: NP_001118.3 GI: 260436862, NP_663782.2 GI: 260436860, NP_001159491.1 GI: 260436864, NP_001365491.1 GI: 1811715268, NP_001365492.1 GI: 1811715217, NP_001365493.1 GI: 1811715175, NP_001365494.1 GI: 1811715287, and NP_001365495.1 GI: 1811715278. Without wishing to be bound by theory, it is believed that in some embodiments, AP1B1 is involved in lipoic acid biosynthesis.

As used herein, the term “KDSR,” “3-ketodihydrosphingosine reductase” refers to the gene KDSR and the gene product encoded by the KDSR gene. It is also known as “DHSR”, “EKVP4”, “FVT1,” or “SDR35C1.” In the human genome, KDSR is located on chromosome 18. Exemplary human KDSR transcript sequences include, but are not limited to, NCBI Reference Sequences: NM_002035.4 GI: 1388035702, XM_005266677.3 GI: 1370473346, and XM_017025690.2 GI: 1370473347. Exemplary human KDSR protein sequences include, but are not limited to, NCBI Reference Sequences: NP_002026.1 GI: 4503817, XP_005266734.1 GI: 530414153, and XP_016881179.1 GI: 1034603820.

As used herein, the term “KIAA2013,” refers to the gene KIAA2013 and the gene product encoded by the KIAA2013 gene. In the human genome, KIAA2013 is located on chromosome 1. Exemplary human KIAA2013transcript sequences include, but are not limited to, NCBI Reference Sequence: NM_138346.3 GI: 1519315727Exemplary human KIAA2013 protein sequences include, but are not limited to, NCBI Reference Sequences: AAH35033.1 GI: 23271560 and NP_612355.1 GI: 25286703.

As used herein, the term “AP2B1,” “adaptor related protein complex 2 subunit beta 1” refers to the gene AP2B1 and the gene product encoded by the AP2B1 gene. It is also known as “ADTB2”, “AP105B”, “AP2-BETA,” or “CLAPB1.” In the human genome, AP2B1 is located on chromosome 17. Exemplary human AP2B1 transcript sequences include, but are not limited to, NCBI Reference Sequences: NM_001030006.2 GI: 1519245384, NM_001282.3 GI: 1676317279, XM_005257937.4 GI: 1370470335, XM_005257938.3 GI: 1370470336, XM_005257941.3 GI: 1370470338, XM_011524448.2 GI: 1370470331, XM_011524449.3 GI: 1370470332, XM_011524450.2 GI: 1370470333, XM_011524451.2 GI: 1370470334, XM_011524452.1 GI: 767993865, XM_011524453.1 GI: 767993867, XM_011524454.1 GI: 767993869, XM_011524455.2 GI: 1034598536, XM_017024284.2 GI: 1370470337, XM_017024285.1 GI: 1034598534, XM_017024286.1 GI: 1034598537, and XM_017024287.2 GI: 1370470339. Exemplary human AP2B1 protein sequences include, but are not limited to, NCBI Reference Sequences: NP_001025177.1 GI: 71773106, NP_001273.1 GI: 4557469, XP_005257994.1 GI: 530410945, XP_005257995.1 GI: 530410947, XP_005257998.1 GI: 530410953, XP_011522750.1 GI: 767993857, XP_011522751.1 GI: 767993859, XP_011522752.1 GI: 767993861, XP_011522753.1 GI: 767993863, XP_011522754.1 GI: 767993866, XP_011522755.1 GI: 767993868, XP_011522756.1 GI: 767993870, XP_011522757.1 GI: 767993872, XP_016879773.1 GI: 1034598533, XP_016879774.1 GI: 1034598535, XP_016879775.1 GI: 1034598538, and XP_016879776.1 GI: 1034598540. Without wishing to be bound by theory, it is believed that in some embodiments, AP2B1 is involved clathrin-coated vesicle trafficking.

As used herein, the term “DENR,” “density regulated re-initiation and release factor” refers to the gene DENR and the gene product encoded by the DENR gene. It is also known as “DRP,” “DRP1” or “SMAP-3.” In the human genome, DENR is located on chromosome 12. Exemplary human DENR transcript sequences include, but are not limited to, NCBI Reference Sequence: NM_003677.5 GI: 1519312566. Exemplary human DENR protein sequences include, but are not limited to, NCBI Reference Sequence: AAH07860.1 GI: 14043821.

As used herein, the term “ARL1,” “ADP ribosylation factor like GTPase 1” refers to the gene ARL1 and the gene product encoded by the ARL1 gene. It is also known as “ARFL1.” In the human genome, ARL1 is located on chromosome 12. Exemplary human ARL1 transcript sequences include, but are not limited to, NCBI Reference Sequences: NM_001177.6 GI: 1519243442 and NM_001301068.1 GI: 666638014. Exemplary human ARL1 protein sequences include, but are not limited to, NCBI Reference Sequences: NP_001168.1 GI: 4502227 and NP_001287997.1 GI: 666638015. Without wishing to be bound by theory, it is believed that in some embodiments, ARL1 is involved in an activity or recruitment of a golgin, arfaptin, and/or Arf-GEF to a trans-Golgi network.

As used herein, the term “PTEN,” “phosphatase and tensin homolog” refers to the gene PTEN and the gene product encoded by the PTEN gene. It is also known as “10q23del”, “BZS”, “CWS1”, “DEC”, “GLM2”, “MHAM”, “MMAC1”, “PTEN1”, “PTENbeta,” or “TEP1.” In the human genome, PTEN is located on chromosome 10. Exemplary human PTEN transcript sequences include, but are not limited to, NCBI Reference Sequences: NM_000314.8 GI: 1732746392, NM_001304717.5 GI: 1520682131, and NM_001304718.2 GI: 1518767609. Exemplary human PTEN protein sequences include, but are not limited to, NCBI Reference Sequences: NP_000305.3 GI: 73765544, NP_001291646.4 GI: 1520682132, and NP_001291647.1 GI: 754502064.

As used herein, the term “RALGAPB,” “Ral GTPase activating protein non-catalytic subunit beta” refers to the gene RALGAPB and the gene product encoded by the RALGAPB gene. It is also known as “KIAA1219,” or “RalGAPbeta.” In the human genome, RALGAPB is located on chromosome 20. Exemplary human RALGAPB transcript sequences include, but are not limited to, NCBI Reference Sequences: NM_020336.4 GI: 1519316374, NM_001282917.2 GI: 1676344148, NM_001282918.2 GI: 1676319944,XM_005260462.3 GI: 1370480751,XM_005260463.3 GI: 1370480752,XM_005260464.3 GI: 1370480753, XM_005260465.3 GI: 1370480755, XM_017027966.2 GI: 1370480754, XM_017027967.2 GI: 1370480756, XM_017027968.2 GI: 1370480757, XM_017027969.2 GI: 1370480758, XM_017027970.2 GI: 1370480760, and XR_001754350.2 GI: 1370480759. Exemplary human RALGAP protein sequences include, but are not limited to, NCBI Reference Sequences: NP_065069.1 GI: 34787409, NP_001269846.1 GI: 544711130, NP_001269847.1 GI: 544711150, XP_005260519.1 GI: 530418210, XP_005260520.1 GI: 530418212, XP_005260521.1 GI: 530418214, XP_005260522.1 GI: 530418216, XP_016883455.1 GI: 1034625493, XP_016883456.1 GI: 1034625496, XP_016883457.1 GI: 1034625498, XP_016883458.1 GI: 1034625500, and XP_016883459.1 GI: 103462550.

As used herein, the term “B3GNT2,” “UDP-GlcNAc:betaGal beta-1,3-N-acetylglucosaminyltransferase 2” refers to the gene B3GNT2 and the gene product encoded by the B3GNT2 gene. It is also known as “3-Gn-Ti”, “3-Gn-T2”, “B3GN-T2”, “B3GNT”, “B3GNT-2”, “B3GNT1”, “BETA3GNT”, “BGNT2”, “BGnT-2”, “beta-1”, “beta3Gn-T1,” or “beta3Gn-T2.” In the human genome, B3GNT2 is located on chromosome 2. Exemplary human B3GNT2 transcript sequences include, but are not limited to, NCBI Reference Sequences: NM_006577.6 GI: 1519246075 and NM_001319075.2 GI: 1889699493. Exemplary human B3GNT2 protein sequences include, but are not limited to, NCBI Reference Sequences: NP_006568.2 GI: 9845238 and NP_001306004.1 GI: 977380204.

As used herein, the term “MCTS1,” “MCTS1 re-initiation and release factor” refers to the gene MCTS1 and the gene product encoded by the MCTS1 gene. It is also known as “MCT-1,” or “MCT.” In the human genome, MCTS1 is located on chromosome X. Exemplary human MCTS1 transcript sequences include, but are not limited to, NCBI Reference Sequences: NM_001137554.2 GI: 1675115521, NM_014060.3 GI: 1519313159, and R_938549.2 GI: 1034674113. Exemplary human MCTS1 protein sequences include, but are not limited to, NCBI Reference Sequences: NP_054779.1 GI: 7662502 and NP_001131026.1 GI: 212276121.

As used herein, the term “NBN,” “nibrin” refers to the gene NBN and the gene product encoded by the NBN gene. It is also known as “AT-V1”, “AT-V2”, “ATV”, “NBS”, “NBS1,” or “P95.” In the human genome, NBN is located on chromosome 8. Exemplary human NBN transcript sequences include, but are not limited to, NCBI Reference Sequences: NM_002485.5 GI: 1780222576, NM_001024688.3 GI: 1889560659, XM_011517045.1 GI: 767953016, XM_011517046.1 GI: 767953019, XM_017013460.1 GI: 1034660370, XM_017013462.2 GI: 1370512401, XM_024447163.1 GI: 1370512395, XM_024447164.1 GI: 1370512397, and XM_024447165.1 GI: 1370512399. Exemplary human NBN protein sequences include, but are not limited to, NCBI Reference Sequences: NP_002476.2 GI: 33356172, NP_001019859.1 GI: 67189945, XP_011515347.1 GI: 767953017, XP_011515348.1 GI: 767953020, XP_016868949.1 GI: 1034660371, XP_016868951.1 GI: 1034660375, XP_024302931.1 GI: 1370512396, XP_024302932.1 GI: 1370512398, and XP_024302933.1 GI: 1370512400.

As used herein, the term “SLC38A10,” “solute carrier family 38 member 10” refers to the gene SLC38A10 and the gene product encoded by the SLC38A10 gene. It is also known as “PP1744.” In the human genome, SLC38A10 is located on chromosome 17. Exemplary human SLC38A10 transcript sequences include, but are not limited to, NCBI Reference Sequences: NM_001037984.3 GI: 1519245429, NM_138570.4 GI: 1675124982, XM_005257019.1 GI: 530411504, XM_011524288.1 GI: 767993473, XM_011524289.1 GI: 767993475, and XM_011524290.1 GI: 767993477. Exemplary human SLC38A10 protein sequences include, but are not limited to, NCBI Reference Sequences: NP_001033073.1 GI: 83921602, NP_612637.1 GI: 20070376, XP_005257076.1 GI: 530411505, XP_011522590.1 GI: 767993474, XP_011522591.1 GI: 767993476, and XP_011522592.1 GI: 767993478.

As used herein, the term “FBXL20,” “F-box and leucine rich repeat protein 20” refers to the gene FBXL20 and the gene product encoded by the FBXL20 gene. It is also known as “Fbl2,” or “Fbl20.” In the human genome, FBXL20 is located on chromosome 17. Exemplary human FBXL20 transcript sequences include, but are not limited to, NCBI Reference Sequences: NM_032875.3 GI: 1519311907, NM_001184906.2 GI: 1628814728, NM_001370208.1 GI: 1628814738, and NM_001370209.1 GI: 1628814869. Exemplary human FBXL20 protein sequences include, but are not limited to, NCBI Reference Sequences: NP_116264.2 GI: 27734755, NP_001171835.1 GI: 296531375, NP001357137.1 GI: 1628814739, and NP_001357138.1 GI: 1628814870.

As used herein, the term “ACP1,” “acid phosphatase 1” refers to the gene ACP1 and the gene product encoded by the ACP1 gene. It is also known as “HAAP”, “LMW-PTP,” or “MWPTP.” In the human genome, ACP1 is located on chromosome 2. Exemplary human ACP1 transcript sequences include, but are not limited to, NCBI Reference Sequences: NM_004300.4 GI: 1519312739, NM_007099.4 GI: 1890251070, NM_001040649.3 GI: 1889567652, and NR_024080.2 GI: 1890391786. Exemplary human ACP1 protein sequences include, but are not limited to, NCBI Reference Sequences: NP_004291.1 GI: 4757714, NP_009030.1 GI: 6005988, and NP_001035739.1 GI: 96304457.

As used herein, the term “Midn,” “midnolin” refers to the gene Midn and the gene product encoded by the Midn gene. It is also known as “Stx.” In the human genome, Midn is located on chromosome 19. Exemplary human Midn transcript sequences include, but are not limited to, NCBI Reference Sequences: NM_001388306.1 GI: 1917984022, NM_177401.5 GI: 1890334238, NM_001388307.1 GI: 1917984028, NM_001388474.1 GI: 1929309600, XM_005259672.3 GI: 1034609937, XM_024451753.1 GI: 1370476142, and XM_024451754.1 GI: 1370476144. Exemplary human Midn protein sequences include, but are not limited to, NCBI Reference Sequences: NP_001375235.1 GI: 1917984023, NP_796375.3 GI: 58761508, NP_001375236.1 GI: 1917984029, NP_001375403.1 GI: 1929309601, XP_005259729.1 GI: 530425485, XP_024307521.1 GI: 1370476143, and XP_024307522.1 GI: 1370476145.

As used herein, the term “Rab7a,” “RAB7A, member RAS oncogene family” refers to the gene Rab7a and the gene product encoded by the Rab7a gene. It is also known as “CMT2B,” “PRO2706,” or “RAB7.” In the human genome, Rab7a is located on chromosome 3. Exemplary human Rab7a transcript sequences include, but are not limited to, NCBI Reference Sequence: NG_008070.1 GI: 192807324. Exemplary human Rab7a protein sequences include, but are not limited to, NCBI Reference Sequences: NP_004628.4 GI: 34147513.

As used herein, the term “Pds5a,” “PDS5 cohesin associated factor A” refers to the gene Pds5a and the gene product encoded by the Pds5a gene. It is also known as “PIG54,” “SCC-112,” or “SCC112.” In the human genome, Pds5a is located on chromosome 4. Exemplary human Pds5a transcript sequences include, but are not limited to, NCBI Reference Sequences: NM_001100399.2 GI: 1519314885, NM_001100400.2 GI: 1890274529, XM_011513672.2 GI: 1034639068, XM_017007928.1 GI: 1034639069, XR_001741184.2 GI: 1370486566, XR_001741185.1 GI: 1034639065, XR_001741186.1 GI: 1034639066, XR_001741187.1 GI: 1034639067, and XR_001741188.1 GI: 1034639071. Exemplary human Pds5a protein sequences include, but are not limited to, NCBI Reference Sequences: NP_001093869.1 GI: 155030216, NP_001093870.1 GI: 155030220, XP_011511974.1 GI: 767930270, and XP_016863417.1 GI: 1034639070.

### AAV Transduction Modulators

The present disclosure provides AAV transduction modulators and methods of modulating AAV transduction using such modulators. In some embodiments, the AAV modulator modulates (e.g., increases or decreases) AAV transduction efficiency. In some embodiments, the AAV modulator modulates a gene or gene product associated with AAV transduction efficiency. Examplary AAV transduction modulators include, but are not limited to, gene editing systems, nucleic acid-based modulators, small molecules, and protein or peptide-based modulators.

### Gene Editing Systems

According to the present disclosure, gene editing systems can be used as AAV transduction modulators. Also contemplated by the present disclosure are nucleic acids encoding one or more components of such gene editing systems. In some embodiments, the gene editing system targets a gene associated with AAV transduction efficiency as described herein. In some embodiments, the gene editing system alters the structure of the gene associated with AAV transduction efficiency. Exemplary gene editing systems include, but are not limited to, CRISPR/Cas gene editing systems, TALEN gene editing systems, and zinc finger nucleases.

### CRISPR/Cas Gene Editing Systems

Naturally occurring CRISPR/Cas systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

The CRISPR/Cas system has been modified for use in gene editing (silencing, enhancing or changing specific genes) in eukaryotes such as mice or primates. Wiedenheft et al. (2012) Nature 482: 331-8. This is accomplished by, for example, introducing into the eukaryotic cell a plasmid containing a specifically designed CRISPR and one or more appropriate Cas.

The CRISPR sequence, sometimes called a CRISPR locus, comprises alternating repeats and spacers. In a naturally occurring CRISPR, the spacers usually comprise sequences foreign to the bacterium such as a plasmid or phage sequence; in an exemplary CRISPR/Cas system targeting a gene associated with AAV transduction efficiency as described herein, the spacers are derived a gene associated with AAV transduction efficiency as described herein, or a sequence of its regulatory elements.

RNA from the CRISPR locus is constitutively expressed and processed into small RNAs. These comprise a spacer flanked by a repeat sequence. The RNAs guide other Cas proteins to silence exogenous genetic elements at the RNA or DNA level. Horvath et al. (2010) Science 327: 167-170; Makarova et al. (2006) Biology Direct 1: 7. The spacers thus serve as templates for RNA molecules, analogously to siRNAs. Pennisi (2013) Science 341: 833-836.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stern et al. (2010) Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836.

The CRISPR/Cas system can thus be used to modify, e.g., delete one or more nucleic acids from, a gene associated with AAV transduction efficiency as described herein, or a gene regulatory element of a gene associated with AAV transduction efficiency as described herein, or introduce a premature stop which thus decreases expression of a functional of a gene associated with AAV transduction efficiency as described herein. The CRISPR/Cas system can alternatively be used like RNA interference, turning off a gene associated with AAV transduction efficiency as described herein in a reversible fashion. In a mammalian cell, for example, the RNA can guide the Cas protein to a promoter of a gene associated with AAV transduction efficiency as described herein, sterically blocking RNA polymerases.

CRISPR/Cas systems for gene editing in eukaryotic cells typically involve (1) a guide RNA molecule (gRNA) comprising a targeting sequence (which is capable of hybridizing to the genomic DNA target sequence), and sequence which is capable of binding to a Cas, e.g., Cas9 enzyme, and (2) a Cas, e.g., Cas9, protein. The targeting sequence and the sequence which is capable of binding to a Cas, e.g., Cas9 enzyme, may be disposed on the same or different molecules. If disposed on different molecules, each includes a hybridization domain which allows the molecules to associate, e.g., through hybridization.

### TALEN Gene Editing Systems

TALENs are produced artificially by fusing a TAL effector DNA binding domain to a DNA cleavage domain. Transcription activator-like effects (TALEs) can be engineered to bind any desired DNA sequence, including a portion of the HLA or TCR gene. By combining an engineered TALE with a DNA cleavage domain, a restriction enzyme can be produced which is specific to any desired DNA sequence, including a HLA or TCR sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

To produce a TALEN, a TALE protein is fused to a nuclease (N), which is, for example, a wild-type or mutated FokI endonuclease. Several mutations to FokI have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96.

The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. (2011) Nature Biotech. 29: 143-8.

A TALEN specific for a gene associated with AAV transduction as described herein can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN, e.g., DNA encoding a CAR, e.g., as described herein; depending on the sequences of the foreign DNA and chromosomal sequence, this process can be used to integrate the DNA encoding the CAR, e.g., as described herein, at or near the site targeted by the TALEN. As shown herein, in the examples, but without being bound by theory, such integration may lead to the expression of the CAR as well as disruption of a gene associated with AAV transduction as described herein. Such foreign DNA molecule is referred to herein as “template DNA.” In embodiments, the template DNA further comprises homology arms 5′ to, 3′ to, or both 5′ and 3′ to the nucleic acid of the template DNA which encodes the molecule or molecules of interest (e.g., which encodes a CAR described herein), wherein said homology arms are complementary to genomic DNA sequence flanking the target sequence.

TALENs specific to sequences in a gene associated with AAV transduction as described herein can be constructed using any method known in the art, including various schemes using modular components. Zhang et al. (2011) Nature Biotech. 29: 149-53; Geibler et al. (2011) PLoS ONE 6: e19509; U.S. Pat. Nos. 8,420,782; 8,470,973, the contents of which are hereby incorporated by reference in their entirety.

### Zinc Finger Nucleases

“ZFN” or “Zinc Finger Nuclease” refer to a zinc finger nuclease, an artificial nuclease which can be used to modify, e.g., delete one or more nucleic acids of, a desired nucleic acid sequence, e.g., a gene associated with AAV transduction as described herein.

Like a TALEN, a ZFN comprises a FokI nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160.

A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair ofZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5.

Also like a TALEN, a ZFN can create a double-stranded break in the DNA, which can create a frame-shift mutation if improperly repaired, leading to a decrease in the expression of a gene associated with AAV transduction as described herein in a cell. ZFNs can also be used with homologous recombination to mutate a gene associated with AAV transduction as described herein, or to introduce nucleic acid encoding a CAR at a site at or near the targeted sequence. As discussed above, the nucleci acid encoding a CAR may be introduced as part of a template DNA. In some embodiments, the template DNA further comprises homology arms 5′ to, 3′ to, or both 5′ and 3′ to the nucleic acid of the template DNA which encodes the molecule or molecules of interest (e.g., which encodes a CAR described herein), wherein said homology arms are complementary to genomic DNA sequence flanking the target sequence.

ZFNs specific to sequences in a gene associated with AAV transduction as described herein, can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; and Guo et al. (2010) J. Mol. Biol. 400: 96; U.S. Patent Publication 2011/0158957; and U.S. Patent Publication 2012/0060230, the contents of which are hereby incorporated by reference in their entirety. In some embodiments, The ZFN gene editing system may also comprise nucleic acid encoding one or more components of the ZFN gene editing system, e.g., a ZFN gene editing system targeted to a gene associated with AAV transduction as described herein.

Without being bound by theory, it is believed that use of gene editing systems (e.g., CRISPR/Cas gene editing systems) which target a gene associated with AAV transduction as described herein, may allow one to modulate (e.g., increase or decrease) one or more functions of a gene associated with AAV transduction as described herein, by, for example, causing an editing event which results in expression of a truncated gene or gene product associated with AAV transduction as described herein. Again, without being bound by theory, such a truncated gene or gene product associated with AAV transduction as described herein may preserve one or more functions of the gene or gene product associated with AAV transduction as described herein, while inhibiting one or more other functions of the gene or gene product associated with AAV transduction as described herein, and as such, may be preferable. Gene editing systems which target a late exon or intron of a gene or gene product associated with AAV transduction as described herein, may be particularly preferred in this regard. In some embodiments, the gene editing system of the disclosure targets a late exon or intron of a gene or gene product associated with AAV transduction as described herein. In some embodiments, the gene editing system of the disclosure targets an exon or intron downstream of exon 8. In some embodiments, the gene editing system targets exon 8 or exon 9, e.g., exon 9, of a gene associated with AAV transduction.

Without being bound by theory, it may also be preferable in other embodiments to target an early exon or intron of a gene associated with AAV transduction as described herein, for example, to introduce a premature stop codon in the targeted gene which results in no expression of the gene product, or expression of a completely non-functional gene product. Gene editing systems which target an early exon or intron of a gene associated with AAV transduction as described herein, may be particularly preferred in this regard. In some embodiments, the gene editing system of the disclosure targets an early exon or intron of a gene associated with AAV transduction as described herein. In some embodiments, the gene editing system of the disclosure targets an exon or intron upstream of exon 4. In some embodiments, the gene editing system targets exon 1, exon 2, or exon 3, e.g., exon 3, of a gene associated with AAV transduction as described herein.

Without being bound by theory, it may also be preferable in other embodiments to target a sequence of a gene associated with AAV transduction as described herein, which is specific to one or more isoforms of the gene but does not affect one or more other isoforms of the gene. In some embodiments, it may be preferable to specifically target an isoform of a gene associated with AAV transduction as described herein which contain a catalytic domain.

### Nucleic Acid Based Modulators

According to the present disclosure, nucleic acids can be used as AAV transduction modulators. In some embodiments, the nucleic acid-based modulator modulates (e.g., increases or decreases) a gene or gene product associated with AAV transduction efficiency as described herein. In some embodiments, the nucleic acid-based modulator is specific for a nucleic acid encoding a gene or gene product associated with AAV transduction efficiency. Exemplary nucleic acid-based modulators include, but are not limited to, double stranded RNAs (dsRNAs) and antisense oligonucleotides (ASOs).

In some embodiments, the nucleic acid-based modualtor is a dsRNA, e.g., siRNA, microRNA or shRNA. Also contemplated by the present disclosure are nucleic acids encoding such dsRNAs.

In some embodiments, the dsRNA, e.g., a siRNA, microRNA or shRNA, comprises at least 15 continguous nucleotides, e.g., 15, 16, 17, 18, 19, 20, 21, 22, 23, 24 or 25 contiguous nucleotides, e.g., 21 contiguous nucleotides, which are complementary (e.g., 100% complementary) to a sequence of a gene or gene product associated with AAV transduction efficiency as described herein. It is understood that some of the target sequences and/or dsRNAs are presented as DNA, but the dsRNAs targeting these sequences or comprising these sequences can be RNA, or any nucleotide, modified nucleotide or substitute disclosed herein and/or known in the art, provided that the molecule can still mediate RNA interference.

In some embodiments, a nucleic acid molecule that encodes a dsRNA that inhibits expression of a gene or gene product associated with AAV transduction efficiency is operably linked to a promoter, e.g., a H1- or a U6-derived promoter such that the dsRNA that inhibits expression of a gene associated with AAV transduction efficiency is expressed within a cell. See e.g., Tiscomia G., “Development of Lentiviral Vectors Expressing siRNA,” Chapter 3, in Gene Transfer: Delivery and Expression of DNA and RNA (eds. Friedmann and Rossi). Cold Spring Harbor Laboratory Press, Cold Spring Harbor, NY, USA, 2007; Brummelkamp T R, et al. (2002) Science 296: 550-553; Miyagishi M, et al. (2002) Nat. Biotechnol. 19: 497-500. In some embodiments, a nucleic acid molecule (e.g., DNA) that encodes a dsRNA that inhibits expression of a gene or gene product associated with AAV transduction efficiency is disposed on a vector, e.g., any conventional expression system, e.g., a viral vector.

Without being bound by theory, a dsRNA (e.g., siRNA, microRNA or shRNA) which targets a gene product (e.g., mRNA) associated with AAV transduction efficiency as described herein, is specific to one or more isoforms of the gene product, but does not affect one or more other isoforms of the gene product (for example, due to targeting a unique splice junction, or targeting a domain which is present in one or more isoforms of the gene product, but is not present in one or more other isoforms of the gene product). In some embodiments, it may be preferable to specifically target an isoform of a gene or gene product associated with AAV transduction, e.g., which contains a catalytic domain.

In some embodiments, the nucleic acid-based modualtor is an antisense oligonucleotide (“ASO”). Also contemplated by the present disclosure are the uses of nucleic acids encoding such nucleic acid-based modulators.

### Small Molecules

According to the present disclosure, small molecules can be used as AAV transduction modulators. In some embodiments, the small molecule modulator modulates (e.g., increases or decreases) a gene or gene product associated with AAV transduction efficiency as described herein. In some embodiments, the small molecule modulator alters (e.g. increases or decreases) the stability of the gene product.

In some embodiments, the small molecule is a protein degrader.

Exemplary small molecule modulators include, but are not limited to, the compounds shown in FIG. 7.

### Protein or Peptide Based Modulators

According to the present disclosure, proteins and peptides can be used as AAV transduction modulators. Also contemplated by the present disclosure are nucleic acids encoding such protein or peptide based modulators. In some embodiments, the protein or peptide based modulator modulates (e.g., increases or decreases) a gene or gene product associated with AAV transduction efficiency as described herein. Examplary protein or peptide-based modulators include, but are not limited to, antibody molecules, binding domains, peptidyl inhibitors, and dominant negative mutant proteins.

In some embodiments, the modulator is an antibody molecule (e.g., an antibody or an antigen-binding fragment thereof). In some embodiments, the antibody molecule is a single chain Fv (scFv). In some embodiments, the antibody molecule is a single-domain antibody (sdAb), also known as a nanobody.

Single-domain antibodies can include antibodies whose complementary determining regions are part of a single-domain polypeptide. Examples include, but are not limited to, heavy chain antibodies, antibodies naturally devoid of light chains, single-domain antibodies derived from conventional 4-chain antibodies, engineered antibodies and single-domain scaffolds other than those derived from antibodies. Single-domain antibodies may be any of the art, or any future single-domain antibodies. Single-domain antibodies may be derived from any species including, but not limited to mouse, human, camel, llama, fish, shark, goat, rabbit, and bovine. According to another aspect of the disclosure, a single-domain antibody is a naturally occurring single-domain antibody known as heavy chain antibody devoid of light chains. Such single-domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the disclosure.

In some embodiments, the modulator is a peptidyl inhibitor.

In some embodiments, the modulator is a dominant negative isoform of a gene product associated with AAV transduction efficiency as described herein. In some embodiments, the modulator is a dominant negative binding partner of a gene product associated with AAV transduction efficiency as described herein.

### Vectors

In another aspect, the disclosure provides vectors comprising a nucleic acid encoding an AAV transduction modulator described herein. In some embodiments, the AAV transduction modulator modulates (e.g., increases or decreases) a gene or gene product associated with AAV transduction efficiency as described herein.

The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Methods of Use of Modulators

In an aspect, the disclosure provides methods of modulating the transduction efficiency of an AAV particle, comprising a step of modulating a gene or gene product associated with AAV transduction efficiency.

In some embodiments, the method comprises contacting an AAV transduction modulator described herein with a cell or subject. In some embodiments, the method alters (e.g., inhibits or activats) expression and/or function of a gene or gene product associated with AAV transduction efficiency. In certain embodiments, the method comprises reducing or eliminating expression and/or function of a gene or gene product associated AAV transduction efficiency in a cell or subject. In other embodiments, the method comprises increasing or activating expression and/or function of a gene or gene product associated with AAV transduction efficiency in a cell or subject. In some embodiments, the method increases the transduction efficiency of an AAV particle. In other embodiments, the method decreases the transduction efficiency of an AAV particle.

In another aspect, the disclosure further provides methods of preparing a subject, comprising the step of altering (e.g., reducing or eliminating, or increasing or activating) the expression and/or function of a gene or gene product associated with AAV transduction efficiency in the subject. In some embodiments, the method comprises contacting the subject with an AAV transduction modulator as described herein. In some embodiments, the contacting is done prior to, simultaneously with, or after said cell is contacted with an AAV particle, e.g., an AAV particle described herein.

In another aspect, the disclosure further provides methods of providing a cell, comprising the step of altering (e.g., reducing or eliminating, or increasing or activating) the expression and/or function of a gene or gene product associated with AAV transduction efficiency in said cell. In some embodiments, the method comprises contacting the cell with an AAV transduction modulator as described herein. In some embodiments, the contacting is done in vivo. In some embodiments, the contacting is done ex vivo. In some embodiments, the contacting is done in vitro. In some embodiments, the contacting is done prior to, simultaneously with, or after said cell is contacted with an AAV particle, e.g., an AAV particle described herein.

In some embodiments, the modulator alters (e.g., increases or decreases) transduction efficiency of an AAV particle, e.g., as determined by an assay described herein (e.g., a FACS-based assay, as described in Example 1 or 2).

In some embodiments, the transduction efficiency is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference level of transduction efficiency.

In some embodiments, the transduction efficiency is decreased by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of transduction efficiency.

In some embodiments, the reference level of transduction efficiency is the level of transduction efficiency in a cell that has not been contacted with the AAV transduction modulator.

In some embodiments, the reference level of transduction efficiency is the level of transduction efficiency before the cell is contacted with the AAV transduction modulator.

### Cells

In one aspect, the disclosure provides cells contacted by an AAV transduction modulator described herein. In some embodiments, the AAV transduction modulator modulates (e.g., increases or decreases) a gene or gene product associated with AAV transduction efficiency as described herein.

In other embodiments, the cell is present in a subject. In some embodiments, the cell is obtained from a subject. The term “subject” is intended to include any living organisms (e.g., mammals) suitable for AAV based gene therapies. Examples of subjects include humans, monkeys, chimpanzees, dogs, cats, mice, rats, and transgenic species thereof.

Examplary type of cells include, but are not limited to, a brain cell, a liver cell, a spinal cord cell, a dorsal root ganglion (DRG) cell, a spleen cell, a lymph node cell, a kidney cell, a lung cell, a heart cell, a muscle cell (e.g., a skeletal muscle cell, e.g., a femur muscle cell), a diaphragm cell, a bone marrow cell, or a gonad cell.

In some embodiments, the cell is a central nervous system (CNS) cell. In some embodiments, the CNS cell is an astrocyte, an oligodendrocyte, a microglial cell, or an ependymal cell. In some embodiments, the cell is a brain cell. In some embodiments, the brain cell is a neuron or glial cell. In some embodiments, the cell is a DRG cell. In some embodiments, the cell is a liver cell. In some embodiments, the liver cell is a hepatocyte, hepatic stellate cell, a Kupffer cell, or a liver sinusoidal endothelial cell. In some embodiments, the cell is a skeletal muscle cell. In some embodiments, the cell is a bone marrow cell.

In some embodiments, the cell is a diseased cell. In some embodiments, the cell is not a diseased cell, e.g., a normal or healthy cell. In some embodiments, the cell is from a subject having, or at risk of having, a disorder, e.g., a disorder described herein.

In some embodiments, the cell is a cancer cell, e.g., a brain cancer cell.

### AAV Gene Therapy

The compositions and methods described herein can be used in AAV-based gene therapies.

Adeno-associated viruses (AAV) are small, single-stranded DNA viruses that can be used as a delivery vehicle for payloads to cells, e.g., for treating diseases or disorders. The AAV genome is flanked by 145 nucleotide-long inverted terminal repeats (ITRs) and encodes a Rep protein and a Cap protein. Binding of AAV to heparan sulphate proteoglycans on the surface of a target cell leads to internalization of the virus via receptor-mediated endocytosis. The virus can, in some instances, be used to permanently insert its genome, or a portion thereof, into the host genome. In some instances, the viral genome may include one or more transgenes that can be inserted into the host genome. In some instances, one or more AAV genes or elements are deleted from the genome. In an embodiment, an AAV genome is modified to comprise only the original ITRs flanking one or more transgenes to be inserted into the host genome.

In some instances, an AAV vector is introduced into a cell along with (e.g., prior to, concurrently with, or subsequent to) a helper virus, e.g., an adenovirus (e.g., an adenovirus comprising a genome comprising at least the adenovirus E2A, E4, and VA1/II regions). In certain embodiments, an AAV vector is produced in a cell comprising a plasmid encoding an AAV rep gene and an AAV cap gene, as well as a plasmid comprising an expression cassette (e.g., an expression cassette comprising two ITRs flanking a transgene of interest). In embodiments, the cell comprises a nucleic acid sequence encoding the E1 region of an adenovirus genome (e.g., an Ad5 genome).

AAV vectors have been used to target diseases including various types of neurodegenerative diseases, cancer (e.g., glioblastoma multiforme), arthritis, muscular dystrophy, cystic fibrosis, Parkinson's Disease, hemophilia B, and many more. AAV vectors can transduce a variety of cell types, including differentiated cells and dividing cells (e.g., cancer cells). AAVs also generally elicit relatively little immune responses, for example, little or no cytotoxic response.

The serotypes of AAVs include, but are not limited to, AAV1, AAV2, AAV3, AAV4, AAV5, AAV6, AAV7, AAV8, AAV9, AAV10, and AAV11.

The tropisms of AAVs include, but are not limited to, the CNS (e.g., AAV1, AAV2, AAV4, AAV5, AAV8, or AAV9), heart (e.g., AAV1, AAV8, or AAV9), kidney (e.g., AAV2), liver (e.g., AAV7, AAV8, or AAV9), lung (e.g., AAV4, AAV5, AAV6, or AAV9), pancreas (e.g., AAV8), photoreceptor cells (e.g., AAV2, AAV5, or AAV8), retinal pigment epithelium (RPE) (e.g., AAV1, AAV2, AAV4, AAV5, or AAV8), and skeletal muscle (e.g., AAV1, AAV6, AAV7, AAV8, or AAV9).

The AAV vectors described herein can include a nucleotide sequence encoding a therapeutic protein, e.g., a protein associated with a disorder described herein, or a nucleotide sequence encoding a therapeutic nucleic acid, e.g., a nucleic acid targeting a nucleotide sequence encoding a protein associated with a disorder described herein.

### Therapeutic Applications

In one aspect, the methods and compositions described herein are indicated for treatment of nervous system diseases or neurodegenerative diseases, e.g., without limitation, Rett Syndrome, Alzheimer's disease, Parkinson's disease, Huntington's disease, or for treatment of nervous system injuries including spinal cord and brain trauma' injury, stroke, and brain cancers.

In some embodiments, the methods and compositions described herein can be used for the treatment of neurodegenerative and/or neurodevelopmental disorders and/or improve clinical trials as shown in Table 1 below.

In some embodiments, the method or composition is used in AAV-mediated delivery of nerve growth factor (NGF) in a subject with Alzheimer's disease (e.g., mild to moderate Alzheimer's disease). In some embodiments, the method or composition is used in AAV-mediated delivery of APOE2 (e.g., human APOE2) in a subject with Alzheimer's disease (e.g., mild to moderate Alzheimer's disease). In some embodiments, the method or composition is used in AAV-mediated delivery of TERT (e.g., human TERT) in a subject with Alzheimer's disease (e.g., mild to moderate Alzheimer's disease) or dementia (e.g., early dementia), e.g., at age greater than or equal to 40 years.

In some embodiments, the method or composition is used in AAV-mediated delivery of MAPT in a subject with Alzheimer's disease (e.g., mild Alzheimer's disease, e.g., at age 50-74). In some embodiments, the method or composition is used in AAV-mediated delivery of GAD in a subject with idiopathic PD, e.g., for at least five years. In some embodiments, the method or composition is used in AAV-mediated delivery of AADC in a subject with PD, e.g., mid-to-late stage PD, e.g., at age of less than or equal to 75 years. In some embodiments, the method or composition is used in AAV-mediated delivery of AADC in a subject with idiopathic PD, e.g., for at least five years. In some embodiments, the method or composition is used in AAV-mediated delivery of NTN in a subject with bilateral idiopathic PD, e.g., for at least five years, e.g., at age 35-75 years. In some embodiments, the method or composition is used in AAV-mediated delivery of GDNF in a subject with idiopathic PD, e.g., for at least five years. In some embodiments, the method or composition is used in AAV-mediated delivery of GDNF in a subject with PD, e.g., at age 35-75 years. In some embodiments, the method or composition is used in AAV-mediated delivery of GCase in a subject with moderate to severe PD, e.g., involving at least one pathogenic GBA1 mutation.

In some embodiments, the method or composition is used in AAV-mediated delivery of HTT in a subject with Huntington's Disease (HD), e.g., early manifest HD (e.g., at age 25-65 years) or manifest HD (e.g., at age 25-65 years).

In some embodiments, the method or composition is used in AAV-mediated delivery of SMN in a subject with Type 1 SMA (e.g., at less than 6 or 9 months of age). In some embodiments, the method or composition is used in AAV-mediated delivery of SMN2 in a subject with Type 1 SMA (e.g., at 2-14 years of age).

In some embodiments, the method or composition is used in AAV-mediated delivery of SOD1 in a subject with ALS (e.g., familial ALS, e.g., involving an SOD1 mutation), e.g., at greater than or equal to 18 years of age. In some embodiments, the method or composition is used in AAV-mediated delivery of C9orf72 in a subject with ALS (e.g., familial ALS, e.g., involving an C9orf72 mutation), e.g., at greater than or equal to 18 years of age.

In some embodiments, the method or composition is used in a therapeutic context. In other embodiments, the method or composition is used in a prophylaxis context.

In some embodiments, the subject has not received, or is not receiving, the therapy, when the AAV transduction modulator is administered. In certain embodiments, the AAV transduction modulator was administered at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy. In certain embodiments, the AAV transduction modulator was administered no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.

In some embodiments, the subject has received, or is receiving, the therapy, when the AAV transduction modulator is administered. In certain embodiments, the subject received the therapy at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered. In certain embodiments, the subject received the therapy no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.

TABLE 1
Gene Therapy for Neurodegenerative Disorders

| Gene therapy | Delivery route | identifier | Phase | Patients | Primary endpoint | Duration |
|---|---|---|---|---|---|---|
| AAV2-NGF | IP (basal forebrain) | NCT00087789 | 1 | Mild-to-moderate AD | Safety and | 2004-2010 |
| | | | | | tolerability | |
| AAV2-NGF | IP (basal forebrain) | NCT00876863 | 2 | Mild-to-moderate AD | Cognition | 2008-2015 |
| AAVrh.10- | IC | NCT03634007 | 1 | MCI or mild-to-severe AD, | Safety | |
| hAPOE2 | | | | APOE4 homozygotes, | | |
| | | | | aged ≥50 years | | |
| AAV-hTERT | IV + IT | NCT04133454 | 1 | AD or early dementia, | Safety and | |
| | | | | aged ≥40 years | tolerability | |
| ASO-MAPT | IT | NCT03186989 | 1/2 | Mild AD, aged 50-74 years | Safety and | |
| | | | | | tolerability | |
| AAV2-GAD | IP (subthalamic | NCT00195143 | 1 | Idiopathic PD, >5 years of | Safety | 2003-2005 |
| | nucleus) | | | disease duration | | |
| AAV2-GAD | IP (subthalamic | NCT00643890 | 2 | Idiopathic PD, >5 years of | Disease severity and | 2008-2010 |
| | nucleus) | | | disease duration | progression | |
| Lentivirus- | IP (striatum) | NCT00627588 | 1/2 | Bilateral idiopathic PD, age | Safety | 2008-2012 |
| TH/AADC/CH1 | | | | 48-65 years, >5 years of | | |
| | | | | disease duration | | |
| Lentivirus- | IP (striatum) | NCT01856439 | 1/2 | From NCT00627588 | Long-term safety | 2011-2022 |
| TH/AADC/CH1 | | | | | andtolerability | |
| AAV2-AADC | IP (striatum) | NCT00229736 | 1 | Mid- to late-stage PD, | Safety and | 2004-2013 |
| | | | | aged ≤75 years | tolerability | |
| AAV2-AADC | IP (striatum) | NCT01973543 | 1 | Idiopathic PD, >5 years of | Safety and | 2013-2020 |
| | | | | disease duration | tolerability | |
| AAV2-AADC | IP (putamen) | NCT02418598 | 1/2 | Idiopathic PD, aged ≤75 years | Safety | |
| AAV2-AADC | IP (striatum) | NCT03065192 | 1 | Idiopathic PD, >5 years of | Safety and suicide | |
| | | | | disease duration | risk | |
| AAV2-AADC | IP (striatum) | NCT03562494 | 2 | PD, aged 40-75 years, >4 | Change in ON time | 2018-2022 |
| | | | | years of disease duration | without | |
| | | | | | troublesome | |
| | | | | | dyskinesia | |
| AAV2-NTN | IP (putamen) | NCT00252850 | 1 | Bilateral idiopathic PD, aged | Safety and | 2005-2007 |
| | | | | 35-75 years, >5 years of | tolerability | |
| | | | | disease duration | | |
| AAV2-NTN | IP (putamen) | NCT00400634 | 2 | Bilateral idiopathic PD, aged | Disease severity and | 2006-2008 |
| | | | | 35-75 years, >5 years of | progression | |
| | | | | disease duration | | |
| AAV2-NTN | IP (substantia | NCT00985517 | 1/2 | Idiopathic PD, age 35-75 | Disease severity and | 2009-2017 |
| | nigra + putamen) | | | | progression | |
| AAV2-GDNF | IP (putamen) | NCT01621581 | 1 | Idiopathic PD, >5 years of | Safety and | 2013-2022 |
| | | | | disease duration | tolerability | |
| AAV2-GDNF | IP (putamen) | NCT04167540 | 1 | PD, aged 35-75 years | Safety and | |
| | | | | | tolerability | |
| AAV9-GCase | IC | NCT04127578 | 1/2 | Moderate-to-severe PD, at least | Safety and | |
| | | | | one pathogenic GBAl mutation | immunogenicity | |
| ASO-HTT | IT | NCT02519036 | 1/2 | Early manifest HD, aged 25- | Safety and | 2015-2017 |
| | | | | 65 years | tolerability | |
| ASO-HTT | IT | NCT03342053 | 2 | From NCT02519036 | Safety and | 2017-2019 |
| | | | | | tolerability | |
| ASO-HTT | IT | NCT04000594 | 1 | Manifest HD, aged 25-65 years | Pharmacokinetics | 2019-2021 |
| | | | | | and | |
| | | | | | pharmacodynamics | |
| ASO-HTT | IT | NCT03761849 | 3 | Manifest HD, aged 25-65 years | Disease progression | 2019-2022 |
| | | | | | andtotal functional | |
| | | | | | capacity | |
| ASO-HTT | IT | NCT03842969 | 3 | Patients who participated in | Long-term safety | 2019-2024 |
| | | | | priorrG6042 (ASO-HTT) | and tolerability, | |
| | | | | studies | suicide risk, | |
| | | | | | cognition | |
| AAV5-miHTT | IP (striatum) | NCT04120493 | 1/2 | Early manifest HD, ≥40 CAG | Safety | 2019-2026 |
| | | | | repeats in HTT | | |
| ASO-mHTT | IT | NCT03225833 | 1/2 | Early manifest HD, | Safety and | 2017-2020 |
| | | | | carrying a targeted SNP | tolerability | |
| | | | | rs362307, aged 25-65 years | | |
| ASO-mHTT | IT | NCT03225846 | 1/2 | Early manifest HD, | Safety and | 2017-2020 |
| | | | | carrying a targeted SNP | tolerability | |
| | | | | rs362331, aged 25-65 years | | |
| AAV9-SMN | IV | NCT02122952 | 1 | Type 1 SMA, <6 or 9 months | Safety | 2014-2019 |
| | | | | ofage | | |
| AAV9-SMN | IV | NCT03306277 | 3 | Type 1 SMA, ≤6 months of | Independent | 2017-2019 |
| | | | | age | sitting, survival | |
| AAV9-SMN | IT | NCT03381729 | 1 | SMA with 3 copies of | Safety and | 2017-2021 |
| | | | | SMN2 (onset at <12 months | tolerability | |
| | | | | of age), aged 6-60 months | | |
| AAV9-SMN | IV | NCT03461289 | 3 | Type 1 SMA, <6 months of | Sitting without | 2018-2020 |
| | | | | age | support | |
| AAV9-SMN | IV | NCT03505099 | 3 | Presymptomatic SMA with 2 | Independent sitting, | 2018-2021 |
| | | | | or 3 copies of SMN2, ≤6 weeks | standing without | |
| | | | | of age | support | |
| AAV9-SMN | IV | NCT03837184 | 3 | Type 1 SMA, <6 months of | Sitting without | 2019-2021 |
| | | | | age | support | |
| ASO-SMN2 | IT | NCT01494701 | 1 | Aged 2-14 years | Safety, | 2011-2013 |
| | | | | | tolerability and | |
| | | | | | pharmacokinetics | |
| ASO-SMN2 | IT | NCT01703988 | 1/2 | Aged 2-15 years | Safety and | 2012-2015 |
| | | | | | tolerability | |
| ASO-SMN2 | IT | NCT01780246 | 1 | From NCT01494701 | Safety and | 2013-2014 |
| | | | | | tolerability | |
| ASO-SMN2 | IT | NCT01839656 | 2 | Age ≤210 days, disease | Motor milestones | 2013-2017 |
| | | | | onset between 21 and 180 | | |
| | | | | days of age | | |
| ASO-SMN2 | IT | NCT02052791 | 1 | From NCT01703988, | Safety and | 2014-2017 |
| | | | | NCT01780246 | tolerability | |
| ASO-SMN2 | IT | NCT02292537 | 3 | Disease onset at >6 months | Motor function | 2014-2017 |
| | | | | ofage, aged 2-12 years | | |
| ASO-SMN2 | IT | NCT02386553 | 2 | SMA with 2 or 3 copies of | respiratory | 2015-2025 |
| | | | | SMN2, ≤6 weeks of age | intervention, | |
| | | | | | survival | |
| ASO-SMN2 | IT | NCT02594124 | 3 | From NCT02193074, | Long-term safety | 2015-2023 |
| | | | | NCT02292537, | and tolerability | |
| | | | | NCT02052791, | | |
| | | | | NCT01839656, | | |
| | | | | NCT02462759 | | |
| ASO-SMN2 | IT | NCT04050852 | 1 | Aged 5-21 years | Pulmonary function | 2019-2022 |
| ASO-SMN2 | IT | NCT04089566 | 2/3 | Child, adult, older adult | Safety and | 2020-2023 |
| | | | | | tolerability with | |
| | | | | | high doses, | |
| | | | | | neuromuscular | |
| | | | | | function | |
| ASO-SOD1 | IT | NCT01041222 | 1 | Familial ALS with SOD1 | Safety, | 2010-2012 |
| | | | | mutation, ≥18 years of age | tolerability and | |
| | | | | | pharmacokinetics | |
| ASO-SOD1 | IT | NCT02623699 | 3 | Familial ALS with SOD1 | Safety, tolerability, | 2016-2021 |
| | | | | mutation, ≥18 years of age | pharmacokinetics | |
| | | | | | and progression of | |
| | | | | | disability | |
| ASO-SOD1 | IT | NCT03070119 | 3 | From NCT02623699 | Long-term safety | 2017-2023 |
| | | | | | andtolerability | |
| ASO-SOD1 | IT | NCT03764488 | 1 | Aged 18-65 years | Drug distribution in | 2018-2021 |
| | | | | | the CNS | |
| ASO-C9orf72 | IT | NCT03626012 | 1 | ALS with C9orf72 | Safety and | 2018-2021 |
| | | | | mutation, ≥18 years of age | tolerability | |
| ASO-C9orf72 | IT | NCT04288856 | 1 | From NCT03626012 | Long-term safety | 2020-2023 |
| | | | | | and tolerability | |

### Combination Therapies

The compositions and methods described herein can be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A composition described herein and the at least one additional therapeutic agent can be administered simultaneously, in the same or in separate compositions, or sequentially. For sequential administration, the composition described herein can be administered first, and the additional agent can be administered second, or the order of administration can be reversed.

The compositions and/or other therapeutic agents, procedures or modalities can be administered during periods of active disorder, or during a period of remission or less active disease. The composition can be administered before the other treatment, concurrently with the treatment, post-treatment, or during remission of the disorder.

When administered in combination, the composition and the additional agent (e.g., second or third agent), or all, can be administered in an amount or dose that is higher, lower or the same than the amount or dosage of each agent used individually, e.g., as a monotherapy. In certain embodiments, the administered amount or dosage of the composition, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the composition, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In an aspect, provided is a method of treating a disorder, the method comprising: administering to a subject in need thereof an effective amount of a therapy comprising an AAV genome or an AAV particle, wherein a gene or gene product associated with AAV transduction efficiency is modulated in the subject, thereby treating the disorder.

In an aspect, provided is a method of treating a disorder, the method comprising: administering to a subject in need thereof an effective amount of (a) an AAV transduction modulator and (b) a therapy comprising an AAV genome or an AAV particle, thereby treating the disorder.

In some embodiments, the subject has not received, or is not receiving, the therapy, when the AAV transduction modulator is administered.

In some embodiments, the AAV transduction modulator was administered at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.

In some embodiments, the AAV transduction modulator was administered no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.

In some embodiments, the subject has received, or is receiving, the therapy, when the AAV transduction modulator is administered.

In some embodiments, the subject received the therapy at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.

In some embodiments, the subject received the therapy no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.

### Pharmaceutical Compositions and Treatments

In an aspect, the disclosure provides pharmaceutical compositions comprising an AAV modulator described herein in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, the compositions of the present disclosure are in one aspect formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an effective amount” or“therapeutic amount” is indicated, the precise amount of the compositions of the present disclosure to be administered can be determined by a physician with consideration of individual differences in age, weight, tumor size, extent of infection or metastasis, and condition of the patient (subject).

### ENUMERATED EMBODIMENT

1. A method of modulating transduction efficiency of an AAV particle, comprising:
   contacting a cell with an AAV transduction modulator,
   thereby modulating the transduction efficiency of the AAV particle.
2. The method of embodiment 2, wherein the modulator modulates (e.g., increases or decreases) a gene or gene product associated with AAV transduction efficiency.
3. The method of embodiment 2 or 3, wherein the gene or gene product is a mammalian (e.g., human) gene or gene product.
4. The method of any of the preceding embodiments, wherein the modulator inhibits the gene or gene product, e.g., a gene or gene product that is associated with a decreased transduction efficiency of an AAV particle.
5. The method of any of the preceding embodiments, wherein the modulator activates the gene or gene product, e.g., a gene or gene product that is associated with an increased transduction efficiency of an AAV particle.
6. The method of any of the preceding embodiments, wherein the gene or gene product is associated with an increased transduction efficiency of an AAV particle (e.g., AAV-R or GPR108).
7. The method of any of the preceding embodiments, wherein the gene or gene product is associated with an increased transduction efficiency of an AAV particle of a first serotype (e.g., AAV2) and an increased transduction efficiency of an AAV particle of a second serotype (e.g., AAV9) (e.g., AAV-R or GPR108).
8. The method of any of the preceding embodiments, wherein the gene or gene product is associated with a decreased transduction efficiency of an AAV particle (e.g., WDR11 or MRE11).
9. The method of any of the preceding embodiments, wherein the gene or gene product is associated with a decreased transduction efficiency of an AAV particle of a first serotype (e.g., AAV2) and a decreased transduction efficiency of an AAV particle of a second serotype (e.g., AAV9) (e.g., WDR11 or MRE11).
10. The method of any of the preceding embodiments, wherein the gene or gene product, when modulated, increases the transduction efficiency of an AAV particle.
11. The method of any of the preceding embodiments, wherein the gene or gene product, when modulated, increases the transduction efficiency of an AAV particle of a first serotype (e.g., AAV2) and the transduction efficiency of an AAV particle of a second serotype (e.g., AAV9).
12. The method of any of the preceding embodiments, wherein the gene or gene product, when modulated, decreases the transduction efficiency of an AAV particle.
13. The method of any of the preceding embodiments, wherein the gene or gene product, when modulated, decreases the transduction efficiency of an AAV particle of a first serotype (e.g., AAV2) and the transduction efficiency of an AAV particle of a second serotype (e.g., AAV9).
14. The method of any of the preceding embodiments, wherein the gene product is an RNA.
15. The method of any of the preceding embodiments, wherein the gene product is a protein.
16. The method of any of the preceding embodiments, wherein the gene or gene product is preferentially expressed in a target tissue (e.g., brain or liver).
17. The method of any of the preceding embodiments, wherein the modulator alters (e.g., increases or decreases) the expression of the gene.
18. The method of any of the preceding embodiments, wherein the modulator alters the structure of the gene.
19. The method of any of the preceding embodiments, wherein the modulator alters (e.g., increases or decreases) an activity (e.g., an enzymatic activity) of the gene product.
20. The method of any of the preceding embodiments, wherein the modulator alters (e.g., increases or decreases) the level (e.g., abundance) of the gene product.
21. The method of any of the preceding embodiments, wherein the modulator alters (e.g., increases or decreases) the stability of the gene product.
22. The method of any of the preceding embodiments, wherein the modulator alters (e.g., increases or decreases) transduction efficiency of an AAV particle, e.g., as determined by an assay described herein.
23. The method of any of the preceding embodiments, wherein the modulator increases transduction efficiency of an AAV particle, optionally wherein the modulator increases transduction to at least 10, 20, 30, 40, 50, or 60 viral copies per genome, optionally wherein the modulator increases transduction to at least 10³, 10⁴, 10⁵, 10⁶, or 10⁷ viral copies per μg DNA.
24. The method of any of the preceding embodiments, wherein the transduction efficiency is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference level of transduction efficiency.
25. The method of any of the preceding embodiments, wherein the modulator decreases transduction efficiency of an AAV particle,
   optionally wherein the modulator decreases transduction to no more than 0, 1, 2, 3, 4, 5, 6, 7, 8, 9, or 10 viral copies per genome,
   optionally wherein the modulator decreases transduction to no more than 10², 10³, or 10⁴ viral copies per μg DNA.
26. The method of any of the preceding embodiments, wherein the transduction efficiency is decreased by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of transduction efficiency.
27. The method of any of the preceding embodiments, wherein the reference level of transduction efficiency is the level of transduction efficiency in a cell that has not been contacted with the AAV transduction modulator.
28. The method of any of the preceding embodiments, wherein the reference level of transduction efficiency is the level of transduction efficiency before the cell is contacted with the AAV transduction modulator.
29. The method of any of the preceding embodiments, wherein the gene or gene product increases the transduction efficiency of an AAV2 particle,
   optionally wherein the gene or gene product does not increase, or does not substantially increase, the transduction efficiency of an AAV9 particle,
   optionally wherein the gene or gene product is AAV-R or GRP108.
30. The method of embodiment 29, wherein the gene or gene product is necessary for the transduction of an AAV2 particle, optionally wherein the gene or gene product is not necessary for the transduction of an AAV9 particle.
31. The method of any of embodiments 29-30, wherein the gene or gene product is selected from the group consisting of: AAV-R, GRP108, EXT1, EXT2, NDST1, BCL10, OAF, PDCD10, LMO4, MALT1, ZNF644, CHUK, GLCE, EP300, ARID4B, RAB21, MED13, DHX15, WASHC5, IKBKB, USP24, HSPA14, CXXC1, UBE2A, INTS8, CDC42, NFKB1, SIN3A, USP7, ARF5, CARD10, JAK1, SLC35C2, PAXK1, ZC3H11A, FAM20B, FAM72A, DHX36, INTS12, and RPRD1B, or a combination thereof.
32. The method of any of embodiments 29-31, wherein the gene or gene product is associated with endosomal sorting (e.g., RAB21, WASHC5, or USP7).
33. The method of any of embodiments 29-31, wherein the gene or gene product is associated with vesicle-mediated transport (e.g., CDC42 or ARF5).
34. The method of any of embodiments 29-31, wherein the gene or gene product is associated with NFKB signaling (e.g., BCL10, MALT1, NFKB1, IKBKB, CHUK, or CARD10).
35. The method of any of embodiments 29-31, wherein the gene or gene product is associated with heparan sulfate biosynthesis (e.g., EXT1, EXT2, NDST1, GLCE, or FAM20B).
36. The method of any of embodiments 29-31, wherein the gene or gene product is associated with H3K9 methylation (e.g., ZNF644).
37. The method of any of embodiments 29-31, wherein the gene or gene product is associated with pre-mRNA processing (e.g., DHX15).
38. The method of any of embodiments 29-31, wherein the gene or gene product is associated with transcription (e.g., LMO4, EP300, MED13, SIN3, ARID4B, ZC3H11A, CXXC1, RPRD1B, or UBE2A).
39. The method of any of embodiments 29-31, wherein the gene or gene product is associated with integrator complex and/or RNA polymerase II function (e.g., INTS8 or INST12).
40. The method of any of embodiments 29-31, wherein the gene or gene product is associated with DNA repair and/or AAV genome resolution (e.g., DHX36, ARID4B, UBE2A, or USP24).
41. The method of any of embodiments 29-31, wherein the gene or gene product is associated with cell proliferation and/or apoptosis (e.g., PDCD10).
42. The method of any of embodiments 29-31, wherein the gene or gene product is associated with spondylocarpotarsal synostosis syndrome (e.g., OAF).
43. The method of any of embodiments 29-31, wherein the gene or gene product is associated with protein folding (e.g., HSPA14).
44. The method of any of embodiments 29-31, wherein the gene or gene product is associated with helicase activity (e.g., DHX36).
45. The method of any of embodiments 29-31, wherein the gene or gene product is associated with fucosylation of Notch and/or transport of GCP-fucose (e.g., SLC35C2).
46. The method of any of embodiments 1-28, wherein the gene or gene product increases the transduction efficiency of an AAV9 particle,
   optionally wherein the gene or gene product does not increase, or does not substantially increase, the transduction efficiency of an AAV2 particle,
   optionally wherein the gene or gene product is AAV-R or GRP108.
47. The method of embodiment 46, wherein the gene or gene product is necessary for the transduction of an AAV9 particle, optionally wherein the gene or gene product is not necessary for the transduction of an AAV2 particle.
48. The method of any of embodiments 46-47, wherein the gene or gene product is selected from the group consisting of: AAV-R, GRP108, TMPRSS11B, HTT, SNRNP70, BRD7, DMXL1, RAB10, CNGA1, KLHDC3, CHP1, CYP3A5, ELOVL4, PNISR, ZCCHC14, AZGP1, TPST1, SC5D, ELP2, ELP3, IP09, RAB14, WDR7, XRCC4, and GDI2, or a combination thereof.
49. The method of any of embodiments 46-48, wherein the gene or gene product is associated with microtubule-mediated transport or vesicle function (e.g., HTT).
50. The method of any of embodiments 46-48, wherein the gene or gene product is an ion channel (e.g., CNGA1).
51. The method of any of embodiments 46-48, wherein the gene or gene product is associated with nuclear protein import (e.g. IP09).
52. The method of any of embodiments 46-48, wherein the gene or gene product is associated with O-sulfation of a tyrosine residue within an acidic motif of a polypeptide (e.g., TPST1).
53. The method of any of embodiments 46-48, wherein the gene or gene product is associated with protection of viral RNA (e.g., ZCCHC14).
54. The method of any of embodiments 46-48, wherein the gene or gene product is associated with RNA binding by an AAV protein (e.g., PNISR).
55. The method of any of embodiments 46-48, wherein the gene or gene product is associated with calcium ion-regulated exocytosis (e.g., CHP1).
56. The method of any of embodiments 46-48, wherein the gene or gene product is associated with intracellular trafficking (e.g., RAB10, RAB14, GDI2, WDR7, or DMXL1).
57. The method of any of embodiments 46-48, wherein the gene or gene product is a subunit (e.g., a catalytic tRNA acetyltransferase subunit) of an elongator complex, e.g., of an RNA polymerase (e.g., RNA polymerase II), e.g., ELP2 or ELP3.
58. The method of any of embodiments 46-48, wherein the gene or gene product binds to chromatin (e.g., KLHDC3).
59. The method of any of embodiments 46-48, wherein the gene or gene product is associated with cholesterol biosynthesis (e.g., SC5D).
60. The method of any of embodiments 1-28, wherein the gene or gene product increases the transduction efficiency of AAV2 and AAV9 particles, optionally wherein the gene or gene product is AAV-R or GRP108.
61. The method of embodiment 60, wherein the gene or gene product is necessary for the transduction of AAV2 and AAV9 particles.
62. The method of any of embodiments 60-61, wherein the gene or gene product is selected from the group consisting of: AAV-R, GRP108, KIAA0319L, BAMBI, TM9SF2, PHIP, DCLRE1C, VPS35, GPR108, CYREN, ACP2, F8A2, F8A1, F8A3, RBPJ, ATP2C1, ICMT, RABIF, IER3IP1, RBM10, SMG7, GTF2I, ELAVL1, MEPCE, RAB4A, IER3IP1, and SAMD1, or a combination thereof.
63. The method of any of embodiments 60-62, wherein the gene or gene product is associated with influenza infection (e.g., ACP2).
64. The method of any of embodiments 60-62, wherein the gene or gene product binds to unmethylated CGIs (e.g., SAMD1).
65. The method of any of embodiments 60-62, wherein the gene or gene product is associated with intracellular vesicular transport (e.g., RABIF).
66. The method of any of embodiments 60-62, wherein the gene or gene product is associated with posttranslational modification of cysteine residues (e.g., C-terminal cysteine residues), e.g., in a target protein (e.g., ICMT).
67. The method of any of embodiments 60-62, wherein the gene or gene product binds to a capsid protein of AAV (e.g., KIAA0319L).
68. The method of any of embodiments 60-62, wherein the gene or gene product is associated with inhibition of a TLR (e.g., TLR9), e.g., GPR108.
69. The method of any of embodiments 60-62, wherein the gene or gene product is associated with endosome trafficking, e.g., of AAV-R (e.g., VPS35).
70. The method of any of embodiments 60-62, wherein the gene or gene product is a calcium ATPase pump (e.g., ATP2C1).
71. The method of any of embodiments 60-62, wherein the gene or gene product is associated with Notch signaling (e.g., RBPJ).
72. The method of any of embodiments 60-62, wherein the gene or gene product is associated with HSPG metabolism (e.g., TM9SF2).
73. The method of any of embodiments 60-62, wherein the gene or gene product is associated with inhibition of NHEJ (e.g., CYREN).
74. The method of any of embodiments 60-62, wherein the gene or gene product is associated with viral hairpin resolution (e.g., DCLRE1C).
75. The method of any of embodiments 60-62, wherein the gene or gene product is associated with Glc transporter translocation, optionally wherein the gene or gene product binds to insulin receptor substrate 1 protein (e.g., PHIP).
76. The method of any of embodiments 1-28, wherein the gene or gene product decreases the transduction efficiency of an AAV2 particle,
   optionally wherein the gene or gene product, when modulated, does not decrease, or does not substantially decrease, the transduction efficiency of an AAV9 particle,
   optionally wherein the gene or gene product is WRD11 or MRE11.
77. The method of embodiment 76, wherein the gene or gene product inhibits or prevents the transduction of an AAV2 particle, optionally wherein the gene or gene product does not inhibit or prevent the transduction of an AAV9 particle.
78. The method of any of embodiments 76-77, wherein the gene or gene product is selected from the group consisting of: WRD11, MRE11, GREB1L, BIRC2, TYMSOS, MSL3, SIK3, GTF2H5, PIGW, ATRAID, PPP4R1, ZFC3H1, SETDB1, SMCHD1, TMEM123, UHRF1, CLUL1, SMAD5, SETX, STEEP1, DENND6A, RTN4RL2, MTMR9, STRADA, PSIP1, BMPR1A, HIKESHI, AP3M1, MEN1, MYL12B, CREB1, RAB12, SMTNL1, MRGBP, WTAP, SUMO2, TCN1, ALDH3B1, ARL14EP, MYL12A, RHOD, PGAP2, EIF4A1, PIGN, DAXX, HDLBP, GOLGA1, SARNP, UBE2L6, TRIM49, SIK2, SOX4, OR6Q1, OR4D6, UNC93B1, CATSPERZ, PIGT, PYM1, MICOS13, RIN1, DOT1L, TRIM49C, MAPK20, COL8A1, FASN, KDM3B, PIGF, PIGA, PIGY, GPC3, TRAPPC2L, RAB31, and ELP3, or a combination thereof.
79. The method of any of embodiments 76-78, wherein the gene or gene product is associated with a retinoic acid receptor activity (e.g., GREB1L).
80. The method of any of embodiments 76-78, wherein the gene or gene product is a thymidylate synthetase (e.g., TYMSOS).
81. The method of any of embodiments 76-78, wherein the gene or gene product is associated with chromatin remodeling (e.g., SMCHD1).
82. The method of any of embodiments 76-78, wherein the gene or gene product is associated with histone H4 acetylation (e.g., MSL3).
83. The method of any of embodiments 76-78, wherein the gene or gene product is associated with transcription and/or DNA repair (e.g., GTF2H5).
84. The method of any of embodiments 76-78, wherein the gene or gene product is a histone methyltransferase (e.g., SETDB1).
85. The method of any of embodiments 76-78, wherein the gene or gene product is associated with exosomal degradation of polyadenylated RNA (e.g., ZFC3H1).
86. The method of any of embodiments 76-78, wherein the gene or gene product is associated with histone deacetylase recruitment to a DNA (e.g., UHRF1).
87. The method of any of embodiments 76-78, wherein the gene or gene product is associated with oxidative stress-induced DNA double strand break response (e.g., SETX).
88. The method of any of embodiments 76-78, wherein the gene or gene product is associated with histone modification (e.g., MEN1).
89. The method of any of embodiments 76-78, wherein the gene or gene product is associated with nucleosome/DNA interaction (e.g., MRGBP).
90. The method of any of embodiments 76-78, wherein the gene or gene product is associated with GPI biosynthesis (e.g., PIGW, PIGN, PIGT, PIGF, PIGA, or PIGY).
91. The method of any of embodiments 76-78, wherein the gene or gene product is associated with maturation of GPI anchors (e.g., PGAP2).
92. The method of any of embodiments 76-78, wherein the gene or gene product is associated with vesicle-mediated endocytosis (e.g., DENND6A, GOLGA1, MTMR9, GPC3, PPP4R1, RAB12, RAB31, RHOD, RIN1, TMEM123, or TRAPPC2L).
93. The method of any of embodiments 76-78, wherein the gene or gene product is associated with clathrin-coated vesicle-mediated endocytosis (e.g., AP3M1).
94. The method of any of embodiments 76-78, wherein the gene or gene product is associated with a BMP receptor kinase (e.g., SMAD5, BMPR1A, or CREB1).
95. The method of any of embodiments 76-78, wherein the gene or gene product is associated with transcription (e.g., PSIP1).
96. The method of any of embodiments 76-78, wherein the gene or gene product is associated with nuclear import of an HSP70 protein (e.g., HIKESHI).
97. The method of any of embodiments 76-78, wherein the gene or gene product is associated with a Ser/Thr kinase (e.g., SIK2 or SIK3).
98. The method of any of embodiments 76-78, wherein the gene or gene product is associated with SUMOylation of a protein (e.g., SUMO2, UBE2L6).
99. The method of any of embodiments 1-28, wherein the gene or gene product decreases the transduction efficiency of an AAV9 particle,
   optionally wherein the gene or gene product does not decrease, or does not substantially decrease, the transduction efficiency of an AAV2 particle,
   optionally wherein the gene or gene product is WRD11 or MRE11.
100. The method of embodiment 99, wherein the gene or gene product inhibits or prevents the transduction of an AAV9 particle, optionally wherein the gene or gene product does not inhibit or prevent the transduction of an AAV2 particle.
101. The method of any of embodiments 99-100, wherein gene or gene product is selected from the group consisting of: WRD11, MRE11, AP2A1, AP2B1, TM9SF4, ACTR5, PTMA, PAPOLA, PDHA1, PDHB, SLC25A19, GAK, AUNIP, FCHO2, ACTB, LIPT1, UCHL5, INO80E, ECHS1, NELFB, EDC4, PRMT1, PDS5B, PELI3, FBXL20, and SLC35A1, or a combination thereof.
102. The method of any of embodiments 99-101, wherein the gene or gene product binds to ATP, a chaperone protein, and/or clathrin (e.g., GAK).
103. The method of any of embodiments 99-101, wherein the gene or gene product is associated with clathrin-coated vesicle trafficking (e.g., AP2B1 or AP2A1).
104. The method of any of embodiments 99-101, wherein the gene or gene product is associated with clathrin-mediated endocytosis (e.g., FCHO2).
105. The method of any of embodiments 99-101, wherein the gene or gene product is associated with vesicular trafficking (e.g., ACTB).
106. The method of any of embodiments 99-101, wherein the gene or gene product is associated with an innate immune response (e.g., PELI3 or FBXL20).
107. The method of any of embodiments 99-101, wherein the gene or gene product is associated with glucose metabolism (e.g., PDHB or PDHA1).
108. The method of any of embodiments 99-101, wherein the gene or gene product is associated with DNA resection and/or homologous recombination, e.g., after DNA damage (e.g., AUNIP).
109. The method of any of embodiments 99-101, wherein the gene or gene product is associated with DNA repair, e.g., after DNA damage (e.g., UCHL5, INO80E, PDS5B, ACTR5, or PRMT1).
110. The method of any of embodiments 99-101, wherein the gene or gene product is associated with transport of CMP-sialic acid from cytosol to a Golgi vesicle (e.g., SLC35A1).
111. The method of any of embodiments 99-101, wherein the gene or gene product is associated with localization of polypeptides comprising glycine-rich transmembrane domains, e.g., to the cell surface (e.g., TM9SF4).
112. The method of any of embodiments 99-101, wherein the gene or gene product is associated with poly(A) tail synthesis (e.g., PAPOLA).
113. The method of any of embodiments 99-101, wherein the gene or gene product is PTMA.
114. The method of any of embodiments 99-101, wherein the gene or gene product is associated with elongation of an mRNA by RNA polymerase II (e.g., NELFB).
115. The method of any of embodiments 99-101, wherein the gene or gene product is associated with mRNA degradation (e.g., EDC4).
116. The method of any of embodiments 99-101, wherein the gene or gene product encodes a mitochondrial lipoyltransferase (e.g., LIPT1).
117. The method of any of embodiments 99-101, wherein the gene or gene product is associated with mitochondrial fatty acid beta-oxidation (e.g., ECHS1).
118. The method of any of embodiments 99-101, wherein the gene or gene product encodes a mitochondrial thiamine pyrophosphate carrier (e.g., SLC25A19).
119. The method of any of embodiments 1-28, wherein the gene or gene product decreases the transduction efficiency of AAV2 and AAV9 particles,
   optionally wherein the gene or gene product is WRD11 or MRE11.
120. The method of embodiment 119, wherein the gene or gene product inhibits or prevents the transduction of AAV2 and AAV9 particles.
121. The method of any of embodiments 119-120, wherein the gene or gene product is selected from the group consisting of: WRD11, MRE11, PITPNB, PITP, FAM91A1, WDR11, AP1G1, AP1M1, AP1S1, AP1S3, HEATR5B, STX16, AP1B1, PIAS1, DENR, ARL1, ZFAT, TBC1D23, LIN37, RALGAPB, B3GNT2, ELOVL1, AP2B1, KIAA2013, PTEN, MCTS1, NBN, HELZ, SLC38A10, FBXL20, TGIF1, KDSR, CPD, CHD7, USP9X, SIMC1, TAF11, VAPA, MTMR6, RAB1B, SLF2, MRE11, VT11A, MBOAT7, and PPP6R3, or a combination thereof.
122. The method of any of embodiments 119-121, wherein the gene or gene product is associated with the long-chain FA elongation cycle (e.g., ELOVL1).
123. The method of any of embodiments 119-121, wherein the gene or gene product is associated with lipoic acid biosynthesis (e.g., PIAS1).
124. The method of any of embodiments 119-121, wherein the gene or gene product is associated with a protein phosphatase catalytic subunit (e.g., PPP6R3).
125. The method of any of embodiments 119-121, wherein the gene or gene product is associated with the WDR11 pathway (e.g., WDR11, FAM91A1, AP1G1, AP1S1, APIM1, APIS3, or TBC1D23).
126. The method of any of embodiments 119-121, wherein the gene or gene product is associated with retrograde transport from the Golgi apparatus to the endoplasmic reticulum, e.g., of PI and/or PC (e.g., PITP).
127. The method of any of embodiments 119-121, wherein the gene or gene product is associated with vesicular transport from a late endosome to a trans-Golgi network (e.g., STX16).
128. The method of any of embodiments 119-121, wherein the gene or gene product is associated with an activity or recruitment of a golgin, arfaptin, and/or Arf-GEF to a trans-Golgi network (e.g., ARL1).
129. The method of any of embodiments 119-121, wherein the gene or gene product encodes a component of a clathrin-dependent vesicle and/or is associated with AP1G1/AP-1-mediated protein trafficking (e.g., HEATR5B).
130. The method of any of the preceding embodiments, wherein the modulator is:
   (a) a gene editing system targeted to one or more sites within the gene or a regulatory element thereof;
   (b) a nucleic acid encoding one or more components of the gene editing system; or
   (c) a combination thereof.
131. The method of embodiment 130, wherein the gene editing system is a CRISPR/Cas system, a zinc finger nuclease system, a TALEN system, and a meganuclease system.
132. The method of any of embodiments 130-131, wherein the gene editing system binds to a target sequence in an early (e.g., the first, second, or third) exon or intron of the gene.
133. The method of any of embodiments 130-132, wherein the gene editing system binds a target sequence of the gene, and the target sequence is upstream of exon 4, e.g., in exon 1, exon 2, or exon 3.
134. The method of any of embodiments 130-133, wherein the gene editing system binds to a target sequence in a late exon or intron of the gene.
135. The method of any of embodiments 130-134, wherein the gene editing system binds a target sequence of the gene, and the target sequence is downstream of a preantepenultimate exon, e.g., is in an antepenultimate exon, a penultimate exon, or a last exon.
136. The method of any of embodiments 130-135, wherein the gene editing system is a CRISPR/Cas system comprising a guide RNA (gRNA) molecule comprising a targeting sequence which hybridizes to a target sequence of the gene.
137. The method of embodiment 136, wherein the CRISPR/Cas system is a CRISPR/Cas9 system.
138. The method of embodiment 136, wherein the CRISPR/Cas system is a CRISPR/Cas12a system.
139. The method of any of embodiments 130-138, wherein the modulator is a small interfering RNA (siRNA), small hairpin (shRNA), or guide RNA (gRNA) specific for the gene, or a nucleic acid encoding the siRNA, shRNA, or gRNA.
140. The method of embodiment 139, wherein the siRNA or shRNA comprises a nucleotide sequence complementary to a nucleotide sequence of an mRNA encoded by the gene.
141. The method of any of embodiments 130-138, wherein the modulator is an antisense oligonucleotide (ASO) specific for the gene.
142. The method of embodiment 141, wherein the ASO comprises a nucleotide sequence complementary to a nucleotide sequence of an mRNA encoded by the gene.
143. The method of any of embodiments 130-138, wherein the modulator is a small molecule.
144. The method of embodiment 143, wherein the small molecule is a protein degrader.
145. The method of any of embodiments 130-138, wherein the modulator is a protein or a peptide, or a nucleic acid encoding the protein or peptide.
146. The method of any of embodiments 130-138, wherein the modulator is an antibody molecule.
147. The method of any of embodiments 130-138, wherein the modulator is a dominant negative binding partner of a protein encoded by the gene, or a nucleic acid encoding the dominant negative binding partner.
148. The method of any of embodiments 130-138, wherein the modulator is a dominant negative variant (e.g., catalytically inactive) of a protein encoded by the gene, or a nucleic acid encoding said dominant negative variant.
149. The method of any of the preceding embodiments, wherein the AAV particle comprises an AAV genome.
150. The method of any of the preceding embodiments, wherein the AAV particle comprises an AAV-like particle.
151. The method of any of the preceding embodiments, wherein the AAV particle comprises a capsid.
152. The method of any of the preceding embodiments, wherein the AAV particle has a serotype of AAV1, AAV2, AAV3, AAV4, AAV5, AAV6, AAV7, AAV8, AAV9, AAV10, AAV11, or a combination thereof.
153. The method of any of the preceding embodiments, wherein the AAV particle is an AAV2 particle.
154. The method of any of the preceding embodiments, wherein the AAV particle is an AAV9 particle.
155. The method of any of the preceding embodiments, wherein the AAV particle has a tropism towards the CNS (e.g., AAV1, AAV2, AAV4, AAV5, AAV8, or AAV9).
156. The method of any of the preceding embodiments, wherein the AAV particle has a tropism towards heart (e.g., AAV1, AAV8, or AAV9).
157. The method of any of the preceding embodiments, wherein the AAV particle has a tropism towards kidney (e.g., AAV2).
158. The method of any of the preceding embodiments, wherein the AAV particle has a tropism towards liver (e.g., AAV7, AAV8, or AAV9).
159. The method of any of the preceding embodiments, wherein the AAV particle has a tropism towards lung (e.g., AAV4, AAV5, AAV6, or AAV9).
160. The method of any of the preceding embodiments, wherein the AAV particle has a tropism towards pancreas (e.g., AAV8).
161. The method of any of the preceding embodiments, wherein the AAV particle has a tropism towards a photoreceptor cell (e.g., AAV2, AAV5, or AAV8).
162. The method of any of the preceding embodiments, wherein the AAV particle has a tropism towards retinal pigment epithelium (RPE) (e.g., AAV1, AAV2, AAV4, AAV5, or AAV8).
163. The method of any of the preceding embodiments, wherein the AAV particle has a tropism towards skeletal muscle (e.g., AAV1, AAV6, AAV7, AAV8, or AAV9).
164. The method of any of the preceding embodiments, wherein the AAV particle comprises a nucleotide sequence encoding a therapeutic protein, e.g., a protein associated with a disorder described herein.
165. The method of any of the preceding embodiments, wherein the AAV particle comprises a nucleotide sequence encoding NGF, APOE2 (e.g., hAPOE2), TERT (e.g., hTERT), MAPT, GAD, AADC, NTN, GDNF, GCase, HIT, SMN, SMN2, SOD1, or C9orf72.
166. The method of any of the preceding embodiments, wherein the AAV particle comprises a nucleotide sequence encoding a therapeutic nucleic acid, e.g., a nucleic acid targeting a nucleotide sequence encoding a protein associated with a disorder described herein.
167. The method of any of the preceding embodiments, wherein the cell is a brain cell, a liver cell, a spinal cord cell, a dorsal root ganglion (DRG) cell, a spleen cell, a lymph node cell, a kidney cell, a lung cell, a heart cell, a muscle cell (e.g., a skeletal muscle cell, e.g., a femur muscle cell), a diaphragm cell, a bone marrow cell, or a gonad cell.
168. The method of any of the preceding embodiments, wherein the cell is a central nervous system (CNS) cell.
169. The method of embodiment 168, wherein the CNS cell is an astrocyte, an oligodendrocyte, a microglial cell, or an ependymal cell.
170. The method of any of the preceding embodiments, wherein the cell is a brain cell.
171. The method of embodiment 170, wherein the brain cell is a neuron or glial cell.
172. The method of any of the preceding embodiments, wherein the cell is a DRG cell.
173. The method of any of the preceding embodiments, wherein the cell is a liver cell;
   optionally wherein the modulator increases transduction to at least 10, 20, 30, 40, 50, or 60 viral copies per liver cell genome,
   optionally wherein the modulator increases transduction to at least 10W, 10⁶, or 10¹ viral copies per μg DNA in the liver cell.
174. The method of embodiment 173, wherein the liver cell is a hepatocyte, hepatic stellate cell, a Kupffer cell, or a liver sinusoidal endothelial cell.
175. The method of any of the preceding embodiments, wherein the cell is contacted with the AAV transduction modulator in vitro.
176. The method of any of the preceding embodiments, wherein the cell is contacted with the AAV transduction modulator ex vivo.
177. The method of any of the preceding embodiments, wherein the cell is contacted with the AAV transduction modulator in vivo.
178. A method of modulating transduction efficiency of an AAV particle, the method comprising:
   administering to a subject in need thereof an effective amount of an AAV transduction modulator,
   thereby modulating the transduction efficiency of the AAV particle.
179. A method of treating a disorder, the method comprising:
   administering to a subject in need thereof an effective amount of a therapy comprising an AAV genome or an AAV particle, wherein a gene or gene product associated with AAV transduction efficiency is modulated in the subject,
   thereby treating the disorder.
180. A method of treating a disorder, the method comprising:
   administering to a subject in need thereof an effective amount of (a) an AAV transduction modulator and (b) a therapy comprising an AAV genome or an AAV particle,
   thereby treating the disorder.
181. The method of embodiment 179 or 180, wherein the subject has not received, or is not receiving, the therapy, when the AAV transduction modulator is administered.
182. The method of any of embodiments 179-181, wherein the AAV transduction modulator was administered at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.
183. The method of any of embodiments 179-182, wherein the AAV transduction modulator was administered no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.
184. The method of any of embodiments 179-183, wherein the subject has received, or is receiving, the therapy, when the AAV transduction modulator is administered.
185. The method of any of embodiments 179-184, wherein the subject received the therapy at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.
186. The method of any of embodiments 179-185, wherein the subject received the therapy no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.
187. The method of any of embodiments 179-186, wherein the subject has a disorder or a symptom thereof, or is at risk of having a disorder or a symptom thereof.
188. The method of embodiment 187, wherein the disorder is a neurodegenerative disorder.
189. The method of embodiment 188, wherein the neurodegenerative disorder is Alzheimer's disease, Parkinson's disease, Huntington's disease, multiple sclerosis (MS), amyotrophic lateral sclerosis (ALS), or Batten disease.
190. The method of embodiment 187, wherein the disorder is mild Alzheimer's disease, mild-to-moderate Alzheimer's disease, mild-to-severe Alzheimer's disease, early dementia, Parkinson's disease (e.g., idiopathic Parkinson's disease, bilateral idiopathic Parkinson's disease, mid-to-late stage Parkinson's disease), Huntington's disease (e.g., manifest Huntington's disease), Type 1 SMA, presymptomatic SMA, or amyotrophic lateral sclerosis (ALS) (e.g., familial ALS, ALS with SOD1 mutation, or ALS with C9orf72 mutation).
191. The method of embodiment 187, wherein the disorder is an eye disorder.
192. The method of embodiment 191, wherein the eye disorder is blindness, e.g., inherited or non-inherited blindness.
193. The method of embodiment 191, wherein the eye disorder is Leber's congenital amaurosis, age-related macular degeneration, choroideremia, or color blindness.
194. The method of any of embodiments 179-193, wherein the method reduces the toxicity of the therapy, enhances the efficacy of the therapy, or both.
195. A method of preparing a subject for a therapy comprising an AAV genome or an AAV particle, the method comprising:
   administering to the subject an effective amount of an AAV transduction modulator,
   thereby preparing the subject for the therapy.
196. A method of reducing the toxicity of a therapy comprising an AAV genome or an AAV particle, the method comprising:
   administering to a subject in need thereof an effective amount of an AAV transduction modulator;
   thereby reducing the toxicity of the therapy.
197. The method of embodiment 196, wherein the subject has not received, or is not receiving, the therapy, when the AAV transduction modulator is administered.
198. The method of embodiment 196 or 197, wherein the AAV transduction modulator was administered at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.
199. The method of any of embodiments 196-198, wherein the AAV transduction modulator was administered no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.
200. The method of any of embodiments 196-199, wherein the subject has received, or is receiving, the therapy, when the AAV transduction modulator is administered.
201. The method of any of embodiments 196-200, wherein the subject received the therapy at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.
202. The method of any of embodiments 196-201, wherein the subject received the therapy no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.
203. The method of any of embodiments 196-202, wherein the toxicity is reduced by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of toxicity.
204. The method of any of embodiments 196-203, wherein the reference level of toxicity is the level of toxicity in a subject that has not been administered to the AAV transduction modulator.
205. The method of any of embodiments 196-204, wherein the reference level of toxicity is the level of toxicity before the subject is administered the AAV transduction modulator.
206. The method of any of embodiments 196-205, wherein the method reduces the toxicity in dorsal root ganglion (DRG).
207. The method of any of embodiments 196-206, wherein the method reduces the toxicity in liver.
208. The method of any of embodiments 196-207, wherein the method reduces the toxicity in cardiomyocytes.
209. The method of any of embodiments 196-208, wherein the method reduces the toxicity in retinal pigment epithelium (RPE).
210. A method of enhancing the efficacy of a therapy comprising an AAV genome or an AAV particle, the method comprising:
   administering to a subject in need thereof an effective amount of an AAV transduction modulator,
   thereby enhancing the efficacy of the therapy.
211. The method of embodiment 210, wherein the subject has not received, or is not receiving, the therapy, when the AAV transduction modulator is administered.
212. The method of embodiment 210 or 211, wherein the AAV transduction modulator was administered at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.
213. The method of any of embodiments 210-212, wherein the AAV transduction modulator was administered no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.
214. The method of any of embodiments 210-213, wherein the subject has received, or is receiving, the therapy, when the AAV transduction modulator is administered.
215. The method of any of embodiments 210-214, wherein the subject received the therapy at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.
216. The method of any of embodiments 210-215, wherein the subject received the therapy no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.
217. The method of any of embodiments 210-216, wherein the efficacy is increased by at least 10/a, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference.
218. The method of any of embodiments 210-217, wherein the reference level of efficacy is the level of efficacy in a subject that has not been administered to the AAV transduction modulator.
219. The method of any of embodiments 210-218, wherein the reference level of efficacy is the level of efficacy before the subject receives the AAV transduction modulator.
220. A method of producing a cell having an increased AAV transduction efficiency, comprising:
   contacting a cell with an AAV transduction modulator,
   thereby producing the cell.
221. A cell produced by a method of embodiment 220.
222. A cell comprising an AAV modulator described herein and an AAV particle.
223. A pharmaceutical composition comprising an AAV modulator described herein and an AAV particle.
224. A kit comprising an AAV modulator described herein and an AAV particle.
225. An AAV transduction modulator (e.g., as described herein) for use in a method of modulating transduction efficiency of an AAV particle in a cell or a subject.
226. An AAV transduction modulator (e.g., as described herein) for use in combination with an AAV genome or an AAV particle in a method of treating a disorder in a subject.
227. An AAV transduction modulator (e.g., as described herein) for use in a method of preparing a subject for a therapy comprising an AAV genome or an AAV particle.
228. An AAV transduction modulator (e.g., as described herein) for use in a method of reducing the toxicity of a therapy comprising an AAV genome or an AAV particle in a subject.
229. An AAV transduction modulator (e.g., as described herein) for use in a method of increasing the efficacy of a therapy comprising an AAV genome or an AAV particle in a subject.
230. Use of an AAV transduction modulator (e.g., as described herein) in the manufacture of a medicament for modulating transduction efficiency of an AAV particle in a cell or a subject.
231. Use of an AAV transduction modulator (e.g., as described herein) in the manufacture of a medicament in combination with an AAV genome or an AAV particle for treating a disorder in a subject.
232. Use of an AAV transduction modulator (e.g., as described herein) in the manufacture of a medicament for preparing a subject for a therapy comprising an AAV genome or an AAV particle.
233. Use of an AAV transduction modulator (e.g., as described herein) in the manufacture of a medicament for reducing the toxicity of a therapy comprising an AAV genome or an AAV particle in a subject.
234. Use of an AAV transduction modulator (e.g., as described herein) in the manufacture of a medicament for increasing the efficacy of a therapy comprising an AAV genome or an AAV particle in a subject.
235. The method of any of embodiments 1-220, the AAV transduction modulator for use of any of embodiments, 225-229, or the use of any of 230-234, wherein the method, AAV transduction modulator for use, or use results in a high gene modulating (e.g., gene editing) efficiency (e.g., at least 60%, 65%, 70%, 75%, 80%, 85%, 90%, 95%, or 98%) in a first tissue (e.g., liver), with limited impact (e.g., a gene modulating (e.g., gene editing) efficiency of 20%, 15%, 10%, 5%, 2%, or less) in a second tissue (e.g., skeletal muscle, bone marrow, or both).
236. The method of any of embodiments 1-220 or 235, the AAV transduction modulator for use of any of embodiments, 225-229 or 235, or the use of any of 230-235, wherein the AAV transduction modulator is contacted or administered intravenously.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present disclosure and practice the claimed methods. The following working examples specifically point out various embodiments of the present disclosure, and are not to be construed as limiting in any way the remainder of the disclosure.

### Example 1. Identification of Genes Impacting AAV Transduction Via Genome-Wide CRISPR/Cas9 Screens

Because of their broad tropism and safety profile, adeno-associated vectors (AAVs) have emerged as viral vectors for the transfer of therapeutic genes to target organs and across various pathological contexts. Two approved drugs are currently on the market (Zolgensma for the treatment of type 1 spinal muscular atrophy and Luxturna for the correction of Leber congenital amaurosis type 2). The efficacy of AAV gene therapy products utilizes the ability of the vector to target the relevant cell population and subsequently deliver its genetic payload to the nucleus. Fundamental knowledge about the transduction process itself remains sparse.

AAVs initially dock onto target cells by binding to glycan residues, their primary receptors, and by interacting with transmembrane proteins co-receptors. Such attachment factors have been characterized for certain AAV serotypes: AAV2 and AAV9, for example, bind to the plasma membranes of mammalian cells via heparan sulphate proteoglycan and non-sialylated N-linked galactose, respectively. In addition, most AAV variants depend upon the presence of the type-I transmembrane protein KIAA0319L (AAVR) to enter the cell. This initial attachment to the plasma membrane corresponds to the first step of a complex process involving the trafficking of the capsid through the endosomal pathway and the Golgi before the AAV particle escapes those intracytosolic organelles, reaches the nucleus and uncoats to free the vector genome. Without wishing to be bound by theory, it is contemplated that each of those events may involve interactions between the viral capsid and a specific set of host factors, the identity of which potentially varying between AAV serotypes and host species. Few of those molecular facilitators or road blockers have been identified and most studies have focused on a single serotype to address this question.

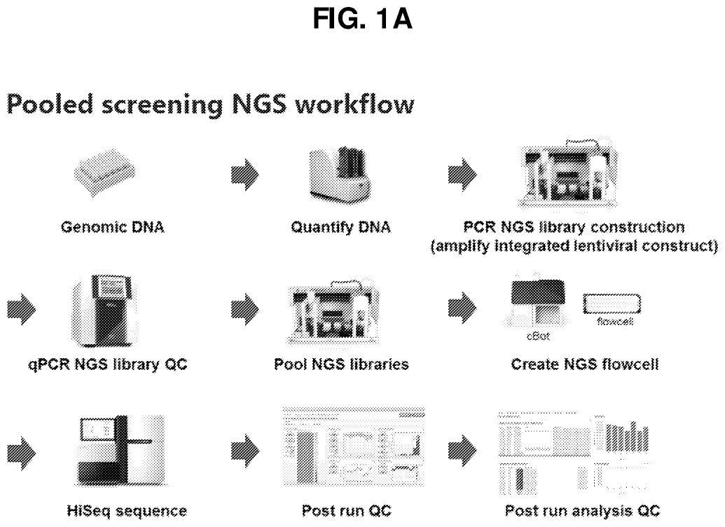

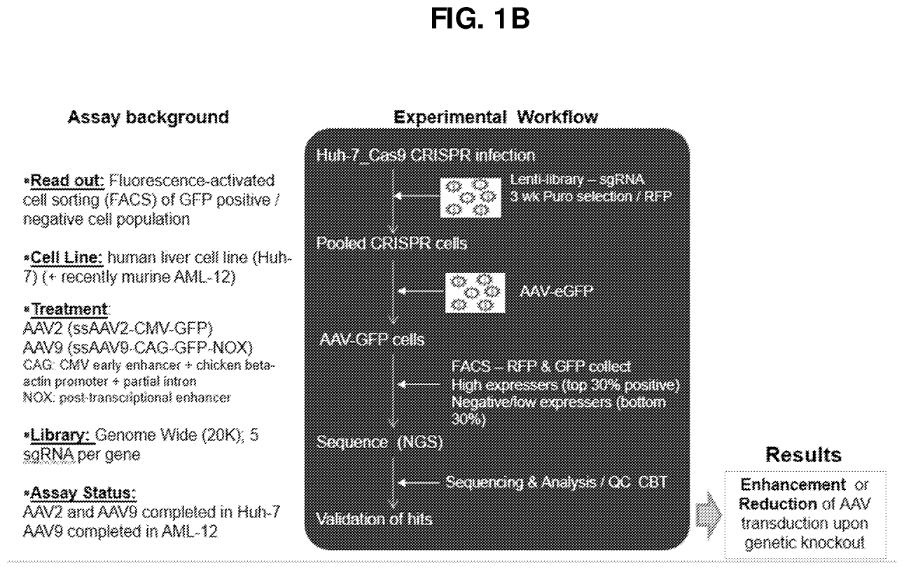

In this example, key genetic modulators of AAV2 and AAV9 transduction were identified through genome-wide CRISPR/Cas9 knockdown library screens in vitro, using either human (Huh-7) or murine (AML-12) cell lines (FIG. 1A). Those studies led to the identification of new genes either promoting or preventing the entry or trafficking of the vector into target cells. Importantly, while some of those genes affect AAV transduction regardless of the serotype, others genetic hits are influencing AAV2 or AAV9 in a specific manner. This Example also highlights key species-specific differences in the context of AAV9 transduction. Pharmacological inhibition or potentiation of these targets (for example, using chemical compounds or siRNA-encapsulating nanoparticles) may be used, for example, to enhance transduction or inhibit transduction of cell types of interest. This may, in some instances, improve efficacy or decrease toxicity of AAV vectors, for example, in specific tissues of interest.

### Methods

### Cell Line and Whole-Genome Editing

A whole-genome CRISPR screen was conducted in HuH-7/Cas9 cell line, a human hepatocyte-derived cellular carcinoma cell line stably expressing Cas9. The cells were cultured in DMEM medium supplemented with Gluta-Max, 10% fetal bovine serum, 10 ug/mL Blasticidin and 100 IU/ml penicillin/streptomycin (all reagents provided by Gibco—ThermoFisher Scientific). To generate genetic knockdowns across the genome, the Huh-7/Cas9 cells were transduced with a library of lentiviral vectors (Cellecta CRISPR human pool) encoding a puromycin resistance gene, a RFP reporter gene and a gRNA targeting each human gene. The library was redundant so that five different gRNAs were targeting one specific gene, for a total of 20,000 genes. The library was divided into two pools, CP1 and CP3. Both pools were run in duplicate, with approximately 600 cells per gRNA. After 3 weeks of selection with 2 ug/mL puromycin, the cells were transduced with either ssAAV2-CMV-GFP (100,000 MOI) or ssAAV9-CAG-GFP-NOX vector (3,300,000 MOI). After 3 days (AAV2) or 4 days (AAV9), the cells were sorted by FACS. The gating strategy removed dead cells and sorted two different GFP populations. The 25-30% high GFP+ population or the 25-30% negative/low GFP+ population. These populations along with an unsorted control were collected for DNA extractions and sent for next generation sequencing.

The whole-genome CRISPR screen was also conducted with the AAV9 seotype in AML-12/Cas9 cell line, a murine hepatocyte-derived cell line stably expressing Cas9. The cells were cultured in DMEM/F12, 10% fetal bovine serum, 15 ug·ml Blasticidin, 100 IU/ml penicillin/streptomycin, ITS, and 40 ng/mL Dexamethasone (all reagents provided by Gibco—ThermoFisher Scientific). To generate genetic knockdowns across the genome, the AML-12/Cas9 cells were transduced with a library of lentiviral vectors (Cellecta CRISPR mouse pool) encoding a puromycin resistance gene, a RFP reporter gene and a gRNA targeting each murine gene. The library was redundant so that five different gRNAs were targeting one specific gene, for a total of 20,000 genes. The library was divided into two pools, MP1005 and MP2004. Both pools were run in duplicate, with approximately 600 cells per gRNA. After 2 weeks of selection with 1 ug/mL puromycin, the cells were transduced with ssAAV9-CAG-GFP-NOX vector (2.6e11 vg/mL). After 4 days, the cells were sorted by FACS. The gating strategy removed dead cells and sorted two different GFP populations. The 25-30% high GFP+ population or the 25-30% negative/low GFP+ population. These populations along with an unsorted control were collected for DNA extractions and sent for next generation sequencing.

### CRISPR Screen Data Analysis

Raw sequencing reads were aligned to the appropriate library using Bowtie (https://genomebiology.biomedcentral.com/articles/10.1186/gb-2009-10-3-r25), allowing for no mismatches, and sgRNA counts were generated. The R software package DESeq2 (https://genomebiology.biomedcentral.com/articles/10.1186/s13059-014-0550-8) was used to evaluate differential sgRNA representation in the form of log 2 fold change between the high GFP and low GFP cell samples. A robust z-score was calculated using the median and mean-absolute deviation for the calculated fold changes across the entire sgRNA library. For gene-based hit calling, the sgRNAs were ranked by the robust z-score, and the statistical significances for each gene enriched toward higher rank (RSA up) and the lower rank (RSA down) were evaluated using the Redundant siRNA Activity (RSA) algorithm (https://www.nature.com/articles/nmeth1089). The RSA score is a statistical score (log 10 (P value)) representing the probability of a gene hit based on the collective activities of multiple sgRNAs per gene. It is a measure of how significantly the rank order of sgRNAs against a given gene differs from the population of other sgRNAs in the library. Gene knock-out activity significance was plotted as the RSA score (assay up or down) in the AAVS2 versus AAVS9 assays.

### Results

Genome-wide CRISPR/Cas9 screens were conducted in vitro to investigate key genetic modulators of AAV transduction (FIG. 1B). Those experiments were performed across two serotypes (AAV2 versus AAV9) and two cell lines (human Huh-7 and murine AML12 cells), therefore allowing for the identification of canonical (across serotypes and species), serotype and/or species-specific cellular host factors impactful to the transduction process.

### Common Genes Affecting AAV2 and AAV9 Transduction in Human Huh-7 Cells

Genes necessary or promoting transduction of both AAV2 and AAV9 in Huh-7 cells (identified from the selection of the GFP-negative cell population after genetic knockdown) included several cellular factors necessary for the entry or trafficking of vectors into target cells, such as KIAA039L (AAV-R; https://pubmed.ncbi.nlm.nih.gov/26814968/; https://pubmed.ncbi.nlm.nih.gov/28679762/; https://pubmed.ncbi.nlm.nih.gov/32280726/), which knockdown had the strongest impact, GPR108 (https://pubmed.ncbi.nlm.nih.gov/26814968/, https://pubmed.ncbi.nlm.nih.gov/29343568/, https://pubmed.ncbi.nlm.nih.gov/31784416/, https://pubmed.ncbi.nlm.nih.gov/32280726/, https://pubmed.ncbi.nlm.nih.gov/32280726/), VPS35 (https://pubmed.ncbi.nlm.nih.gov/26814968/), ATP2C1 (https://pubmed.ncbi.nlm.nih.gov/26814968/, https://pubmed.ncbi.nlm.nih.gov/32817219/) and TM9SF2 (https://pubmed.ncbi.nlm.nih.gov/26814968/, https://pubmed.ncbi.nlm.nih.gov/32280726/) (FIGS. 2A-2B). Of interest, AAV-R is the only hit for which a potential function of cell surface receptor can be inferred, as it is a transmembrane protein detectable at the plasma membrane (even though it is continuously recycling from the surface to intracellular compartments). This result demonstrates that the initial docking of the capsid at the surface of the cell dependents on very few proteins overall and that most of the host factors required for successful transduction are involved in the intracellular trafficking of the AAV capsids after their entry into target cells. Interestingly, a few identified genes encoded for proteins predominantly located into the nucleus and known to participate in the regulation of DNA repair (NEHJ) or methylation (CYREN, DCLRE1C, SAMD1). It is possible that those host factors play a role in the fate of the vector genome after capsid uncoating. Because the screen was conducted with single-stranded AAVs, one can hypothesize that some of those genes participate in the synthesis of the complementary strand of the vector genome, a necessary step for the transgene to be expressed (if this is the case, those genes may not be as impactful in the context of transduction by a self-complementary vector).

The sorting and analysis of GFP-positive Huh-7 cells (cells permissive to transduction after genetic knockdown) also allowed the identification of host factors that normally negatively affect AAV2 and AAV9 transduction. Genes identified to inhibit the entry or trafficking of the vector were more numerous than permissive host factors described above, therefore emphasizing the many roadblocks the AAV capsid needs to overcome for successful transduction and transgene expression. Because most of the previously published screens have generally focused on the detection of cellular factors promoting transduction, the vast majority the genes identified in that part of the screen have not yet been reported to be associated with AAVs. Interestingly, several members of the WDR11 pathway happen to greatly diminish transduction, including WDR11 itself but also FAM91A1, AP1G1, AP1S1, AP1M1, AP1S3 and TBC1D23 (FIGS. 2C-2D). The WDR11 complex facilitates the tethering of AP-1-derived vesicles that cycle between the trans-Golgi network and the endosomes and plasma membrane. A key function of this molecular complex is the sorting of acidic cluster-containing proteins, which includes AAV-R (https://pubmed.ncbi.nlm.nih.gov/29426865/). It is therefore possible that the activity of the WRD11 complex limits the stabilization of AAV-R at the plasma membrane at baseline, therefore decreasing the probability of interaction between the AAV capsid and its main protein receptor. Another remarkable set of genes identified participate in the MRN nuclear complex (MRE11, NBN) which is known as a DNA double-strand break repair machinery and has been previously demonstrated to regulate the fate of the AAV genome into the nucleus (https://pubmed.ncbi.nlm.nih.gov/25903339/; https://pubmed.ncbi.nlm.nih.gov/25320294/; https://pubmed.ncbi.nlm.nih.gov/17898048/). While the underlying molecular mechanisms remain largely unknown, it is possible that the activity of this complex regulates the sequestration, degradation, maturation (concatemerization of vector genomes) and integration of the AAV vector genomes into the host DNA.

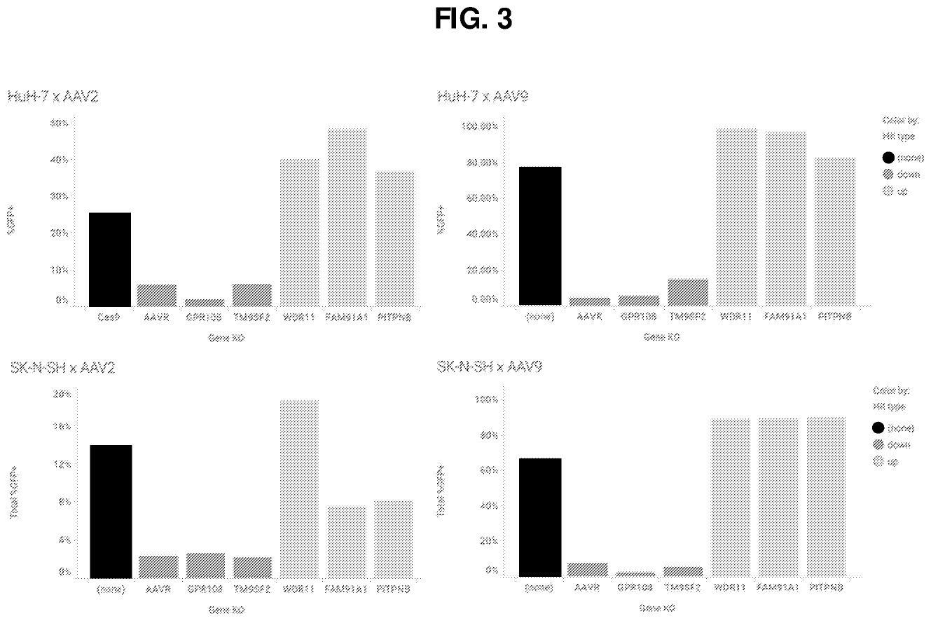

To follow-up on those results, a series of in vitro validation experiments were initiated for a small subset of a key identified genes (either promoting or inhibiting transduction, FIG. 3). Six genes (AAV-R, GPR108, TM9SF2, WDR11, FAM91A1 and PITPNB) were individually edited in both Huh-7 (hepatoma) and SK-N-HS (neuroblastoma) cell lines. The impact of those genetic knockdowns was evaluated on AAV2 and AAV9 transduction. AAV-R, GRP108 and TM9SF2 were confirmed as key facilitators of this process (their knockdown resulting in a decreased % of GFP-transduced cells), but the effect of the other three genes was less robust across serotypes and cell types. For example, the disruption of the WRD11 gene enhanced AAV2 transduction to a greater extend as compared to AAV9 in Huh-7 or in SK-N-SH cells. In addition, while the knockdown of FAM91A1 and PITPNB significantly promoted AAV2 transduction in Huh-7 cells, an opposite effect was observed in SK-N-SH cells. Those data suggest that, while the key host factors necessary for AAV transduction may be shared across cell types and serotypes, the effect of cellular inhibitors may be cell type- and serotype-specific. Those results could be of importance for the field to get a better understanding about how host factors eventually dictate the tropism of AAV vectors.

### Genes Specifically Affecting AAV9 Transduction of Huh-7 Cells

The comparison of the sets of genes identified in the genetic screens conducted with AAV2 and AAV9 serotypes allowed identifying the cellular host factors that modulate transduction in a serotype-specific manner.

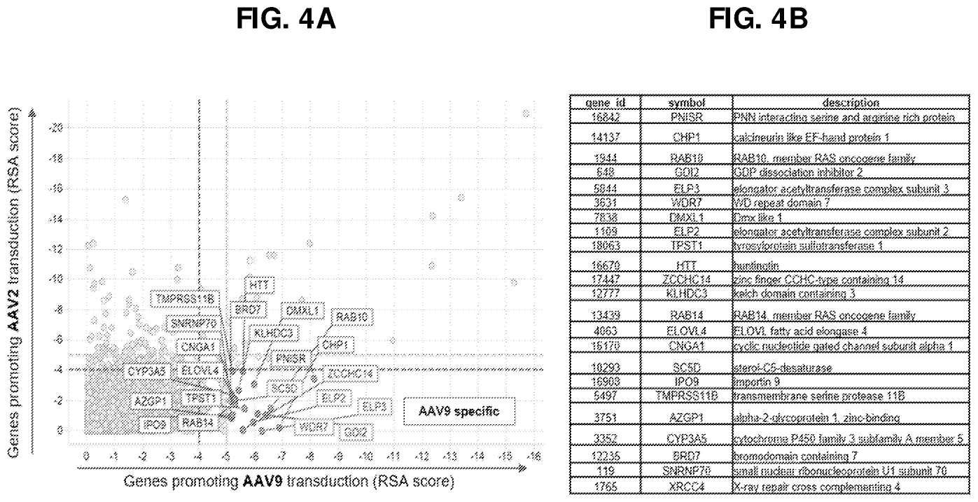

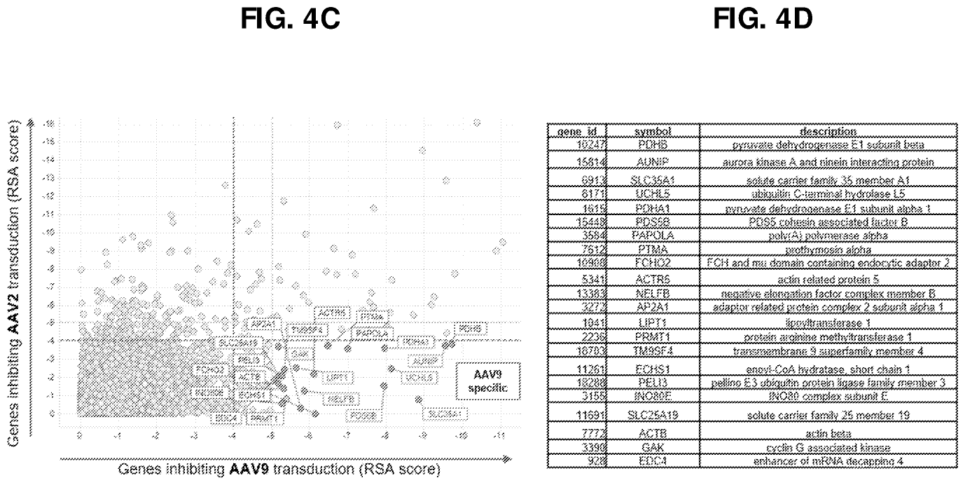

AAV9-specific genetic enhancers (FIGS. 4A-4B) included cytosolic and nuclear proteins involved in vesicular trafficking (CHP1, RAB10, RAB14, GDI2) and in the regulation of mRNA homeostasis and transcription (PNISR, ELP2 and ELP3). It is possible, however, that this latter group of genes are only impactful when a specific genetic payload is being packaged in the AAV9 capsid, and therefore do not consistently promote AAV9 transduction. Of interest, CHP1 has been shown to promote micropinocytosis, a function that could be related to the unique ability of the AAV9 capsid to cross biological barrier such as the blood brain barrier (https://pubmed.ncbi.nlm.nih.gov/27718541/).

Among AAV9-specific inhibitors (FIGS. 4C-4D), several genes important for mitochondrial function were noticeable (PDHB, PDHA1, LIPT1) and not observed in the AAV2 screen. In addition, SLC35A1 is a key enzyme transporting CMP-sialic acid from the cytosol into Golgi, controlling the terminal sialylation of glycan structures. Considering that the presence of sialic acid residues has been shown to inhibit the binding of AAV9 to cells (https://www.jbc.org/article/S0021-9258(20)51734-1/fulltext, https://pubmed.ncbi.nlm.nih.gov/34069698/, https://pubmed.ncbi.nlm.nih.gov/30101537/), the relevance of this gene is high. Other genes that negatively affected AAV9 transduction participate in DNA repair/DNA maintenance (AUNIP, UCHL5, INO80E, PDS5B, ACTR5, PRMT1), in the regulation of vesicular trafficking (AP2B1, AP2A1, FCHO2) and innate immunity (PELI3, FBXL20).

### Genes Specifically Affecting AAV2 Transduction of Huh-7 Cells

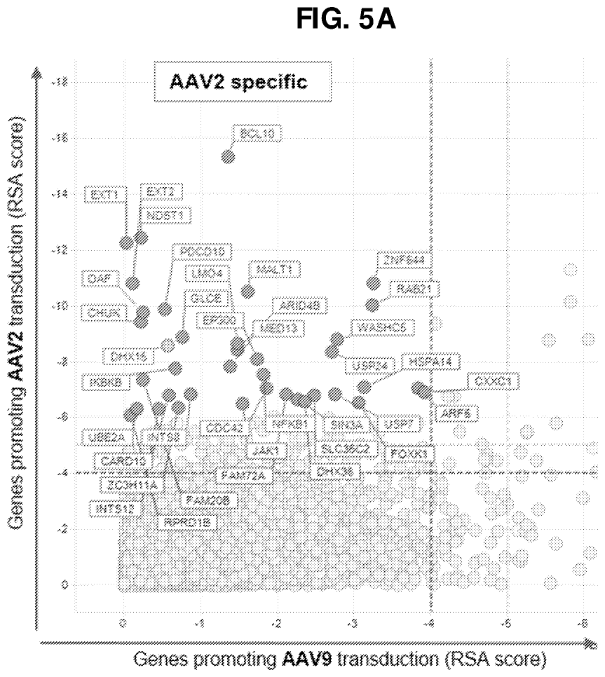

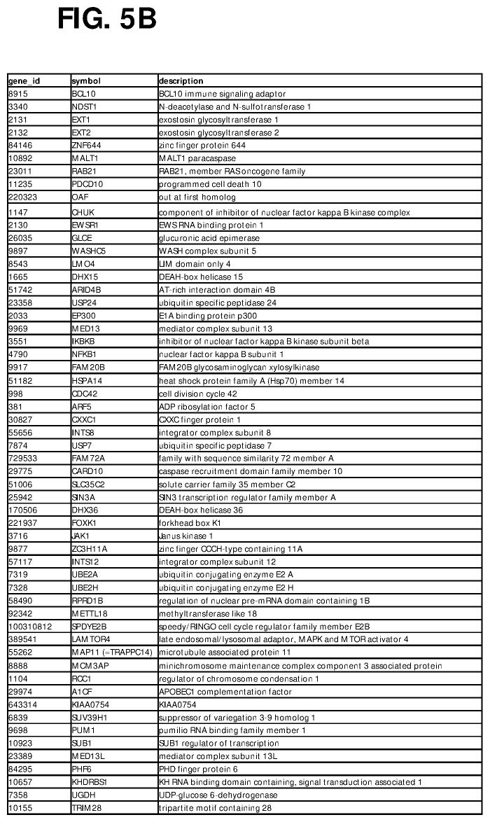

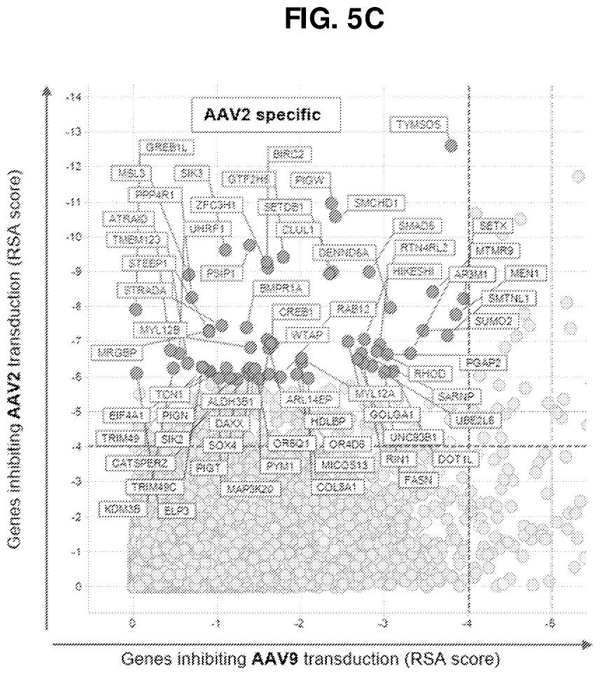

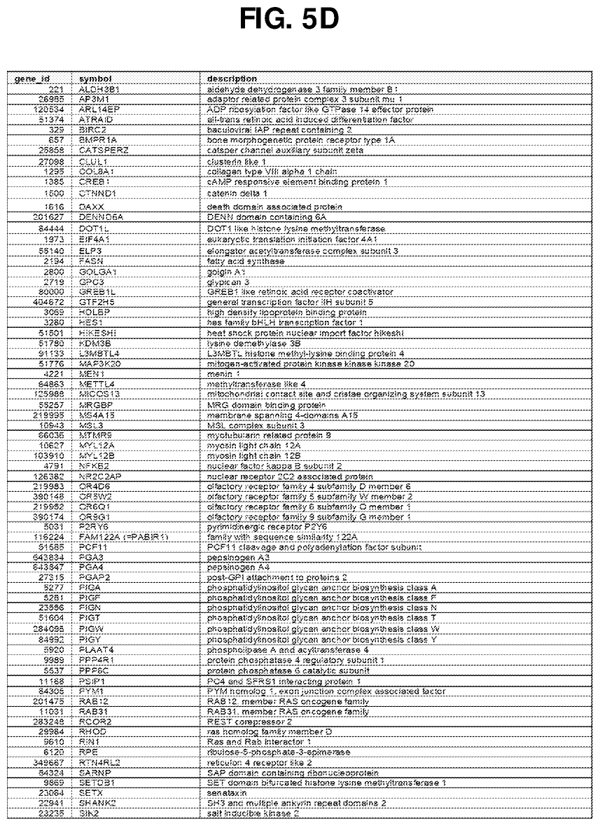

Genes promoting AAV2 transduction (FIGS. 5A-5B) included several members of the heparan sulfate biosynthesis pathway (EXT1, EXT2, NDST1, GLCE, FAM20B). This is a relevant finding considering that proteoglycan heparan sulfate residues have been characterized as the primary surface receptor for this serotype (https://pubmed.ncbi.nlm.nih.gov/9445046/, https://journals.asm.org/doi/10.1128/JVI.01939-15, https://academic.oup.con/glycob/article/14/11/969/627363). The NFKB pathway also appeared to promote AAV2 transduction (BCL10, MALT1, NFKB1, IKBKB, CHUK, CARD10), but this effect may be due to the choice of promoter driving expression of the GFP transgene (CMV promoter), as it contains several NFKB binding site. It is therefore unclear whether this molecular cascade is directly involved in the transduction process. Other identified AAV2-specific enhancers included transcriptional (ZNF644, DHX15, LMO4, EP300, MED13, MED13, and others) and translational (HSP414) factors. Proteins involved in DNA repair (DHX36, ARID4B, UBE2A) could be of relevance in the resolution of the AAV genome after entry into the nucleus.

Remarkably, most of the genes identified to have a negative impact on AAV2 transduction (FIGS. 5C-5D) play a role in DNA repair (SETX, GTF2H5), chromatin remodeling and histone post-translational modification (SMCHD1, MSL3, SETDB1, UHRF1, MEN1, MRGBP). It is possible that those host factors participate in inducing epigenetic changes onto the AAV genome, preventing expression of the transgene. Those findings also suggest that the identity of the capsid itself may dictate which intranuclear proteins will eventually interact with the AAV genome itself. Among AAV2-specific host inhibitors, TYMSOS was the gene having the strongest effect on AAV2 transduction, but the underlying mode of action of this long non-coding RNA with the regards to viral vectors is unknown.

### Limited Overlap Between Human and Murine Host Factors Modulating AAV9 Transduction

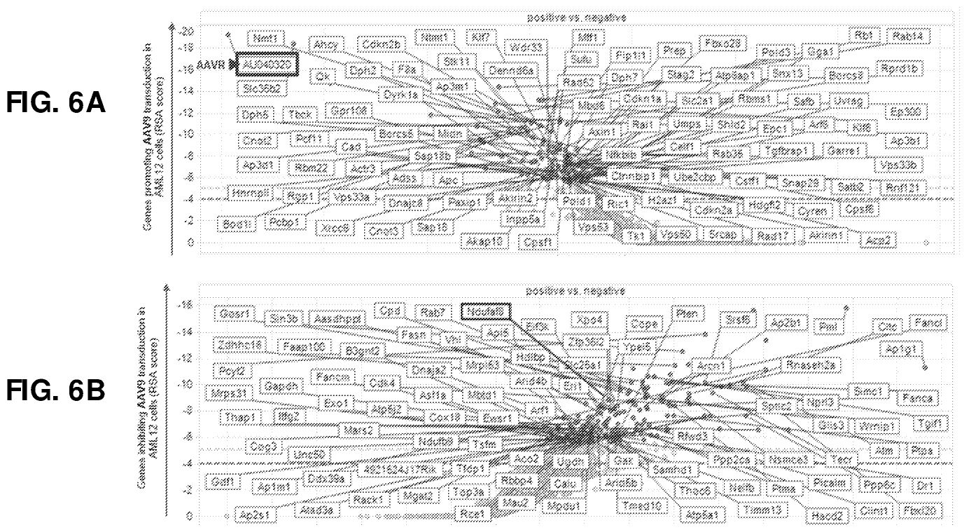

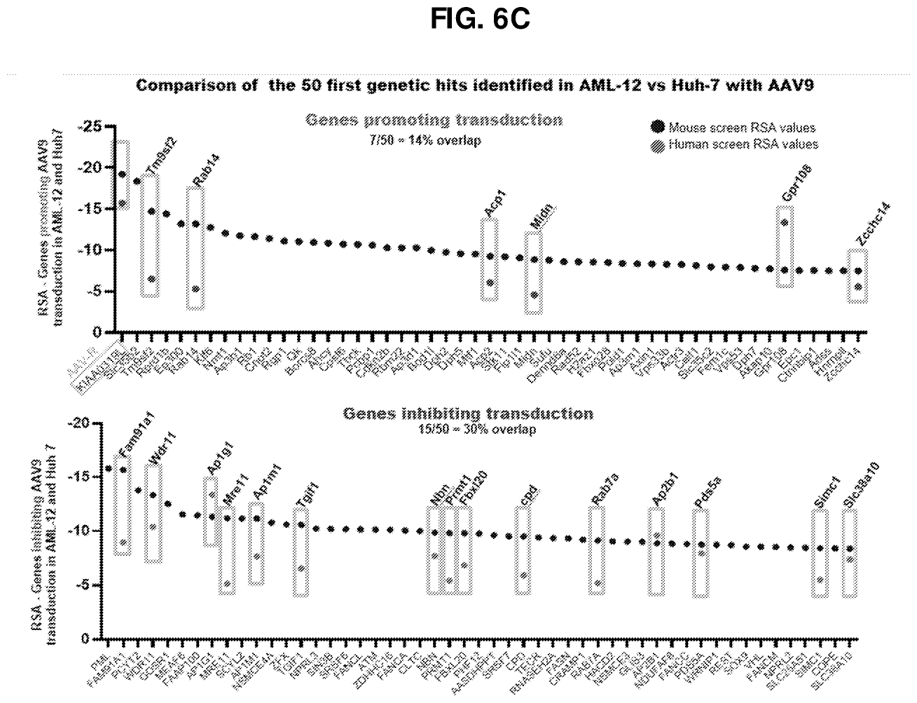

A similar whole-genome CRISPR/Cas9 screen was conducted with AAV9 in the murine hepatocyte cell line AML12 (FIGS. 6A-6B). Surprisingly, the overlap between the human and murine screens was very limited. Among the 50 first host factors that either enhanced or reduced AAV9 transduction in the murine cells, only 7 and 15 genes had previously been identified in the human Huh-7 cells, respectively. There was more overlap between the human and mouse screens for genes preventing transduction (=‘road blockers’) than genes promoting transduction (also referred to herein as facilitators). While AAV9 targets liver cells across species very efficiently, those results demonstrate that distinct species-specific host factors control the entry and trafficking of the capsid from the plasma membrane to the nucleus.

### Example 2. Pharmacological, sgRNA, and siRNA Modulation of Genes Affecting AAV Transduction

Investigation of the Mode of Action of Specific Genes Identified from the Whole-Genome CRISPR/Cas9 Screens on AAV Transduction: Identification of Key Transduction Steps being Modulated by Host Factors

As described above, key genes have been identified that either allow or prevent transduction in Huh-7 human cells. The genes that appear to impact AAV transduction most significantly from this initial screen (15-20 top ‘up’ and ‘down’ hits) are selected for further validation. Using molecular biology (qPCR for vector genome and transgene mRNA) and imaging (FACS/IHC/cell painting) techniques, whether the genetic knockdown of each target affects the initial entry of the vector, its trafficking to the nucleus, or the expression of the transgene itself is each be assessed. Those experiments will be performed across several cell lines to assess potential cell-type specific effects. It is also conceivable that a subset of those genes may have a primary impact on the subcellular localization of the AAV-R itself (a key entry factor for most AAV serotypes and the most prominent hits from our initial screens) rather than on the AAV capsid entry/trafficking. This hypothesis is tested by analyzing each genetic knockdown potentially disrupt the subcellular localization of AAV-R (either after direct immunolabeling of the receptor by IHC or after expression of a tagged AAV-R to facilitate its detection).

### Pharmacological Modulation of Identified Targets on AAV Transduction

Druggable genes were identified that appear necessary for transduction (e.g., SC5D, ICMT) or prevent efficient gene delivery (e.g., ARF1, PDHB, GAK, and MRE11) into target cells. A set of compounds were selected to be tested for their impact on AAV transduction in vitro (FIG. 7), across different cell lines. Transduction is assessed by qPCR for the vector genome and transgene mRNA, as well as by FACS/IHC. The two most potent compounds with a favorable safety profile are selected for in vivo validation (2-3 doses per compound, n=5 mice/group).

In Vivo Validation of Specific Genetic Modulators of AAV Transduction—Potential Translatability Using siRNA GalNAc Particles

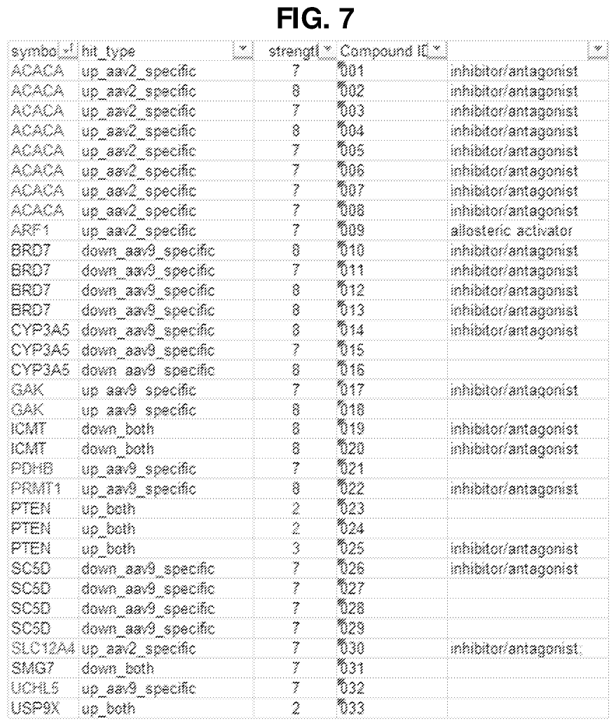

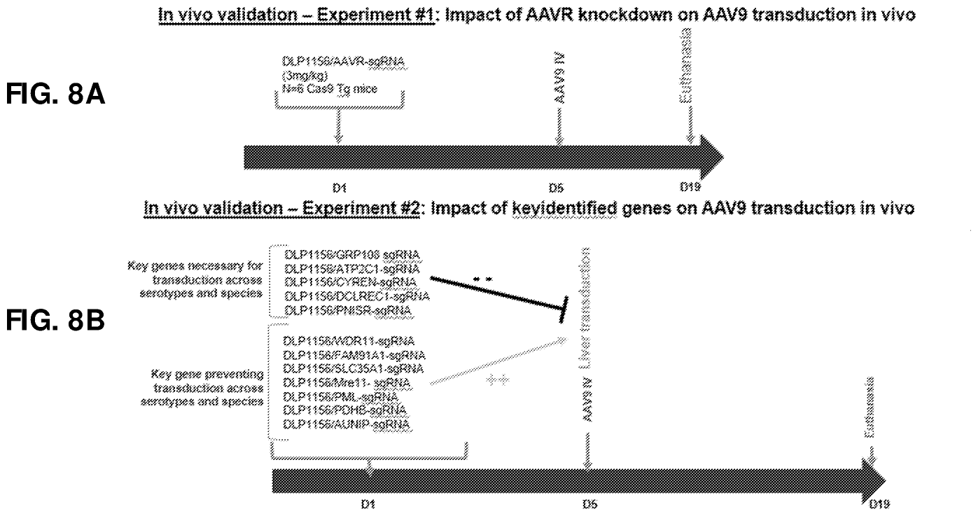

For key genetic targets identified from the in vitro screens, in vivo follow-up experiments will be conducted (FIG. 8). To mimic the initial experimental in vitro setting, nanoparticles (DLP1156, dosed at 3 mg/kg) encapsulating sgRNA targeting each gene will be systemically injected to Cas9 transgenic mice. Five days after administrating those nanoparticles, animals will be dose intravenously with AAV9. Transduction in liver is primarily evaluated considering that liver is the primary target of both nanoparticles and AAV9. An initial experiment targeting AAV-R (as positive control, FIG. 8A) is performed before testing a few other key genetic modulators (FIG. 8B).

In a second stage, whether downregulating a subset of genes using GalNac-siRNA nanoparticles can be achieved and can also efficiently modulate AAV9 transduction is evaluated. The translatability of this approach would be more optimal as it is hypothesized that the transduction process happens within the first few hours post-administration, and as such transiently modifying the expression level of relevant genes may be enough to have long-lasting effect on AAV gene transfer.

Those in vivo follow-up experiments may offer novel strategies to prevent liver transduction and improve the safety profile of AAV gene products. Indeed, liver toxicity remains one of the key safety concerns associated with systemically administered AAV vectors. Alternatively, such approaches may enhance AAV9 transduction in tissues the vector does not target efficiently in baseline conditions.

### Example 3: In Vivo Validation of Specific Genetic Modulators of AAV Transduction

Several in vitro CRISPR screens were conducted to identify key genes or pathways promoting or inhibiting AAV2 and AAV9 transduction in human or mouse liver cell lines (study 1920007). The purpose of this Example was to assess the translatability of key in vitro findings in vivo. Single guide RNA (sgRNA) targeting either the AAV receptor (‘AAV-R’, corresponding to the murine gene AU040320), GPR108, WDR11 or MRE11 murine genes were encapsulated in lipid nanoparticles and administered to Cas9 transgenic mice, prior to systemic administration of AAV9. Two weeks after dosing with AAV, AAV biodistribution was assessed in key tissues. Those 4 genes were chosen as they were identified in the previous in vitro CRISPR screens as significant modulators of AAV transduction across species (mouse and human cell lines).

### Methods

### Preliminary In Vitro Validation of the Impact of Gene AU040320 (AAV-R), GRP108, MRE11 and WDR11 Knockdown on AAV Transduction Efficiency

The murine hepatocyte-derived cell line stably expressing Cas9, AML-12/Cas9, was cultured in DMEM/F12, 10% fetal bovine serum, 15 ug·ml Blasticidin, 100 IU/ml penicillin/streptomycin, ITS, and 40 ng/mL Dexamethasone (all reagents provided by Gibco-ThermoFisher Scientific). To generate knockdowns, the AML-12/Cas9 were transduced with lentiviral vectors encoding a UbiC promoter to drive puromycin resistance plus an RFP reporter gene and an U6 promotor driving a gRNA targeting AAV-R, GFP108, WDR11, or MRE11 (Table 2). After 3 weeks of selection with 0.4 ug/mL puromycin, the cells were transduced with either ssAAV2-CMV-GFP (5×10¹⁰ vg/mL) or ssAAV9-CAG-GFP-NOX (1×10¹¹ vg/mL). After 4 days, the cells were imaged on the ImageXpress micro-confocal (Molecular devices) and analyzed with the MetaXpress cell scoring algorithm adjusted to on-plate controls.

TABLE 2
sgRNA design

| gRNA | | Name of | | |
|---|---|---|---|---|
| name | sgRNA sequence | gene | Gene ID | Species |
| AAV-R | CTTCCAGCCACCATAGAGCG | AU040320 | 100317_2 | mouse |
| GPR108 | GGTCCAAGTCCGAAAATATG | GPR108 | 78308 | mouse |
| WDR11 | TCTGCCGTCGCTTACTATGA | WDR11 | 207425 | mouse |
| MRE11 | TTCTATGAAGAACCGCCGCA | MRE11 | 17535 | mouse |

### Animals

In-house derived spCAS9 Tg mice (B6-Rosa26,tm7.1,1-5C.Npa) were chosen for the in vivo validation study (sent from Charles River Laboratories, Wilmington, MA). Thirteen weeks-old males and females were used. Animals deemed acceptable for use on study were chosen based on pretest examinations and randomized to groups via cage order.

gRNA Encapsulated Lipid Nanoparticles

Lipid nanoparticles (DLP) were prepared using microfluids (Nanoassembler, Precision Nanosystem). The nanoparticles comprised of ionizable lipid, cholesterol, DSPC and pegylated lipid. The lipid, dissolved in ethanol and three volumes of mRNA (see Table 3 for sequence) in citrate buffer with flow rate of 12 ml/min. Post formulation, the nanoparticles were buffer exchanged with PBS using tangential flow filtration system. The nanoparticle, underwent characterization, which included determining size and pdi using Zetaizer (Malvern instruments). mRNA encapsulation inside the nanoparticles were determined using spectrophotometer.

Nanoparticles were incubated with SyberGold dye with or without breaking them using Triton-X 100 (0.02%) to measure free mRNA vs all mRNA in the solution.

TABLE 3
gRNA sequences

| gRNA | |
|---|---|
| name | Annotation of full gRNA sequence |
| AAV-R | 5′(cs)(us)(us)CCAGCCACCAUAGAGCGGUUUUAGAgcuagaaauagcAAGUUAAAAUA |
| | AGGCUAGUCCGUUAUCAacuugaaaaaguggcaccgagucggugc(us)(us)(us)u-3′ |
| GPR108 | 5′(gs)(gs)(us)CCAAGUCCGAAAAUAUGGUUUUAGAgcuagaaauagcAAGUUAAAAUA |
| | AGGCUAGUCCGUUAUCAacuugaaaaaguggcaccgagucggugc(us)(us)(us)u-3′ |
| WDR11 | 5′(us)(cs)(us)GCCGUCGCUUACUAUGAGUUUUAGAgcuagaaauagcAAGUUAAAAUA |
| | AGGCUAGUCCGUUAUCAacuugaaaaaguggcaccgagucggugc(us)(us)(us)u-3′ |
| MRE11 | 5′(us)(us)(cs)UAUGAAGAACCGCCGCAGUUUUAGAgcuagaaauagcAAGUUAAAAUA |
| | AGGCUAGUCCGUUAUCAacuugaaaaaguggcaccgagucggugc(us)(us)(us)u-3′ |
| scramble | 5′(gs)(cs)(as)GGCGGUUCAAGACUCAUGUUUUAGAgcuagaaauagcAAGUUAAAAUA |
| guide | AGGCUAGUCCGUUAUCAacuugaaaaaguggcaccgagucggugc(us)(us)(us)u-3′ |

* RNA residues: A, C, G, U;
2′-O-methyl residues: a, c, g, u;
2′-F residues: Af, Cf, Gf, Uf;
Phosphorothioate linkage: s

### AAV Vector

The AAV9-CAG-GFP-NOX vector was manufactured as follows. AAV9-CAG-GFP-NOX is an AAV9 serotype vector encoding the reporter gene Green Fluorescent Protein expressed under the strong ubiquitous promoter CAG. The large-scale production was generated using nano bioreactor process 2.0. The downstream processing includes clarification through depth filtration and polish filtration. The batch was loaded onto cation exchanger (CEX), with gradient elution 500 mM NaCl to 2M NaCl. The CEX pool was then neutralized from pH 3.5. A tangential flow filtration (TFF) step was used to exchange CEX buffer to 3M CsCl. The TFF pool was ultra-centrifugated to separate empty and full capsids. The full AAVs were resuspended in PBS. The final titer of the vector was determined by ddPCR and reached 4.4×10¹³ vg/ml.

Systemic Delivery of sgRNA/DLP and AAV9

Table 4 summarizes the study design and test articles used for this study. Nanoparticles (DLP1156, dosed at 3 mg/kg) encapsulating sgRNA targeting the murine AAV-R, WDR11, GPR108 or MRE11 genes were intravenously injected to Cas9 transgenic mice through the lateral tail vein. A control sgRNA/DLP1156 (‘scramble sgRNA’ or sgSCR) targeting no endogenous murine gene was included in group 2. Group 7 included a slightly different nanoparticle formulation (DLP1620) engineered by Novartis Biological Center (NBC), for exploratory purposes. Seven days after sgRNA nanoparticle administration (on Day8), animals received an intravenous injection of AAV9-CAG-GFP-NOX at a dose of 4e13 vg/kg. Animals included in Group 1 received 2 consecutive injections pf vehicle (Day 1 and Day 8). Two weeks after dosing with AAV9, the animals were euthanized. Blood and key tissues were collected at necropsy and either fixed in 10% NBF or snap-frozen for further analyses.

TABLE 4
Summary of study design
Formulation: Phosphate buffered saline (PBS) with PF68

| | | | Doses | Study | Animal |
|---|---|---|---|---|---|
| Group | Test Article | Animal ID | (DL1156/1620 and AAV9) | length | N |
| 1 | Vehicle | 1501/03 | — | Day 21 | 3 |
| 2 | DLP1156/sgSCR (Day 1) + | 2001/02 | DLP1156/sgSCR, dosed at 3 mg/kg + | Day 21 | 4 |
| | AAV9 (Day 8) | 2501/02 | AAV9-CAG-GFP: 1 × 10¹² (4 × 10¹³vg/kg) | | |
| 3 | DLP1156/AAV-R (Day 1) + | 3001/02 | DLP1156/sgAAV-R, dosed at 3 mg/kg + | Day 21 | 4 |
| | AAV9 (Day 8) | 3501/02 | AAV9-CAG-GFP: 1 × 10¹² (4 × 10¹³vg/kg) | | |
| 4 | DLP1156/sgGPR108 (Day 1) + | 4001/02 | DLP1156/sgGPR108, dosed at 3 mg/kg + | Day 21 | 4 |
| | AAV9 (Day 8) | 4501/02 | AAV9-CAG-GFP: 1 × 10¹² (4 × 10¹³vg/kg) | | |
| 5 | DLP1156/sgWDR11 (Day 1) + | 5001/02 | DLP1156/sgWDR11, dosed at 3 mg/kg + | Day 21 | 4 |
| | AAV9 (Day 8) | 5501/02 | AAV9-CAG-GFP: 1 × 10¹² (4 × 10¹³vg/kg) | | |
| 6 | DLP1156/sgMRE11 (Day 1) + | 6001/02 | DLP1156/sgMRE11, dosed at 3 mg/kg + | Day 21 | 4 |
| | AAV9 (Day 8) | 650102 | AAV9-CAG-GFP: 1 × 10¹² (4 × 10¹³vg/kg) | | |
| 7 | DLP1620/sgAAV-R (Day 1) + | 7501/03 | DLP1156/sgMRE11, dosed at 3 mg/kg + | Day 21 | 3 |
| | AAV9 (Day 8) | | AAV9-CAG-GFP: 1 × 10¹² (4 × 10¹³vg/kg) | | |

IV, bolus

Evaluation of Gene Editing Efficiency Approximately 100 mg (weight not documented) of liver, skeletal muscle and bone marrow (BM—from left femur) tissue samples were collected and frozen in liquid nitrogen, stored at approximately −70° C. for analysis of AAV-R, GPR18, WDR11 and MRE11 gene editing efficiency using a ddPCR drop-off assay. Genomic DNA (gDNA) isolation from samples is described under “Viral copy number and mRNA expression”. Each drop-off ddPCR assay was carried out using a reaction of 50 ng of gDNA, Bio-Rad ddSuperMix for probes (No UTP) (#186-3024), EcoRI (NEB #R3101 S) and an assay of interest (Table 5). An assay for TFRC by life technologies (4458367) was used as a copy number reference. Bio-Rad droplet generator was used to generate droplets, followed by thermocycling (Table 6, thermal condition), then read on Bio-Rad QX200Droplet reader with the QuantaSoft software plus Excel to analyze the samples.

TABLE 5
Primers* and MGB-probes sequences

| Assay of | Sequence/ | | Length | | | Amplicon |
|---|---|---|---|---|---|---|
| interest | Probe Name | Sequence | (bp) | Tm | Dye | length |
| AAV-R | AAV-R_Fw_Prim | CTTGAGAACTGAGGGAGAAAATCAC | 25 | 58.5 | NA | 186 |
| | AAV-R_Rev_Prim | GAATTGGAAGAGTCTGTCCTAAAAGG | 26 | 58.8 | NA | |
| | AAV-R_sgPro | CTGCCACGCTCTAT | 14 | 68.0 | 6FAM | NA |
| | AAV-R_RefPro | CTTGCAGGAAGCCT | 14 | 68.0 | VIC | |
| GPR108 | GPR108_Fw_Prim | TGACCTCCCCCTTCCTGATT | 20 | 59.3 | NA | 213 |
| | GPR108_Rev_Prim | CCCAGTAGTGTTAAAGCTGGTCTTT | 25 | 58.1 | NA | |
| | GPR108_sgPro | CAAGTCCGAAAATATG | 16 | 70.0 | 6FAM | NA |
| | GPR108_RefPro | TTCTCCCTGAAGCTC | 15 | 69.0 | VIC | |
| MRE11 | MRE11_Fw_Prim | TGTGCAGACATGTAGGCTTGCT | 22 | 59.5 | NA | 128 |
| | MRE11_Rev_Prim | GTTGAACAGGTTTGGGTGGTTAG | 23 | 58.5 | NA | |
| | MRE11_sgPro | CCGTGCGGCGGTT | 13 | 70.0 | 6FAM | NA |
| | MRE11_RefPro | CGCATTAAAGGGAGAAAG | 18 | 69.0 | VIC | |
| WDR11 | WDR11_Fw_Prim | GAAAACTGTCCGTCCCTTCAGT | 22 | 58.2 | NA | 138 |
| | WDR11_Rev_Prim | CCTCACCTGTTCCGTGCAT | 19 | 58.1 | NA | |
| | WDR11_sgPro | CAGCTGCCCTCATAGTA | 17 | 70.0 | 6FAM | NA |
| | WDR11_RefPro | TGGTGTGCTGTCCTGT | 16 | 68.0 | VIC | |

*Customized primers using Primer express 3.0

TABLE 6
Thermal conditions for (Targets and TFRC assays)

| Step | Temps (° C.) | Duration | Repeat |
|---|---|---|---|
| Enzyme activation | 95 | 10′ | 1 |
| Denaturation | 95 | 15″ | 40 |
| Annealing | * | 30″ | |
| Enzyme deactivation | 98 | 10′ | 1 |
| End | 4 | ∞ | 1 |

*AAV-R assay: 57.5° C./GPR108 assay: 58.9° C./WDR11 assay: 58.9° C./MRE11 assay: 58.9° C./TFRC assay: 60° C.

Viral Copy Number and mRNA Expression

Approximately 25 mg (weight not documented) of each frozen tissue sample was used for the quantification of vector genome and qPCR. Homogenization of tissue was completed using Precellys Evolution prior to DNA and RNA isolation.

For vector genome copies in the bone marrow, heart, liver, muscle, and spinal cord, DNA was isolated using Qiagen's (69582) DNeasy 96 Blood & Tissue kit. The extracted DNA was quantified using a Nanodrop and normalized to 10 ng/ul using the Qiagility robot. 30 ng of DNA was assayed for eGFP (Table 5) and mouse CFTR (Table 5) genes using a 7900HT Fast real-time PCR machine. The eGFP results were analyzed to determine the vial copies per cell and per ng gDNA.

To assess eGFP transgene mRNA expression, RNA was isolated using RNeasy mini kit (Qiagen, #74106) with the additional on-column DNase digestion step according to manufacturer. Extracted RNA was quantified using the Nanodrop and normalized to 300 ng for the liver and 150 ng in the muscles for reverse transcription using applied biosystems high-capacity cDNA kit (4368813). 2 ul of the cDNA was used for qPCR reaction, looking at eGFP (Table 7) and 18s expression (Life technologies #4333760F). Fold change in eGFP expression was determined using Delta C_{T} (C_{T} obtained for eGFP-C_{T} obtained for 18S) and the formula 2\hat{ }(−DC_{T}). Final data sets compared eGFP expression to sgRNA scrambled control.

TABLE 7
PCR assays

| Assay | | | Internal |
|---|---|---|---|
| name | 5′ primer | 3′ primer | oligo |
| eGFP | GAACCGCATC | TAGACGTTGTG | ATCGACTTCAAG |
| | GAGCTGAA | GCTGTTGTAG | GAGGACGGCAAC |
| Murine | AGAAAAGAGA | GTGAACGTCAT | CCCACATTTCCA |
| CFTR | CTGTCCCTAG | GAGATCCAAA | GGCAGAAG |

### Protein Expression Via Western Blot Analysis and Direct Fluorescent Measurements

Approximately 100-150 mg (weight not documented) of each frozen tissue sample was used for the quantification of western blot. Protein expression of AAV-R, MRE11, and GFP were analyzed from liver tissue. Cellular proteins of snap frozen liver tissues were extracted in RIPA buffer (RIPA buffer (150 mM NaCl, 50 mM Tris, 1% NP-40, 0.5% sodium deoxycholate, 0.1% SDS, pH 7.4), supplemented with protease inhibitor cocktail. Total protein levels in liver homogenates were measured using the Pierce bicinchoninic acid (BCA) protein assay kit (Thermo Fisher Scientific, 23227), and were normalized to equal protein concentration per sample. Proteins (30 ug) were resolved by SDS-PAGE, transferred to a nitrocellulose membrane, and probed with anti-AAV-R antibody (ProteinTech, 21016-1-AP), anti-Mre11 antibody (Thermo Fisher Scientific, MA5-35465), or anti-GFP antibody (GenScript, A01704) Following incubation with secondary horseradish peroxidase conjugated anti-rabbit antibody (Jackson ImmunoResearch, 111-035-003), detections were performed using the ECL detection reagents and Li-Cor C-DiGit Blot Scanner. Equal loading for each sample was confirmed using an anti-GAPDH (GeneTex, GTX100118) antibody. Densitometry analysis was performed with Image Studio Digits Ver 5.2 (LI-COR, Lincoln, Nebraska USA).

For direct measurement of fluorescence intensity, liver protein extracts were diluted to 0.5 mg/ml protein content in PBS, fluorescence intensity was measured in 100 μl volume in a 96-well plate using a fluorescence reader at excitation 485 nm, emission 538 nm wavelength (SpectraMax M5, Molecular Devices). Results were expressed as Relative Fluorescence Units (RFUs).

### Molecular Localization Studies

Liver samples were processed and analyzed by immunohistochemistry (IHC) and in situ hybridization (ISH) analyses on paraffin-embedded tissues. Immunohistochemistry staining for GFP including the deparaffinization and antigen retrieval steps, were performed on a Ventana Discovery XT autostainer using standard Ventana Discovery XT reagents (Ventana, Indianapolis, IN). Slides were deparaffinized then submitted to heat-induced antigen retrieval by covering them with Cell Conditioning 1 (CC1/pH8) solution according to the standard Ventana retrieval protocol. Slides were incubated with the primary antibody (rabbit monoclonal anti-GFP antibody clone EPR14104-89 at 0.372 ug/mL) or a non-immune isotype-matched control (Rabbit monoclonal IgG clone DA1E at 0.372 ug/ml) as indicated in Table 8 for one hour. Visualization was obtained by incubation with the appropriate Ventana Discovery OmniMap HRP reagent as indicated below followed by Ventana Discovery ChromoMap 3,3′-Diaminobenzidine (DAB). Counterstaining was performed using Ventana Hematoxylin and Ventana Bluing reagent for 4 minutes each. Slides were dehydrated, cleared and cover slipped with a synthetic mounting medium.

TABLE 8
Immunohistochemistry probes

| | | Dilution/ |
|---|---|---|
| Antibody supplier/code | Clone | Concentration |
| Abcam/ab183735 | EPR14104-89 | 0.372 ug/ml |
| Cell signaling Technology/3900s | DA1E | 0.372 ug/ml |

In situ hybridization to detect GFP antisense (AS) and sense (S) sequences encoded in the AAV vector as well as Macaca fascicularis (Mm)-PPIB (AS) (positive control and tissue quality control) and DapB (AS) (negative control) genes (Table 7) was performed on select blocks using reagents and equipment supplied by Advanced Cell Diagnostics (ACD) (Hayward, CA) and Ventana Medical Systems (Roche, Tucson AZ). The in situ hybridization RNAScope® probes were designed by ACD. A list of probes is presented in Table 9. Positive PPIB and negative DAPB control probe sets were included to ensure mRNA quality and specificity, respectively. The hybridization method followed protocols established by ACD and Ventana systems using Ventana mRNA Red chromogens. Briefly, 5 μm sections were baked at 60 degrees for 60 minutes and used for hybridization. The deparaffinization and rehydration protocol was performed using a Sakura Tissue-Tek DR5 Stainer with the following steps: 3 times xylene for 5 minutes each; 2 times 100% alcohol for 2 minutes; air dried for 5 minutes. Off-line manual pretreatment in 1× retrieval buffer at 98 to 104 degrees C. for 15 minutes. Optimization was performed by first evaluating PPIB and DAPB hybridization signal and subsequently using the same conditions for all slides. Following pretreatment, the slides were transferred to a Ventana Ultra autostainer to complete the ISH procedure including protease pretreatment; hybridization at 43 degrees C. for 2 hours followed by amplification; and detection with HRP and hematoxylin counter stain.

TABLE 9
In situ hybridization probes

| Probe | Name | Vendor | Cat # | Comment |
|---|---|---|---|---|
| DapB | Dihydrodipicolinate reductase | ACD | 312039 | Negative control probe |
| Mm-PPIB | Mus musculus peptidylprolyl | ACD | 313919 | Positive control |
| | isomerase B | | | housekeeping gene |
| | | | | probe |
| GFP-antisense | Green Fluorescent Protein | ACD | 400289 | Vector antisense probe |
| GFP-sense | Green Fluorescent Protein | ACD | 409979 | Vector sense probe |

### Results

An in vivo study was conducted to investigate key genetic modulators of AAV transduction. To do so, four different murine genes (AAV-R, GPR108, WDR11 and MRE11) were initially knocked down using a CRISPR/Cas9 approach by systemic delivery of sgRNA-conjugated lipid nanoparticles in Cas9 transgenic mice. After 7 days, a single intravenous injection of single-stranded AAV9-CAG-GFP-NOX was performed. Editing efficiency, vector biodistribution and transgene product quantification was assessed at necropsy 14 days post-AAV administration.

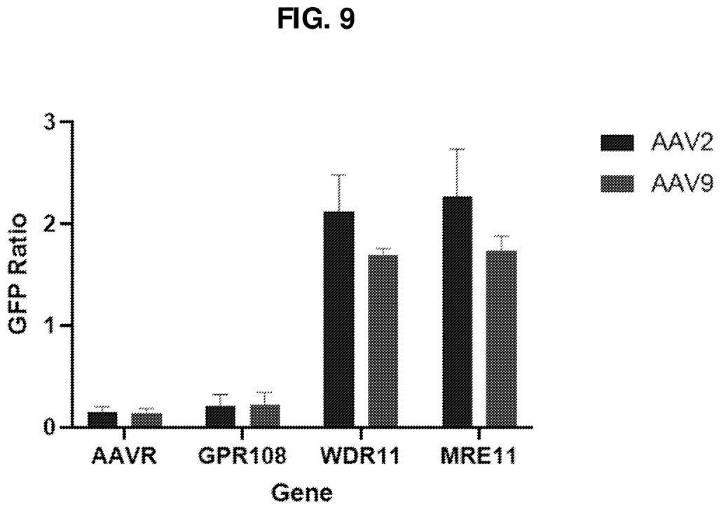

### In Vitro Validation of AAV-R, GPR108, WDR11 and MRE11 as Key Host Factors Modulating AAV2 and AAV9 Transduction in Murine AML-12 Cells (FIG. 9)

A series of in vitro validation experiments were initiated for a small subset of a key identified genes (either promoting or inhibiting transduction, Table 4. Four genes (AU040320—further referred to as ‘AAV-R’, GPR108, WDR11, and MRE11) were individually edited in AML-12 cell line. The impact of those genetic knockdowns was evaluated on AAV2 and AAV9 transduction. AAV-R and GRP108 were confirmed as key ‘enablers’ of this process, with their knockdown resulting in a decreased of 100× fold of GFP-transduced cells, respectively. By contrast, the genetic disruption of the WRD11 or MRE11 genes enhanced AAV transduction 2-fold. Those in vitro results therefore confirm the sgRNA sequences for further use in vivo and the effect of those key genes in the AML-12 cells to modulate AAV transduction.

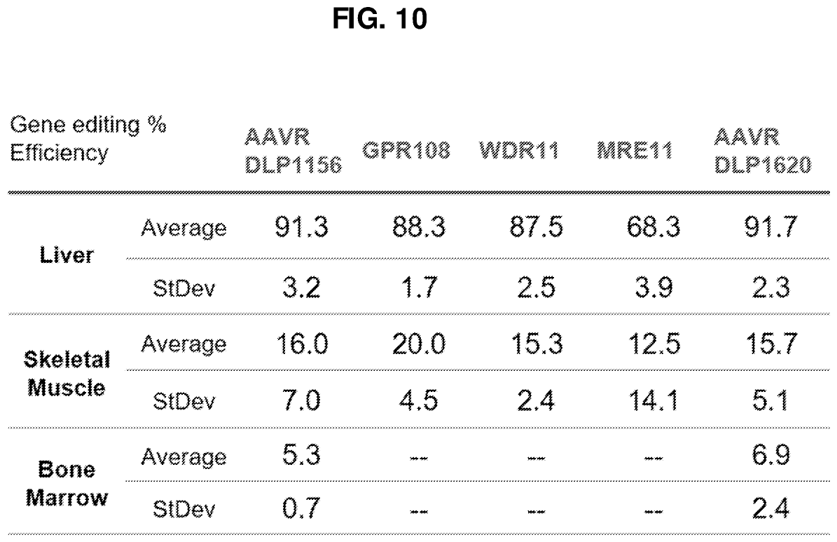

Intravenous Administration of sgRNA-Conjugated Lipid Nanoparticles Targeting AA V-R, GPR108, WDR11 and MRE11 in Cas9 Transgenic Mice Leads to Efficient Gene Editing in Liver (FIG. 10)

A drop-off ddPCR assay was used to examine the gene editing efficiency in the liver, skeletal muscle and select bone marrow samples. High editing efficiency of the respective targets was detected in liver tissue after delivery of DLP1156/sgRNA-(AAV-R, GPR108, WDR11, and MRE11) and DLP1162/sgRNA-AAV-R, ranging from 68.3t3.9% for MRE11 to 91.7±2.3% for AAV-R. No editing was observed when using scrambled sgRNA sequence. Gene editing in skeletal muscle tissues was less efficient, ranging from 12.5±14.1 for MRE11 to 20t4.5% for GPR108. Bone marrow had even lower editing efficiency (5.3±0.7% DLP1156/sgRNA-AAV-R and 6.9±2.4% DLP1620/sgRNA-AAV-R). The difference in editing efficiencies across tissues likely reflect the biodistribution of sgRNA-conjugated lipid nanoparticles in vivo following systemic dosing. Based on current observation and fluorescence values, unprecise editing is unlikely to happen with AAV-R, GPR108 and MRE11 due to no difference in Reference-probe fluorescent signals: Scramble versus treated groups. Small amplicons may be more sensitive to pick up the unprecise editing events as the reference probe is closer to the cutting site.

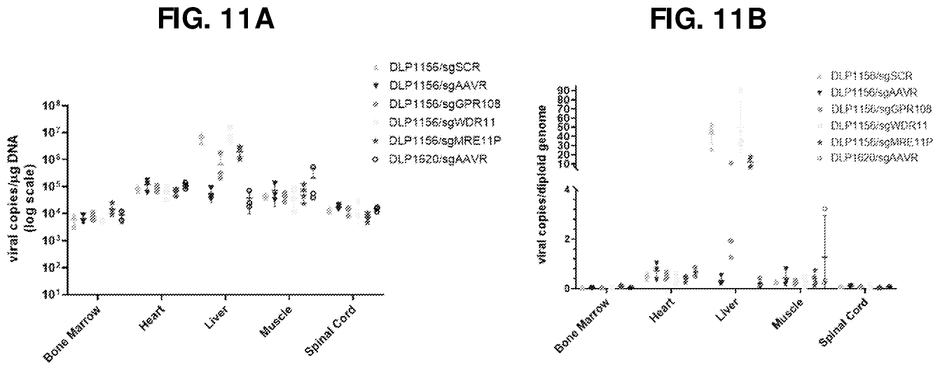

### AAV9 Biodistribution In Vivo is Significantly Impacted by the Knockdown of Key Host Entry Factors of the Vector (FIG. 11)

AAV9 biodistribution was examined in bone marrow, heart, liver, muscle, and spinal cord. For the mice dosed with the control sgRNA (‘sgSCR’) prior AAV9, liver had the highest viral copies per ug of DNA (43±11.5 vg/dg), followed by heart/muscle, and bone marrow/spinal cord. The knockdown of AAV-R (DLP1156 & DLP1620) reduced AAV9 levels in the liver, with the number of vector genomes per diploid genome (vg/dg) decreasing to 0.3±0.18 vg/dg and 0.2±0.17 vg/dg after intravenous delivery of sgAAV-R/DLP1156 and sgAAV-R/DLP1620 respectively. GPR108 KO also reduced AAV9 levels in the liver (4.0±4.7 vg/dg), but not as robust. A single GPR108 mouse, number 4001, had higher vg/dg than the other mice at 11 vg/dg. If we remove this mouse from the group of four, the average AAV9 levels drops to 1.7±0.38 vg/dg. MRE11 KO showed a slight reduction in the liver and no change in the amount of viral genome in that tissue was observed after gene editing of WDR11. Of note, the biodistribution of the vector genome in other tissues analyzed (heart, skeletal muscle and bone marrow) did not significantly differ across experimental groups.

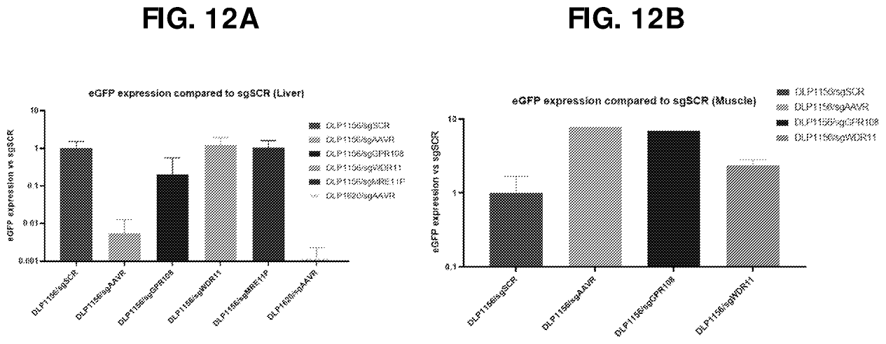

GFP mRNA Expression Parallels the Biodistribution of AAV9 Vector Genomes (FIGS. 12A-12B)

The levels of GFP mRNA expression in the liver and select muscle samples (only the following animals had sufficient RNA 2002, 2501, 3002, 4002, 5001, & 5002) were analyzed. The knockdown of AAV-R, whether delivery via DLP1156 or DLP1620, reduced GFP expression in the liver by a factor of 100-fold as compared to the control group (sgSCR+AAV9). Genetic editing of GPR108 prior AAV9 dosing also reduced GFP expression, about 10-fold as compared to scramble gRNA treated animals. The WDR11 and MRE11 genes knockdown had no change on GFP expression as compared to the control group. The analysis of GFP expression levels in skeletal muscle revealed a slight increase in transgene expression after WDR11 knockdown. A larger increase in GFP expression was observed with AAV-R and GPR108 knockdown in that tissue, but only single animal was analyzed.

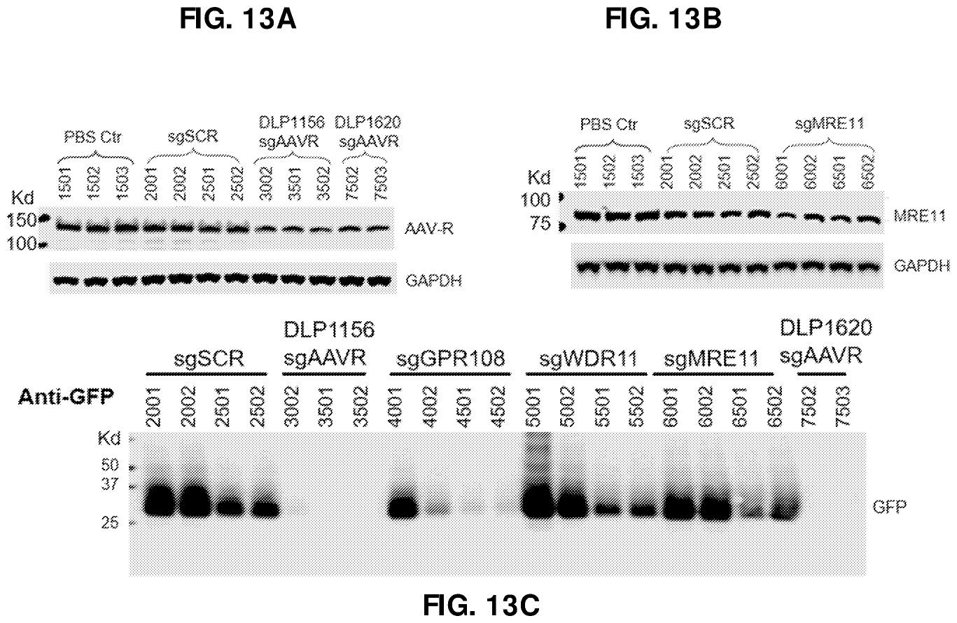

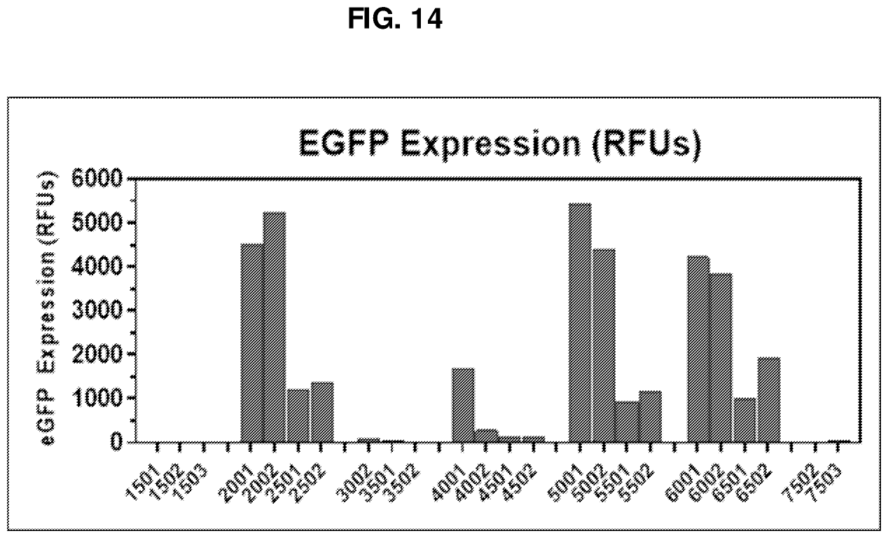

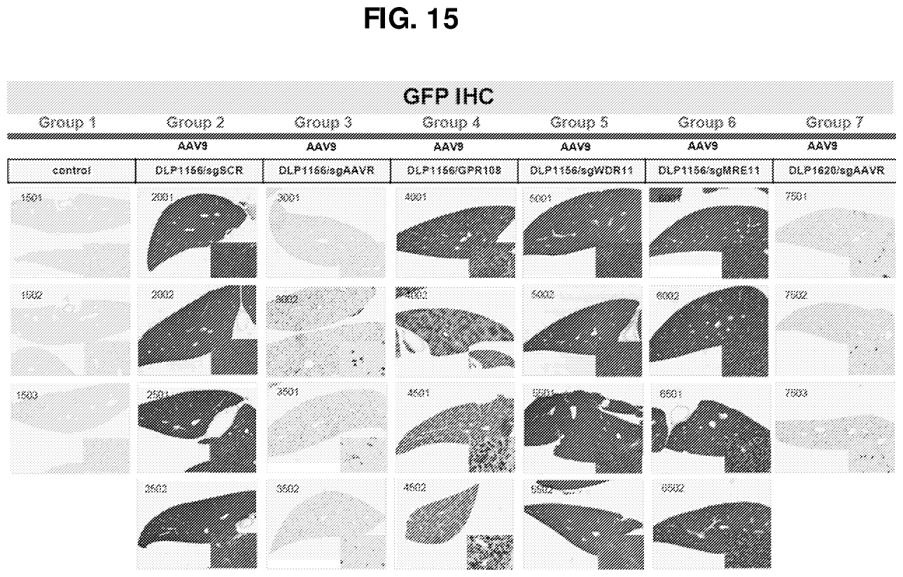

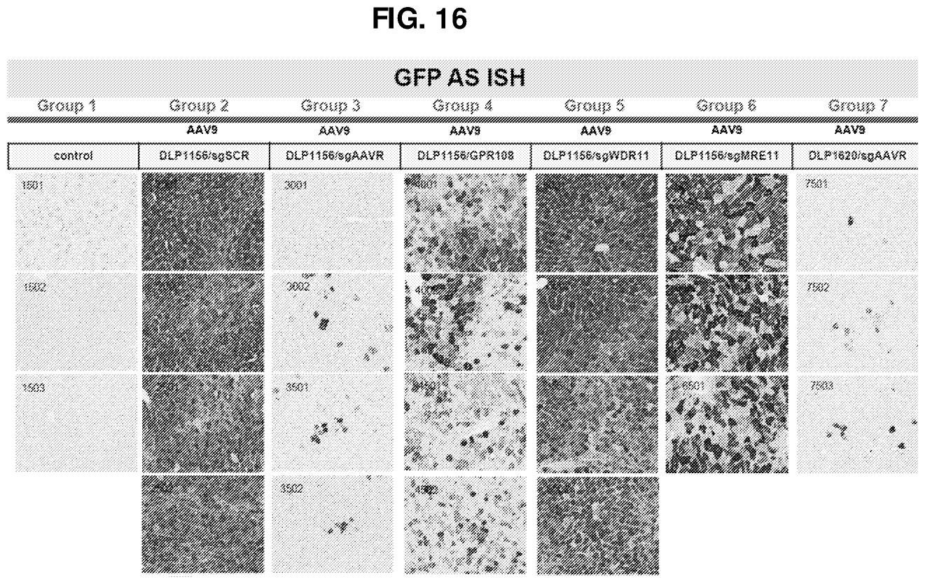

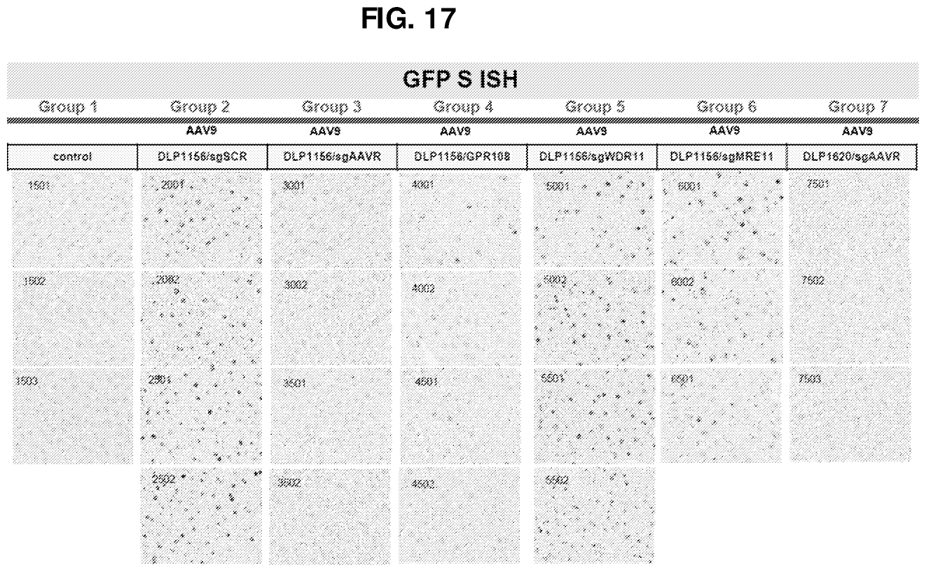

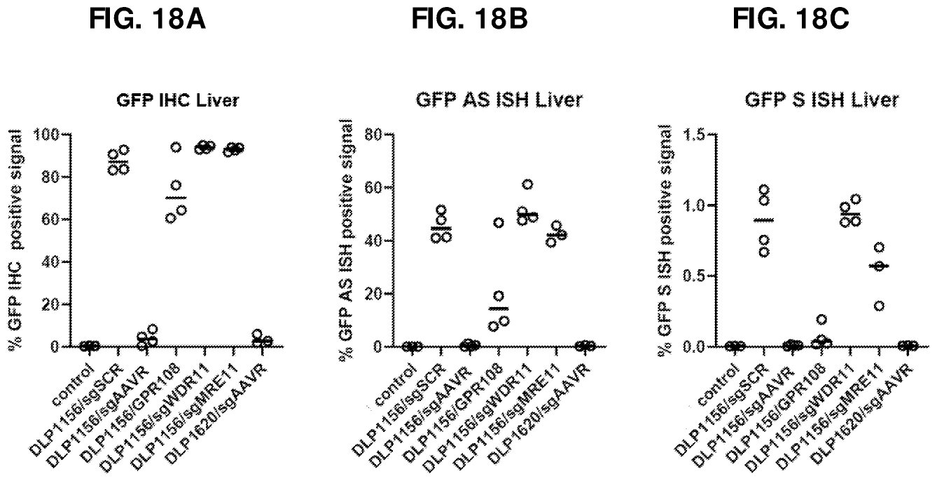

GFP Protein Quantification in Liver Using Western Blot and Direct Fluorescence Confirms that the Knockdown of AAV-R and GPR108 Effectively Decreases the Amount of Transgene Product in that Tissue (FIGS. 13 and 14)

Using a western blot approach to detect AAV-R and MRE11 at the protein level, it was confirmed that the genetic knockdown of AAV-R or MRE11 murine genes reduces the amount of those target proteins in the liver (FIG. 12). Due to a lack of reliable anti-WDR11 and anti-GPR108 antibodies, such analysis remained unconclusive for those two other targets. Detection of the AAV9 transgene product by western blot confirmed that the knockdown of either AAV-R or GPR108 resulted in a significant decrease of GFP protein expression in liver, even though the amplitude of this change was lower in the GPR108 group. The MRE11 or WDR11 gene knockdown did not affect the levels of GFP protein in liver as compared with the control group. Of note, female mice had lower GFP protein than the male mice. Direct measurement of fluorescence intensity from liver homogenates confirmed the western blot results, with the AAV-R KO having the highest reduction effect.

### The Genetic Knockdown of AAV-R or GPR108 Significantly Reduces the Amount of GFP Protein, Transcript and Vector Genome Detected in Liver Tissue by Immunohistology and In Situ Hybridization

Both DLP1156/sgAAV-R and DLP1620/sgAAV-R resulted in a reduction in GFP IHC, and GFP AS and S ISH signal compared to DLP1156/sgSCR. With both AAV-R KO groups, scattered individual hepatocytes showed evidence of high GFP protein and mRNA expression despite low intranuclear sense signal; these findings suggest that this pattern may be independent of individual hepatocyte transduction level. DLP1156/sgGPR108 resulted in more modest reduction in GFP IHC, and GFP AS and S ISH signal compared to DLP1156/sgAAV-R with greater reduction in the periportal regions. DLP1156/sgWDR11 produced no detectable effect on GFP protein on nucleic acid expression. DLP1156/sgMRE11 had no effect on GFP protein expression but did reduce GFP AS and S signal. There was minimal GFP expression in skeletal muscle myocytes of the DLP1156/sgSCR group and no difference discernable in other groups.

### CONCLUSIONS

Our in vitro CRISPR screens have allowed the identification of key host factors necessary for efficient AAV transduction (such as AAR and GPR108) or limiting this complex process (such as WDR11 and MRE11). The present study was designed to assess the potential translatability of such findings in vivo. AAV-R is a cell surface protein that rapidly cycles from the plasma membrane to the endosomal network and has been shown to enable the initial attachment and entry of many AAV serotypes (including AAV9) into target cells. While primary located in the endoplasmic reticulum, GPR108 also has been recognized as an important enabler of AAV transduction, most probably impacting the trafficking of the viral capsid from the cell surface to the nucleus. By contrast, WDR11 (potentially via regulation of the amount of AAV-R at the cell surface) and MRE11 (involved in the resolution of the vector genome in the nucleus) appear to be host factors limiting transduction in vitro.

Systemic delivery of sgRNAs-conjugated DLPs in the SpCas9_Mouse models leads to high gene editing efficiency in liver, with limited impact in skeletal muscle and bone marrow.

The present in-vivo study demonstrated that the genetic knockdown of AAV-R or, to a lesser extend GPR108, in the liver significantly reduces AAV transduction, as seen in the reduction in viral copies per cell and the decreased GFP mRNA & protein expression. WDR11 and MRE11 knockdown had been found to increase AAV transgene expression in in vitro systems, but a similar effect was not observed in liver tissue in vivo. WDR11 KO was like scramble sgRNA in all endpoints and MRE11 had a reduced in AAV transduction (AAV biodistribution and GFP AS and S signal). The liver already saturated with AAVs, may not have been sensitive to slight increases in AAV transduction. It is conceivable that the knockdown of these genes could have a beneficial impact on the transduction of other tissues, but the preferential biodistribution of the nanoparticles in liver did not allow to assess such hypothesis.

Of importance, while the use of a CRISPR/Cas9 approach in conjunction with AAVs is most likely not translatable to the clinical setting, a similar outcome (i.e. AAV liver detargeting after AAV-R or GPR108 knockdown) could be achieved using a nanoparticles-conjugated siRNA strategy targeting either AAV-R or GPR108 (among which GalNac-siRNAs could be a promising path forward). Such transient reduction of AAV-R or GPR108 would potentially decrease the viral load in the patients' liver, lifting some of the hepatotoxicity concerns for this novel therapeutic modality.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A method of modulating transduction efficiency of an AAV particle, comprising:
contacting a cell with an AAV transduction modulator,
thereby modulating the transduction efficiency of the AAV particle.

2. The method of claim 2, wherein the modulator modulates (e.g., increases or decreases) a gene or gene product associated with AAV transduction efficiency.

3. The method of claim 2 or 3, wherein the gene or gene product is a mammalian (e.g., human) gene or gene product.

4. The method of any of the preceding claims, wherein the modulator inhibits the gene or gene product, e.g., a gene or gene product that is associated with a decreased transduction efficiency of an AAV particle.

5. The method of any of the preceding claims, wherein the modulator activates the gene or gene product, e.g., a gene or gene product that is associated with an increased transduction efficiency of an AAV particle.

6. The method of any of the preceding claims, wherein the gene or gene product is associated with an increased transduction efficiency of an AAV particle (e.g., AAV-R or GPR108).

7. The method of any of the preceding claims, wherein the gene or gene product is associated with an increased transduction efficiency of an AAV particle of a first serotype (e.g., AAV2) and an increased transduction efficiency of an AAV particle of a second serotype (e.g., AAV9) (e.g., AAV-R or GPR108).

8. The method of any of the preceding claims, wherein the gene or gene product is associated with a decreased transduction efficiency of an AAV particle (e.g., WDR11 or MRE11).

9. The method of any of the preceding claims, wherein the gene or gene product is associated with a decreased transduction efficiency of an AAV particle of a first serotype (e.g., AAV2) and a decreased transduction efficiency of an AAV particle of a second serotype (e.g., AAV9) (e.g., WDR11 or MRE11).

10. The method of any of the preceding claims, wherein the gene or gene product, when modulated, increases the transduction efficiency of an AAV particle.

11. The method of any of the preceding claims, wherein the gene or gene product, when modulated, increases the transduction efficiency of an AAV particle of a first serotype (e.g., AAV2) and the transduction efficiency of an AAV particle of a second serotype (e.g., AAV9).

12. The method of any of the preceding claims, wherein the gene or gene product, when modulated, decreases the transduction efficiency of an AAV particle.

13. The method of any of the preceding claims, wherein the gene or gene product, when modulated, decreases the transduction efficiency of an AAV particle of a first serotype (e.g., AAV2) and the transduction efficiency of an AAV particle of a second serotype (e.g., AAV9).

14. The method of any of the preceding claims, wherein the gene product is an RNA.

15. The method of any of the preceding claims, wherein the gene product is a protein.

16. The method of any of the preceding claims, wherein the gene or gene product is preferentially expressed in a target tissue (e.g., brain or liver).

17. The method of any of the preceding claims, wherein the modulator alters (e.g., increases or decreases) the expression of the gene.

18. The method of any of the preceding claims, wherein the modulator alters the structure of the gene.

19. The method of any of the preceding claims, wherein the modulator alters (e.g., increases or decreases) an activity (e.g., an enzymatic activity) of the gene product.

20. The method of any of the preceding claims, wherein the modulator alters (e.g., increases or decreases) the level (e.g., abundance) of the gene product.

21. The method of any of the preceding claims, wherein the modulator alters (e.g., increases or decreases) the stability of the gene product.

22. The method of any of the preceding claims, wherein the modulator alters (e.g., increases or decreases) transduction efficiency of an AAV particle, e.g., as determined by an assay described herein.

23. The method of any of the preceding claims, wherein the modulator increases transduction efficiency of an AAV particle,
optionally wherein the modulator increases transduction to at least 10, 20, 30, 40, 50, or 60 viral copies per genome,
optionally wherein the modulator increases transduction to at least 10³, 10⁴, 10⁵, 10⁶, or 10⁷ viral copies per μg DNA.

24. The method of any of the preceding claims, wherein the transduction efficiency is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference level of transduction efficiency.

25. The method of any of the preceding claims, wherein the modulator decreases transduction efficiency of an AAV particle,
optionally wherein the modulator decreases transduction to no more than 0, 1, 2, 3, 4, 5, 6, 7, 8, 9, or 10 viral copies per genome,
optionally wherein the modulator decreases transduction to no more than 10², 10³, or 10⁴ viral copies per μg DNA.

26. The method of any of the preceding claims, wherein the transduction efficiency is decreased by at least 10%, 15%, 20%, 25%, 30%, 35%, 40%, 45%, 50%, 55%, 60%, 65%, 70%, 750/, 80/, 85/, 90%, 95%, 96%, 97%, 98%, 99%, or 100%, compared to a reference level of transduction efficiency.

27. The method of any of the preceding claims, wherein the reference level of transduction efficiency is the level of transduction efficiency in a cell that has not been contacted with the AAV transduction modulator.

28. The method of any of the preceding claims, wherein the reference level of transduction efficiency is the level of transduction efficiency before the cell is contacted with the AAV transduction modulator.

29. The method of any of the preceding claims, wherein the gene or gene product increases the transduction efficiency of an AAV2 particle,
optionally wherein the gene or gene product does not increase, or does not substantially increase, the transduction efficiency of an AAV9 particle,
optionally wherein the gene or gene product is AAV-R or GRP108.

30. The method of claim 29, wherein the gene or gene product is necessary for the transduction of an AAV2 particle, optionally wherein the gene or gene product is not necessary for the transduction of an AAV9 particle.

31. The method of any of claims 29-30, wherein the gene or gene product is selected from the group consisting of: AAV-R, GRP108, EXT1, EXT2, NDST1, BCL10, OAF, PDCD10, LMO4, MALT1, ZNF644, CHUK, GLCE, EP300, ARID4B, RAB21, MED13, DHX15, WASHC5, IKBKB, USP24, HSPA14, CXXC1, UBE2A, INTS8, CDC42, NFKB1, SIN3A, USP7, ARF5, CARD10, JAK1, SLC35C2, PAXK1, ZC3H11A, FAM20B, FAM72A, DHX36, INTS12, and RPRD1B, or a combination thereof.

32. The method of any of claims 29-31, wherein the gene or gene product is associated with endosomal sorting (e.g., RAB21, WASHC5, or USP7).

33. The method of any of claims 29-31, wherein the gene or gene product is associated with vesicle-mediated transport (e.g., CDC42 or ARF5).

34. The method of any of claims 29-31, wherein the gene or gene product is associated with NFKB signaling (e.g., BCL10, MALT1, NFKB1, IKBKB, CHUK, or CARD10).

35. The method of any of claims 29-31, wherein the gene or gene product is associated with heparan sulfate biosynthesis (e.g., EXT1, EXT2, NDST1, GLCE, or FAM20B).

36. The method of any of claims 29-31, wherein the gene or gene product is associated with H3K9 methylation (e.g., ZNF644).

37. The method of any of claims 29-31, wherein the gene or gene product is associated with pre-mRNA processing (e.g., DHX15).

38. The method of any of claims 29-31, wherein the gene or gene product is associated with transcription (e.g., LMO4, EP300, MED13, SIN3, ARID4B, ZC3H11A, CXXC1, RPRD1B, or UBE2A).

39. The method of any of claims 29-31, wherein the gene or gene product is associated with integrator complex and/or RNA polymerase II function (e.g., INTS8 or INST12).

40. The method of any of claims 29-31, wherein the gene or gene product is associated with DNA repair and/or AAV genome resolution (e.g., DHX36, ARID4B, UBE2A, or USP24).

41. The method of any of claims 29-31, wherein the gene or gene product is associated with cell proliferation and/or apoptosis (e.g., PDCD10).

42. The method of any of claims 29-31, wherein the gene or gene product is associated with spondylocarpotarsal synostosis syndrome (e.g., OAF).

43. The method of any of claims 29-31, wherein the gene or gene product is associated with protein folding (e.g., HSPA14).

44. The method of any of claims 29-31, wherein the gene or gene product is associated with helicase activity (e.g., DHX36).

45. The method of any of claims 29-31, wherein the gene or gene product is associated with fucosylation of Notch and/or transport of GCP-fucose (e.g., SLC35C2).

46. The method of any of claims 1-28, wherein the gene or gene product increases the transduction efficiency of an AAV9 particle,
optionally wherein the gene or gene product does not increase, or does not substantially increase, the transduction efficiency of an AAV2 particle,
optionally wherein the gene or gene product is AAV-R or GRP108.

47. The method of claim 46, wherein the gene or gene product is necessary for the transduction of an AAV9 particle, optionally wherein the gene or gene product is not necessary for the transduction of an AAV2 particle.

48. The method of any of claims 46-47, wherein the gene or gene product is selected from the group consisting of: AAV-R, GRP108, TMPRSS11B, HTT, SNRNP70, BRD7, DMXL1, RAB10, CNGA1, KLHDC3, CHP1, CYP3A5, ELOVL4, PNISR, ZCCHC14, AZGP1, TPST1, SC5D, ELP2, ELP3, IPO9, RAB14, WDR7, XRCC4, and GDI2, or a combination thereof.

49. The method of any of claims 46-48, wherein the gene or gene product is associated with microtubule-mediated transport or vesicle function (e.g., HTT).

50. The method of any of claims 46-48, wherein the gene or gene product is an ion channel (e.g., CNGA1).

51. The method of any of claims 46-48, wherein the gene or gene product is associated with nuclear protein import (e.g. IP09).

52. The method of any of claims 46-48, wherein the gene or gene product is associated with O-sulfation of a tyrosine residue within an acidic motif of a polypeptide (e.g., TPST1).

53. The method of any of claims 46-48, wherein the gene or gene product is associated with protection of viral RNA (e.g., ZCCHC14).

54. The method of any of claims 46-48, wherein the gene or gene product is associated with RNA binding by an AAV protein (e.g., PNISR).

55. The method of any of claims 46-48, wherein the gene or gene product is associated with calcium ion-regulated exocytosis (e.g., CHP1).

56. The method of any of claims 46-48, wherein the gene or gene product is associated with intracellular trafficking (e.g., RAB10, RAB14, GDI2, WDR7, or DMXL1).

57. The method of any of claims 46-48, wherein the gene or gene product is a subunit (e.g., a catalytic tRNA acetyltransferase subunit) of an elongator complex, e.g., of an RNA polymerase (e.g., RNA polymerase II), e.g., ELP2 or ELP3.

58. The method of any of claims 46-48, wherein the gene or gene product binds to chromatin (e.g., KLHDC3).

59. The method of any of claims 46-48, wherein the gene or gene product is associated with cholesterol biosynthesis (e.g., SC5D).

60. The method of any of claims 1-28, wherein the gene or gene product increases the transduction efficiency of AAV2 and AAV9 particles,
optionally wherein the gene or gene product is AAV-R or GRP108.

61. The method of claim 60, wherein the gene or gene product is necessary for the transduction of AAV2 and AAV9 particles.

62. The method of any of claims 60-61, wherein the gene or gene product is selected from the group consisting of: AAV-R, GRP108, KIAA0319L, BAMBI, TM9SF2, PHIP, DCLRE1C, VPS35, GPR108, CYREN, ACP2, F8A2, F8A1, F8A3, RBPJ, ATP2C1, ICMT, RABIF, IER3IP1, RBM10, SMG7, GTF2I, ELAVL1, MEPCE, RAB4A, IER3IP1, and SAMD1, or a combination thereof.

63. The method of any of claims 60-62, wherein the gene or gene product is associated with influenza infection (e.g., ACP2).

64. The method of any of claims 60-62, wherein the gene or gene product binds to unmethylated CGIs (e.g., SAMD1).

65. The method of any of claims 60-62, wherein the gene or gene product is associated with intracellular vesicular transport (e.g., RABIF).

66. The method of any of claims 60-62, wherein the gene or gene product is associated with posttranslational modification of cysteine residues (e.g., C-terminal cysteine residues), e.g., in a target protein (e.g., ICMT).

67. The method of any of claims 60-62, wherein the gene or gene product binds to a capsid protein of AAV (e.g., KIAA0319L).

68. The method of any of claims 60-62, wherein the gene or gene product is associated with inhibition of a TLR (e.g., TLR9), e.g., GPR108.

69. The method of any of claims 60-62, wherein the gene or gene product is associated with endosome trafficking, e.g., of AAV-R (e.g., VPS35).

70. The method of any of claims 60-62, wherein the gene or gene product is a calcium ATPase pump (e.g., ATP2C1).

71. The method of any of claims 60-62, wherein the gene or gene product is associated with Notch signaling (e.g., RBPJ).

72. The method of any of claims 60-62, wherein the gene or gene product is associated with HSPG metabolism (e.g., TM9SF2).

73. The method of any of claims 60-62, wherein the gene or gene product is associated with inhibition of NHEJ (e.g., CYREN).

74. The method of any of claims 60-62, wherein the gene or gene product is associated with viral hairpin resolution (e.g., DCLRE1C).

75. The method of any of claims 60-62, wherein the gene or gene product is associated with Glc transporter translocation, optionally wherein the gene or gene product binds to insulin receptor substrate 1 protein (e.g., PHIP).

76. The method of any of claims 1-28, wherein the gene or gene product decreases the transduction efficiency of an AAV2 particle,
optionally wherein the gene or gene product, when modulated, does not decrease, or does not substantially decrease, the transduction efficiency of an AAV9 particle,
optionally wherein the gene or gene product is WRD11 or MRE11.

77. The method of claim 76, wherein the gene or gene product inhibits or prevents the transduction of an AAV2 particle, optionally wherein the gene or gene product does not inhibit or prevent the transduction of an AAV9 particle.

78. The method of any of claims 76-77, wherein the gene or gene product is selected from the group consisting of: WRD11, MRE11, GREB1L, BIRC2, TYMSOS, MSL3, SIK3, GTF2H5, PIGW, ATRAID, PPP4R1, ZFC3H1, SETDB1, SMCHD1, TMEM123, UHRF1, CLUL1, SMAD5, SETX, STEEP1, DENND6A, RTN4RL2, MTMR9, STRADA, PSIP1, BMPR1A, HIKESHI, AP3M1, MEN1, MYL12B, CREB1, RAB12, SMTNL1, MRGBP, WTAP, SUMO2, TCN1, ALDH3B1, ARL14EP, MYL12A, RHOD, PGAP2, EIF4A1, PIGN, DAXX, HDLBP, GOLGA1, SARNP, UBE2L6, TRIM49, SIK2, SOX4, OR6Q1, OR4D6, UNC93B1, CATSPERZ, PIGT, PYM1, MICOS13, RIN1, DOT1L, TRIM49C, MAPK20, COL8A1, FASN, KDM3B, PIGF, PIGA, PIGY, GPC3, TRAPPC2L, RAB31, and ELP3, or a combination thereof.

79. The method of any of claims 76-78, wherein the gene or gene product is associated with a retinoic acid receptor activity (e.g., GREB1L).

80. The method of any of claims 76-78, wherein the gene or gene product is a thymidylate synthetase (e.g., TYMSOS).

81. The method of any of claims 76-78, wherein the gene or gene product is associated with chromatin remodeling (e.g., SMCHD1).

82. The method of any of claims 76-78, wherein the gene or gene product is associated with histone H4 acetylation (e.g., MSL3).

83. The method of any of claims 76-78, wherein the gene or gene product is associated with transcription and/or DNA repair (e.g., GTF2H5).

84. The method of any of claims 76-78, wherein the gene or gene product is a histone methyltransferase (e.g., SETDB1).

85. The method of any of claims 76-78, wherein the gene or gene product is associated with exosomal degradation of polyadenylated RNA (e.g., ZFC3H1).

86. The method of any of claims 76-78, wherein the gene or gene product is associated with histone deacetylase recruitment to a DNA (e.g., UHRF1).

87. The method of any of claims 76-78, wherein the gene or gene product is associated with oxidative stress-induced DNA double strand break response (e.g., SETX).

88. The method of any of claims 76-78, wherein the gene or gene product is associated with histone modification (e.g., MEN1).

89. The method of any of claims 76-78, wherein the gene or gene product is associated with nucleosome/DNA interaction (e.g., MRGBP).

90. The method of any of claims 76-78, wherein the gene or gene product is associated with GPI biosynthesis (e.g., PIGW, PIGN, PIGT, PIGF, PIGA, or PIGY).

91. The method of any of claims 76-78, wherein the gene or gene product is associated with maturation of GPI anchors (e.g., PGAP2).

92. The method of any of claims 76-78, wherein the gene or gene product is associated with vesicle-mediated endocytosis (e.g., DENND6A, GOLGA1, MTMR9, GPC3, PPP4R1, RAB12, RAB31, RHOD, RIN1, TMEM123, or TRAPPC2L).

93. The method of any of claims 76-78, wherein the gene or gene product is associated with clathrin-coated vesicle-mediated endocytosis (e.g., AP3M1).

94. The method of any of claims 76-78, wherein the gene or gene product is associated with a BMP receptor kinase (e.g., SMAD5, BMPR1A, or CREB1).

95. The method of any of claims 76-78, wherein the gene or gene product is associated with transcription (e.g., PSIP1).

96. The method of any of claims 76-78, wherein the gene or gene product is associated with nuclear import of an HSP70 protein (e.g., HIKESHI).

97. The method of any of claims 76-78, wherein the gene or gene product is associated with a Ser/Thr kinase (e.g., SIK2 or SIK3).

98. The method of any of claims 76-78, wherein the gene or gene product is associated with SUMOylation of a protein (e.g., SUMO2, UBE2L6).

99. The method of any of claims 1-28, wherein the gene or gene product decreases the transduction efficiency of an AAV9 particle,
optionally wherein the gene or gene product does not decrease, or does not substantially decrease, the transduction efficiency of an AAV2 particle,
optionally wherein the gene or gene product is WRD11 or MRE11.

100. The method of claim 99, wherein the gene or gene product inhibits or prevents the transduction of an AAV9 particle, optionally wherein the gene or gene product does not inhibit or prevent the transduction of an AAV2 particle.

101. The method of any of claims 99-100, wherein gene or gene product is selected from the group consisting of: WRD11, MRE11, AP2A1, AP2B1, TM9SF4, ACTR5, PTMA, PAPOLA, PDHA1, PDHB, SLC25A19, GAK, AUNIP, FCHO2, ACTB, LIPT1, UCHL5, INO80E, ECHS1, NELFB, EDC4, PRMT1, PDS5B, PELI3, FBXL20, and SLC35A1, or a combination thereof.

102. The method of any of claims 99-101, wherein the gene or gene product binds to ATP, a chaperone protein, and/or clathrin (e.g., GAK).

103. The method of any of claims 99-101, wherein the gene or gene product is associated with clathrin-coated vesicle trafficking (e.g., AP2B1 or AP2A1).

104. The method of any of claims 99-101, wherein the gene or gene product is associated with clathrin-mediated endocytosis (e.g., FCHO2).

105. The method of any of claims 99-101, wherein the gene or gene product is associated with vesicular trafficking (e.g., ACTB).

106. The method of any of claims 99-101, wherein the gene or gene product is associated with an innate immune response (e.g., PELI3 or FBXL20).

107. The method of any of claims 99-101, wherein the gene or gene product is associated with glucose metabolism (e.g., PDHB or PDHA1).

108. The method of any of claims 99-101, wherein the gene or gene product is associated with DNA resection and/or homologous recombination, e.g., after DNA damage (e.g., AUNIP).

109. The method of any of claims 99-101, wherein the gene or gene product is associated with DNA repair, e.g., after DNA damage (e.g., UCHL5, INO80E, PDS5B, ACTR5, or PRMT1).

110. The method of any of claims 99-101, wherein the gene or gene product is associated with transport of CMP-sialic acid from cytosol to a Golgi vesicle (e.g., SLC35A1).

111. The method of any of claims 99-101, wherein the gene or gene product is associated with localization of polypeptides comprising glycine-rich transmembrane domains, e.g., to the cell surface (e.g., TM9SF4).

112. The method of any of claims 99-101, wherein the gene or gene product is associated with poly(A) tail synthesis (e.g., PAPOLA).

113. The method of any of claims 99-101, wherein the gene or gene product is PTMA.

114. The method of any of claims 99-101, wherein the gene or gene product is associated with elongation of an mRNA by RNA polymerase II (e.g., NELFB).

115. The method of any of claims 99-101, wherein the gene or gene product is associated with mRNA degradation (e.g., EDC4).

116. The method of any of claims 99-101, wherein the gene or gene product encodes a mitochondrial lipoyltransferase (e.g., LIPT1).

117. The method of any of claims 99-101, wherein the gene or gene product is associated with mitochondrial fatty acid beta-oxidation (e.g., ECHS1).

118. The method of any of claims 99-101, wherein the gene or gene product encodes a mitochondrial thiamine pyrophosphate carrier (e.g., SLC25A19).

119. The method of any of claims 1-28, wherein the gene or gene product decreases the transduction efficiency of AAV2 and AAV9 particles,
optionally wherein the gene or gene product is WRD11 or MRE11.

120. The method of claim 119, wherein the gene or gene product inhibits or prevents the transduction of AAV2 and AAV9 particles.

121. The method of any of claims 119-120, wherein the gene or gene product is selected from the group consisting of: WRD11, MRE11, PITPNB, PITP, FAM91A1, WDR11, AP1G1, AP1M1, AP1S1, AP1S3, HEATR5B, STX16, AP1B1, PIAS1, DENR, ARL1, ZFAT, TBC1D23, LIN37, RALGAPB, B3GNT2, ELOVL1, AP2B1, KIAA2013, PTEN, MCTS1, NBN, HELZ, SLC38A10, FBXL20, TGIF1, KDSR, CPD, CHD7, USP9X, SIMC1, TAF11, VAPA, MTMR6, RAB1B, SLF2, MRE11, VT11A, MBOAT7, and PPP6R3, or a combination thereof.

122. The method of any of claims 119-121, wherein the gene or gene product is associated with the long-chain FA elongation cycle (e.g., ELOVL1).

123. The method of any of claims 119-121, wherein the gene or gene product is associated with lipoic acid biosynthesis (e.g., PIAS1).

124. The method of any of claims 119-121, wherein the gene or gene product is associated with a protein phosphatase catalytic subunit (e.g., PPP6R3).

125. The method of any of claims 119-121, wherein the gene or gene product is associated with the WDR11 pathway (e.g., WDR11, FAM91A1, AP1G1, AP1S1, APIM1, APIS3, or TBC1D23).

126. The method of any of claims 119-121, wherein the gene or gene product is associated with retrograde transport from the Golgi apparatus to the endoplasmic reticulum, e.g., of PI and/or PC (e.g., PITP).

127. The method of any of claims 119-121, wherein the gene or gene product is associated with vesicular transport from a late endosome to a trans-Golgi network (e.g., STX16).

128. The method of any of claims 119-121, wherein the gene or gene product is associated with an activity or recruitment of a golgin, arfaptin, and/or Arf-GEF to a trans-Golgi network (e.g., ARL1).

129. The method of any of claims 119-121, wherein the gene or gene product encodes a component of a clathrin-dependent vesicle and/or is associated with AP1G1/AP-1-mediated protein trafficking (e.g., HEATR5B).

130. The method of any of the preceding claims, wherein the modulator is:
(a) a gene editing system targeted to one or more sites within the gene or a regulatory element thereof;
(b) a nucleic acid encoding one or more components of the gene editing system; or
(c) a combination thereof.

131. The method of claim 130, wherein the gene editing system is a CRISPR/Cas system, a zinc finger nuclease system, a TALEN system, and a meganuclease system.

132. The method of any of claims 130-131, wherein the gene editing system binds to a target sequence in an early (e.g., the first, second, or third) exon or intron of the gene.

133. The method of any of claims 130-132, wherein the gene editing system binds a target sequence of the gene, and the target sequence is upstream of exon 4, e.g., in exon 1, exon 2, or exon 3.

134. The method of any of claims 130-133, wherein the gene editing system binds to a target sequence in a late exon or intron of the gene.

135. The method of any of claims 130-134, wherein the gene editing system binds a target sequence of the gene, and the target sequence is downstream of a preantepenultimate exon, e.g., is in an antepenultimate exon, a penultimate exon, or a last exon.

136. The method of any of claims 130-135, wherein the gene editing system is a CRISPR/Cas system comprising a guide RNA (gRNA) molecule comprising a targeting sequence which hybridizes to a target sequence of the gene.

137. The method of claim 136, wherein the CRISPR/Cas system is a CRISPR/Cas9 system.

138. The method of claim 136, wherein the CRISPR/Cas system is a CRISPR/Cas12a system.

139. The method of any of claims 130-138, wherein the modulator is a small interfering RNA (siRNA), small hairpin (shRNA), or guide RNA (gRNA) specific for the gene, or a nucleic acid encoding the siRNA, shRNA, or gRNA.

140. The method of claim 139, wherein the siRNA or shRNA comprises a nucleotide sequence complementary to a nucleotide sequence of an mRNA encoded by the gene.

141. The method of any of claims 130-138, wherein the modulator is an antisense oligonucleotide (ASO) specific for the gene.

142. The method of claim 141, wherein the ASO comprises a nucleotide sequence complementary to a nucleotide sequence of an mRNA encoded by the gene.

143. The method of any of claims 130-138, wherein the modulator is a small molecule.

144. The method of claim 143, wherein the small molecule is a protein degrader.

145. The method of any of claims 130-138, wherein the modulator is a protein or a peptide, or a nucleic acid encoding the protein or peptide.

146. The method of any of claims 130-138, wherein the modulator is an antibody molecule.

147. The method of any of claims 130-138, wherein the modulator is a dominant negative binding partner of a protein encoded by the gene, or a nucleic acid encoding the dominant negative binding partner.

148. The method of any of claims 130-138, wherein the modulator is a dominant negative variant (e.g., catalytically inactive) of a protein encoded by the gene, or a nucleic acid encoding said dominant negative variant.

149. The method of any of the preceding claims, wherein the AAV particle comprises an AAV genome.

150. The method of any of the preceding claims, wherein the AAV particle comprises an AAV-like particle.

151. The method of any of the preceding claims, wherein the AAV particle comprises a capsid.

152. The method of any of the preceding claims, wherein the AAV particle has a serotype of AAV1, AAV2, AAV3, AAV4, AAV5, AAV6, AAV7, AAV8, AAV9, AAV10, AAV11, or a combination thereof.

153. The method of any of the preceding claims, wherein the AAV particle is an AAV2 particle.

154. The method of any of the preceding claims, wherein the AAV particle is an AAV9 particle.

155. The method of any of the preceding claims, wherein the AAV particle has a tropism towards the CNS (e.g., AAV1, AAV2, AAV4, AAV5, AAV8, or AAV9).

156. The method of any of the preceding claims, wherein the AAV particle has a tropism towards heart (e.g., AAV1, AAV8, or AAV9).

157. The method of any of the preceding claims, wherein the AAV particle has a tropism towards kidney (e.g., AAV2).

158. The method of any of the preceding claims, wherein the AAV particle has a tropism towards liver (e.g., AAV7, AAV8, or AAV9).

159. The method of any of the preceding claims, wherein the AAV particle has a tropism towards lung (e.g., AAV4, AAV5, AAV6, or AAV9).

160. The method of any of the preceding claims, wherein the AAV particle has a tropism towards pancreas (e.g., AAV8).

161. The method of any of the preceding claims, wherein the AAV particle has a tropism towards a photoreceptor cell (e.g., AAV2, AAV5, or AAV8).

162. The method of any of the preceding claims, wherein the AAV particle has a tropism towards retinal pigment epithelium (RPE) (e.g., AAV1, AAV2, AAV4, AAV5, or AAV8).

163. The method of any of the preceding claims, wherein the AAV particle has a tropism towards skeletal muscle (e.g., AAV1, AAV6, AAV7, AAV8, or AAV9).

164. The method of any of the preceding claims, wherein the AAV particle comprises a nucleotide sequence encoding a therapeutic protein, e.g., a protein associated with a disorder described herein.

165. The method of any of the preceding claims, wherein the AAV particle comprises a nucleotide sequence encoding NGF, APOE2 (e.g., hAPOE2), TERT (e.g., hTERT), MAPT, GAD, AADC, NTN, GDNF, GCase, HIT, SMN, SMN2, SOD1, or C9orf72.

166. The method of any of the preceding claims, wherein the AAV particle comprises a nucleotide sequence encoding a therapeutic nucleic acid, e.g., a nucleic acid targeting a nucleotide sequence encoding a protein associated with a disorder described herein.

167. The method of any of the preceding claims, wherein the cell is a brain cell, a liver cell, a spinal cord cell, a dorsal root ganglion (DRG) cell, a spleen cell, a lymph node cell, a kidney cell, a lung cell, a heart cell, a muscle cell (e.g., a skeletal muscle cell, e.g., a femur muscle cell), a diaphragm cell, a bone marrow cell, or a gonad cell.

168. The method of any of the preceding claims, wherein the cell is a central nervous system (CNS) cell.

169. The method of claim 168, wherein the CNS cell is an astrocyte, an oligodendrocyte, a microglial cell, or an ependymal cell.

170. The method of any of the preceding claims, wherein the cell is a brain cell.

171. The method of claim 170, wherein the brain cell is a neuron or glial cell.

172. The method of any of the preceding claims, wherein the cell is a DRG cell.

173. The method of any of the preceding claims, wherein the cell is a liver cell;
optionally wherein the modulator increases transduction to at least 10, 20, 30, 40, 50, or 60 viral copies per liver cell genome,
optionally wherein the modulator increases transduction to at least 10⁵, 10⁶, or 10⁷ viral copies per μg DNA in the liver cell.

174. The method of claim 173, wherein the liver cell is a hepatocyte, hepatic stellate cell, a Kupffer cell, or a liver sinusoidal endothelial cell.

175. The method of any of the preceding claims, wherein the cell is contacted with the AAV transduction modulator in vitro.

176. The method of any of the preceding claims, wherein the cell is contacted with the AAV transduction modulator ex vivo.

177. The method of any of the preceding claims, wherein the cell is contacted with the AAV transduction modulator in vivo, optionally wherein the method results in a high gene modulating (e.g., gene editing) efficiency (e.g., at least 60%, 65%, 70%, 75%, 80%, 85%, 90%, 95%, or 98%) in a first tissue (e.g., liver), with limited impact (e.g., a gene modulating (e.g., gene editing) efficiency of 20%, 15%, 10%, 5%, 2%, or less) in a second tissue (e.g., skeletal muscle, bone marrow, or both).

178. A method of modulating transduction efficiency of an AAV particle, the method comprising:
administering to a subject in need thereof an effective amount of an AAV transduction modulator,
thereby modulating the transduction efficiency of the AAV particle.

179. A method of treating a disorder, the method comprising:
administering to a subject in need thereof an effective amount of a therapy comprising an AAV genome or an AAV particle, wherein a gene or gene product associated with AAV transduction efficiency is modulated in the subject,
thereby treating the disorder.

180. A method of treating a disorder, the method comprising:
administering to a subject in need thereof an effective amount of (a) an AAV transduction modulator and (b) a therapy comprising an AAV genome or an AAV particle,
thereby treating the disorder.

181. The method of claim 179 or 180, wherein the subject has not received, or is not receiving, the therapy, when the AAV transduction modulator is administered.

182. The method of any of claims 179-181, wherein the AAV transduction modulator was administered at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.

183. The method of any of claims 179-182, wherein the AAV transduction modulator was administered no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.

184. The method of any of claims 179-183, wherein the subject has received, or is receiving, the therapy, when the AAV transduction modulator is administered.

185. The method of any of claims 179-184, wherein the subject received the therapy at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.

186. The method of any of claims 179-185, wherein the subject received the therapy no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.

187. The method of any of claims 179-186, wherein the subject has a disorder or a symptom thereof, or is at risk of having a disorder or a symptom thereof.

188. The method of claim 187, wherein the disorder is a neurodegenerative disorder.

189. The method of claim 188, wherein the neurodegenerative disorder is Alzheimer's disease, Parkinson's disease, Huntington's disease, multiple sclerosis (MS), amyotrophic lateral sclerosis (ALS), or Batten disease.

190. The method of claim 187, wherein the disorder is mild Alzheimer's disease, mild-to-moderate Alzheimer's disease, mild-to-severe Alzheimer's disease, early dementia, Parkinson's disease (e.g., idiopathic Parkinson's disease, bilateral idiopathic Parkinson's disease, mid-to-late stage Parkinson's disease), Huntington's disease (e.g., manifest Huntington's disease), Type 1 SMA, presymptomatic SMA, or amyotrophic lateral sclerosis (ALS) (e.g., familial ALS, ALS with SOD1 mutation, or ALS with C9orf72 mutation).

191. The method of claim 187, wherein the disorder is an eye disorder.

192. The method of claim 191, wherein the eye disorder is blindness, e.g., inherited or non-inherited blindness.

193. The method of claim 191, wherein the eye disorder is Leber's congenital amaurosis, age-related macular degeneration, choroideremia, or color blindness.

194. The method of any of claims 179-193, wherein the method reduces the toxicity of the therapy, enhances the efficacy of the therapy, or both.

195. A method of preparing a subject for a therapy comprising an AAV genome or an AAV particle, the method comprising:
administering to the subject an effective amount of an AAV transduction modulator,
thereby preparing the subject for the therapy.

196. A method of reducing the toxicity of a therapy comprising an AAV genome or an AAV particle, the method comprising:
administering to a subject in need thereof an effective amount of an AAV transduction modulator;
thereby reducing the toxicity of the therapy.

197. The method of claim 196, wherein the subject has not received, or is not receiving, the therapy, when the AAV transduction modulator is administered.

198. The method of claim 196 or 197, wherein the AAV transduction modulator was administered at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.

199. The method of any of claims 196-198, wherein the AAV transduction modulator was administered no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.

200. The method of any of claims 196-199, wherein the subject has received, or is receiving, the therapy, when the AAV transduction modulator is administered.

201. The method of any of claims 196-200, wherein the subject received the therapy at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.

202. The method of any of claims 196-201, wherein the subject received the therapy no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.

203. The method of any of claims 196-202, wherein the toxicity is reduced by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, compared to a reference level of toxicity.

204. The method of any of claims 196-203, wherein the reference level of toxicity is the level of toxicity in a subject that has not been administered to the AAV transduction modulator.

205. The method of any of claims 196-204, wherein the reference level of toxicity is the level of toxicity before the subject is administered the AAV transduction modulator.

206. The method of any of claims 196-205, wherein the method reduces the toxicity in dorsal root ganglion (DRG).

207. The method of any of claims 196-206, wherein the method reduces the toxicity in liver.

208. The method of any of claims 196-207, wherein the method reduces the toxicity in cardiomyocytes.

209. The method of any of claims 196-208, wherein the method reduces the toxicity in retinal pigment epithelium (RPE).

210. A method of enhancing the efficacy of a therapy comprising an AAV genome or an AAV particle, the method comprising:
administering to a subject in need thereof an effective amount of an AAV transduction modulator,
thereby enhancing the efficacy of the therapy.

211. The method of claim 210, wherein the subject has not received, or is not receiving, the therapy, when the AAV transduction modulator is administered.

212. The method of claim 210 or 211, wherein the AAV transduction modulator was administered at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.

213. The method of any of claims 210-212, wherein the AAV transduction modulator was administered no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the subject receives the therapy.

214. The method of any of claims 210-213, wherein the subject has received, or is receiving, the therapy, when the AAV transduction modulator is administered.

215. The method of any of claims 210-214, wherein the subject received the therapy at least 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.

216. The method of any of claims 210-215, wherein the subject received the therapy no more than 1, 2, 3, 4, 5, 6, 7, 8, 9, 10, 11, 12, 13, 14, 15, 16, 17, 18, 19, 20, 21, 22, o 23 hours, 1, 2, 3, 4, 5, or 6 days, 1, 2, 3, or 4 weeks, 1, 2, 3, 4, 5, or 6 months, before the AAV transduction modulator is administered.

217. The method of any of claims 210-216, wherein the efficacy is increased by at least 10%, 20%, 30%, 40%, 50%, 60%, 70%, 80%, or 90%, or at least 1, 2, 3, 4, 5, 6, 7, 8, 9, 10, 20, 30, 40, 50, 60, 70, 80, 90, 100, 200, 300, 400, 500, 600, 700, 800, 900, or 1000-fold, compared to a reference.

218. The method of any of claims 210-217, wherein the reference level of efficacy is the level of efficacy in a subject that has not been administered to the AAV transduction modulator.

219. The method of any of claims 210-218, wherein the reference level of efficacy is the level of efficacy before the subject receives the AAV transduction modulator.

220. A method of producing a cell having an increased AAV transduction efficiency, comprising:
contacting a cell with an AAV transduction modulator,
thereby producing the cell.

221. A cell produced by a method of claim 220.

222. A cell comprising an AAV modulator described herein and an AAV particle.

223. A pharmaceutical composition comprising an AAV modulator described herein and an AAV particle.

224. A kit comprising an AAV modulator described herein and an AAV particle.

225. An AAV transduction modulator (e.g., as described herein) for use in a method of modulating transduction efficiency of an AAV particle in a cell or a subject.

226. An AAV transduction modulator (e.g., as described herein) for use in combination with an AAV genome or an AAV particle in a method of treating a disorder in a subject.

227. An AAV transduction modulator (e.g., as described herein) for use in a method of preparing a subject for a therapy comprising an AAV genome or an AAV particle.

228. An AAV transduction modulator (e.g., as described herein) for use in a method of reducing the toxicity of a therapy comprising an AAV genome or an AAV particle in a subject.

229. An AAV transduction modulator (e.g., as described herein) for use in a method of increasing the efficacy of a therapy comprising an AAV genome or an AAV particle in a subject.

230. Use of an AAV transduction modulator (e.g., as described herein) in the manufacture of a medicament for modulating transduction efficiency of an AAV particle in a cell or a subject.

231. Use of an AAV transduction modulator (e.g., as described herein) in the manufacture of a medicament in combination with an AAV genome or an AAV particle for treating a disorder in a subject.

232. Use of an AAV transduction modulator (e.g., as described herein) in the manufacture of a medicament for preparing a subject for a therapy comprising an AAV genome or an AAV particle.

233. Use of an AAV transduction modulator (e.g., as described herein) in the manufacture of a medicament for reducing the toxicity of a therapy comprising an AAV genome or an AAV particle in a subject.

234. Use of an AAV transduction modulator (e.g., as described herein) in the manufacture of a medicament for increasing the efficacy of a therapy comprising an AAV genome or an AAV particle in a subject.

235. The method of any of claims 1-220, the AAV transduction modulator for use of any of claims, 225-229, or the use of any of 230-234, wherein the method, AAV transduction modulator for use, or use results in a high gene modulating (e.g., gene editing) efficiency (e.g., at least 60%, 65%, 70%, 75%, 80%, 85%, 90%, 95%, or 98%) in a first tissue (e.g., liver), with limited impact (e.g., a gene modulating (e.g., gene editing) efficiency of 20%, 15%, 10%, 50%, 20%, or less) in a second tissue (e.g., skeletal muscle, bone marrow, or both).

236. The method of any of claims 1-220 or 235, the AAV transduction modulator for use of any of claims, 225-229 or 235, or the use of any of 230-235, wherein the AAV transduction modulator is contacted or administered intravenously.
